(19) United States
(12) Patent Application Publication
(10) Pub. No.: US 2026/0248724 A1
(43) Pub. Date: Aug. 27, 2026

(54) INJECTABLE HIGH CONCENTRATION PHARMACEUTICAL FORMULATIONS AND METHODS OF MANUFACTURING AND USE THEREOF

(71) Applicant: Xeris Pharmaceuticals, Inc., Chicago, IL (US)

(72) Inventors: Martin DONOVAN, Chicago, IL (US); Steve PIPPIN, Chicago, IL (US); Glen KLEINSCHMIDT, Chicago, IL (US); Diana BOWMAN, Chicago, IL (US); Alicia ZAYAS GARCIA, Chicago, IL (US); Laura KELLY, Chicago, IL (US)

(73) Assignee: Xeris Pharmaceuticals, Inc., Chicago, IL (US)

(21) Appl. No.: 19/550,070

(22) Filed: Feb. 25, 2026

Related U.S. Application Data
(60) Provisional application No. 63/763,178, filed on Feb. 25, 2025.
(60) Provisional application No. 63/813,987, filed on May 29, 2025.

(51) Int. Cl. A61K 9/14; A61J 1/22; A61K 9/00; A61K 39/00; A61K 45/06; A61K 47/14; A61K 47/20; A61K 47/26; C07K 16/32

(52) U.S. Cl. CPC A61K 9/145; A61J 1/22; A61K 9/0019; A61K 45/06; A61K 47/14; A61K 47/20; A61K 47/26; C07K 16/32; A61K 2039/54

(57) ABSTRACT

The present invention provides compositions comprising one or more active pharmaceutical ingredients, wherein the compositions are in the form of high solids content concentrated suspensions capable of being injected in relatively low volumes into an animal using standard commercially available syringes. The invention also provides methods of making such compositions, particularly those compositions comprising high molecular weight active ingredients (e.g., antibodies, enzymes and other proteins and peptides) at relatively high therapeutic concentrations in the high solids content concentrated suspensions. The invention further provides methods of using such formulations in treating, preventing, and/or ameliorating certain diseases and physical disorders in animals, including humans, in need thereof.

### CROSS REFERENCE TO RELATED APPLICATIONS

This application claims the priority benefit of U.S. Provisional Application No. 63/763,178, filed Feb. 25, 2025, and of U.S. Provisional Application No. 63/813,987, filed May 29, 2025, the disclosures of which are incorporated by reference herein in their entireties.

### STATEMENT REGARDING PRIOR DISCLOSURES BY THE INVENTOR OR A JOINT INVENTOR

Some of the material disclosed herein was disclosed in U.S. Pat. Nos. 8,110,209, 8,790,679, and 9,314,424, and in U.S. Published Application Nos. 2017/0216529 and 2023/0085357.

### BACKGROUND OF THE INVENTION

### Field of the Invention

The present invention relates generally to parenteral, i.e., intracutaneous, subcutaneous, intradermal, and/or intramuscular, injection of pharmaceutical formulations including those containing at least one active pharmaceutical ingredient at high concentrations, particularly in the form of pastes, and provides such formulations, methods of manufacturing and use of such formulations and kits comprising such formulations.

### Description of Related Art

Parenteral injection refers to the administration of drugs, medications or vaccines via injection under or through one or more layers of skin or mucus membranes of an animal. Standard injections are given into the subcutaneous or intramuscular region of an animal, e.g., a human patient. These deep locations are targeted because the tissue expands more easily, relative to shallow dermal sites, to accommodate the 0.1-3.0 cc (ml) injection volumes required to deliver most therapeutic agents.

Generally, injections have been classified into different categories, including (1) solutions ready for injection; (2) dry soluble products (solutes) ready to be combined with a solvent just prior to being injected into a patient; (3) dry, insoluble products ready to be combined with a suitable injection medium prior to administration; (4) suspensions ready for injection; and (5) emulsions ready for injection. Such injectable formulations are administered by routes including·intravenous, subcutaneous, intradermal, intramuscular, intraspinal, intracisternal, and intrathecal. The nature of the therapeutic agent and of the disease or disorder being treated quickly determines the route of administration. However, the desired route of administration places constraints on the therapeutic formulation itself. For example, solutions for subcutaneous administration require strict attention to tonicity adjustment in order to avoid irritation to the nerves and tissue in the surrounding area of injection. Likewise, suspensions are not administered directly into the blood stream in view of the potential of insoluble particles blocking capillaries.

In comparison to other dosage forms and routes of administration (e.g., oral, transdermal), injectables possess certain advantages, including immediate physiological action (e.g., via intravenous injection), avoidance of intestinal absorption problems attended with many drugs, and the accurate administration of the desired dose into the blood stream of a patient. On the other hand, one of the disadvantages of injectables is the pain and discomfort present at the site of administration associated with certain pharmaceutically active agents, as well as the trauma of having a needle inserted under the skin or into a vein. There is a degree of discomfort for the patient with each injection which is administered.

Currently, biopharmaceutical agents are typically reconstituted into sterile solutions and are administered into the subcutaneous or intramuscular space using a large gauge needle, e.g., in the range 18-30 gauge. Pain is caused by the depth of the penetration of the needle, the size “gauge” of the needle, the large volume of injection, and the diffusion of drug away from the site of injection, among other things. In addition to problems with administration of injectables due to pain associated with the same, there are other drawbacks of current practices with respect to injections. For example, many proteins and sustained release drugs require reconstitution immediately prior to administration. Dosing of drugs can be inflexible and inaccurate. Further, many formulations need to be refrigerated to protect the drug(s) from physical and/or chemical degradation (e.g., hydrolysis). Further, current administration systems are wasteful in that the injection device retains a significant amount of the drug product. Further, to effect delivery of the necessary dose required, an injectable formulation typically must be concentrated and stabilized. Standard injections are given in the liquid form. Products that are sold as liquids or a lyophilized powder require reconstitution in an aqueous carrier prior to injection. Many therapeutic protein and vaccine products are produced in a dry, solid form to promote stability while on the shelf. These formulations are diluted/reconstituted to a solution or suspension prior to injection in a pharmaceutically acceptable medium, including sterile water for injection (SWFI), phosphate buffer solution, or isotonic saline.

More recently, the preparation and use of high concentration pharmaceutical formulations in the form of concentrated suspensions, including viscoelastic suspensions (VES), which may also be referred to as “low-moisture suspensions,” or “pastes” in certain prior disclosures, has been described (see, e.g., U.S. Pat. Nos. 8,110,209, 8,790,679, and 9,314,424, and US Patent Publication No. 2017/0216529, the disclosures of all of which are incorporated herein by reference in their entireties). Such formulations may contain active pharmaceutical ingredients at substantially higher concentrations than found in traditional aqueous pharmaceutical formulations. A VES, comprising a two-phase composition of one or more solid-phase components (i.e., powders) dispersed in a continuous liquid-phase comprising one or more diluents that are non-solvents, substantially non-solvents, or only partial solvents with respect to the powder, may be an effective dosage form for delivering medicament(s) parenterally (e.g., subcutaneously, intramuscularly, intradermally, or otherwise intracutaneously). For example, VES formulations may be able to achieve much higher solids (e.g., drug) concentrations than typical solutions (e.g., water-based solutions) while also providing greater stability relative to aqueous solutions as the active ingredient in a VES may be formulated in the solid state (e.g., as a powder). This approach may be particularly advantageous for formulating active pharmaceutical ingredients that are not very soluble in aqueous solutions or that are prone to chemical degradation (e.g., hydrolysis), and/or physical instability (e.g., aggregation) upon being formulated into aqueous formulations containing lower concentrations of the active pharmaceutical ingredient for delivery to a patient.

VES compositions, as described above, may be characterized by their viscoelastic behavior, exhibiting both viscous (fluid-like) and elastic (solid-like) properties, and may contain a high percentage of finely dispersed solids (e.g., powder particles) in a pharmaceutically acceptable diluent system (e.g., oils, triglycerides, or other biocompatible carriers) with a consistency that may range from relatively soft to relatively stiff depending on the solids content and/or the powder and diluent characteristics. The actual solids content and solids concentration of the VES may primarily depend on the properties of the constituent powder and diluent system, and may range both below and above conventional pharmaceutical dosage form ranges, as described in U.S. Pat. Nos. 8,110,209, 8,790,679, 9,314,424, and 11,129,940, the disclosures of all of which are incorporated herein by reference in their entireties. Various approaches to preparing concentrated suspensions (e.g., VES) are described herein. In one approach, a quantity of diluent may be added to a powder that is sufficient to wet the powder particles and disrupt powder-powder contacts, while any partial dissolution of the powder in the diluent should not substantially compromise the two-phase nature or viscoelastic properties of the resulting composition. It is understood that while complete disruption of all direct powder-powder contacts may be desirable, in practice many micronized powders are highly cohesive and complete disruption of all direct powder-powder contacts may not be achievable despite the application of high-shear mixing techniques and other agglomerate disruption methods as described herein. The goal of agglomerate disruption, as described herein, may be to reduce the number and/or size of agglomerates to a level that is compatible with delivery through the intended delivery flow path, rather than to eliminate all particle-particle contacts. Additional diluent may then be added to the mixture to fill in the interstitial spaces between the powder particles (i.e., the void volume) and thus enable the composition to flow under applied force. In an alternative approach described herein, a dilute suspension may be prepared first using high-shear mixing to disrupt agglomerates at low solids content, and the suspension may then be concentrated by removing a portion of the diluent through a separation process to achieve the target solids content. Powders having very low density (i.e., those having a high surface area-to-volume ratio) may require a greater volume of diluent to form a concentrated suspension compared to powders with a lower surface area-to-volume ratio. Therefore, the percent solids content of a concentrated suspension may vary greatly and may depend on multiple factors, including the process by which the powder was prepared (e.g., non-limiting examples include freeze drying, spray drying, spray freeze-drying, thin-film freezing, solvent extraction/exchange, coacervation, and additional particle engineering techniques that are known in the art), the composition and properties of the diluent system including any additives, and the methods employed to disrupt agglomerates and optimize syringeability.

Though being a two-phase system (containing both a solid particulate phase dispersed in a continuous liquid phase comprising one or more diluents) and thus encompassed within the broad category of suspensions, VES formulations may be physically distinct from dilute suspensions and other pharmaceutical formulations in several respects. The concentration of the particulate matter (e.g., powder) in a VES may be sufficiently high such that the particles may not settle in the continuous liquid phase over storage conditions and storage periods relevant to commercial pharmaceutical drug products. Additionally, the cohesive interparticle network that characterizes a VES, as described herein, may impart viscoelastic properties and elevated viscosity to the composition, distinguishing VES formulations from dilute suspensions that lack such a network. As described herein, VES formulations may further be distinguished from gels, creams, foams and other ‘semi-solid’ pharmaceutical dosage forms that may contain lower solids concentrations and that may rely on dissolved polymers or other mechanisms to achieve their consistency.

Accordingly, parenteral (e.g., intracutaneous, subcutaneous, intradermal, and/or intramuscular) delivery (e.g., injection) of such concentrated suspensions (e.g., VES formulations) may pose difficulties. In particular, such compositions may have elevated apparent viscosity when compared with dilute suspensions or traditional aqueous solutions, and injection of concentrated suspensions with elevated viscosity using traditional syringes may be difficult (e.g., potentially requiring excessive force and/or causing excessive pain due, for example, to the use of larger bore needles). Further, being two-phase compositions comprising solid particulate matter dispersed in a continuous liquid phase, these compositions may be particularly susceptible to either partial and/or complete occlusion of the delivery flow path, for example due to the presence of powder agglomerates that may bridge, interlock, accumulate, or otherwise impede flow at constriction points in the delivery flow path, which may impose a further limitation on the potential for parenterally delivering therapeutic concentrated suspensions.

Methods of injecting concentrated suspensions (e.g., VES formulations) have been previously disclosed. For example, U.S. Pat. Nos. 8,790,679, 8,110,209 and 9,314,424, and U.S. Patent Publication Nos. US 2017/0007675 and US 2017/0216529 (the disclosures of all of which are incorporated herein by reference in their entireties) disclose the preparation of therapeutic concentrated suspensions for intracutaneous administration and indicate that because such compositions may display elevated viscosity and reduced flow characteristics in standard syringes, novel needle/syringe designs may be required to deliver such compositions. To accomplish delivery, the injection device may preferably incorporate a plunger that may fit into the lumen of the needle, and that may act in a way such that the full amount of the therapeutic composition loaded into the device may be loaded into the lumen of the needle and may then be pushed out into the patient upon administration using a positive displacement design. Notably, however, this type of configuration may require a plunger that fits within the lumen of a needle and that may be displaced toward the end of the needle upon activation in such a manner that a substantial portion (e.g., greater than about 90%) of the loaded therapeutic composition may be dispensed through the needle and into the injection site.

As is well known in the field, commercially available syringes possess internal barrel diameters that may be several times larger than the internal diameter of the lumen of a needle. For example, the standard 1-mL long syringes used in many commercial injectable drug products may have an internal diameter of approximately 6.4 mm (compared to approximately 0.26 mm for a 25G needle). Moreover, the injection device described in the prior art may only be capable of delivering a very small volume of composition through a standard needle. As an example, a typical needle used for subcutaneous injection is a 27-gauge (or 27G), ultra-thin wall (UTW) 6-mm (exposed length) needle. This needle has an internal diameter of approximately 300 μm (0.300 mm). Modeling the internal volume of the needle as a cylinder of height 6 mm and diameter 0.300 mm, the volume of composition that may be contained within such a geometry is 4.24×10⁻⁴ cm³, or approximately 0.42 μL. Typical injection volumes for intracutaneous delivery often may range from 100-1000 μL (0.1-1.0 mL), and depending on the indication, drug, etc., the delivered volume may be even larger (e.g., 2000 or 3000 μL or more). Thus, delivery of most therapeutically relevant volumes may require very long and very large (with respect to the internal diameter) needles. As is further discussed in the art, “the needle portion of the injection device is from about 6 to about 8 cm in length, thereby providing a lumen having a sufficient interior volume to contain the dose of semi-solid therapeutic formulation and the plunger.” US Patent Publication 2006/0211982, paragraph

Typical needle lengths for intradermal (I.D.) and subcutaneous (S.C.) administration are ≥0.5 inches (or 1.3 cm). Even deeper intramuscular (I.M.) injections commonly employ needles only between 1.0 and 1.5 inches (or between 2.5-3.8 cm). Accordingly, the needles envisioned for the administration of viscous therapeutic pastes or concentrated suspensions would have to be at least twice as long as commercially available needles. However, even using these long and specially designed needles, and also assuming a relatively large internal diameter, the volume that can be placed within the lumen may still be well below that required to achieve a therapeutic dose. For example, the internal volume of an 8-cm long, 18G needle (internal diameter of 0.84 mm) is only 4.4×10⁻² cm³, or approximately 44 μL.

As is further discussed in the art, “the needle portion of the injection device is from about 6 to about 8 cm in length, thereby providing a lumen having a sufficient interior volume to contain the dose of semi-solid therapeutic formulation and the plunger.” US Patent Publication 2006/0211982, paragraph [0115]. Typical needle lengths for intradermal (I.D.) and subcutaneous (S.C.) administration are ≥0.5 inches (or 1.3 cm). Even deeper intramuscular (I.M.) injections commonly employ needles only between 1.0 and 1.5 inches (or between 2.5-3.8 cm). Accordingly, the needles envisioned in the prior art for the administration of concentrated suspensions (e.g., VES formulations) may have to be at least twice as long as commercially available needles. However, even using these long and specially designed needles, and also assuming a relatively large internal diameter, the volume that may be placed within the lumen may still be well below that required to achieve a therapeutic dose. For example, the internal volume of an 8-cm long, 18G needle (internal diameter of 0.84 mm) is only about 4.4×10⁻² cm³, or approximately 44 μL.

In addition to the small volumes that can be administered from an arrangement where the entire dose is contained within the lumen of the needle, such long needles may typically have to be specially manufactured and may be frightening or repulsive to certain patients due to their length. Moreover, as injection pain can be related to the overall diameter (i.e., gauge) of the needle, such large needles may be very painful, and thus may adversely affect patient compliance with a dosing regimen that requires multiple injections with such large needles.

Accordingly, there may be a need in the art for storage-stable compositions, methods, kits and devices for use in parenteral delivery of concentrated suspensions comprising one or more therapeutic agents. Such therapeutic agents may include, without limitation, small molecule therapeutics such as kinase inhibitors, immunomodulatory agents, proteasome inhibitors, targeted protein degraders, and other small molecules as described herein, as well as biologic molecules such as monoclonal antibodies, bispecific antibodies, antibody-drug conjugates, fusion proteins, peptides, proteins, enzymes, oligonucleotides, and other biologics as described herein. There may be an additional need for compositions, methods, kits and/or devices that may facilitate delivery of such compositions using a variety of delivery systems, including but not limited to prefilled syringes, autoinjectors, pen injectors, on-body delivery systems, and subcutaneous infusion pumps, that may accommodate volumes ranging from less than about 1 mL to greater than about 10 mL, and that may provide enhanced syringeability, improved patient comfort, and reliable dose delivery across this range of therapeutic agents, concentrations, and delivery modalities.

### BRIEF SUMMARY OF THE INVENTION

The present invention provides compositions suitable for the parenteral, i.e., intracutaneous, subcutaneous and/or intramuscular administration of concentrated suspensions (e.g., VES), and may provide such compositions, methods of manufacturing and use of such compositions and kits comprising such compositions. Certain aspects of the invention described herein may be directed to the discovery that concentrated suspensions comprising elevated concentrations of active pharmaceutical ingredients may be readily delivered parenterally from a standard (i.e., commercially available) syringe/needle combination. In this way, the present invention may provide pharmaceutical compositions comprising an elevated mass of an active pharmaceutical ingredient in a relatively low volume of diluent or carrier (compared to traditional aqueous pharmaceutical formulations), particularly wherein the compositions may be manufactured in a way that may permit administration of the composition to a patient other than via intravenously, e.g., subcutaneously, intradermally or parenterally, in a way that may provide a ready-to-use composition (i.e., one that may not require reconstitution or dilution prior to being administered to a patient), and that may additionally provide longer-term storage stability than has been previously achieved. The present invention thus may facilitate the manufacturing, storage and delivery of medications parenterally which previously were only delivered intravenously—i.e., the ability to take a large volume medication (regardless of the route of administration) and deliver the same therapeutic effect using a smaller volume intracutaneous, subcutaneous or intramuscular injection. In certain embodiments, this approach may be coupled with a reduction in adverse injection site reactions that may often accompany parenteral injection of large-volume pharmaceutical compositions.

In one aspect, the present invention may provide methods of producing concentrated suspensions having solids contents and rheological properties suitable for parenteral injection of therapeutic agents or active pharmaceutical ingredients. Such methods may comprise preparing one or more powders comprising the therapeutic agent, for example by spray-drying or lyophilizing aqueous formulations comprising one or more active pharmaceutical ingredients, and optionally further processing (e.g., micronizing, grinding, sieving, etc.) the resulting powders to yield powder particles of relatively small diameter and a size distribution such that they may be delivered through small diameter needles appropriate for administration by parenteral injection. In certain aspects, such powders may then be dispersed in one or more diluents at a relatively low solids content to form a dilute suspension, subjected to high-shear mixing to disrupt powder agglomerates, and subsequently concentrated by removing a portion of the diluent through a separation process as described herein to produce a concentrated suspension having a target solids content and a reduced population of agglomerates. In certain other aspects, such powders may be blended with one or more diluents that are non-solvents, substantially non-solvents, or only partial solvents with respect to the powder to produce concentrated suspensions with elevated solids content and elevated active ingredient concentration that may be suitable for injection in relatively low volumes into an animal (e.g., a human or a veterinary animal) in order to treat, ameliorate, prevent or diagnose a disease or physical disorder in the animal. The invention also may provide such concentrated suspensions produced by such methods of the invention.

In additional aspects, the present invention may provide concentrated suspensions for parenteral (e.g., intracutaneous, subcutaneous and/or intramuscular) administration of therapeutic agents or active pharmaceutical ingredients, and methods of manufacturing such compositions in a way that may result in the production of storage-stable compositions that may be ready-to-use (i.e., that may not require reconstitution and/or dilution prior to use). In certain such compositions, the therapeutic agent itself may be complexed or conjugated with one or more excipients or copolymers. In additional aspects, the compositions of the invention generally may comprise one or more excipients, carriers or buffers, and other pharmaceutically acceptable carriers, excipients and fillers that may be readily familiar to those of ordinary skill in the relevant arts. In certain embodiments, the concentrated suspensions of the invention may comprise one or more pharmaceutically acceptable excipients, carriers, or buffers. Such excipients may include, without limitation, one or more monosaccharides such as dextrose, glucose, mannose, fructose, galactose, and the like; one or more disaccharides such as sucrose, lactose, maltose, trehalose, and the like; one or more sugar alcohols such as mannitol, xylitol, glycerol, erythritol, maltitol, sorbitol, and the like; one or more buffering agents such as histidine, citrate, succinate, lactate, phosphate, acetate, and the like; one or more surfactants such as polysorbate 20, polysorbate 80, poloxamer 188, Kolliphor® HS15, sorbitan monolaurate (Span 20), and the like; one or more lipophilic fluids such as medium-chain triglycerides (e.g., Miglyol® 810 (caprylic/capric triglyceride), Miglyol® 812 (caprylic/capric triglyceride)), propylene glycol diesters (e.g., Miglyol® 829 (caprylic/capric/succinic triglyceride), Miglyol® 840 (propylene glycol dicaprylate/dicaprate), mixed lipid systems (e.g., Miglyol® 818 (caprylic/capric/linoleic triglyceride)), triacetin, benzyl benzoate, ethyl oleate, sesame oil, cottonseed oil, and the like; one or more hydrophilic fluids such as propylene glycol, polyethylene glycol 400, and the like; one or more amino acids such as proline, glycine, methionine, tryptophan, phenylalanine, arginine, cysteine, and the like; one or more chelating agents such as ethylenediaminetetraacetic acid (EDTA) and its pharmaceutically acceptable salts, citric acid, tartaric acid, and the like; one or more antioxidants such as ascorbic acid, sodium metabisulfite, and the like; one or more tonicity modifiers such as sodium chloride, and the like; one or more preservatives such as benzyl alcohol, metacresol, phenol, and the like; and other pharmaceutically acceptable carriers, excipients, and fillers that may be readily familiar to those of ordinary skill in the relevant arts. The foregoing lists are non-limiting examples, and other suitable excipients may be used either alone or in combination with those described above.

Concentrated suspensions provided by the invention may be stable and may not require reconstitution prior to use, comprising an effective amount of at least one therapeutic agent (and in some embodiments, more than one, e.g., two, three, four or more therapeutic agents in an admixture, particularly such coformulations in which two or more therapeutic agents are present that are not compatible with each other in a typical aqueous formulation) homogeneously contained within a pharmaceutically acceptable carrier. In certain such aspects, the compositions may comprise from about 10% to about 95% by weight (i.e., solids content), from about 15% to about 90% solids content, or from about 20% to about 85% solids content, and in certain embodiments from about 40% to about 75% by weight, particularly about 40%, about 42%, about 45%, about 50%, about 55%, about 60%, about 65%, about 67% or about 70%, by weight. In certain such aspects, the powder comprising the therapeutic or pharmaceutically active agent may have primary powder particles with a mean particle size range of from about 10 nanometers (0.01 micrometers) to about 100 micrometers, with no primary particles being larger than about 1 mm, and in certain such embodiments the powder may have a mean primary particle size from about 0.1 micrometers to about 25 micrometers, with no primary particles being larger than about 25 micrometers, and in certain other embodiments the powder may have a mean primary particle size of from about 0.5 to about 15 micrometers, particularly wherein at least about half of the primary powder particles may range in size from about 1 micrometers to about 10 micrometers. It is understood that powders may contain agglomerates comprising clusters of two or more primary particles, and such agglomerates may be substantially larger than the primary particles and may require disruption through processing methods such as high-shear mixing and separation processes as described herein to achieve improved deliverability of the concentrated suspension through the intended delivery flow path. In particular, the processes used to produce the present compositions may result in compositions wherein the primary powder particles are relatively uniform in size, though not necessarily considered monodisperse. For example, the measured size distribution of the primary powder particles (e.g., measured by standard techniques such as laser diffraction and reported as the D₁₀, D₅₀ and D₉₀) may yield a span (typically defined in the field as ((D₉₀−D₁₀)/D₅₀)) ranging from 0.5-5.0, or 1.0-3.0, or 1.25-2.5. Ideally, the primary powder particles of the therapeutic agent may be of a size and of a size distribution such as to promote efficient packing, characteristics that can be controlled using the manufacturing processes provided by the invention as described elsewhere herein.

In certain embodiments, the composition may further comprise one or more carriers (e.g., one or more diluents, additives and/or polymers) which may impart thixotropic properties to the composition. The therapeutic agent may be homogeneously incorporated into the pharmaceutically acceptable carrier(s), and said composition may be in a thixotropic or non-Newtonian state in the form of a concentrated suspension. In certain preferred such embodiments, the therapeutic agent is present in powder form and is homogeneously contained within a pharmaceutically acceptable carrier. The carrier is preferably biocompatible and is a non-solvent to the therapeutic agent powder (such that no or minimal dissolution of the powder occurs in the carrier), and in certain preferred embodiments fills the spaces between the particles of the therapeutic agent powder in a way that makes them flow. In certain such embodiments, the carrier is selected from the group consisting of alkyl benzoates, aryl benzoates, aralkyl benzoates, triacetin, aprotic polar solvents (e.g., N-methyl-2-pyrrolidine 5 (NMP), dimethyl sulfoxide (DMSO)), medium chain triglycerides (MCTs, e.g., Miglyol®810, Miglyol®812 N (caprylic/capric triglyceride), Miglyol®818, Miglyol®829, Miglyol®840, and the like), alkanes, cyclic alkanes, chlorinated alkanes, fluorinated alkanes, perfluorinated alkanes and mixtures thereof. The carrier can be a single fluid or semi-solid, or a mixture of two or more fluids (or semi-solids) that may be either partially or fully miscible with each other, or that are immiscible with each other such as mixtures of two or more fluids that form an emulsion.

In certain embodiments, the therapeutic agent may be present in powder form and may be homogeneously contained within a pharmaceutically acceptable carrier. The carrier may be biocompatible and may be a nonsolvent, substantially nonsolvent, or only partial solvent to the therapeutic agent powder (such that no or minimal dissolution of the powder occurs in the carrier), and in certain embodiments may fill the spaces between the particles of the therapeutic agent powder in a way that may enable them to flow. In certain such embodiments, the carrier may be selected from the group consisting of lipophilic diluents as described herein, including but not limited to alkyl benzoates, aryl benzoates, aralkyl benzoates, benzyl benzoate, triacetin, aprotic polar solvents (e.g., N-methyl-2-pyrrolidine 5 (NMP), dimethyl sulfoxide (DMSO)), medium chain triglycerides (e.g., Miglyol® 810, Miglyol® 812), propylene glycol diesters (e.g., Miglyol® 829, Miglyol® 840), mixed lipid systems (e.g., Miglyol® 818), alkanes, cyclic alkanes, chlorinated alkanes, fluorinated alkanes, perfluorinated alkanes and mixtures thereof. The carrier may be a single diluent, or a mixture of two or more diluents that may be either partially or fully miscible with each other, or that are immiscible with each other such as mixtures of two or more diluents that may form an emulsion.

In certain embodiments, the injectable composition may provide controlled (slow) or sustained release. In such embodiments, for example, the composition may comprise a pharmaceutically acceptable polymer in an amount effective to slow the release of the therapeutic agent from said composition upon administration via injection into the epidermal, dermal or subcutaneous layer of an animal. The agent(s) promoting controlled or sustained release of the active pharmaceutical (therapeutic) ingredient in such compositions may be incorporated into the continuous (diluent) phase and/or the dispersed (particulate matter) phase of the composition. Additionally, or alternatively, the therapeutic agent may be incorporated into liposomes or conjugated to or incorporated with polysaccharides and/or other polymers to provide a controlled release of the therapeutic agent from said composition upon administration via injection into the epidermal, dermal or subcutaneous layer of an animal. In certain embodiments, the therapeutic agent may be incorporated into a biocompatible polymer and a biocompatible solvent having low water miscibility that may form a viscous gel with the polymer and may limit water uptake by the composition. Such compositions may be disclosed, for example, in U.S. Pat. No. 6,130,200, which is incorporated by reference herein in its entirety, and for example may utilize a PEG polymer or a PLGA copolymer together with an effective plasticizing amount of a solvent (e.g., comprising a lower alkyl or aralkyl ester of benzoic acid) to form a gel with the polymer. Exemplary processes for producing such formulations are provided elsewhere herein, particularly in the Examples hereinbelow.

In additional embodiments, the present invention also may provide methods of administration of an injectable composition into an animal (e.g., a human or a veterinary or agricultural animal) parenterally, e.g., intracutaneously (into the epidermis or dermis), subcutaneously, intradermally, or intramuscularly, to deliver elevated concentrations or amounts of the active pharmaceutical ingredient into the animal in a lower volume than would be possible with traditional aqueous formulations, or that may effect pain-free or substantially pain-free administration of a therapeutic agent, comprising injecting a concentrated suspension (e.g., a VES) comprising from about 20 to about 85% solids content, by weight, and comprising an effective amount of a therapeutic agent into the epidermal, dermal or subcutaneous skin layer of an animal. The present invention is further directed in part to methods of treating animals, e.g., human patients or veterinary or agricultural animals, utilizing the injectable formulations, injection devices and methods of preparation of the present invention.

In certain embodiments, the therapeutic agent may be processed, e.g., via spray drying or lyophilization, to produce a particle size suitable for injection through a fine-gauge needle (e.g., 21 to 30 gauge). The therapeutic agent may be processed into a powder alongside one or more excipients that may be included, for example, to promote stability, achieve a desired pharmacokinetic profile and/or improve manufacturability of the therapeutic agent.

Exemplary processes for producing such compositions are provided elsewhere herein, particularly in the Examples herein. The term “intramuscular” means administration into the muscle layer of an animal, e.g., a human or veterinary or agricultural animal.

In certain embodiments, the powder comprising the therapeutic agent may be incorporated into a nonaqueous or semi-aqueous pharmaceutically acceptable diluent. In further embodiments, the composition may exhibit shear-thinning properties upon delivery from a delivery device.

The present invention may be further directed in part to methods of treating animals, e.g., human patients or veterinary or agricultural animals, utilizing the injectable compositions, delivery devices and methods of preparation of the present invention.

In certain aspects, the present invention provides syringes that have been pre-loaded with a high concentration/high viscosity pharmaceutical paste formulation of the invention. In certain such embodiments, pre-loaded syringes comprise a syringe body defining a reservoir, a paste disposed within the reservoir, the paste having a solids concentration of at least about, about, or greater than 100-1000 mg/mL (particularly about 100 mg/mL, about 200 mg/mL, about 300 mg/mL, about 350 mg/mL, about 400 mg/mL, about 425 mg/mL, about 450 mg/mL, about 475 mg/mL, about 500 mg/mL, about 525 mg/mL, about 550 mg/mL, about 575 mg/mL, about 600 mg/mL, about 625 mg/mL, about 650 mg/mL, about 675 mg/mL, about 700 mg/mL, about 750 mg/mL, about 800 mg/mL, about 850 mg/mL, about 900 mg/mL, about 950 mg/mL and about 1000 mg/mL, and most particularly about 300 mg/mL to about 950 mg/mL), a plunger and/or piston disposed within the reservoir and configured to be moved to dispense paste from the reservoir, a Luer fitting disposed on the syringe body and in fluid communication with the reservoir, and a sealing cap disposed on the Luer fitting to seal the reservoir. Some embodiments comprise a needle defining a lumen, the needle configured to be coupled to the syringe body via the Luer fitting to allow intracutaneous delivery of the paste, where the reservoir has an internal first transverse dimension larger than an internal second transverse dimension of the lumen. Embodiments of the present pre-loaded syringes may have the needle affixed to the syringe via a Luer-lock or Luer-slip (“slip-tip”) fitting. Alternative embodiments of the present invention may have the needle permanently affixed to the syringe body using, for example, a staked-needle configuration, wherein needle is not removable from the syringe body as with a Luer fitting.

In certain embodiments, the pre-loaded syringes comprise a syringe body defining a reservoir having an internal first transverse dimension, a paste disposed within the reservoir, the paste having a solids concentration of at least about, about, or greater than about 300-950 mg/mL, a needle defining a lumen having an internal second transverse dimension that is smaller than the first transverse dimension, the needle configured to be in fluid communication with the reservoir to allow intracutaneous delivery of the paste, and a plunger disposed within the reservoir and configured to be moved to dispense paste from the reservoir through the lumen.

In some embodiments of the present pre-loaded syringes, the paste has a volume of between 15, 40, 50, 100, 150, 250 or 500 μL and 1000, 2000, or 3000 μL. In certain aspects the paste can have a volume of between 15 μL and 1000 μL. In some embodiments, the paste has a volume of up to about 40 μL. In some embodiments, the paste has a volume of up to about 50 μL. In some embodiments, the paste has a volume of up to about 100 μL or about 150 μL. In some embodiments, the paste has a volume of up to about 200 μL to about 1000 μL, e.g., about 200 μL, about 300 μL, about 350 μL, about 400 μL, about 450 μL, about 500 μL, about 550 μL, about 600 μL, about 650 μL, about 700 μL about 750 μL, about 800 μL, about 850 μL, about 900 μL, about 950 μL or about 1000 μL.

Some embodiments of the present pre-loaded syringes are configured to dispense paste at a flow rate of at least about, about, or greater than 15 microliters per second (μL/s) under a force applied to the plunger having a magnitude of about or at most 50, 60, or 70 newtons (N). In certain aspects, the force applied to the plunger can be below 5, 10, 20, 25, 30, 35, 40, 45, 50, 55, 60, 65, or 70 N. In a further aspect, the force applied to the plunger can be below 25 N. Some embodiments are configured to dispense paste at a flow rate of greater than 65 μL/s under a force applied to the plunger having a magnitude of about or at most 50 to 70 N. In other aspects, particularly those using autoinjectors or other assisted delivery devices (e.g., reusable autoinjectors), the pre-loaded syringes or devices are configured to dispense paste at a flow rate of the above-described forces or even at higher forces than about 70 N, e.g., about 75 N, about 80 N, about 85 N, about 90 N, about 95 N or about 100 N.

In some embodiments, the formulation is contained within a prefilled syringe. A prefilled syringe (PFS) suitable for use with the present compositions comprises a barrel defining a reservoir configured to contain the formulation, a plunger disposed within the barrel and configured to be advanced to dispense the formulation, and a needle in fluid communication with the reservoir. In certain aspects, the prefilled syringe is a glass prefilled syringe, such as a borosilicate glass syringe. In other aspects, the prefilled syringe comprises a cyclic olefin polymer (COP), also referred to as cyclo olefin polymer, or a cyclic olefin copolymer (COC) barrel, which may reduce extractables and leachables relative to glass and provide break resistance during shipping and handling. Non-limiting examples of prefilled syringes suitable for use with the present compositions include the BD Hypak™ for Biotech glass prefillable syringe (Becton, Dickinson and Company), the BD Neopak™ glass prefillable syringe (Becton, Dickinson and Company), the SCHOTT syriQ® prefillable syringe (SCHOTT Pharma AG), the Gerresheimer Gx® RTF ready-to-fill syringe (Gerresheimer AG), the Ompi EZ-fill® prefillable syringe (Stevanato Group S.p.A.), the DAIKYO Crystal Zenith® cyclic olefin polymer syringe (West Pharmaceutical Services, Inc.), and Nipro prefillable syringes (Nipro Corporation), and others that are known in the art.

In some embodiments, the prefilled syringe has a nominal fill volume suitable for the intended dose and route of administration. By way of example and not limitation, in certain aspects, the nominal fill volume is from about 0.5 mL to about 5 mL. In some embodiments, the nominal fill volume is from about 1 mL to about 2.25 mL. In some embodiments, the prefilled syringe has a nominal fill volume of about 1 mL. In other embodiments, the prefilled syringe has a nominal fill volume of about 2.25 mL. In still other embodiments, the prefilled syringe has a nominal fill volume of about 3 mL or greater.

In some embodiments, the prefilled syringe comprises a staked needle. In certain aspects, the staked needle is a thin-wall needle or an ultra-thin-wall needle or an extra-thin-wall needle. As used herein, an ultra-thin-wall needle refers to a needle having a wall thickness that is reduced relative to a standard-wall needle of the same gauge, thereby providing a larger internal lumen diameter and reduced resistance to fluid flow. Non-limiting examples include the BD Neopak™ XtraFlow™ glass prefillable syringe (Becton, Dickinson and Company), which incorporates ultra-thin-wall needle technology to reduce glide force and injection time. In some embodiments, the prefilled syringe comprises a needle having a gauge suitable for the intended formulation and route of administration. By way of example and not limitation, the needle gauge may be from about 21G to about 30G, such as from about 23G to about 29G, such as from about 25G to about 27G. In certain aspects, the needle has a length of about 8 mm to about 12.7 mm (about ½ inch).

In some embodiments, the prefilled syringe is compatible with one or more autoinjector platforms described herein. In some embodiments, the prefilled syringe is provided as a standalone device for manual injection by a patient or healthcare provider.

In some embodiments, the formulation is administered using an autoinjector. As used herein, an autoinjector refers to a handheld, self-contained device configured to automatically insert a needle and deliver a predetermined dose of a formulation subcutaneously or intramuscularly upon activation by the user. In certain aspects, the autoinjector is a single-use, disposable autoinjector. In other aspects, the autoinjector comprises a reusable power unit and a disposable drug cassette containing a prefilled syringe or prefilled cartridge.

In some embodiments, the autoinjector is a spring-driven autoinjector comprising a housing, a prefilled syringe disposed within the housing, and a spring mechanism configured to advance the plunger of the prefilled syringe to dispense the formulation upon activation. Non-limiting examples of spring-driven autoinjectors suitable for use with the present compositions include the Molly® autoinjector and the Molly® 2.25 autoinjector (SHL Medical AG), the YpsoMate® autoinjector and the YpsoMate® 2.25 autoinjector (Ypsomed AG), the BD Physioject™ disposable autoinjector (Becton, Dickinson and Company), the Safelia® autoinjector (Nemera S.A.S.), the Aidaptus® autoinjector (Owen Mumford Ltd.), the eCAP™ autoinjector (Elcam Drug Delivery Devices Ltd.), and the ARAI™ auto-injector (Aktiv Pharma Group), and others that are known in the art.

In some embodiments, the autoinjector is configured to deliver the formulation in a volume and within a delivery time suitable for the intended dose and patient population. By way of example and not limitation, the autoinjector may be configured to deliver the formulation in a volume of up to about 1 mL, or up to about 2.25 mL, or greater.

In some embodiments, the autoinjector comprises an enhanced spring force, a torsion spring with a regulator to control the application of force over time, or a pressure-assisted mechanism to manage increased glide force associated with the formulation. Non-limiting examples of autoinjectors suitable for use with the present compositions include the YpsoMate® 2.25 Pro autoinjector (Ypsomed AG), and the VIBEX® and VIBEX® QuickShot® pressure-assisted auto injectors (Halozyme, Inc., formerly Antares Pharma, Inc.), and others that are known in the art.

In some embodiments, the autoinjector employs a torsion spring mechanism with a regulator to control the application of force over time during the injection stroke, thereby maintaining a more consistent injection force profile relative to a linear compression spring. A non-limiting example of such a technology is the Rotaject® technology (SHL Medical AG), which employs a patented clock spring mechanism for constant force delivery of high-volume drug formulations. In other embodiments, the autoinjector employs a pressure-assisted mechanism in which a gas cartridge or other stored energy source provides the driving force for plunger advancement. In still other embodiments, the autoinjector is an electromechanical autoinjector comprising a motor-driven actuation system. A non-limiting example of an electromechanical autoinjector is the Elexy™ autoinjector (SHL Medical AG), which comprises a reusable electronic power unit compatible with disposable drug cassettes containing a prefilled syringe or cartridge.

In some embodiments, the autoinjector further comprises a needle safety feature configured to shield the needle before and/or after injection. Non-limiting examples of needle safety devices include the BD UltraSafe™ passive needle guard (Becton, Dickinson and Company) and the Safe'n' Sound® needle safety device (Nemera S.A.S.). In certain aspects, the autoinjector provides audible, visual, or tactile feedback to the user indicating the start and/or completion of the injection.

In some embodiments, the formulation is administered using a large-volume handheld autoinjector configured to deliver the formulation in a volume greater than that of a standard autoinjector. By way of example and not limitation, the large-volume handheld autoinjector may be configured to deliver the formulation in a volume of greater than about 2.25 mL, such as from about 3 mL to about 10 mL, such as from about 3 mL to about 5 mL, or greater. Large-volume handheld autoinjectors may employ cartridge-based primary containers rather than prefilled syringes to accommodate the increased fill volume, and may utilize enhanced actuation mechanisms including, without limitation, high-force spring mechanisms, torsion springs, gas-assisted drives, or electromechanical motor-driven systems to deliver the formulation within a delivery time suitable for patient comfort.

Non-limiting examples of large-volume handheld autoinjectors suitable for use with the present compositions include the Maggie® 3.0 mL cartridge-based autoinjector and the Maggie® 5.0 mL cartridge-based autoinjector (SHL Medical AG), each of which incorporates Needle Isolation Technology (NIT®). In some embodiments, the handheld form factor is preferred by the patient over a wearable on-body device.

In some embodiments, the large-volume handheld autoinjector is an electromechanical autoinjector comprising a reusable electronic drive unit and a single-use drug cassette. The electronic drive unit may be programmed to deliver the formulation at a controlled flow rate, a variable flow rate, or a ramping flow rate profile. In certain aspects, the electromechanical autoinjector is the Elexy™ autoinjector (SHL Medical AG), which is compatible with drug cassettes containing either a prefilled syringe or a cartridge.

In some embodiments, the formulation is administered using a pen injector. As used herein, a pen injector refers to a device having a generally cylindrical, pen-like form factor configured to deliver one or more doses of a formulation from a replaceable or integrated cartridge. Pen injectors may be disposable (prefilled, single-patient-use) or reusable (accepting replaceable cartridges). In certain aspects, the pen injector is a variable-dose pen injector configured to allow the user to select a dose prior to injection. In other aspects, the pen injector is a fixed-dose pen injector.

Non-limiting examples of pen injectors suitable for use with the present compositions include the PENDURA™ AD reusable pen injector (Nemera S.A.S.), which integrates an automatic spring-driven dose release feature coupled with a side-activation button; the Axis-D™ disposable pen system and the i-Pen™ reusable pen injector (Wilhelm Haselmeier GmbH & Co. KG); the Autopen® reusable pen injector (Owen Mumford Ltd.); the UnoPen® fixed-dose disposable pen injector and the YpsoMate® Pen variable-dose disposable pen injector (Ypsomed AG); pen injector platforms offered by SHL Medical AG; and the Companion™ safety syringe system (Credence MedSystems, Inc.), which may be configured with auto-retract needle and dual-chamber reconstitution capabilities, and others that are known in the art.

In some embodiments, the pen injector is configured to deliver a dose volume suitable for the intended formulation and dosing regimen. By way of example and not limitation, the dose volume may be from about 0.1 mL to about 3 mL per injection, or greater. In certain aspects, the pen injector is a multi-dose device configured to deliver a plurality of injections from a single cartridge. Pen injectors are particularly suitable for formulations requiring dose titration or frequent administration, such as daily, every-other-day, or weekly dosing regimens.

In some embodiments, the formulation is administered using an on-body delivery system (OBDS), also referred to as a wearable injector, a body-worn injector, or an on-body injector. As used herein, an on-body delivery system refers to a device configured to be adhered to the skin of a patient-such as on the abdomen, upper thigh, or upper arm-using an adhesive, and to deliver the formulation subcutaneously at a controlled flow rate over an extended period of time.

On-body delivery systems may employ a mechanical, electromechanical, or electrochemical actuation mechanism to advance a plunger or piston to dispense the formulation from a reservoir through a cannula or needle into the subcutaneous tissue. In certain aspects, the OBDS autonomously handles needle or cannula insertion, formulation delivery at a predetermined or variable flow rate, and post-dose needle retraction. In some embodiments, the OBDS is configured to deliver the formulation at a constant flow rate. In other embodiments, the OBDS is configured to deliver the formulation at a variable or ramping flow rate, which may be programmed to accommodate the rheological properties of the formulation and to minimize injection-site pain and tissue back-pressure.

In some embodiments, the OBDS is configured to deliver a volume suitable for the intended dose. By way of example and not limitation, the volume may be from about 2 mL to about 50 mL, such as from about 3 mL to about 20 mL, such as from about 3 mL to about 10 mL, or other volumes as appropriate.

Non-limiting examples of on-body delivery systems suitable for use with the present compositions include the SmartDose® electronic wearable bolus injector (West Pharmaceutical Services, Inc.), which has been configured to deliver a volume of about 3.5 mL; the Onpro® on-body injector (Amgen Inc.), which has been used for delivery of pegfilgrastim (Neulasta®); the YpsoDose® large-volume patch injector (Ypsomed AG), which is a prefilled, spring-driven system configured to deliver volumes of up to about 10 mL; and the BD Libertas™ wearable autoinjector (Becton, Dickinson and Company), which is an electromechanical system configured to deliver volumes of from about 2 mL to about 5 mL, and others that are known in the art.

In some embodiments, the OBDS is an electromechanical wearable injector comprising a programmable electronic controller and a miniaturized motor or pump. Non-limiting examples include the Sorrel™ on-body injection platform (LTS LOHMANN Device Technologies), which comprises a reusable electromechanical smart controller paired with a single-use, prefilled cassette and is configured to deliver volumes of from about 1 mL to about 50 mL with variable delivery profiles accommodating a range of formulations; the Symbioze® reusable on-body injector platform (Nemera S.A.S.), which employs a miniaturized motor capable of sustaining controlled flow rates and comprises a reusable electronic portion and a disposable drug-containing element for delivery of volumes up to about 20 mL; the enFuse® on-body delivery platform (Enable Injections, Inc.), which is configured to deliver volumes of up to about 50 mL; and the Sonceboz wearable injection platforms (Sonceboz SA), which employ micro-motor-based electromechanical actuation, and others that are known in the art.

In some embodiments, the OBDS further comprises one or more connectivity features selected from Bluetooth, near-field communication (NFC), and wireless data transmission, configured to provide patient feedback, therapy tracking, dose confirmation, or integration with electronic health records or companion mobile applications. In certain aspects, the OBDS provides audible, visual, or haptic feedback to the user indicating device status, injection progress, and dose completion.

In some embodiments, the formulation is administered using a bolus injector, which may also be referred to as a patch pump or a high-volume injector. In certain aspects, a patch pump may be employed for prolonged delivery of formulations. Devices may be worn on the body and may provide automated subcutaneous or intra-cutaneous delivery of a high-concentration formulation at a slower infusion rate relative to a traditional autoinjector or manually operated syringe.

Non-limiting examples of bolus injectors and patch pumps suitable for use with the present compositions include the Lapas® bolus injector (Bespak, a Recipharm company), which is a mechanical patch pump configured to deliver volumes of up to about 10 mL for prolonged subcutaneous delivery of pastes; the Omnipod® tubeless patch pump system (Insulet Corporation), which is an electromechanical wearable pump primarily developed for insulin delivery but adaptable to other therapeutic proteins; and the MiniMed™ infusion pump system (Medtronic, Inc.), which provides highly precise flow control for subcutaneous infusion, and others that are known in the art.

In some embodiments, the formulation is administered using a subcutaneous infusion pump, also referred to as a syringe-driving pump or an ambulatory infusion pump.

As used herein, a subcutaneous infusion pump refers to a device configured to deliver a formulation from a syringe or reservoir through tubing and a subcutaneous needle or cannula at a controlled, programmable flow rate over an extended period of time. Subcutaneous infusion pumps are suitable for delivery of large-volume formulations. By way of example and not limitation, the volume delivered may be greater than about 10 mL, such as from about 10 mL to about 60 mL, or greater.

Non-limiting examples of subcutaneous infusion pumps suitable for use with the present compositions include the Freedom60® syringe driver (EMED Technologies, Inc.), which is a spring-driven, ambulatory infusion pump configured to deliver volumes of up to about 60 mL; the SCIg infusion systems offered by RMS Medical Products; and the Sapphire™ multi-therapy infusion pump (Eitan Medical Ltd.), which provides precision electromechanical flow control, and others that are known in the art.

In some embodiments, the subcutaneous infusion pump is configured to deliver the formulation to one or more subcutaneous infusion sites simultaneously. In certain aspects, the infusion pump is configured to deliver the formulation at a flow rate that does not exceed a subcutaneous tissue back-pressure threshold associated with pain or tissue damage. In some embodiments, the formulation is co-administered with a permeation enhancer, such as recombinant human hyaluronidase (rHuPH20), to facilitate dispersion and absorption of large volumes in the subcutaneous space.

Some embodiments of the present kits comprise a device selected from the group consisting of a prefilled syringe, an autoinjector, a large-volume handheld autoinjector, a pen injector, an on-body delivery system, a bolus injector, a patch pump, and a subcutaneous infusion pump, wherein the device contains the formulation described herein and is configured to deliver the formulation subcutaneously or intramuscularly to a patient.

In some embodiments, the device comprises a plunger or piston disposed within a reservoir and configured to be moved to dispense the formulation from the reservoir through a lumen under a force applied to the plunger. The device may be configured to deliver the formulation in a volume, at a flow rate, and over a delivery time appropriate for the device type, formulation, and intended use.

Some embodiments of the present kits comprise a syringe body defining a reservoir having an internal first transverse dimension and a needle configured to be coupled to the syringe body and defining a lumen having an internal second transverse dimension that is smaller than the first transverse dimension. In some embodiments, the paste is disposed within the reservoir. In some embodiments, the syringe body comprises a Luer fitting (e.g. Luer-lock or Luer-slip fitting) in communication with the reservoir and a sealing cap disposed on the Luer fitting to seal the reservoir, where the needle is configured to be coupled to the syringe body via the Luer fitting. In other embodiments, the needle is integral to the syringe body without being detachably connected. In some embodiments, the reservoir has a volume of between 50, 75, or 100 μL and 1000, 2000, or 3000 μL.

Some embodiments of the present kits comprise a plunger disposed within the reservoir and configured to be moved to dispense paste from the reservoir through the lumen at a flow rate of greater than 30 μL/s under a force applied to the plunger having a magnitude such as those described elsewhere herein. Some embodiments comprise a plunger disposed within the reservoir and configured to be moved to dispense paste from the reservoir through the lumen at a flow rate of greater than 65 μL/s under a force applied to the plunger having a magnitude such as those described elsewhere herein.

An alternative embodiment is the use of bolus injectors, which are alternatively known as patch pumps or high-volume injectors. In certain aspects, a patch pump can be employed for prolonged delivery of viscous pastes to a patient. Examples of these injectors include the SmartDose™ electronic wearable bolus injector (West Pharmaceutical Services, Inc.) and the Lapas® bolus injector (Bespak), and others that are known in the art (see, e.g., Badkar A. V. et al., Drug Des. Devel. Ther. 15:159-170 (2021), doi: 10.2147/DDDT.S287323). These devices can be worn on the body and can provide automated sub- or intra-cutaneous delivery of a high concentration paste at a slower infusion rate relative to a traditional auto-injector or manually operated syringe. In these devices the paste is filled in an internal reservoir and slowly infused into the patient at a low volumetric flow rate (relative to manual syringes and auto-injector devices). These devices may be worn like a patch adhered to the skin, delivering the medicament over the course of several minutes, or up to about an hour. As a non-limiting example of the volumetric flow rates that may be employed in these systems, delivery of 3 mL of a therapeutic paste over the course of 10 minutes would entail a delivery rate of 5 μL/second. Delivery of a 3 mL volume of paste over the course of 1 hour would entail a delivery rate of 0.83 μL/second.

Some embodiments of the present methods for intracutaneously injecting a volume of paste comprise moving a plunger of a syringe to dispense paste from a reservoir of the syringe through a lumen of a needle of the syringe, the reservoir having an internal first transverse dimension that is larger than an internal second transverse dimension of the lumen, e.g., where the second transverse dimension is between 0.1 and 0.9 mm, where the paste has a solids content of about 20% to about 99%, including all values and ranges therebetween, a solids concentration of greater than about 100 mg/mL, e.g., of about 300 to about 950 mg/mL including all values and ranges therebetween, and particularly an active pharmaceutical ingredient concentration of about 300 to about 800 mg/ml including all values and ranges therebetween, and where the paste is dispensed at a flow rate of greater than 30 μL/s as the plunger is moved at a rate of between 0.5 and 50 millimeters per second (mm/s). Some embodiments comprise disposing the needle into and/or through cutaneous tissue of a patient. Some embodiments comprise removing a sealing cap from a Luer fitting of the reservoir. Some embodiments comprise coupling the needle to the reservoir via a Luer fitting disposed on at least one of the needles and the reservoir. In some embodiments, the flow rate of the paste is substantially linearly proportional to the rate of plunger movement.

In some embodiments of the present methods, the injected volume of paste is greater than about 1 μL. In some embodiments, the injected volume of paste is between 15, 30, or 100 μL and 1200, 2000, or 3000 μL for single injection doses, and up to about 10 mL for infusion use. In some embodiments of the present syringes, kits, and/or methods, the first transverse dimension is larger than the second transverse dimension. In some embodiments, the first transverse dimension is between 1, 2, 3, 4 and 5, 6, 7, 8, 9, 10, 11, 12 mm, including all values and ranges there between. In some embodiments, the second transverse dimension is between 0.1, 0.2, 0.3, or 0.4 and 0.5, 0.6, 07, 0.8, or 0.9 mm, including all values and ranges there between.

In some embodiments of the present syringes, kits, and/or methods, the needle is a size of 18 Gauge or of higher gauge (where higher gauge represents a physically smaller needle in terms of needle external and/or internal diameter). In some embodiments, the needle has a size of 23 Gauge or smaller. In some embodiments, the needle has a size of 25 Gauge or 27 G or smaller (i.e., higher gauge). In some embodiments, the needle has an exposed length smaller than or about 50 mm. In some embodiments, the needle has an exposed length smaller than or about 40 mm. In some embodiments, the needle has an exposed length smaller than or about 13 mm. In some embodiments, the needle has an exposed length of approximately 8 mm. In some embodiments, the needle has an exposed length of approximately 6 mm.

In some embodiments of the present syringes, kits, and/or methods, the paste has a solids concentration of greater than 200 mg/mL. In some embodiments, the paste has a solids concentration of between 200 and 950 mg/mL. In some embodiments, the paste has a solids concentration of between 300 and 800 mg/mL. In some embodiments, the paste has a solids content of between 1% and 99%. In some embodiments, the paste has a solids content of between 30% and 95%. In some embodiments, the paste has a solids content of between 40% and 80% or between 50% and 70% or between 50% and 70%. In some embodiments, the paste has a density of between about 0.5, 0.7, 0.75, 1.0, 1.1, 1.2, 1.3, to about 1.4, 1.5, 1.6, 1.7, 1.8, 1.9, 2.0 g/mL, including all values and ranges there between.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Other objects, advantages, and features of the present invention will be readily apparent to those of ordinary skill in the art upon review of the description, drawings, examples and claims presented herein.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 is a chart showing four different exemplary and non-limiting methods of the present invention (Options 1 through 4) to prepare the high solids content concentrated suspensions of the invention.

FIG. 2 is a pair of scanning electron photomicrographs showing particles of therapeutic protein powders (in this case, monoclonal antibodies (mAbs)) prepared according to the spray-drying methods of the invention using different spray-dryer (Buchi B290) apparatus settings. FIG. 2A: inlet temperature 90° C., aspirator 65% (27 m³/hr), nozzle gas rate 473 L/hour (pressure drop 0.41 bar), feed solution pump rate 3% (about 1 mL/min), and no secondary drying or processing (e.g., sieving) after spray-drying. FIG. 2B: inlet temperature 70° C., aspirator 85% (34 m³/hr), nozzle gas rate 473 L/hour (pressure drop 0.41 bar), feed solution pump rate 10% (about 3 mL/min), and secondary drying (lyophilization) and sieving were performed after spray-drying.

FIG. 3 is a series of scanning electron photomicrographs showing particles of therapeutic protein powders (in this case, monoclonal antibodies) prepared according to the spray-drying methods of the invention using different spray-dryer apparatus settings, as set forth in Table 4 hereinbelow. FIG. 3A: Formulation 1; FIG. 3B: Formulation 2; FIG. 3C: Formulation 3; FIG. 3D: Formulation 4; FIG. 3E: Formulation 5; FIG. 3F: Formulation 6; FIG. 3G: Formulation 7; FIG. 3H: Formulation 8.

FIG. 4 is series of bar graphs showing the size distribution (assessed by visual examination via scanning electron microscopy) of particles of therapeutic protein powders (in this case, monoclonal antibodies) prepared according to the spray-drying methods of the invention using different spray-dryer apparatus process settings. Formulation numbers and spray-dryer settings correspond to those set forth in the description of FIG. 3, above. FIG. 4A: Formulation 1; FIG. 4B: Formulation 2; FIG. 4C: Formulation 3; FIG. 4D: Formulation 4; FIG. 4E: Formulation 5; FIG. 4F: Formulation 6; FIG. 4G: Formulation 7; FIG. 4H: Formulation 8.

FIG. 5 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 6 is a chart (top) showing the percent aggregation of certain formulations prepared according to the methods of the invention prior to and after spray-drying (at time zero after spray-drying and after one day storage at 50° C. after spray-drying), and a bar graph depicting these results graphically. Secondary drying was performed after spray drying under reduced pressure in a lyophilizer (lyo).

FIG. 7 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 8 is a chart showing the percent aggregation of certain formulations prepared according to the methods of the invention prior to and after spray-drying (at time zero (T=0) after spray-drying and after five days storage at 40° C. post-drying).

FIG. 9 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 10 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 11 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 12 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 13 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 14 is set of charts showing the main peak (FIG. 14A), acidic variants (FIG. 14B) and basic variants (FIG. 14C) observed upon ion exchange chromatography of cysteine-containing formulations prepared according to the methods of the invention prior to (“pre-SD”) and after spray-drying (at time zero (t0) after spray-drying and after one day storage at 50° C. after spray-drying (50 C×1d)).

FIG. 15 is a comparison of representative tracings of two formulations, one containing 1.5 mg/mL cysteine (FIG. 15A) and the other containing 6 mg/mL cysteine (FIG. 15B) prepared according to the methods of the invention prior to (“pre-SD”) and after spray-drying (at time zero (“t0”) after spray-drying and after one day storage at 50° C. (“50 C×1d”) after spray-drying).

FIG. 16 is a chart (top) showing the percent aggregation of certain formulations prepared according to the methods of the invention prior to and after spray-drying at different inlet temperatures (at time zero after spray-drying and after one day storage at 50° C. after spray-drying), and a bar graph depicting these results graphically.

FIG. 17 is a series of scanning electron photomicrographs showing particles of therapeutic peptide powders (in this case, monoclonal antibodies) spray-dried from the commercial formulations, showing particles observed in spray-dried formulations of Herceptin (TmAb) (FIG. 17A), Erbitux (cetuximab) (FIG. 17B) and Privigen (immune globulin) (FIG. 17C).

FIG. 18 is a chart showing the particle size distribution in the three formulations depicted in FIG. 16, as measured using laser diffraction. D₁₀: tenth percentile particle size; D₅₀: fiftieth percentile particle size; D₉₀: ninetieth percentile particle size. Span=(D₉₀−D₁₀/D₅₀).

FIG. 19 is a line graph depicting the injection force required (measured using a texture analyzer) to dispense 1 mL of paste formulations of the invention using a 1 mL long syringe with a 23G needle (bottom tracings) or a 27G needle (top tracings).

FIG. 20 is an ion exchange chromatogram showing the peaks obtained in formulations of trastuzumab (Herceptin®) in the commercial aqueous form (red tracing), a powder prepared by the spray-drying methods of the invention and then reconstituted in water (green tracing), or a XeriJect® paste formulation of the invention (pink tracing).

FIG. 21 is a line graph (top) showing the pharmacokinetics (PK) of formulations of trastuzumab (Herceptin®) injected into test animals and assessed for plasma antibody concentration in the commercial aqueous form injected intravenously (blue tracing), and two XeriJect® paste formulations of the invention injected subcutaneously into test animals, using a dose of 10 mg/Kg and the other 20 mg/Kg (FIG. 21A); and a chart showing certain PK parameters in tabular form (FIG. 21B).

FIG. 22 is a pair of scanning electron photomicrographs of a human enzyme formulation powder prepared from a commercial aqueous formulation by lyophilization (FIG. 22A) or by the spray-drying methods of the present invention (FIG. 22B).

FIG. 23 is a series of line graphs showing pharmacokinetic (PK) results of intravenous injection (FIG. 23A) of a commercial aqueous formulation of the enzyme used in FIG. 22, or of subcutaneous injection of the aqueous enzyme formulation (FIG. 23B, “Group 2”)) or a XeriJect® paste of the enzyme prepared according to the methods of the present invention (FIG. 23B, “Group 3”).

FIG. 24 is a series of line graphs showing pharmacodynamic results of intravenous injection (FIG. 24A) of a commercial aqueous formulation of the enzyme used in FIG. 22, or of subcutaneous injection of the aqueous enzyme formulation (FIG. 24B, “Group 2”)) or a XeriJect® paste of the enzyme prepared according to the methods of the present invention (FIG. 24B, “Group 3”).

FIG. 25 is series of line graphs showing pharmacokinetic (FIG. 25A) and pharmacodynamic (FIG. 25B) results of subcutaneous injection of a commercially available aqueous glucagon formulation (“GEK” in FIGS. 25A and 25B) or a XeriJect® paste of glucagon prepared according to the methods of the present invention (“Xeris Paste” in FIGS. 25A and 25B).

FIG. 26 is a pair of scanning electron photomicrographs of a human recombinant protein formulation powder prepared from a commercial aqueous formulation by the spray-drying methods of the present invention. FIG. 26A: low-concentration feed solution; FIG. 26B: high-concentration feed solution.

FIG. 27 is a bar graph depicting the injection force required (measured using a texture analyzer) to inject about 150 μL of a recombinant protein paste prepared from the powders shown in FIG. 26, using commercially available large and small syringes affixed with either regular wall or thin wall 27G needles, at a volumetric flow rate of 30 μL per second.

FIG. 28 is a pair of scanning electron photomicrographs of a human monoclonal antibody (bevacizumab, BmAb) formulation powder prepared by the spray-drying methods of the present invention. FIG. 28A: formulation XJ-1 (pH 4.0); FIG. 28B: Formulation XJ-2 (pH 6.0).

FIG. 29 is a pair of pharmacokinetic line graphs showing plasma concentrations over time of various formulations of XeriJect bevacizumab (BmAb) after injection into minipigs. FIG. 29A: linear scale; FIG. 29B: same results, but on a semilogarithmic scale.

FIG. 30 is a bar graph showing the time to maximum plasma concentration (Tₘₐₓ) for various formulations of XeriJect bevacizumab after injection into minipigs.

FIG. 31 is a pair of bar graphs showing the maximum plasma concentration (Cₘₐₓ) for various formulations of XeriJect bevacizumab after injection into minipigs, either uncorrected (FIG. 31A) or corrected for dose (FIG. 31B).

FIG. 32 is a bar graph showing the plasma half-life (T_{1/2}) for various formulations of XeriJect bevacizumab after injection into minipigs.

FIG. 33 is a pair of bar graphs showing dose-corrected total animal exposure for various formulations of XeriJect bevacizumab after injection into minipigs. FIG. 33A: dose-corrected AUCₗₐₛₜ; FIG. 33B: dose-corrected AUC_{∞}.

FIG. 34 is a bar graph showing the dose-corrected partial animal exposure, 14 days post-injection (AUC₃₃₆), for various formulations of bevacizumab after injection into minipigs.

FIG. 35 is a line graph showing the mean (±SEM) plasma insulin concentration after subcutaneous administration of Humulin R and XeriJect insulin formulations in Yucatan minipigs.

FIG. 36 is a line graph showing the mean (±SEM) blood glucose concentration after subcutaneous administration of Humulin R and XeriJect insulin formulations in Yucatan minipigs.

FIG. 37 is a pair of scanning electron photomicrographs of an exemplary spray dried IgG powder formulation produced by the methods of the present invention. Images are provided at different magnifications. Two different magnifications are depicted, at higher (FIG. 37A; scale bar=10 μm) and lower (FIG. 37B; scale bar=20 μm) magnification.

FIG. 38 is a line graph showing the particle size distribution analysis of an exemplary spray dried IgG powder formulation produced by the methods of the present invention.

FIG. 39 is a series of scanning electron photomicrographs of an exemplary spray dried IgG paste produced by the methods of the present invention. Images are provided at different magnifications. Three different magnifications are depicted, at higher (FIG. 39A; scale bar=10 μm and 39B, scale bar=8 μm) and lower (FIG. 39C; scale bar=20 μm) magnification.

FIG. 40 is a line graph showing the particle size distribution analysis of an exemplary spray dried IgG powder formulation produced by the methods of the present invention. X axis: distance (mm); Y axis: force (N).

FIG. 41 is a line graph showing the stability of spray dried bevacizumab (BmAb) powders stored in a 2-8° C. stability chamber, evaluated by size exclusion HPLC. Data are provided as the % Monomer (native) content measured for n=1 replicate samples per timepoint.

FIG. 42 is a series of scanning electron photomicrographs of exemplary spray dried bevacizumab (BmAb) powder formulations produced by the methods of the present invention, at the BmAb percentage (% w/w of total solids in the formulation) shown above each panel. All samples were imaged at the same scale (corresponding to 5000× magnification on the SEM screen used to perform the analysis).

FIG. 43 is a graph showing the injection force profiles of 44% (w/w) lactose-triacetin pastes prepared using unscreened powders that had been manually mixed or mixed with a THINKY mixer, vs. powders that were screened through a 75 μm pore size sieve prior to being mixed in a THINKY mixer.

FIG. 44 is a depiction of the use of a three-roll mill to prepare pastes of the invention, showing the milling apparatus itself (FIG. 44A), the operation of the rollers (FIG. 44B), and the pastes resulting from hand mixing vs. those obtained by passing the paste through the three-roll mill twice (FIG. 44C).

FIG. 45 are scanning electron micrographs of unmilled pastes (FIG. 45A) or pastes that had been passed through the three-roll mill twice (FIG. 45B). Circles in FIG. 45A show examples of large aggregates of particles as are typically seen in unprocessed pastes.

FIG. 46 is a line graph showing the injection force profiles of pastes that were not processed to reduce the number of aggregates (“Unprocessed”) and pastes that had been passed through the three-roll mill twice (“TRM (2×)).

FIG. 47 is a schematic cross-sectional view of an apparatus 200 configured in accordance with one embodiment of the present disclosure, shown in an operational configuration during an active separation process for concentrating a suspension from a dilute suspension comprising a powder and a diluent. The apparatus 200 comprises weighted plates 210, a plunger rod 220, a plunger 230 (functioning as the pushing device), a main body 240, a suspension 250, a separation medium 260, expelled diluent 270, a collection vessel 280, a support 290, and a base 295. A directional arrow labeled “Direction of Travel” indicates the direction of force applied during the separation process.

FIG. 48 is a series of photographs showing the assembly of the Aeropress extraction cassette. FIG. 48A: exploded view showing all components including the pharmaceutical grade membrane filter. FIG. 48B: combination of the filter components results in a stacked membrane filter which is then assembled into the bottom portion of the Aeropress device. FIG. 48C: the assembled filtration cassette.

FIG. 49 is a pair of scanning electron micrographs of the particles in a suspension used to make a paste of the invention, following high-shear mixing and pressing (diluent extraction). Particles are shown at 10,000× (FIG. 49A) and 5,000× (FIG. 49B) magnifications.

FIG. 50 is a pair of graphs showing the injection force profiles for viscoelastic suspensions prepared according to the standard method, stainless steel bead additive method, or press extrusion method (as described in Example 13). In each case, VES was delivered through 2.25 mL glass LL syringes having attached 23G, 8 mm UTW needles at a volumetric flow rate of 50 μL/sec. FIG. 50A: injection force profiles for standard and steel bead methods. FIG. 50B: injection force profile for press extrusion method.

FIG. 51 is a photograph showing the physical properties (including the presence of agglomerates) in a spray-dried trehalose powder that was tumbled. The diameter of the cylinder was 2.2 inches (~56 mm).

FIG. 52 is a pair of photographs comparing the appearance of pastes (VES) prepared by the standard mixing (FIG. 52A) and high-shear mixing (FIG. 52B) approaches described in Example 13.

FIG. 53 is a line graph showing the injection force profiles observed for three samples of pastes (VES) prepared by the press extrusion method. Pastes were delivered from 2.25 mL glass syringes with attached 25G 0.5-inch UTW needles at a volumetric flow rate of 50 μL/sec.

FIG. 54 is a scanning electron micrograph of particles in a paste prepared from a powder of spray-dried IgG particles mixed with Miglyol® 840 as the diluent (scale bar=10 μm).

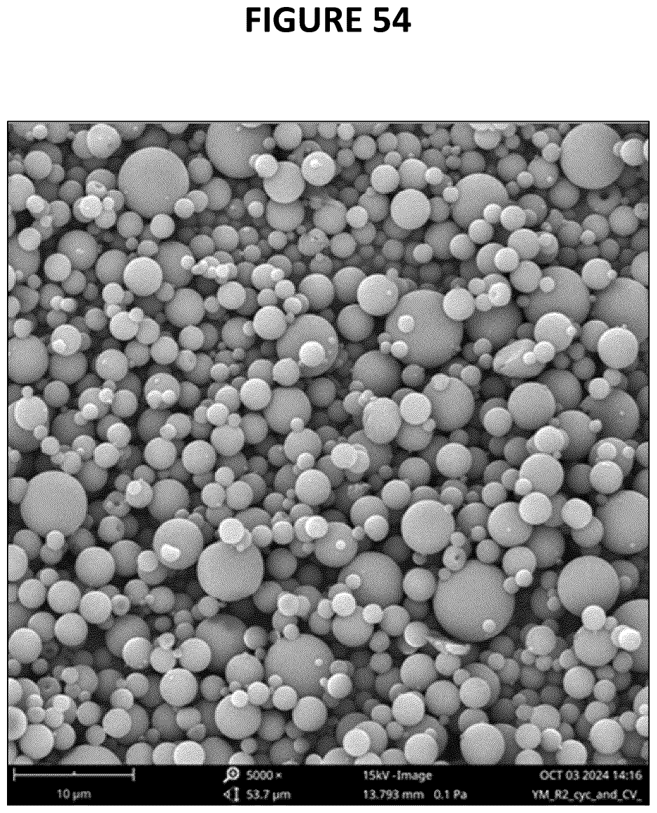

FIG. 55 is a table showing the relative liquid content of a mixture, along with a description of its state, a schematic diagram, and its physical description.

FIG. 56 is a photograph from a scanning electron microscope of micronized lactose prepared according to the methods of the present invention.

FIG. 57 is a graph comparing injection forces for an experimental sample of a formulation containing PS80 and a formulation without PS80.

FIG. 58 is a scanning electron microscope photograph of a spray dried IgG powder formulation prepared according to the methods of the present invention.

FIG. 59 is a series of photographs depicting the flowability and other physical characteristics (top row), and microscopic appearance by SEM (bottom row), of VES formulations of the present invention containing the indicated amounts of PS80.

FIG. 60 is a bar graph showing the mean glide injection forces of IgG VES prepared according to the methods of the present invention and containing 0.10%, 0.25%, 0.50%, 1.00%, or control (0.0%) amounts of PS80.

FIG. 61 is a schematic cross-sectional view of a separation medium assembly in accordance with one embodiment of the apparatus, depicting one non-limiting example of the arrangement of individual components in a layered configuration. A directional arrow labeled “Direction of Flow” indicates the direction of flow of the liquid phase through the assembly during the separation process.

### DETAILED DESCRIPTION OF THE INVENTION

### Definitions

In the present application, certain words, terms, and phrases are defined for reference and clarity only. Other definitions that are known in the art for these words, terms, and phrases may similarly apply, provided that such art-known definitions do not conflict with those provided specifically herein. Other words, terms, and phrases that are not specifically defined herein have their commonly understood definitions.

For purposes of the present invention, the term “therapeutic agent” or “active pharmaceutical ingredient” or “pharmaceutically active ingredients” encompasses small molecule therapeutics and biologic molecules as described herein, including but not limited to those explicitly described herein. In certain embodiments, the compositions may comprise one or more polymer or copolymer carriers which may provide for sustained release of the therapeutic compound, e.g., poly(ethylene glycol) (“PEG”), poly(lactic-co-glycolic acid) (“PLGA”), and the like.

As used herein, a “paste” is a two-phase mixture of a solid (e.g., a powder containing a medicament and, if necessary, stabilizing agents and/or excipients) dispersed in a liquid (e.g., a biocompatible diluent), which is a non-solvent to, or which only minimally solubilizes, the solid (e.g., and thus, the diluent is typically, but not always, lipophilic in nature). A paste behaves as a solid until a sufficiently large load or stress is applied (typically referred to as the ‘yield stress’), at which point the paste flows like a liquid (e.g., pastes may be defined as semi-solids). Pastes may exhibit non-Newtonian fluid behavior, specifically shear-thinning flow characteristics and/or viscoelastic behavior.

As used herein, the terms “viscoelastic suspension,” “viscoelastic composition,” and “VES” are used interchangeably to refer to a suspension comprising one or more powders dispersed in one or more diluents, wherein the diluent is a nonsolvent or substantially a nonsolvent with respect to the powder, and wherein the composition exhibits both viscous (fluid-like) and elastic (solid-like) behavior. The viscoelastic character of a VES arises from a cohesive interparticle network, such as a capillary network formed between adjacent particles of the powder, wherein the network imparts structural integrity and cohesive uniformity to the composition. The viscoelastic character is manifested by the composition's ability to store a portion of applied deformation energy as recoverable elastic energy while simultaneously dissipating energy through viscous flow. A VES is distinguished from a dilute suspension of the same powder(s) and diluent(s) in that the interparticle network of a VES contributes elastic solid-like behavior that may be absent in a more dilute suspension, and is distinguished from a dry or friable solid, wetted solid, or clay-like material in that the cohesive interparticle network of a VES is substantially uniform throughout the composition, imparting cohesive continuity rather than the crumbling, fragmentation, or non-uniform cohesive behavior that may be exhibited by compositions in which the solids content exceeds the range at which a uniform cohesive network is maintained.

The viscoelastic character of a VES may be demonstrated or confirmed by any suitable measurement or combination of measurements, including without limitation: oscillatory shear rheometry (e.g., measurement of storage modulus (G′) and loss modulus (G″), wherein G′exceeds G″ over at least a portion of the tested frequency or strain range); yield stress measurement (e.g., determination of the shear stress at which G′ and G″ cross over in an oscillatory amplitude sweep, or by other methods such as controlled stress ramp, tangent intersection, or model extrapolation); tack measurement (e.g., probe tack testing to determine the work-of-cohesion and/or work-of-adhesion of the composition, which may be indicative of the strength of the cohesive interparticle network); assessment of cohesive uniformity (e.g., the composition maintains structural coherence and may extrude as a substantially continuous, cohesive mass rather than as discrete crumbles, or fragments, or may not flow in a steady, continuous stream under the force of gravity alone); or measurement of complex viscosity or apparent viscosity at shear rates relevant to the intended application.

The foregoing characterization methods are not exhaustive; a VES may be identified by any measurable property or combination of properties that is indicative of the presence of a cohesive interparticle network arising from the interactions between the powder and the diluent at the solids content of the composition. The specific values of any rheological parameter at which a given powder-diluent combination exhibits viscoelastic suspension behavior are dependent on the physicochemical properties of the particular powder and diluent, the solids content of the composition, and the conditions of measurement, and are not limited to any particular numerical range or threshold.

A VES may exhibit shear-rate dependent viscosity, broadly characterized as non-Newtonian behavior. In many embodiments, the VES may exhibit shear-thinning behavior, wherein the apparent viscosity of the composition decreases with increasing shear rate. Such shear-thinning behavior may be further described as thixotropic, wherein the apparent viscosity decreases under sustained shear and recovers, partially or fully, upon cessation or reduction of shear. However, the compositions described herein are not limited to shear-thinning or thixotropic behavior, and a VES that exhibits shear-thickening, rheopectic, or other non-Newtonian rheological behavior is within the scope of the present disclosure. The term “viscoelastic suspension” is not limited to any particular solids content, viscosity, storage modulus, loss modulus, yield stress, or other rheological parameter, and encompasses compositions across a broad range of consistencies, from relatively thin, slowly flowing compositions to relatively thick compositions of high viscosity.

It is further noted that within the range of solids contents at which a given powder and diluent combination exhibits viscoelastic behavior, the consistency of the VES may vary considerably. At solids contents near the lower boundary of the viscoelastic range for a given powder and diluent combination, the VES may exhibit a relatively thin consistency, a lower yield stress, a lower apparent viscosity, and may be described as soft, spreadable, or flowable under moderate applied force, while still exhibiting measurable viscoelastic character (i.e., both a storage modulus (G′) and a loss modulus (G″), with the storage modulus exceeding the loss modulus at rest). At higher solids contents within the viscoelastic range, the VES may exhibit a progressively thicker consistency, a higher yield stress, a higher apparent viscosity, and may be described as stiff, dense, or resistant to deformation. At solids contents above the viscoelastic range, the composition may transition to a state in which the cohesive interparticle network is no longer maintained uniformly throughout the composition, and the material may exhibit behavior characteristic of a wetted solid, granular paste, or clay-like material rather than of a viscoelastic suspension. In all cases within the viscoelastic range, the composition remains a viscoelastic suspension as defined herein, provided that it exhibits both viscous and elastic behavior. The term “viscoelastic suspension” or “VES” as used herein is therefore not limited to compositions of any particular consistency, stiffness, yield stress, apparent viscosity, or other rheological property, and encompasses the full continuum of consistencies from relatively thin to relatively thick that a given powder and diluent combination may exhibit across the range of solids contents at which viscoelastic behavior is present. The target solids content and consistency of a VES for a particular application may be selected based on the intended end use of the composition, including but not limited to the route of administration, the delivery device (e.g., syringe and needle gauge), the acceptable injection force, the desired dose volume, and the stability and shelf-life requirements of the composition, and may be adjusted by the addition or removal of diluent using the methods and apparatus described herein.

As used herein, the term “dilute suspension” refers to a suspension at a solids content at which the composition behaves predominantly as a fluid, flows readily under its own weight or upon the application of minimal force, and in which the rheological behavior of the composition is dominated by the properties of the diluent rather than by interparticle interactions between the powder particles. A dilute suspension may serve as a precursor to a VES; for example, a dilute suspension may be converted to a VES by the removal of a portion of the diluent (i.e., by increasing the solids content through the separation process described herein), or a VES may be converted to a dilute suspension by the addition of diluent. The terms “dilute” and “concentrated” as used herein are relative terms referring to different solids content levels of the same powder(s) and diluent(s), and the solids content at which a given powder-diluent combination transitions from dilute suspension behavior to viscoelastic suspension behavior is specific to the physicochemical properties of that particular system and is not limited to any particular solids content or range of solids contents.

As used herein, the term “concentrated suspension” refers to a suspension having a solids content that is elevated relative to a more dilute suspension of the same powder(s) and diluent(s). The methods and apparatus described herein may be used to prepare concentrated suspensions across a range of solids contents and rheological behaviors. Where the concentrated suspension exhibits viscoelastic behavior as described herein, the composition may be referred to as a viscoelastic suspension (VES). The use of the term “concentrated suspension” in connection with the methods and apparatus described herein is not intended to limit the applicability of those methods and apparatus to any particular rheological profile, and encompasses the preparation of compositions that may or may not exhibit viscoelastic behavior. The use of “concentrated suspension (e.g., VES)” throughout this specification is intended to indicate that the described methods and apparatus are applicable to concentrated suspensions generally, including but not limited to those that exhibit viscoelastic behavior, while recognizing that the viscoelastic suspensions described herein represent a principal application of the disclosed methods and apparatus.

While the methods and apparatus described herein are applicable to concentrated suspensions generally as described above, it is noted that the preparation of viscoelastic suspensions (VES) for delivery through delivery devices having constrained flow paths, such as but not limited to fine-gauge needles, may present challenges with respect to powder agglomerates that may be less consequential in the preparation of concentrated suspensions that do not exhibit viscoelastic behavior. Without wishing to be bound by theory, the cohesive interparticle network that characterizes a VES may cause powder agglomerates present in the composition to interact with one another and with the surfaces defining the delivery flow path in a manner that promotes partial or complete occlusion, for example by bridging, interlocking, accumulating, or otherwise impeding flow at constriction points in the delivery flow path. The sensitivity of the delivery process to the presence of powder agglomerates may be influenced by, among other factors, the strength and uniformity of the cohesive interparticle network, the solids content of the composition, the size and size distribution of the agglomerates relative to the dimensions of the delivery flow path, and the rheological properties of the composition under the shear conditions encountered during delivery. The methods described herein, including the high-shear mixing and separation approach, may therefore be of particular utility in the preparation of VES compositions intended for delivery through fine-gauge needles or other delivery devices, where the rheological properties of the VES may make the composition sensitive to the presence of powder agglomerates. It is understood that the foregoing does not limit the applicability of the disclosed methods and apparatus to the preparation of VES compositions, and that the methods and apparatus described herein may be used to prepare any concentrated suspension, whether or not the resulting composition exhibits viscoelastic behavior.

The solids content at which a composition transitions from a dilute suspension (i.e., a predominantly fluid composition in which rheological behavior is dominated by the diluent) to a viscoelastic suspension (i.e., a composition exhibiting both viscous and elastic behavior, as defined herein) is not a fixed or universal value and may vary over a wide range depending on the physicochemical properties of the powder and the diluent. Without limitation, factors that may influence this transition include the particle size and particle size distribution of the powder, the specific surface area of the powder, the particle morphology (e.g., spherical, irregular, elongated, porous, or collapsed), the true density of the powder, the packing behavior and packing fraction of the powder, the surface chemistry and surface energy of the powder particles, the viscosity and surface tension of the diluent, the presence or absence of surface-active agents (e.g., surfactants such as polysorbates) in the diluent that may modify the interparticle interactions, and the nature and magnitude of the interparticle forces (e.g., van der Waals forces, capillary forces, electrostatic forces, and other surface interactions) between adjacent powder particles in the presence of the diluent. By way of example and not limitation, a powder comprising small particles having a relatively high surface area-to-volume ratio (e.g., micronized or spray dried particles having a mean particle size in the range of approximately 1 to 10 micrometers) may exhibit viscoelastic behavior at a relatively different solids content than a comparatively denser powder having markedly lower surface area-to-volume ratio (e.g., coarser granulated or crystalline particles). The compositions, methods, and apparatus described herein are not limited to any particular solids content or range of solids contents at which the transition from dilute suspension to VES occurs, and encompass all powder and diluent combinations in which a VES may be formed at any solids content.

The compositions described herein may, in certain respects, resemble compositions referred to in the pharmaceutical art as “pastes.” The United States Pharmacopeia (USP), in General Chapter <1151> (“Pharmaceutical Dosage Forms”), defines pastes as semisolid preparations of stiff consistency that contain a high percentage of finely dispersed solids, typically in the range of 20% to 50% by weight, and that are intended for application to the skin, oral cavity, or mucous membranes. The compositions, e.g., VES compositions comprising concentrated suspensions, of the present invention share certain characteristics with the USP definition of pastes, in that they are semisolid compositions containing finely dispersed solid particles. However, the VES compositions described herein are distinct from the USP definition of pastes in several respects. First, the VES compositions of the present disclosure are generally intended for parenteral administration (e.g., subcutaneous or intramuscular injection) rather than for topical application to the skin or mucous membranes, and their rheological properties are accordingly optimized for delivery through syringes and needles rather than for surface application. Second, the VES compositions described herein may or may not exhibit the “stiff consistency” described in the USP definition; the consistency of a VES is dependent on the properties of the powder (e.g., particle size, morphology, specific surface area, and composition) and the solids content of the composition, and may range from relatively soft or flowing at lower solids contents or when prepared from powders having particular characteristics (e.g., spray-dried powders having a significant protein content) to relatively stiff at higher solids contents or when prepared from powders having different characteristics (e.g., micronized crystalline powders). Third, the solids content of the VES compositions described herein is not limited to the 20% to 50% range recited in the USP definition, and may be lower (e.g., below 20%) or substantially higher (e.g., above 50%, above 60%, above 70%, or higher, depending on the powder and diluent employed). In prior disclosures by one or more of the present inventors, compositions within the scope of the present disclosure have been referred to as “pastes.” For the avoidance of doubt, the term “viscoelastic suspension” or “VES” as used herein encompasses any composition that would be described as a “paste” in the context of such prior disclosures, and the two terms may be considered generally equivalent for the purposes of the present application. The use of the term “viscoelastic suspension” herein is intended to more accurately describe the rheological characteristics of the compositions and is not intended to disclaim or narrow the scope of compositions previously disclosed or claimed as “pastes” by any of the present inventors.

As used herein, the term “suspension” refers to a composition comprising one or more powders dispersed in one or more diluents. The term encompasses compositions across the full range of solids contents at which the powder remains dispersed in the diluent, and is not limited to any particular solids content, viscosity, rheological profile, powder type, diluent type, or method of preparation. The term encompasses compositions that may alternatively be referred to in the art as dispersions, slurries, or other terms denoting a solid-in-liquid mixture.

As used herein, the term “slurry” is used interchangeably with “suspension” unless the context clearly indicates otherwise. The term is not limited to any particular solids content, viscosity, particle size, or degree of homogeneity. The terms “controlled-release” and “sustained release” are defined for purposes of the present invention as the release of the therapeutic agent at such a rate that blood (e.g., plasma) concentrations are maintained within the therapeutic range but below toxic concentrations over a period of time of about one hour or longer, preferably 12 hours or longer.

As used herein, the term “intracutaneous injection” encompasses administration into the epidermal or dermal skin layers.

As used herein, the term “parenteral injection” refers to the administration of one or more therapeutic agents such as those described herein, or one or more of the compositions of the invention comprising one or more of such therapeutic agents, via a route other than the alimentary canal—any administration that is not by way of the digestive tract—for example, intravenous infusion, intranasal administration, buccal administration, transdermal administration, intracutaneous, subcutaneous, intradermal, intramuscular, intrathecal, intraocular, intravitreal, periocular, intra-articular or injection under or through one or more layers of skin or mucus membranes of an animal, such as a human.

As used herein, a “phase” is defined as a homogeneous, physically distinct portion of a system that is separated from other portions of the system by bounding surfaces. It is known that there are three primary phases of matter (solid, liquid and gaseous). As an example, a system containing particulate matter suspended in a liquid that is a non-solvent to the particulate matter is considered a two-phase system. Conversely, a system consisting of organic macromolecules uniformly distributed throughout a liquid such that no apparent boundaries exist between the macromolecules and the liquid molecules is considered a single-phase solution.

As used herein, a “semi-solid” is an attribute of a material that exhibits plastic flow behavior. A semi-solid material is not pourable, does not readily conform to its container at room temperature, and does not flow at low shear stress. Accordingly, semi-solids have a yield stress that must be exceeded before plastic (i.e., non-reversible) deformation occurs. Semi-solids typically have a viscoelastic rheological flow profile.

Accordingly, a semi-solid is not a specific physical composition or pharmaceutical dosage form, but rather refers to a physical property of the material. Thus, a variety of materials can be considered semi-solids, as they will possess the attribute of a semi-solid material, despite being physically distinct compositions. For example, the USP-NF describes both a cream and a medicated foam as having a semi-solid consistency, and thus both may be considered semi-solid fluids, or semi-solids, despite being otherwise physically distinct compositions. Similarly, gels and pastes are often both termed semi-solids, despite being physically distinct. Gels are defined by the USP-NF as a dosage form that is a semi-solid dispersion of small particles or a solution of large molecules interpenetrated by a solution containing a gelling agent to provide stiffness. Thus, gels may be either single-phase or two-phase systems. As defined in Remington: The Science and Practice of Pharmacy (2006), gel systems may be either clear or turbid, as the ingredients comprising the gel may not be completely soluble or insoluble, or they may form aggregates and disperse light. Gels are defined “as semi-rigid systems in which the movement of the dispersing medium is restricted by an interlacing three-dimensional network of particles or solvated macromolecules in the dispersed phase . . . the interlacing and consequential internal friction is responsible for increased viscosity and the semi-solid state.”

Gels in which the macromolecules are distributed throughout the liquid in such a manner that no apparent boundaries exist between them and the liquid are called single-phase gels. In instances in which the gel mass consists of floccules of small distinct particles, the gel is classified as a two-phase system and frequently called a magma or a milk. Gels and magmas are considered colloidal dispersions since they each contain particles of colloidal dimension. The generally accepted size range for a substance “colloidal” is when particles fall between 1 nm and 0.5 μm.

By contrast, pastes may be defined as a semi-solid dosage form containing a high percentage of finely dispersed solids with a stiff consistency. As discussed earlier, the actual solids content of the paste will primarily depend on the properties of the constituent powder. To prepare a paste, the minimum quantity of fluid that is added to a powder must be sufficient to coat and produce a monolayer of fluid around each individual powder particle. Note that this is an idealized situation where all powder-powder contacts have been fully disrupted, though in reality many micronized powders are highly cohesive, and complete disruption of all direct powder-powder contacts may not be possible, despite the application of high-shear mixing techniques. Additional fluid is then added to the mixture to fill in the interstitial spaces between the powder particles (i.e., the void volume) and thus enable the particles to flow as a fluid when the yield stress of the paste has been exceeded. Accordingly, powders possessing very low density (i.e., high surface area-to-volume ratio) and/or poor packing (i.e. larger interstitial spaces between particles) will require a greater volume/mass of fluid to form a paste compared to powders with a lower surface area-to-volume ratio and/or good packing. Thus, gels and pastes may both possess the semi-solid character, and may both be referred to as semi-solids, but they are physically distinct dosage forms. In particular, the solids concentration of a paste is typically much greater, and the particles are often much larger than the upper limit of the colloidal region (0.5 μm). Overall, the USP-NF defines at least six different dosage forms as being semi-solids, including creams, foams, gels, jellies, ointments, and pastes. However, it will be readily known and understood by the ordinarily skilled technician that these pharmaceutical dosage forms are distinct physical compositions having distinct physical properties, despite all having the semi-solid rheological attribute and thus being broadly termed semi-solids.

“Non-Newtonian,” as used herein, defines a fluid where the viscosity is dependent on the shear rate or shear rate history. This contrasts with a Newtonian fluid, where the viscosity is typically independent of the applied shear rate.

“Thixotropic,” as used herein, defines a fluid that exhibits a shear-thinning property. More specifically, a thixotropic fluid exhibits a time-dependent shear-thinning property, which contrasts with a pseudoplastic fluid, which may characterize a fluid that exhibits time-independent shear-thinning. However, for the purpose of this application, a thixotropic fluid describes shear-thinning fluids in general.

As used herein, the term “parenteral administration” refers to the administration of compositions via injection under or through one or more layers of skin or mucus membranes, including but not limited to intracutaneous, subcutaneous, intradermal, intramuscular, intravenous, intrathecal, intraocular, intravitreal, periocular, intra-articular, and other injection routes suitable for the intended therapeutic application.

As used herein, the term “powder” refers to any solid material in particulate form, regardless of particle size, particle size distribution, particle shape, morphology, crystallinity, porosity, surface area, or chemical composition. The term encompasses single-component powders substantially composed of a single chemical entity, as well as multi-component powders containing two or more chemical entities within individual particles or as combinations of different powder types. The term includes, without limitation, active pharmaceutical ingredients, excipients, fillers, binders, disintegrants, lubricants, surfactants, stabilizers, polymeric particles, biological materials, and combinations thereof. The term is not limited by the method of powder production and encompasses powders produced by any process known in the art, including but not limited to milling, grinding, spray drying, lyophilization, precipitation, crystallization, granulation, fluid bed processing, thin film freezing, co-precipitation, coacervation, electrospinning, electrospraying, emulsification, supercritical fluid processing, or other particle formation techniques. The term “powder” may refer to a single powder or to any blend, mixture, or combination of powders, and is used interchangeably with “solid phase” unless the context clearly indicates otherwise.

As used herein, the term “diluent” refers to any liquid or combination of liquids in which the powder is suspended, dispersed, or otherwise distributed, regardless of whether the liquid dissolves, partially dissolves, or does not dissolve any component of the powder. This term shall also be used regardless of how well distributed into the diluent the powder is dispersed. The term encompasses, without limitation, water, aqueous solutions, hydrophobic liquids, organic solvents, oils, liquid polymers, and any mixture thereof. The term “diluent” is used interchangeably with “liquid phase” unless the context clearly indicates otherwise.

As used herein, the term “carrier” refers to the liquid phase of a composition, and may comprise one or more diluents as defined herein, optionally together with one or more dissolved excipients, polymers, surfactants, or other additives. Where the carrier consists of one or more diluents without additional dissolved components, the terms “carrier” and “diluent” may be used interchangeably. Where the carrier further comprises one or more dissolved components, the term “carrier” refers to the complete liquid phase including such dissolved components, while “diluent” refers to the liquid component(s) of the carrier in which the powder is not dissolved or is only partially dissolved

As used herein, the term “agglomerate” refers to a cluster, assemblage, or grouping of two or more primary particles of the powder that are held together by any interparticle force or combination of forces, including but not limited to van der Waals forces, electrostatic forces, capillary forces, liquid bridging, hydrophobic interactions, mechanical interlocking, sintering, fusion, or any combination thereof. Agglomerates may range in size from slightly larger than the primary particle size to many times the primary particle size, and may be regular or irregular in shape. The term encompasses structures that may be referred to in the art as aggregates (in the powder science context), flocculates, flocs, conglomerates, clusters, granules, coagulates, lumps, clumps, accretions, or any other term denoting a multi-particle assembly. The term “agglomerate” is not limited by the composition, size, or method of formation of the primary particles, and encompasses agglomerates formed from any powder as defined herein. Notwithstanding the foregoing, the term “agglomerate” as used herein is distinct from “aggregates” as that term is used in biopharmaceutical protein characterization. As used herein, “aggregate” or “protein aggregate” refers specifically to molecular-level associations of active pharmaceutical ingredients through chemical bonding, non-covalent interactions, misfolding, or other molecular association mechanisms. The distinction between “agglomerates” (physical multi-particle assemblies) and “aggregates” (molecular-level associations) is maintained throughout this specification.

For the avoidance of doubt, the term “agglomerate” as used herein does not require that the agglomerate be larger than any particular dimension, such as the lumen diameter of a needle, infusion set tubing, or other delivery device through which the composition is intended to be administered. Agglomerates that are smaller than the lumen diameter of the intended delivery device may nevertheless contribute to partial or complete occlusion of the delivery flow path, for example by converging at a constriction point (such as the transition from a syringe barrel to a needle hub), bridging across a portion of the flow path, or otherwise impeding the flow of the composition during administration. The disruption of agglomerates as described herein is therefore directed to reducing the number and/or size of agglomerates across the entire size distribution of the powder, and is not limited to the disruption of agglomerates above any particular size threshold.

As used herein, the term “primary particle” refers to an individual, discrete particle that represents the fundamental unit of the powder before any agglomeration or multi-particle assembly occurs. Primary particles may vary widely in size, shape, morphology, crystallinity, density, porosity, surface area, and chemical composition depending on the powder and the method by which the powder was produced. The term encompasses particles of any size, from nanoscale to microscale dimensions, and is not limited by particle geometry, surface characteristics, or internal structure. Primary particles may be solid, porous, hollow, crystalline, amorphous, spherical, irregular, or any combination thereof. The term is not limited by the composition of the particle and encompasses single-component particles composed substantially of one chemical entity as well as multi-component particles containing two or more chemical entities within the individual particle structure.

As used herein, the term “high-shear” when used to describe processing refers to any process, equipment, or conditions capable of generating sufficient energy to disrupt agglomerates present in a powder or suspension through the application of mechanical forces, fluid shear, or other energy forms. High-shear processing includes, but is not limited to, mixing, screening, sieving, milling, homogenization, sonication, or any other process capable of breaking or weakening interparticle forces that hold agglomerates together, including but not limited to van der Waals forces, electrostatic forces, capillary forces, liquid bridging, hydrophobic interactions, and mechanical interlocking. Disruption may result in complete breakdown of agglomerates to primary particles, partial size reduction of agglomerates, weakening of agglomerate structure, or any combination thereof, and does not require that all agglomerates present be affected or that any particular degree of size reduction be achieved. The effectiveness of high-shear processing may be demonstrated by improvements in one or more functional characteristics of the composition, including but not limited to particle size distribution, syringeability, flow properties, or other performance parameters relevant to the intended application. The term is not limited to any particular equipment type, energy source, operating parameter, rotational speed, tip speed, power input, energy density, shear rate, or other quantitative measure, and encompasses any processing approach that achieves a functional improvement in the composition through agglomerate modification.

As used herein, the term “syringeability” refers to the overall suitability of a composition for delivery through any injection or infusion device, including but not limited to syringe and needle assemblies, cartridge-based systems, pump-driven devices, autoinjectors, pen injectors, pre-filled delivery devices, and other parenteral delivery systems, and encompasses one or more of the following characteristics, individually or in combination: (a) the force required to expel the composition from the delivery device through the delivery conduit at a given volumetric flow rate (referred to herein as “injection force,” “extrusion force,” or “delivery force”); (b) the consistency and uniformity of the delivery force over the course of the injection, including the absence or reduction of transient peaks, spikes, or fluctuations in the force profile that may be indicative of partial or complete occlusion of the delivery flow path by powder agglomerates or other obstructions; (c) the ability to deliver the intended dose volume within a clinically and/or commercially acceptable time and with clinically and/or commercially acceptable force; and (d) any other characteristic relevant to the reliable, reproducible, and clinically acceptable delivery of the composition through the intended delivery system. An improvement in syringeability may be evidenced by a reduction in the mean injection force, a reduction in the variability of the injection force, a reduction in the frequency or magnitude of injection force peaks or spikes, an increase in the proportion of syringes that deliver the intended dose without clogging, or any combination thereof, relative to a comparable composition that has not been subjected to the same processing.

As used herein, the term “solids loading” refers to the concentration of non-volatile components in a solution or suspension that is used to produce a powder, such as through spray drying, lyophilization, or other powder formation techniques. Solids loading encompasses all components that remain after removal of volatile solvents or carriers and that are intended to be incorporated into the final powder, including active pharmaceutical ingredients, excipients, buffers, surfactants, and other non-volatile additives, but excludes volatile components such as water or organic solvents that are removed during the powder formation process. Ions added through pH adjustment (e.g., chloride, sodium) may end up in the final powder formulation but are typically present in trace amounts and are generally not factored into the solids loading calculation of the solution. The determination of which components to include in the solids loading calculation may depend on their intended function and relative concentration in the solution. Solids loading is typically expressed in units of concentration such as mg/mL.

As used herein, the term “solids content” refers to the weight percentage of the solid phase relative to the total weight of the composition (solids plus liquid), typically expressed as percent (w/w).

As used herein, the term “solids concentration” refers to the mass of the solid phase per unit volume of the composition, typically expressed in units such as mg/mL or g/mL. The solids concentration may be calculated from the solids content (expressed as a weight fraction) and the density of the composition. Where both “solids content” and “solids concentration” are used in this specification, “solids content” refers to the weight-based measure (% w/w) and “solids concentration” refers to the volume-based measure (mg/mL or g/mL).

As used herein, the term “powder concentration” is used interchangeably with “solids concentration” and refers to the mass of powder per unit volume of composition, typically expressed in units such as mg/mL. The powder concentration provides a measure of how much total powder is present in a given volume of composition, regardless of the API content of that powder.

As used herein, the term “powder API content” refers to the weight fraction or weight percentage of active pharmaceutical ingredient present in the powder, expressed as % w/w of the powder. For example, a spray-dried powder containing 80% (w/w) active pharmaceutical ingredient would have the remaining 20% (w/w) comprised of excipients, residual moisture, ions, and/or other non-API components. The powder API content is dependent on the composition of the solution used to prepare the powder and the processing conditions employed during powder formation. The powder API content is a parameter that may be relevant for determining the overall API concentration in a final composition, such as a concentrated suspension (e.g., VES).

As used herein, the term “composition API content” refers to the concentration or weight fraction of active pharmaceutical ingredient in the final composition, suspension, or VES. The composition API content is determined by both the solids content of the composition and the API content of the powder according to the relationship: Composition API Content=(Solids Content)× (Powder API Content). The composition API content may be expressed as a weight percentage (% w/w), a concentration (e.g., mg/mL), or other suitable units. For example, a concentrated suspension (e.g., VES) having 60% (w/w) solids content prepared from a powder containing 80% (w/w) API would have a composition API content of approximately 48% (w/w). When expressed as a concentration in mg/mL, the composition API content may be calculated using the density of the composition.

As used herein, the term “fine-gauge needle” refers to any hollow needle, cannula, or other delivery conduit having an internal lumen diameter suitable for the parenteral or percutaneous administration of the compositions described herein. The term encompasses, without limitation, needles sized for intradermal, subcutaneous, intramuscular, intraocular, intravitreal, periocular, intra-articular, intrathecal, or other routes of parenteral or localized injection or infusion, as may be appropriate for the intended therapeutic application. By way of example and not limitation, fine-gauge needles may include needles commonly designated in the art by gauge numbers, wherein a higher gauge number corresponds to a smaller internal lumen diameter, and wherein the internal lumen diameter may range from several hundred microns to less than one hundred microns depending on the gauge designation and wall thickness of the needle. The term is not limited to any particular gauge designation, lumen diameter, needle length, wall thickness (e.g., regular wall, thin wall, ultra-thin wall, or extra-thin wall), or needle geometry. It is noted that the selection of a particular needle gauge and configuration for the delivery of a concentrated suspension (e.g., VES) may be influenced by the route of administration, the viscosity and rheological properties of the composition, the intended injection volume, the acceptable injection time and force, and the clinical requirements of the intended therapeutic application.

As used herein, the term “excess diluent” refers to the portion of the diluent in a suspension that is in excess of the amount required to achieve the target solids content of the concentrated suspension (e.g., VES) for the intended application. The amount of diluent that constitutes “excess” is determined by the target solids content and, where applicable, the target rheological properties of the final concentrated suspension, and is specific to the particular combination of powder and diluent employed. The excess diluent is the diluent that is removed during the separation process to increase the solids content of the composition from its starting solids content toward the target solids content, whether or not the resulting composition exhibits viscoelastic behavior. It is understood that the determination of what constitutes excess diluent may vary depending on the characteristics of the powder (e.g., particle size, morphology, specific surface area, packing behavior), the properties of the diluent, the starting amount of diluent prior to separation, the interactions between the powder and diluent, and the intended application of the resulting concentrated suspension (e.g., VES).

As used herein, the term “separation” refers to any process by which a portion of a liquid phase is removed from a composition comprising the liquid phase and a solid phase, such that the solid phase becomes concentrated relative to its concentration in the composition prior to the separation. The term “separation” is not limited to any particular mechanism, driving force, or instrument, or apparatus, and encompasses any process in which the liquid phase is caused to pass through, around, or away from the solid phase while the solid phase is substantially retained. By way of example and not limitation, “separation” as used herein includes processes that may be referred to in the art as filtration, expression, pressing, straining, decanting, draining, dewatering, desiccation, squeezing, wringing, centrifugation, sedimentation, drying, or any combination thereof. The term further encompasses processes driven by any suitable force, including but not limited to mechanical force, pneumatic pressure, hydraulic pressure, gravitational force, centrifugal force, magnetic force, capillary action, vacuum, osmotic pressure, or any combination thereof. The use of the term “separation” herein is intended to be construed broadly and is not limited by the specific examples, element or body configurations, or process descriptions provided elsewhere in the specification. For the avoidance of doubt, the term “separation” as used herein is directed to the concentration of the solid phase and does not require that the removed liquid phase be collected, recovered, or otherwise retained.

As used herein, the terms “separation medium” and “separation media” refer to any materials, structures, barriers, and/or elements that, when interposed between the suspension and an exit path for the liquid phase, facilitates the concentration of the solid phase of the suspension by permitting passage to a greater proportion of the liquid phase than the solid phase upon the application of a force or pressure. The terms are not limited to media that retain all of the solid phase and encompass media through which a portion of the solid phase may pass, provided that the net effect of the separation is the concentration of the solid phase relative to its concentration in the suspension prior to the separation. The terms encompass, without limitation, any number of filter papers, filter plates, semi-permeable membranes, woven and non-woven filter cloths, woven wire mesh screens, sieves, sintered plates or ceramic elements, perforated plates, and any combination thereof, regardless of the material of construction, pore size, aperture size, placement, or mesh opening size.

As used herein, the term “separated diluent” refers to the portion of the liquid phase (diluent) that has been removed from the suspension through the separation process and has passed through the separation medium. The separated diluent may be collected in a collection vessel or other receptacle, or may be discarded, diverted, or otherwise directed away from the apparatus. The term is used interchangeably with “removed diluent” and, in contexts where the liquid phase is urged through the separation medium by an applied force or pressure, with “expressed diluent.” For the avoidance of doubt, the separated diluent may contain minor amounts of solid phase (e.g., fine particles that have passed through the separation medium), and the presence of such fine particles in the separated diluent does not alter the characterization of the liquid as “separated diluent.” The term “expelled diluent” is used interchangeably with “separated diluent” unless the context clearly indicates otherwise.

As used herein, the term “pushing device(s)” refers to any number of elements, components, or any number of assemblies configured to apply force to the suspension, regardless of its shape, geometry, or mechanism of action. The term encompasses, without limitation, pistons, plungers, plates, discs, diaphragms, bladders, bellows, screw/progressive cavity mechanisms, rams, stamps, or any other structure capable of transmitting force to the suspension. The pushing device(s) may be any combination of rigid, semi-rigid, flexible, or inflatable components and/or parts, may apply force to the suspension directly or indirectly through an intermediate element and/or any number of bodies, may supply and/or control their own sources of force and/or pressure, and may be used to move the suspension in conjunction with or separately from the separation process.

As used herein, the term “apparatus” refers to the entirety of the physical system that performs the separation process along with all supporting and supplementary activities. The term “separation apparatus” is used interchangeably with “apparatus” when used in the context of the separation process described herein, and encompasses all components, bodies, and systems described in the detailed description of the apparatus, including but not limited to the main body, any auxiliary bodies, any interchangeable bodies, any separation media, any pushing devices, any collection vessels, any support structures, and any feedback systems.

As used herein, the term “main body” refers to a unique body where the suspension is first placed during the separation procedure, and where part or all of the separation process will occur. The term is not limited to any particular shape, size or material.

As used herein, the term “auxiliary body” refers to any component, fitting, attachment, adapter, conduit, manifold, valve, port, cap, or assembly that is configured to attach to the main body and/or to any number of auxiliary bodies and that performs or aids in performing one or more functions associated with the operation of the apparatus, including but not limited to providing a connection to a source of force or pressure, providing a connection to a source of one or more materials, supporting any number of separation media, relieving or introducing pressure, venting, sampling, sealing, or providing connection to one or more other bodies that support another action of the apparatus. The term is not limited to any particular shape, size, material, or spatial orientation relative to the main body, and is applicable to bodies that are immovable or that can be removed, replaced, modified, or otherwise reconfigured as needed.

As used herein, the term “interchangeable body” refers to any component, fitting, attachment, adapter, or assembly that is configured to attach to the main body and/or to one or more other interchangeable bodies or auxiliary bodies, and that may be removed, replaced, substituted, or reconfigured to modify the flow, shape, cross-sectional area of flux, direction, or exit point of the suspension, the separated diluent, and/or the concentrated suspension as it passes through or exits the apparatus. The term encompasses, without limitation, bodies configured to support, retain, and/or secure one or more separation media within the apparatus, bodies configured to modify the flow path or exit geometry of the apparatus (e.g., by substituting an extrusion or dispensing geometry such as a nozzle, orifice, or port for a separation medium assembly), and bodies configured to accommodate different collection vessels, clearance requirements, or downstream processing equipment. The interchangeable nature of these bodies allows the operator to adapt the flow characteristics and separation configuration of the apparatus to the requirements of the particular operation being performed, including reconfiguration between or during processing steps, without requiring replacement of the entire apparatus. The term is not limited to any particular shape, size, material, or spatial orientation relative to the main body.

As used herein, the term “feedback system” refers to any system, mechanism, or method, whether electronic, mechanical, pneumatic, hydraulic, optical, manual, or any combination thereof, by which one or more parameters indicative of the status of the separation process and/or of the status of any part of the apparatus are measured, observed, shared and/or otherwise determined, and by which the resulting information is used to direct, adjust, monitor, and/or terminate the separation process. The feedback system may be fully automated, semi-automated, or manually operated. The term encompasses systems comprising any number of electronic sensors and controllers, mechanical indicators and gauges, motors and gears, pneumatic or hydraulic control elements, visual, haptic or auditory indicators, physical or remote data sharing or storage, or any combination thereof. The term also encompasses any algorithms that may be developed by the collection of data, and/or applied to modify the function of the feedback system or any body or function of the apparatus.

As used herein, the term “target solids content” refers to a predetermined or desired proportion of the solid phase in the composition (e.g., a concentrated suspension or viscoelastic suspension), and may be expressed as a weight fraction (w/w), percentage, or any other suitable measure. The target solids content may be selected based on the intended end use of the resulting concentrated suspension (e.g., VES), and may vary or be changed at any time depending on the particular powder, diluent, application, and any measured parameter of the separation process. The target solids content need not be a single precise value, and may encompass one or more ranges or tolerance bands.

As used herein, the term “compatible,” when used to describe a material of construction of the apparatus or any component thereof in relation to the suspension or its components, means that the material does not adversely affect the intended function of the apparatus, the function of any body of the apparatus, the separation process, or the suitability of the resulting composition for its intended end use. Minor or trace-level interactions between the material and the suspension that do not compromise the intended performance or specifications are encompassed within this definition. The determination of compatibility may be made with reference to applicable regulatory requirements, standards, or product specifications, or by a person of ordinary skill in the art.

As used herein, the term “separated diluent” refers to the portion of the liquid phase (diluent) that has been removed from the suspension through the separation process and has passed through the separation medium. The separated diluent may be collected in a collection vessel or other receptacle, or may be discarded, diverted, or otherwise directed away from the apparatus. The term is used interchangeably with “removed diluent” and, in contexts where the liquid phase is urged through the separation medium by an applied force or pressure, with “expressed diluent.” For the avoidance of doubt, the separated diluent may contain minor amounts of solid phase (e.g., fine particles that have passed through the separation medium), and the presence of such fine particles in the separated diluent does not alter the characterization of the liquid as “separated diluent.” The term “expelled diluent” is used interchangeably with “separated diluent” unless the context clearly indicates otherwise.

As used herein, the term “approximately,” when applied to a numerical value, means within a range of reasonable variation as would be understood by a person of ordinary skill in the art in the context in which the term is used. Unless a different tolerance is expressly recited or is apparent from the context, “approximately” encompasses a variation of ±10% of the stated value. The term is intended to accommodate the inherent variability in measurements, process parameters, equipment tolerances, and material properties encountered in the methods, apparatus, and compositions described herein. For the avoidance of doubt, the terms “approximately” and “substantially” may overlap in meaning in certain contexts but are not necessarily interchangeable; “substantially” as defined hereinbelow relates to a degree of completeness or achievement of a condition, whereas “approximately” relates to numerical precision.

As used herein, the term “nonsolvent” refers to a liquid (i.e., a diluent as defined herein) in which the powder, or the principal component(s) of the powder, does not dissolve to a significant or material extent under the conditions of formulation, storage, and use. A diluent may be considered a nonsolvent with respect to a given powder even if minor, incidental, or trace-level dissolution of one or more components of the powder occurs in the diluent, provided that the powder remains substantially in particulate form and that the dissolution does not materially alter the particle size distribution, the solids content, or the rheological properties of the resulting composition. The term encompasses liquids in which the powder is completely insoluble as well as liquids in which the solubility of the powder is sufficiently low that dissolution does not meaningfully affect the composition under the conditions of use. The determination of whether a diluent is a nonsolvent with respect to a given powder may be made by a person of ordinary skill in the art based on the known solubility characteristics of the powder components in the diluent, and does not require that the solubility be precisely zero. The term “substantially a nonsolvent” as used herein is synonymous with “nonsolvent” as defined in this paragraph.

As used herein, the term “low-shear mixing” or “low-shear processing” refers to any mixing, blending, stirring, kneading, folding, tumbling, or other mechanical processing that is performed under conditions that do not generate sufficient energy to disrupt agglomerates to the extent achievable by high-shear processing as defined herein. Low-shear mixing may be used, for example, to disperse one or more powders in one or more diluents to form a suspension, to distribute diluent throughout a composition, to improve the homogeneity or uniformity of a concentrated suspension or VES following separation, to incorporate additional diluent into an over-pressed composition, to incorporate one or more additives (e.g., stabilizers, or other excipients) or additional liquids into a suspension or concentrated suspension, or to blend two or more compositions. Low-shear mixing includes, without limitation, processing performed using planetary-centrifugal mixers (e.g., THINKY or equivalent), paddle mixers, ribbon blenders, tumble blenders, spatula mixing, and any other equipment or technique operated under conditions that are primarily intended to distribute or homogenize rather than to disrupt agglomerates. The distinction between high-shear and low-shear processing is functional rather than equipment-specific; a given piece of equipment may be capable of operating in either a high-shear or low-shear mode depending on the operating parameters (e.g., speed, duration, configuration) and the properties of the composition being processed.

As used herein, the term “dispersion mixer” refers to any mixer, blender, or processing equipment that is effective for distributing a powder throughout a diluent to form a suspension, but that may not generate sufficient shear to disrupt all agglomerates present in the powder to the extent achievable by high-shear processing as defined herein. The term encompasses, without limitation, planetary-centrifugal mixers (e.g., THINKY or equivalent), paddle mixers, impeller mixers, orbital mixers, and other mixing equipment that functions primarily to distribute and incorporate the powder into the diluent. For the avoidance of doubt, a dispersion mixer may partially reduce agglomerate size during the mixing process, and the use of the term “dispersion mixer” does not imply that no agglomerate disruption occurs; rather, the term indicates that the primary function of the mixer in the context described is the formation of a suspension by distributing the powder in the diluent, and that additional high-shear processing may be employed to achieve agglomerate disruption beyond that achievable by the dispersion mixer alone.

As used herein, the term “delivery flow path” or “delivery path” refers to the entirety of the internal passage or passageway through which a composition travels during delivery from a delivery device to the site of administration. The delivery flow path encompasses, without limitation, the internal lumen of the container (e.g., a syringe barrel, cartridge, or reservoir), any transition region between the container and the delivery conduit (e.g., the syringe barrel-to-hub transition, a needle hub, a connector, or a fitting), the internal lumen of the delivery conduit itself (e.g., a needle, cannula, or tubing), and any other internal surface, constriction, orifice, junction, or passage through which the composition must pass during delivery. The term is not limited to any particular device type, geometry, material, or dimension. It is noted that the delivery flow path may include one or more regions of reduced cross-sectional area (i.e., constrictions) at which the flow path narrows relative to adjacent regions, and that such constrictions may be particularly susceptible to partial or complete occlusion by powder agglomerates, even where the agglomerates are individually smaller than the nominal lumen diameter of the delivery conduit, as described in the definition of “agglomerate” herein.

As used herein, the term “delivery device” refers to any device, system, or assembly configured to deliver a composition to a subject or to an intended site of administration. The term encompasses, without limitation, syringes (including pre-filled syringes, luer-lock syringes, and luer-slip syringes), syringe and needle assemblies, cartridges, autoinjectors, pen injectors, wearable injectors, on-body delivery systems, pump-driven devices (including syringe pumps, peristaltic pumps, and infusion pumps), implantable delivery devices, microneedle arrays, jet injectors, needle-free injection systems, and any other device through which the composition may be administered to a subject. The term is not limited to devices for parenteral administration and may encompass devices for any route of delivery for which the compositions described herein may be suitable. A delivery device comprises one or more delivery flow paths as defined herein.

As used herein, the term “yield stress” refers to the stress at which a composition transitions from predominantly elastic, solid-like behavior to predominantly viscous, fluid-like behavior. The yield stress may be determined by any suitable method known in the art, including without limitation: (a) the shear stress at which the storage modulus (G′) and loss modulus (G″) cross over in an oscillatory amplitude sweep (i.e., the crossover stress); (b) the tangent intersection method applied to a flow curve; (c) a controlled stress ramp in which the onset of flow is identified; (d) extrapolation to zero shear rate using models such as Bingham, Herschel-Bulkley, or Casson; or (e) any other method recognized by a person of ordinary skill in the art for determining the stress threshold at which a material yields. The yield stress may also be referred to in the art as the “flow point,” particularly when determined by the G′/G″ crossover method in an oscillatory amplitude sweep, and the terms may be used interchangeably herein unless the context clearly indicates otherwise. The term is not limited to any particular measurement method, geometry, instrument, temperature, or testing protocol. It is noted that different measurement methods may yield different numerical values for the yield stress of the same composition. The presence or absence of a yield stress is not, by itself, determinative of whether a composition is a viscoelastic suspension as defined herein.

As used herein, the term “interior volume” refers to the internal space defined by the walls of the main body that is configured to contain the suspension during the separation process. The interior volume encompasses the space available to the suspension and any headspace (e.g., air or gas space) above the suspension within the main body. When a pushing device is present within the main body, the interior volume refers to the total internal space of the main body; the volume occupied by the pushing device reduces the effective volume available to the suspension but does not alter the interior volume of the main body itself. The interior volume is in fluid communication with one or more openings in the main body as described herein. The interior volume is not limited to any particular shape, cross-sectional geometry, or dimension, and may be cylindrical, conical, or of any other geometry suitable for containing the suspension during the separation process.

The term “physical stability” means that with respect to the therapeutic agent, an acceptable percentage of aggregates (e.g., dimers, trimers and larger forms) is formed. In particular, a formulation is considered physically stable if no more than about 15%, and preferably no more than about 1-10% or about 1-5%, aggregates are formed after one year of storage at the intended storage temperature of the product (e.g., room temperature); or storage of the product at 30° C./60% relative humidity for one year; or storage of the product at 40° C./75% relative humidity for one month, and preferably three to six months.

The term “chemical stability” means that with respect to the therapeutic agent, an acceptable percentage of degradation products produced by chemical pathways such as oxidation or hydrolysis is formed. In particular, a formulation is considered chemically stable if no more than up to about 50%, e.g., no more than about 10%, about 20%, about 30%, about 40%, or about 50%, breakdown products are formed after one year of storage at the intended storage temperature of the product (e.g., room temperature); or storage of the product at 30° C./60% relative humidity for one year; or storage of the product at 40° C./75% relative humidity for one month, and preferably three to six months.

The term “stable formulation” means that at least about 65% chemically and physically stable therapeutic agent remains after two months of storage at room temperature. Particularly preferred formulations are those which retain at least about 80% chemically and physically stable therapeutic agent under these conditions.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Pharmaceutically acceptable carrier” means a pharmaceutically acceptable solvent, suspending agent, or vehicle for delivering a drug compound of the present invention to a mammal such as a human.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements but is not limited to possessing only those elements. Likewise, a method that “comprises,” “has,” “includes,” or “contains” one or more steps possesses those one or more steps but is not limited to possessing only those one or more steps. Further, a device or system that is configured in a certain way is configured in at least that way, but it can also be configured in other ways than those specifically described.

As used herein, a “co-formulation” is a formulation that contains two or more therapeutic agents dissolved in an aprotic polar solvent system. The therapeutic agents may belong to the same class (for example, a co-formulation comprising two or more therapeutic peptides, such as insulin and pramlintide, or glucagon and GLP-1), or the therapeutic agents may belong to different classes (for example a co-formulation comprising one or more therapeutic small molecules and one or more therapeutic peptide molecules, such as GLP-1 and lisofylline).

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%.

As used herein, “analogue” and “analog,” when referring to a peptide or protein, refers to a modified peptide or protein wherein one or more amino acid residues of the peptide or protein have been substituted by other amino acid residues, or wherein one or more amino acid residues have been deleted from the peptide or protein, or wherein one or more amino acid residues have been added to the peptide or protein, or any combination of such modifications. Such addition, deletion, or substitution of amino acid residues can take place at any point, or multiple points, along the primary structure comprising the peptide, including at the N-terminal of the peptide or protein and/or at the C-terminal of the peptide or protein.

As used herein, “derivative,” in relation to a parent peptide or protein, refers to a chemically modified parent peptide or protein or an analog thereof, wherein at least one substituent is not present in the parent peptide or protein an analog thereof. One such non-limiting example is a parent peptide or protein which has been covalently modified. Typical modifications are amides, carbohydrates, alkyl groups, acyl groups, esters, pegylations and the like.

The term “coupled” is defined as connected, although not necessarily directly, and not necessarily mechanically; two items that are “coupled” may be unitary with each other.

The terms “a” and “an” are defined as one or more unless this disclosure explicitly requires otherwise.

The term “stable formulation” means that at least about 65% chemically and physically stable therapeutic agent remains after two months of storage at room temperature. Particularly preferred formulations are those which retain at least about 80% chemically and physically stable therapeutic agent under these conditions. Especially preferred stable formulations are those which do not exhibit degradation after sterilizing irradiation (e.g., gamma, beta or electron beam).

The term “bioavailability” is defined for purposes of the present invention as the extent to which the therapeutic agent is absorbed from the formulation into the bloodstream and/or tissues of an animal or human to whom the formulation has been administered.

The term “systemic” means, with respect to delivery or administration of a beneficial agent to a subject, that beneficial agent is detectable at a biologically significant level in the blood plasma of the subject.

The term “therapeutic agent,” which is used interchangeably herein with the terms “pharmaceutically active ingredient,” “active ingredient,” or “active pharmaceutical ingredient,” means an agent that effects a desired, beneficial, often pharmacological, effect upon administration to a human or an animal, whether alone or in combination with other pharmaceutical excipients or inert ingredients. In certain aspects of the present invention, a therapeutic agent encompasses drugs (e.g., small molecules, peptides, proteins, biologics), vaccines, oligonucleotides, gene therapy vehicles/vectors, and the like used in the prevention, diagnosis, alleviation, treatment or cure of a condition, ailment or disease.

As used herein, the term “separation” refers to any process by which a portion of a liquid phase is removed from a composition comprising the liquid phase and a solid phase, such that the solid phase is concentrated relative to its concentration in the composition prior to the separation. The term “separation” is not limited to any particular mechanism, driving force, or apparatus, and encompasses any process in which the liquid phase is caused to pass through, around, or away from the solid phase while the solid phase is substantially retained. By way of example and not limitation, “separation” as used herein includes processes that may be referred to in the art as filtration, expression, pressing, straining, decanting, draining, dewatering, desiccation, squeezing, wringing, centrifugation, sedimentation, or any combination thereof. The term further encompasses processes driven by any suitable force, including but not limited to mechanical force, pneumatic pressure, hydraulic pressure, gravitational force, centrifugal force, magnetic force, capillary action, vacuum, osmotic pressure, or any combination thereof. The use of the term “separation” herein is intended to be construed broadly and is not limited by the specific examples, apparatus configurations, or process descriptions provided elsewhere in the specification. For the avoidance of doubt, the term “separation” as used herein is directed to the concentration of the solid phase and does not require that the removed liquid phase be collected, recovered, or otherwise retained.

As used herein, the terms “separation medium” and “separation media” refer to any materials, structures, barriers, and/or elements that, when interposed between the suspension and an exit path for the liquid phase, facilitates the concentration of the solid phase of the suspension by permitting passage to a greater proportion of the liquid phase than the solid phase upon the application of a force or pressure. The terms are not limited to media that retain all of the solid phase and encompass media through which a minor portion of the solid phase may pass, provided that the net effect of the separation is the concentration of the solid phase relative to its concentration in the suspension prior to the separation. The terms encompass, without limitation, any number of filter papers, filter plates, semi-permeable membranes, woven and non-woven filter cloths, woven wire mesh screens, sieves, sintered metal or ceramic elements, perforated plates, and any combination thereof, regardless of the material of construction, pore size, aperture size, placement, or mesh opening size.

As used herein, the term “substantially” means a degree that is within practical tolerances recognized by a person of ordinary skill in the art. When applied to a numerical value, “substantially” means within ±10% of the stated value unless a different tolerance is expressly recited. The term is not intended to require mathematical exactness, but rather to accommodate the inherent variability in the processes and compositions described herein.

As used herein, the term “pushing device(s)” refers to any number of elements, components, or any number of assemblies configured to apply force to the suspension within the interior volume of the main body, regardless of its shape, geometry, or mechanism of action. The term encompasses, without limitation, pistons, plungers, plates, discs, diaphragms, bladders, bellows, screw/progressive cavity mechanisms, rams, stamps, or any other structure capable of transmitting force to the suspension. The pushing device may be any combination of rigid, semi-rigid, flexible, or inflatable components and/or parts, may apply force to the suspension directly or indirectly through an intermediate element, and may supply and/or control its own source of force and/or pressure.

As used herein, the term “auxiliary body” refers to any component, fitting, attachment, adapter, conduit, manifold, valve, port, cap, or assembly that is configured to attach to the main body and/or to another auxiliary body and that performs or aids in performing one or more functions associated with the operation of the apparatus, including but not limited to providing a connection to a source of force or pressure, providing a connection to a source of one or more materials, supporting any number of separation media, relieving or introducing pressure, venting, sampling, sealing, or providing connection to more or more other bodies that support another action of the suspension, including to but limited to. The term is not limited to any particular shape, size, material, or spatial orientation relative to the main body.

As used herein, the term “interchangeable body” refers to any component, fitting, attachment, adapter, support, holder, cradle, or assembly that is configured to attach to the main body or to another interchangeable body and that is removable, replaceable, or reconfigurable to accommodate different separation requirements. The term encompasses, without limitation, components configured to support, retain, or secure one or more separation media, and components configured to modify the flow, shape, cross-sectional area of flux, direction, or exit point of the suspension or the separated liquid. The term is not limited to any particular shape, size, material, or spatial orientation relative to the main body.

As used herein, the term “feedback system” refers to any system, mechanism, or method, whether electronic, mechanical, pneumatic, hydraulic, optical, manual, or any combination thereof, by which one or more parameters indicative of the status of the separation process are measured, observed, or otherwise determined, and by which the resulting information is used to direct, adjust, monitor, or terminate the separation process. The feedback system may be fully automated, semi-automated, or manually operated. The term encompasses systems comprising any number of electronic sensors and controllers, mechanical indicators and gauges, motors and gears, pneumatic or hydraulic control elements, visual, haptic or auditory indicators, physical or remote data sharing or storage, or any combination thereof.

As used herein, the term “diluent” refers to any liquid or combination of liquids in which the powder is suspended, dispersed, or otherwise distributed, regardless of whether the liquid dissolves, partially dissolves, or does not dissolve any component of the powder. This term shall also be used regardless of how well distributed into the diluent the powder is dispersed. The term encompasses, without limitation, water, aqueous solutions, hydrophobic liquids, organic solvents, oils, liquid polymers, and any mixture thereof. The term “diluent” is used interchangeably with “liquid phase” unless the context clearly indicates otherwise.

As used herein, the term “powder” refers to any solid material in particulate form, regardless of particle size, particle size distribution, particle shape, morphology, crystallinity, porosity, surface area, or chemical composition. The term encompasses single-component powders that are substantially composed of a single chemical entity, as well as multi-component or composite powders in which two or more chemical entities are present within individual particles. This term also includes powders that are comprised of a combination of single-component and multi-component powders, in any degree of mixture, along with powders that may alternate between identifying as single- and multi-component powders. By way of example and not limitation, a multi-component or composite powder may comprise an active pharmaceutical ingredient and one or more excipients co-formulated within individual particles at any ratio, or may comprise multiple active pharmaceutical ingredients, multiple excipients, or any combination thereof. The term further encompasses, without limitation, active pharmaceutical ingredients, excipients, fillers, binders, disintegrants, lubricants, surfactants, stabilizers, polymeric particles, biological materials, and any combination thereof. The term “powder” is not limited by the method by which the powder is produced, and encompasses powders produced by any process known in the art, including but not limited to milling, grinding, micronization, jet milling, spray drying, electrostatic spray drying, thin film freezing, lyophilization, freeze drying, precipitation, crystallization, granulation, spray granulation, fluid bed processing, hot melt extrusion, co-precipitation, coacervation, electrospinning, electrospraying, emulsification, solvent evaporation, supercritical fluid processing, atomization, or any combination thereof. The term “powder” as used herein may refer to a single powder or to a blend, mixture, or combination of two or more powders, whether of the same or different compositions, particle sizes, or origins. The term “powder” is used interchangeably with “solid phase” unless the context clearly indicates otherwise.

As used herein, the terms “viscoelastic suspension,” “viscoelastic composition,” and “VES” are used interchangeably to refer to a suspension comprising one or more powders dispersed in one or more diluents, wherein the diluent is a nonsolvent or substantially a nonsolvent with respect to the powder, and wherein the composition exhibits both viscous (fluid-like) and elastic (solid-like) behavior. The viscoelastic character of a VES arises from a cohesive interparticle network, such as a capillary network formed between adjacent particles of the powder, wherein the network imparts structural integrity and cohesive uniformity to the composition. The viscoelastic character is manifested by the composition's ability to store a portion of applied deformation energy as recoverable elastic energy while simultaneously dissipating energy through viscous flow. A VES is distinguished from a dilute suspension of the same powder(s) and diluent(s) in that the interparticle network of a VES contributes elastic solid-like behavior that may be absent in a more dilute suspension, and is distinguished from a dry or friable solid, wetted solid, or clay-like material in that the cohesive interparticle network of a VES is substantially uniform throughout the composition, imparting cohesive continuity rather than the crumbling, fragmentation, or non-uniform cohesive behavior that may be exhibited by compositions in which the solids content exceeds the range at which a uniform cohesive network is maintained.

The present invention provides methods of producing certain high solids content suspensions, particularly viscoelastic suspensions (“VES”) comprising one or more active pharmaceutical ingredients (“APIs”) in the solids, that remain flowable and capable of being injected into a patient in need of treatment with the one or more APIs. The invention also provides compositions, particularly VESs, produced by these methods. In other embodiments, the invention provides an apparatus useful in carrying out the present methods and in producing the present compositions. In other methods, the invention also provides methods of treating certain diseases, disorders, and/or medical conditions in mammals, particularly humans and veterinary animals, comprising administering one or more of the compositions of the present invention to a mammal suffering from or predisposed to such diseases, disorders, and/or medical conditions, thereby treating, curing, or preventing the disease, disorder, and/or medical condition in the mammal.

As shown in Table 1, in some exemplary embodiments syringes can be used to administer one or more of the compositions of the present invention to the mammal.

TABLE 1
Plunger Velocities at Two Illustrative Flow Rates for
Reservoirs Consistent with Certain Syringes

| | 33.3 μL/s | 67.0 μL/s |
|---|---|---|
| Reservoir Volume | Plunger Velocity | Plunger Velocity |
| (μL) | (mm/s) | (mm/s) |
| 100 | 19.91 | 40.00 |
| 250 | 8.02 | 16.12 |
| 500 | 3.90 | 7.83 |
| 1000 | 2.00 | 4.01 |

In certain such embodiments, the syringes are configured to dispense paste at a flow rate of greater than 30 μL/s as the plunger is moved at a rate of between 2 and 40 mm/s. Also, as shown in the non-limiting Examples hereinbelow, flow rates of the VESs provided by the invention are substantially linearly proportional to the rate of plunger movement.

Some embodiments of the present methods for parenterally (e.g., intracutaneously, intramuscularly, intradermally, or intrathecally) injecting a volume of paste comprise moving a plunger of a syringe to dispense paste from a reservoir of the syringe through a lumen of a needle of the syringe, the reservoir having an internal first transverse dimension that is larger than an internal second transverse dimension of the lumen, where the second transverse dimension is between 0.1 and 0.9 mm, where the paste has a solids concentration of greater than 100 mg/L, and where the paste is dispensed at a flow rate of greater than 30 μL/s as the plunger is moved at a rate of between 2 and 40 mm/s. Some methods comprise removing a sealing cap from a fitting (e.g., a Luer fitting) of the reservoir. Some methods comprise coupling the needle to the reservoir via a Luer fitting disposed on at least one of the needle and the reservoir. Some methods comprise disposing the needle into and/or through cutaneous tissue of a patient.

In some methods, the injected volume of paste is greater than 10 μL. In some methods, the injected volume of paste is between 15, 500, or 1000 μL to 1200, 2000, or 3000 μL. In some methods, the injected volume of paste is between 30 μL and 100 μL.

### Pharmaceutically Active Ingredients

The compositions of the present invention suitably comprise one or more (e.g., 1, 2, 3, 4, 5 or more) pharmaceutically active ingredients (used interchangeably herein with “active pharmaceutical ingredients” or “therapeutic ingredients”). By a “pharmaceutically active ingredient” is intended an ingredient in the composition which has a physiological, metabolic, physical, or mechanical effect when introduced into an animal (e.g., a human or veterinary animal) and is therefore useful in therapeutic and diagnostic methods for treating, ameliorating, preventing and/or diagnosing a disease or disorder in the animal into which the pharmaceutically active ingredient is introduced. Examples of suitable pharmaceutically active ingredients for use in preparing the paste formulations provided by the present invention include, but are not limited to, peptides, oligonucleotides, radiopharmaceuticals, biologics, proteins, and small molecule therapeutic or diagnostic agents.

In some embodiments, the small molecule is a kinase inhibitor, or a pharmaceutically acceptable salt thereof. Kinase inhibitors include, without limitation, receptor tyrosine kinase inhibitors, non-receptor tyrosine kinase inhibitors, serine/threonine kinase inhibitors, lipid kinase inhibitors, and dual- or multi-kinase inhibitors.

Non-limiting examples include: BCR-ABL inhibitors such as imatinib, dasatinib, nilotinib, bosutinib, ponatinib, and asciminib; EGFR inhibitors such as erlotinib, gefitinib, afatinib, and osimertinib; ALK inhibitors such as crizotinib, ceritinib, alectinib, brigatinib, and lorlatinib; BRAF inhibitors such as vemurafenib, dabrafenib, and encorafenib; MEK inhibitors such as trametinib, cobimetinib, binimetinib, and selumetinib; VEGFR/multi-kinase inhibitors such as sorafenib, sunitinib, pazopanib, axitinib, cabozantinib, lenvatinib, and regorafenib; BTK inhibitors such as ibrutinib, acalabrutinib, zanubrutinib, and pirtobrutinib; JAK inhibitors such as ruxolitinib, tofacitinib, baricitinib, upadacitinib, and fedratinib; CDK4/6 inhibitors such as palbociclib, ribociclib, and abemaciclib; FGFR inhibitors such as erdafitinib, pemigatinib, futibatinib, and infigratinib; RET inhibitors such as selpercatinib and pralsetinib; KRAS G12C inhibitors such as sotorasib and adagrasib; PI3K inhibitors such as idelalisib, copanlisib, alpelisib, and duvelisib; mTOR inhibitors such as everolimus and temsirolimus; FLT3 inhibitors such as midostaurin, gilteritinib, and quizartinib; TRK inhibitors such as larotrectinib and entrectinib; MET inhibitors such as capmatinib and tepotinib; and other kinase inhibitors, or pharmaceutically acceptable salts thereof.

In some embodiments, the small molecule is an immunomodulatory agent. Non-limiting examples include: thalidomide analogs (IMiDs) such as thalidomide, lenalidomide, pomalidomide, and iberdomide; calcineurin inhibitors such as cyclosporine and tacrolimus; mTOR inhibitors such as sirolimus and the mTOR inhibitors described above under Kinase Inhibitors; sphingosine-1-phosphate (S1P) receptor modulators such as fingolimod, siponimod, ozanimod, ponesimod, and etrasimod; PDE4 inhibitors such as apremilast; DHODH inhibitors such as leflunomide and teriflunomide; IMPDH (inosine monophosphate dehydrogenase) inhibitors such as mycophenolate mofetil and mycophenolic acid; and other immunomodulatory or immunosuppressive small molecules, or pharmaceutically acceptable salts thereof.

In some embodiments, the small molecule is a proteasome inhibitor. Non-limiting examples include: bortezomib, carfilzomib, ixazomib, and other proteasome inhibitors, or pharmaceutically acceptable salts thereof.

In some embodiments, the small molecule is a targeted protein degrader or a molecular glue. Targeted protein degraders include, without limitation, proteolysis-targeting chimeras (PROTACs), molecular glues, and other heterobifunctional or monovalent degrader compounds that induce ubiquitin-mediated degradation of a target protein. Non-limiting examples of molecular glues include the cereblon-modulating agents described above under Immunomodulatory Agents (e.g., lenalidomide, pomalidomide, iberdomide) and other E3 ligase-recruiting degraders. The present formulations are applicable to any targeted protein degrader regardless of the target protein, E3 ligase engaged, or degrader format.

In some embodiments, the small molecule is an epigenetic modulator. Non-limiting examples include: HDAC inhibitors such as vorinostat, romidepsin, belinostat, panobinostat, and tucidinostat; DNMT inhibitors such as azacitidine and decitabine; IDH1 inhibitors such as ivosidenib; IDH2 inhibitors such as enasidenib; EZH2 inhibitors such as tazemetostat; BET inhibitors; and other epigenetic modulators, or pharmaceutically acceptable salts thereof.

In some embodiments, the small molecule is a BCL-2 family inhibitor or other apoptosis modulator. Non-limiting examples include: venetoclax (BCL-2 inhibitor); navitoclax (BCL-2/BCL-XL inhibitor); and other inhibitors of anti-apoptotic proteins, or pharmaceutically acceptable salts thereof.

In some embodiments, the small molecule is a poly(ADP-ribose) polymerase (PARP) inhibitor. Non-limiting examples include: olaparib, niraparib, rucaparib, talazoparib, and other PARP inhibitors, or pharmaceutically acceptable salts thereof.

In some embodiments, the small molecule is a hormonal or endocrine therapeutic agent. Non-limiting examples include: aromatase inhibitors such as letrozole, anastrozole, and exemestane; selective estrogen receptor modulators (SERMs) such as tamoxifen and raloxifene; selective estrogen receptor degraders (SERDs) such as fulvestrant and elacestrant; androgen receptor inhibitors such as enzalutamide, apalutamide, darolutamide, and bicalutamide; CYP17 inhibitors such as abiraterone; GnRH receptor antagonists such as relugolix; thyroid hormone and analogs such as levothyroxine and liothyronine; anti-thyroid agents such as methimazole and propylthiouracil; and other hormonal or endocrine small molecules, or pharmaceutically acceptable salts thereof.

In some embodiments, the small molecule is an anti-inflammatory or analgesic agent. Non-limiting examples include: non-steroidal anti-inflammatory drugs (NSAIDs) such as ibuprofen, naproxen, diclofenac, celecoxib, and meloxicam; corticosteroids such as prednisone, prednisolone, dexamethasone, budesonide, methylprednisolone, and hydrocortisone; colchicine; and other anti-inflammatory or analgesic small molecules, or pharmaceutically acceptable salts thereof.

In some embodiments, the small molecule is a cardiovascular therapeutic agent. Non-limiting examples include: HMG-COA reductase inhibitors (statins) such as atorvastatin, rosuvastatin, and simvastatin; anticoagulants such as warfarin, apixaban, rivaroxaban, edoxaban, and dabigatran; antiplatelet agents such as clopidogrel, ticagrelor, and prasugrel; antiarrhythmic agents such as amiodarone, flecainide, and dronedarone; beta-adrenergic receptor blockers such as metoprolol, carvedilol, and propranolol; calcium channel blockers such as amlodipine, diltiazem, and verapamil; angiotensin-converting enzyme (ACE) inhibitors such as enalapril, lisinopril, and ramipril; angiotensin II receptor blockers (ARBs) such as losartan, valsartan, and irbesartan; angiotensin receptor-neprilysin inhibitors (ARNIs) such as sacubitril/valsartan; SGLT2 inhibitors such as empagliflozin, dapagliflozin, and canagliflozin; soluble guanylate cyclase (sGC) stimulators such as vericiguat and riociguat; endothelin receptor antagonists such as bosentan, ambrisentan, and macitentan; and other cardiovascular small molecules, or pharmaceutically acceptable salts thereof.

In some embodiments, the small molecule is a CNS therapeutic agent. Non-limiting examples include: selective serotonin reuptake inhibitors (SSRIs) such as fluoxetine, sertraline, and escitalopram; serotonin-norepinephrine reuptake inhibitors (SNRIs) such as venlafaxine and duloxetine; atypical antipsychotics such as risperidone, olanzapine, quetiapine, aripiprazole, and brexpiprazole; anticonvulsants such as levetiracetam, lamotrigine, valproic acid, carbamazepine, and lacosamide; dopaminergic agents such as levodopa, carbidopa, pramipexole, ropinirole, and entacapone; cholinesterase inhibitors such as donepezil, rivastigmine, and galantamine; NMDA receptor antagonists such as memantine; benzodiazepines such as diazepam, lorazepam, and midazolam; orexin receptor antagonists such as suvorexant and lemborexant; CGRP receptor antagonists (gepants) such as ubrogepant, rimegepant, and atogepant; and other CNS small molecules, or pharmaceutically acceptable salts thereof.

In some embodiments, the small molecule is an anti-infective agent. Non-limiting examples include: antibacterials such as beta-1actams (e.g., amoxicillin, piperacillin, meropenem, ceftriaxone), fluoroquinolones (e.g., ciprofloxacin, levofloxacin, moxifloxacin), macrolides (e.g., azithromycin, clarithromycin), tetracyclines (e.g., doxycycline, omadacycline), aminoglycosides (e.g., gentamicin, tobramycin), glycopeptides (e.g., vancomycin), oxazolidinones (e.g., linezolid, tedizolid), and lipoglycopeptides (e.g., dalbavancin); antivirals such as nucleos(t)ide analogs (e.g., tenofovir, entecavir, acyclovir, remdesivir), protease inhibitors (e.g., nirmatrelvir, ritonavir), polymerase inhibitors (e.g., sofosbuvir, molnupiravir), NS5A inhibitors (e.g., ledipasvir, velpatasvir), integrase inhibitors (e.g., dolutegravir, bictegravir, cabotegravir), NNRTIs (e.g., efavirenz, doravirine), and cap-dependent endonuclease inhibitors (e.g., baloxavir marboxil); antifungals such as azoles (e.g., fluconazole, voriconazole, posaconazole, isavuconazonium), echinocandins (e.g., caspofungin, micafungin, anidulafungin, rezafungin), and polyenes (e.g., amphotericin B); antiparasitics such as artemisinin derivatives, ivermectin, and nitazoxanide; and other anti-infective small molecules, or pharmaceutically acceptable salts thereof.

In some embodiments, the small molecule is a metabolic or endocrine therapeutic agent. Non-limiting examples include: biguanides such as metformin; sulfonylureas such as glipizide and glimepiride; DPP-4 inhibitors such as sitagliptin, saxagliptin, and linagliptin; SGLT2 inhibitors as described above under Cardiovascular Agents; thiazolidinediones such as pioglitazone; urate-lowering agents such as allopurinol, febuxostat, and lesinurad; and other metabolic small molecules, or pharmaceutically acceptable salts thereof.

In some embodiments, the small molecule is a respiratory therapeutic agent. Non-limiting examples include: inhaled corticosteroids such as fluticasone, budesonide, and mometasone; long-acting beta-agonists (LABAs) such as salmeterol, formoterol, and vilanterol; long-acting muscarinic antagonists (LAMAs) such as tiotropium, umeclidinium, and glycopyrrolate; phosphodiesterase inhibitors such as roflumilast; leukotriene receptor antagonists such as montelukast; and other respiratory small molecules, or pharmaceutically acceptable salts thereof.

In some embodiments, the small molecule is a gastrointestinal therapeutic agent. Non-limiting examples include: proton pump inhibitors (PPIs) such as omeprazole, esomeprazole, lansoprazole, and pantoprazole; H2 receptor antagonists such as famotidine; 5-ASA agents such as mesalamine; ursodeoxycholic acid and obeticholic acid; and other gastrointestinal small molecules, or pharmaceutically acceptable salts thereof.

In some embodiments, the small molecule is a dermatologic therapeutic agent. Non-limiting examples include: retinoids such as tretinoin, isotretinoin, acitretin, and tazarotene; topical calcineurin inhibitors such as tacrolimus and pimecrolimus; topical JAK inhibitors such as ruxolitinib; topical PDE4 inhibitors such as crisaborole; and other dermatologic small molecules, or pharmaceutically acceptable salts thereof.

In some embodiments, the small molecule is an ophthalmic therapeutic agent. Non-limiting examples include: prostaglandin analogs such as latanoprost, travoprost, and bimatoprost; rho kinase inhibitors such as netarsudil; beta-adrenergic blockers such as timolol; carbonic anhydrase inhibitors such as dorzolamide; and other ophthalmic small molecules, or pharmaceutically acceptable salts thereof.

In some embodiments, the small molecule is not encompassed by the foregoing categories. Non-limiting examples include: CFTR modulators such as ivacaftor, lumacaftor, tezacaftor, and elexacaftor; SMN2 splicing modulators such as risdiplam; sickle cell disease agents such as hydroxyurea; complement inhibitors such as iptacopan (factor B inhibitor) and danicopan (factor D inhibitor); and other small molecule therapeutics, or pharmaceutically acceptable salts thereof.

The formulations described herein are not limited to the specific small molecules enumerated above. The foregoing examples are provided to illustrate the breadth and diversity of small molecule therapeutics that may benefit from the present formulation platform and are not intended to be exhaustive.

In some embodiments, the small molecule is any organic compound having a molecular weight of less than about 2,000 Da that (i) is intended for oral, subcutaneous, intramuscular, intravenous, intravitreal, intrathecal, intraarticular, intradermal, topical, transdermal, inhaled, intranasal, rectal, or other administration, and (ii) may benefit from a formulation that provides one or more of: enhanced solubility, improved bioavailability, enhanced stability, controlled release, sustained release, modified release, reduced food effect, improved taste masking, reduced dosing frequency, or improved patient compliance relative to a reference formulation.

In some embodiments, the small molecule has a concentration in the formulation of from about 0.01 mg/mL to about 700 mg/mL, such as from about 0.1 mg/mL to about 500 mg/mL, such as from about 1 mg/mL to about 200 mg/mL. In some embodiments, the small molecule has a concentration in the formulation greater than about 700 mg/mL.

In some embodiments, the small molecule is a generic equivalent, an authorized generic, a 505(b) (2) product, or an ANDA product of any of the foregoing small molecules. In some embodiments, the small molecule is a deuterated analog, a prodrug, a soft drug, an extended-release formulation, a co-crystal, or a novel salt form of any of the foregoing small molecules.

In some embodiments, the small molecule is any compound that falls within the scope of the classes described above but that is not specifically named herein, including compounds that are developed after the filing date of the present application and that would have been recognized by a person of ordinary skill in the art as belonging to one or more of the foregoing classes.

Exemplary monoclonal antibody alternatives may include, but are not limited to, one or more of a fragment-based antibody, an antibody fragment, a multi-specific antibody, an Fc-engineered antibody, a fusion antibody, a conjugated antibody, a multimeric antibody, an antibody based on a non-immunoglobulin scaffold, a specialized engineered antibody, or a synthetic antibody-like molecule. In some embodiments, the antibody fragment is selected from one or more of a Fab fragment, a scFv fragment, a scFab fragment, a Fv fragment, a VHH fragment, a dAb fragment, and a VH-only fragment. Exemplary multi-specific antibodies include a BsAb IgG-like antibody, a BsAb non-IgG-like antibody, a BiTE antibody, a DART antibody, an ImmTAC antibody, and a MATCH antibody. In some embodiments, the formulations include one or more Fc-engineered antibodies such as a mAb with a modified Fc, a silent Fc-mAb, an immunocytokine, or a peptibody. Fusion antibodies used in certain embodiments of the invention may include, but are not limited to, an Fc-fusion antibody. Conjugated antibodies used in certain embodiments of the invention include an ADC, a radioimmunoconjugate, an antibody-oligonucleotide conjugate, or an antibody-enzyme conjugate. In certain embodiments, a multimeric antibody is used, such as a diabody, a triabody, a tetrabody, a pentabody, a hexabody, a heptabody, or an octabody. Certain additional embodiments may include an antibody based on a non-immunoglobulin scaffold, such as an affibody, a DARPin, an anticalin, a monobody, an adnectin, a fynomer, a Kunitz domain-containing antibody, an avimer, or a knottin. Specialized engineered antibodies used in certain embodiments of the invention may be an intrabody, a CAR, a CAR-T, a biparatopic antibody, a conditionally active antibody, a pH-switchable antibody, a protease-activated antibody, an orthogonal Fab antibody, or a zippered antibody. In some embodiments, a synthetic antibody-like molecule is used, such as a cyclic peptide with antibody-like binding, a DNA/RNA aptamer, or a siRNA. In certain embodiments, the pharmaceutically active ingredient is a peptide or protein therapeutic.

Exemplary peptide or protein therapeutics include those that have been approved for use in human and/or veterinary animal therapeutic and/or diagnostic use, such as those therapeutic peptides and proteins listed in the online “THPdb” database (available at http://crdd.osdd.net/raghava/thpdb/). Such peptide and protein therapeutics include, but are not limited to, an enzyme (such as dornase alpha, velaglucerase alpha, taliglucerase alpha, asparaginase, glucarpidase, asfotase alpha, elosulfase alpha, sebelipase alpha, sacrosidase and pegloticase), an antithrombin agent (such as lepirudin, bivalirudin, defibrotide and sulodexide), a thrombolytic agent (such as reteplase, anistreplase, tenecteplase, streptokinase and urokinase), a peptide or protein hormone such as parathyroid hormone, thyroxine (or an analogue thereof such as levothyroxine), prolactin, mammatrophic hormone, vasopressin, oxytocin, cortisol, and the like, amylin, angiotensin, growth hormone (including human growth hormone), growth hormone-releasing factor, glatiramer, exenatide, insulin-like growth factor, cosinotropin, chorionic gonadotropin (e.g., human chorionic gonadotropin) and somatotropin), a bone-active peptide or protein (such as calcitonin, e.g., salmon calcitonin), a diabetic-active peptide or protein (such as insulin (which may be human or porcine) and analogues thereof (including insulin lispro, insulin glargine, insulin aspart, insulin detemir, and insulin glulisine), pramlintide, and glucagon and analogues thereof (including dasiglucagon), an antibody or a fragment thereof (which may be a monoclonal antibody or a fragment thereof such as cetuximab, trastuzumab, bevacizumab, rituximab, obinutuzumab, gemtuzumab, canakinumab, ipilimumab, daratumumab, vedolizumab, ustekinumab, siltuximab, ramucirumab, pembrolizumab, ofatumumab, nivolumab, mepolizumab, brodalumab, pertuzumab, denosumab, golimumab, belimumab, raxibacumab, blinatuomab, dinutuximab, and ibritumomab), a non-antibody antineoplastic agent (such as leuprolide, denileukin diftitox, aldesleukin, asparaginase, pegasparagase, interferon beta, aflibercept, lenograstim and sipuleucel-T), a fertility agent (such as leuprolide, a menotropin, lutropin alpha, follitropin beta, urofollitropin, and choriogonadotropin alpha), and an immunosuppressive agent (such as etanercept, peginterferon alpha, an interferon alpha, filgrastim, pegfilgrastim, sargramostim, anakinra, an interferon beta, an interferon gamma, adalimumab, infliximab, basiliximab, muromonab, efalizumab, daclizumab, abatacept, rilonacept, belatacept, natalizumab, blintumomab, ustekinumab and human immune globulin). Other protein and peptide therapeutics suitable for use in the compositions and methods of the present invention will be familiar to those of ordinary skill in the art. Protein and peptide therapeutics advantageously used in accordance with the present invention may be naturally derived, synthetic or produced recombinantly, using methods of peptide and protein production that are well-known in the art.

The formulations, methods, kits, and devices described herein are suitable for use with a wide variety of biologic molecules. As used herein, a “biologic molecule” refers broadly to any protein, peptide, polypeptide, antibody, antibody fragment, antibody conjugate, fusion protein, glycoprotein, nucleoprotein, lipoprotein, PEGylated protein, albumin-conjugated protein, lipidated peptide, polyclonal antibody, polyclonal antibody fragment, oligonucleotide, aptamer, or other biologic macromolecule suitable for parenteral administration. The biologic molecule may be, without limitation, a monoclonal antibody, a polyclonal antibody, a bispecific antibody, a multispecific antibody, an antibody fragment, a nanobody, a single-domain antibody, an antibody-drug conjugate, a radioimmunoconjugate, a fusion protein, a recombinant protein, a peptide, a polypeptide, a cytokine, a growth factor, an enzyme, a hormone, a blood factor, an immunoglobulin preparation, a vaccine antigen, a virus-like particle, an oligonucleotide, a peptide-drug conjugate, a radiolabeled peptide conjugate, or any combination thereof. In some embodiments, the biologic molecule is a therapeutic protein. In some embodiments, the biologic molecule is a protein or mixture of proteins having a molecular weight or mean molecular weight of from about 1 kDa to about 250 kDa, such as from about 5 kDa to about 200 kDa, such as from about 10 kDa to about 160 kDa. The following paragraphs provide non-limiting examples of biologic molecules within each class that may benefit from the present formulation platform. The inclusion of any specific biologic molecule herein does not imply any particular regulatory status, and the omission of any specific biologic molecule does not imply exclusion from the scope of the present formulations. The present formulations are applicable to biologic molecules at any stage of development, manufacture, or commercialization.

In some embodiments, the biologic molecule is a monoclonal antibody (mAb). As used herein, a “monoclonal antibody” refers to an antibody produced by a single clone of B cells or a recombinant cell line, wherein substantially all antibody molecules in the preparation share the same primary amino acid sequence and bind to the same epitope. The monoclonal antibody may be a murine antibody, a chimeric antibody, a humanized antibody, or a fully human antibody. In certain aspects, the monoclonal antibody is an immunoglobulin G (IgG) antibody, such as an IgG1, IgG2, or IgG4 antibody. The monoclonal antibody may have a molecular weight of from about 140 kDa to about 160 kDa, such as about 150 kDa.

In some embodiments, the monoclonal antibody is an anti-TNF-alpha antibody. Non-limiting examples of anti-TNF-alpha antibodies include infliximab, adalimumab, golimumab, and certolizumab pegol, and biosimilars thereof.

In some embodiments, the monoclonal antibody is an anti-interleukin antibody. Non-limiting examples of anti-interleukin antibodies include: anti-IL-1β antibodies such as canakinumab; anti-IL-2 receptor antibodies such as basiliximab; anti-IL-4 receptor alpha antibodies such as dupilumab; anti-IL-5 antibodies such as mepolizumab, reslizumab, and depemokimab; anti-IL-5 receptor alpha antibodies such as benralizumab; anti-IL-6 antibodies such as siltuximab; anti-IL-6 receptor antibodies such as tocilizumab and sarilumab; anti-IL-12/IL-23 p40 antibodies such as ustekinumab; anti-IL-13 antibodies such as tralokinumab; anti-IL-17A antibodies such as secukinumab and ixekizumab; anti-IL-17 receptor A antibodies such as brodalumab; anti-IL-23 p19 antibodies such as guselkumab, risankizumab, tildrakizumab, and mirikizumab; anti-IL-31 receptor alpha antibodies such as nemolizumab; anti-IL-33 antibodies such as itepekimab and tozorakimab; anti-IL-36 receptor antibodies such as spesolimab; anti-thymic stromal lymphopoietin (TSLP) antibodies such as tezepelumab; and antibodies targeting other interleukins or interleukin receptors, and biosimilars thereof.

In some embodiments, the monoclonal antibody targets a cluster of differentiation (CD) antigen or other cell surface antigen. Non-limiting examples include: anti-CD2 antibodies such as siplizumab; anti-CD3 antibodies such as teplizumab; anti-CD19 antibodies such as tafasitamab; anti-CD20 antibodies such as rituximab, ofatumumab, obinutuzumab, ocrelizumab, and ublituximab; anti-CD22 antibodies such as epratuzumab; anti-CD30 antibodies; anti-CD38 antibodies such as daratumumab and isatuximab; anti-CD47 antibodies such as magrolimab; anti-CD52 antibodies such as alemtuzumab; anti-CD79b antibodies; anti-P-selectin antibodies such as crizanlizumab; anti-SLAMF7 antibodies such as elotuzumab; anti-GD2 antibodies such as dinutuximab and naxitamab; anti-CCR4 antibodies such as mogamulizumab; and antibodies targeting other CD antigens or cell surface markers, and biosimilars thereof.

In some embodiments, the monoclonal antibody is an immune checkpoint inhibitor antibody. Non-limiting examples include: anti-PD-1 antibodies such as nivolumab, pembrolizumab, cemiplimab, dostarlimab, retifanlimab, toripalimab, tislelizumab, and zimberelimab; anti-PD-L1 antibodies such as atezolizumab, avelumab, and durvalumab; anti-CTLA-4 antibodies such as ipilimumab and tremelimumab; anti-LAG-3 antibodies such as relatlimab; anti-TIGIT antibodies such as tiragolumab and domvanalimab; anti-TIM-3 antibodies; and antibodies targeting other immune checkpoint receptors or ligands, including without limitation VISTA, B7-H3, BTLA, ICOS, OX40, 4-1BB, GITR, and CD27, and biosimilars thereof.

In some embodiments, the monoclonal antibody targets human epidermal growth factor receptor 2 (HER2), epidermal growth factor receptor (EGFR), or another member of the ErbB receptor family. Non-limiting examples include: anti-HER2 antibodies such as trastuzumab, pertuzumab, and margetuximab; anti-EGFR antibodies such as cetuximab, panitumumab, and necitumumab; anti-HER3 antibodies; and antibodies targeting other members of the ErbB family, and biosimilars thereof.

In some embodiments, the monoclonal antibody targets a component of the angiogenesis signaling pathway. Non-limiting examples include: anti-VEGF antibodies such as bevacizumab; anti-VEGFR2 antibodies such as ramucirumab; anti-angiopoietin antibodies; and other antibodies targeting components of the angiogenesis pathway, and biosimilars thereof.

In some embodiments, the biologic molecule targets a component of the complement system. Non-limiting examples include: anti-C5 antibodies such as eculizumab and ravulizumab; anti-C1s antibodies such as sutimlimab; complement C3 inhibitors such as pegcetacoplan; complement C5 peptide inhibitors such as zilucoplan; complement C5 aptamer inhibitors such as avacincaptad pegol; and other antibodies or inhibitors targeting complement components, including without limitation C1q, C1r, C2, C3, C4, factor B, factor D, factor H, properdin, and the membrane attack complex, and biosimilars thereof.

In some embodiments, the monoclonal antibody targets the neonatal Fc receptor (FcRn). Non-limiting examples include: rozanolixizumab; nipocalimab; and other anti-FcRn antibodies, and biosimilars thereof. FcRn antagonists that are not monoclonal antibodies, such as efgartigimod (an engineered IgG1 Fc fragment), are described below. In some embodiments, the monoclonal antibody targets other immunomodulatory or inflammatory mediators. Non-limiting examples include: anti-IgE antibodies such as omalizumab; anti-BLyS/BAFF antibodies such as belimumab; anti-alpha-4 integrin antibodies such as natalizumab; anti-alpha-4-beta-7 integrin antibodies such as vedolizumab; anti-CGRP antibodies such as galcanezumab, fremanezumab, and eptinezumab; anti-CGRP receptor antibodies such as erenumab; anti-RANKL antibodies such as denosumab; anti-sclerostin antibodies such as romosozumab; anti-NGF antibodies such as tanezumab and fasinumab; anti-PCSK9 antibodies such as evolocumab and alirocumab; anti-factor XIa antibodies such as abelacimab and osocimab; anti-TFPI antibodies such as marstacimab and concizumab; anti-amyloid beta antibodies such as lecanemab, donanemab, and aducanumab; and antibodies targeting other immunomodulatory, inflammatory, metabolic, neurological, or musculoskeletal mediators, and biosimilars thereof.

In some embodiments, the biologic molecule is a polyclonal antibody or a polyclonal antibody preparation. As used herein, a “polyclonal antibody” or “polyclonal antibody preparation” refers to a mixture of antibody molecules derived from the plasma or serum of one or more human or animal donors, wherein the mixture comprises antibodies recognizing a plurality of epitopes. Polyclonal antibody preparations may be derived from pooled human plasma, from hyper-immunized human donors, or from immunized animals. Polyclonal antibody preparations may comprise intact immunoglobulin molecules or antibody fragments generated by enzymatic digestion of intact immunoglobulins.

In some embodiments, the polyclonal antibody preparation is an immune globulin intravenous (IVIG) product prepared from pooled human plasma and indicated for the treatment of primary immunodeficiency, immune thrombocytopenia, chronic inflammatory demyelinating polyneuropathy, Kawasaki disease, and other immune-mediated conditions.

In some embodiments, the polyclonal antibody preparation is an immune globulin subcutaneous (SCIG) product. In some embodiments, the SCIG product is co-formulated with recombinant human hyaluronidase (rHuPH20) to enhance subcutaneous dispersion and absorption.

In some embodiments, the polyclonal antibody preparation is a hyperimmune globulin derived from donors with high titers of antibodies against a specific pathogen or antigen. Non-limiting examples include: anti-D immunoglobulin (Rho(D) immune globulin); hepatitis B immune globulin; rabies immune globulin; tetanus immune globulin; varicella-zoster immune globulin; cytomegalovirus immune globulin; botulism antitoxin; vaccinia immune globulin; and other hyperimmune globulins directed against other pathogens, toxins, or antigens.

In some embodiments, the polyclonal antibody preparation comprises antibody fragments produced by enzymatic digestion of intact polyclonal immunoglobulins, yielding Fab or F(ab′)2 fragments. Non-limiting examples include: antithymocyte globulin (rabbit); antithymocyte globulin (equine); crotalidae polyvalent immune Fab (ovine); crotalidae immune F(ab′)2 (equine); Centruroides immune F(ab′)2 (equine); digoxin immune Fab (ovine); and other polyclonal antibody fragment preparations.

In some embodiments, the polyclonal antibody preparation is an animal-derived polyclonal antiserum or antitoxin. Non-limiting examples include: diphtheria antitoxin (equine); black widow spider antivenin (equine); North American coral snake antivenin (equine); and other animal-derived antisera and antitoxins for the treatment or prophylaxis of envenomation, intoxication, or infectious diseases.

In some embodiments, the biologic molecule is a bispecific antibody or a multispecific antibody. As used herein, a “bispecific antibody” refers to an antibody or antibody-like molecule capable of specifically binding to two different epitopes or antigens. A “multispecific antibody” refers to an antibody or antibody-like molecule capable of specifically binding to three or more different epitopes or antigens. Bispecific and multispecific antibodies may employ any format, including without limitation bispecific T-cell engagers (BiTE), dual-variable-domain immunoglobulins (DVD-Ig), knobs-into-holes formats, CrossMAb formats, dual-affinity retargeting (DART) molecules, tandem diabodies, trispecific killer cell engagers (TrikEs), and other heterodimeric or multidomain constructs. Multispecific antibodies may have a molecular weight of from about 50 kDa to about 250 kDa, depending on format.

Non-limiting examples of bispecific and multispecific antibodies include: blinatumomab (anti-CD19×CD3); mosunetuzumab (anti-CD20×CD3); glofitamab (anti-CD20×CD3); epcoritamab (anti-CD20×CD3); teclistamab (anti-BCMA×CD3); elranatamab (anti-BCMA×CD3); talquetamab (anti-GPRC5D×CD3); tarlatamab (anti-DLL3×CD3); linvoseltamab (anti-BCMA×CD3); tebentafusp (gp100×CD3); ivonescimab (anti-PD-1×VEGF); cadonilimab (anti-PD-1×CTLA-4); zanidatamab (anti-HER2 biparatopic); emicizumab (anti-factor IXa×factor X); faricimab (anti-VEGF-A×Ang-2); amivantamab (anti-EGFR×c-MET); zenocutuzumab (anti-HER2×HER3); sonelokimab (anti-IL-17A/IL-17F trivalent nanobody construct); and other bispecific and multispecific antibodies targeting any combination of antigens, and biosimilars thereof.

In some embodiments, the biologic molecule is an antibody fragment, a single-domain antibody, or a nanobody. As used herein, an “antibody fragment” refers to a portion of an antibody that retains antigen-binding capacity, including without limitation Fab fragments, F(ab′)2 fragments, single-chain variable fragments (scFv), diabodies, minibodies, and single-domain antibodies (sdAb or VHH, also known as nanobodies). Antibody fragments typically have a molecular weight of from about 12 kDa to about 110 kDa. Non-limiting examples include: certolizumab pegol (PEGylated anti-TNF Fab′); ranibizumab (anti-VEGF-A Fab); brolucizumab (anti-VEGF-A scFv); caplacizumab (anti-von Willebrand factor nanobody); ozoralizumab (anti-TNF-alpha trivalent nanobody); and other antibody fragments and nanobodies, and biosimilars thereof.

In some embodiments, the biologic molecule is a fusion protein. As used herein, a “fusion protein” refers to a recombinant protein comprising two or more protein domains or functional moieties joined by a peptide bond or linker, including Fc-fusion proteins, receptor-Fc fusions, enzyme-Fc fusions, cytokine-Fc fusions, albumin-fusion proteins, and engineered Fc fragments. Non-limiting examples include: etanercept (TNFR2-Fc fusion); abatacept (CTLA-4-Ig Fc fusion); belatacept (CTLA-4-Ig Fc fusion); aflibercept (VEGFR1/VEGFR2-Fc fusion); rilonacept (IL-1R/IL-1RAcP-Fc fusion); romiplostim (thrombopoietin receptor agonist peptibody); dulaglutide (GLP-1-Fc fusion); efgartigimod (engineered IgG1 Fc fragment, FcRn antagonist); efmoroctocog alfa (factor VIII-Fc fusion); alprolix (factor IX-Fc fusion); luspatercept (activin receptor IIB-Fc fusion); sotatercept (activin receptor IIA-Fc fusion); eftrenonacog alfa (factor IX-Fc fusion); albutrepenonacog alfa (factor IX-albumin fusion); and other fusion proteins, and biosimilars thereof.

In some embodiments, the biologic molecule is a peptide or polypeptide, or a pharmaceutically acceptable salt, solvate, hydrate, ester, or prodrug thereof. As used herein, a “peptide” refers to a molecule comprising from about 2 to about 100 amino acid residues, and a “polypeptide” refers to a molecule comprising more than about 100 amino acid residues but that is not a full-length antibody or antibody fragment. Peptides and polypeptides may be natural, synthetic, or recombinant, and may be modified by PEGylation, lipidation, glycosylation, acetylation, cyclization, stapling, or other post-translational or chemical modifications.

In some embodiments, the peptide is a GLP-1 receptor agonist or a multi-receptor incretin agonist, or a pharmaceutically acceptable salt thereof. Non-limiting examples include: exenatide; lixisenatide; liraglutide; semaglutide; tirzepatide (a dual GIP/GLP-1 receptor agonist); retatrutide (a triple GIP/GLP-1/glucagon receptor agonist); survodutide (a dual glucagon/GLP-1 receptor agonist); and other single-receptor, dual-receptor, or multi-receptor incretin agonists.

In some embodiments, the peptide is insulin or an insulin analog.

In some embodiments, the peptide is glucagon or a glucagon analog.

In some embodiments, the peptide is selected from, without limitation, one or more of the following: calcitonin; teriparatide (parathyroid hormone [1-34]); palopegteriparatide; abaloparatide; vosoritide; octreotide; lanreotide; pasireotide; enfuvirtide; icatibant; teduglutide; pramlintide; ziconotide; linaclotide; plecanatide; setmelanotide; bremelanotide; afamelanotide; etelcalcetide; difelikefalin; terlipressin; zilucoplan; motixafortide; trofinetide; pegcetacoplan; and other therapeutic peptides, peptide analogs, cyclic peptides, stapled peptides, peptibodies, and peptide-drug conjugates, or a pharmaceutically acceptable salt, solvate, or prodrug thereof.

In some embodiments, the biologic molecule is a cytokine or an immunomodulatory protein. Non-limiting examples include: interferons, such as interferon alfa-2a, interferon alfa-2b, interferon beta-1a, interferon beta-1b, interferon gamma-1b, peginterferon alfa-2a, peginterferon alfa-2b, peginterferon beta-1a, and ropeginterferon alfa-2b; interleukins, such as aldesleukin (interleukin-2), oprelvekin (interleukin-11), and engineered IL-2 variants; colony-stimulating factors, such as filgrastim, pegfilgrastim, eflapegrastim, and sargramostim; interleukin-1 receptor antagonists such as anakinra; and other recombinant cytokines, chemokines, or immunomodulatory proteins.

In some embodiments, the biologic molecule is a growth factor. Non-limiting examples include: erythropoietin (epoetin alfa, epoetin beta, epoetin zeta); darbepoetin alfa; methoxy polyethylene glycol-epoetin beta; romiplostim; platelet-derived growth factor; fibroblast growth factors such as FGF-21 analogs; palifermin (keratinocyte growth factor); dibotermin alfa (BMP-2); vascular endothelial growth factor; and analogs, variants, and PEGylated forms thereof.

In some embodiments, the biologic molecule is an enzyme, including enzymes used in enzyme replacement therapy (ERT) for lysosomal storage disorders and other metabolic diseases. Non-limiting examples include: imiglucerase, velaglucerase alfa, and taliglucerase alfa (for Gaucher disease); agalsidase alfa and agalsidase beta (for Fabry disease); laronidase (for MPS I); idursulfase (for MPS II); galsulfase (for MPS VI); elosulfase alfa (for MPS IVA); vestronidase alfa (for MPS VII); cerliponase alfa (for CLN2 disease); avalglucosidase alfa and alglucosidase alfa (for Pompe disease); sebelipase alfa (for LAL deficiency); olipudase alfa (for Niemann-Pick disease); pegunigalsidase alfa (for Fabry disease); cipaglucosidase alfa (for Pompe disease); asfotase alfa (for hypophosphatasia); and other enzyme replacement therapies.

In some embodiments, the enzyme is used for a therapeutic indication other than enzyme replacement therapy. Non-limiting examples include: pegaspargase and calaspargase pegol; rasburicase; pegloticase; hyaluronidase (including rHuPH20); collagenase clostridium histolyticum; alpha-1 proteinase inhibitor; C1 esterase inhibitor; ecallantide; dornase alfa; and other therapeutic enzymes.

In some embodiments, the biologic molecule is a hormone or a hormone analog. Non-limiting examples include: somatropin and long-acting growth hormone analogs such as somapacitan and lonapegsomatropin; follitropin alfa, follitropin beta, and corifollitropin alfa; lutropin alfa; choriogonadotropin alfa; GnRH analogs such as leuprolide, goserelin, triptorelin, buserelin, nafarelin, histrelin, and degarelix; teriparatide, palopegteriparatide, and abaloparatide; dasiglucagon; calcitonin; oxytocin; desmopressin and terlipressin; cosyntropin; thyrotropin alfa; and other recombinant, synthetic, or naturally derived hormones or hormone analogs.

In some embodiments, the biologic molecule is a coagulation factor or a protein involved in hemostasis. Non-limiting examples include: factor VIII products such as octocog alfa, turoctocog alfa, simoctocog alfa, damoctocog alfa pegol, rurioctocog alfa pegol, and efmoroctocog alfa; factor IX products such as nonacog alfa, nonacog beta pegol, albutrepenonacog alfa, and eftrenonacog alfa; factor VIIa (eptacog alfa); factor XIII (catridecacog); von Willebrand factor (vonicog alfa); antithrombin III; protein C concentrate; fibrinogen concentrate; thrombin; and other recombinant or plasma-derived coagulation factors.

In some embodiments, the biologic molecule for hemostasis is a non-factor replacement therapy. Non-limiting examples include: emicizumab (anti-factor IXa×factor X bispecific antibody); marstacimab (anti-TFPI antibody); concizumab (anti-TFPI antibody); fitusiran (antithrombin-targeting siRNA); abelacimab (anti-factor XI antibody); and other non-factor replacement therapies.

In some embodiments, the biologic molecule is a vaccine antigen or a component of a vaccine composition. Non-limiting examples include: recombinant protein subunit antigens (e.g., recombinant hepatitis B surface antigen, recombinant influenza hemagglutinin, SARS-CoV-2 spike protein or receptor-binding domain, recombinant varicella-zoster glycoprotein E, respiratory syncytial virus prefusion F protein); virus-like particles (VLPs) such as human papillomavirus L1 protein VLPs and hepatitis B surface antigen VLPs; toxoids such as tetanus toxoid, diphtheria toxoid, and pertussis toxoid; conjugated polysaccharide antigens such as pneumococcal and meningococcal conjugate vaccine antigens; nanoparticle-based antigen formulations; and other vaccine antigens for infectious diseases, oncology, or other prophylactic or therapeutic applications. In certain aspects, the vaccine composition further comprises one or more adjuvants.

In some embodiments, the biologic molecule is an oligonucleotide therapeutic. Non-limiting examples include: antisense oligonucleotides (ASOs) such as nusinersen, inotersen, eplontersen, mipomersen, volanesorsen, tofersen, and olezarsen; phosphorodiamidate morpholino oligomers (PMOs) such as eteplirsen, golodirsen, viltolarsen, and casimersen; small interfering RNAs (siRNAs) such as patisiran, givosiran, lumasiran, inclisiran, fitusiran, vutrisiran, and nedosiran; oligonucleotide telomerase inhibitors such as imetelstat; aptamers such as pegaptanib and avacincaptad pegol; and other oligonucleotide therapeutics, including anti-microRNA oligonucleotides, splice-switching oligonucleotides, mRNA therapeutics, and circular RNA therapeutics. In certain aspects, the oligonucleotide is conjugated to N-acetylgalactosamine (GalNAc) for hepatocyte targeting, is formulated in a lipid nanoparticle, or employs a phosphorodiamidate morpholino, phosphorothioate, locked nucleic acid (LNA), 2′-O-methoxyethyl (2′-MOE), or other chemically modified backbone.

In some embodiments, the biologic molecule is a radiolabeled peptide conjugate or a peptide-drug conjugate. Non-limiting examples include: lutetium Lu 177 dotatate (a somatostatin receptor-targeting radiolabeled peptide); lutetium Lu 177 vipivotide tetraxetan (a PSMA-targeting radiolabeled peptide); and other radiolabeled peptide conjugates or peptide-drug conjugates.

In some embodiments, the biologic molecule is a recombinant protein not encompassed by the foregoing categories. Non-limiting examples include nesiritide and recombinant human alpha-1 antitrypsin.

In some embodiments, the biologic molecule is a recombinant toxin or immunotoxin, such as: botulinum toxin type A (onabotulinumtoxinA, abobotulinumtoxinA, incobotulinumtoxinA, prabotulinumtoxinA, daxibotulinumtoxinA); and botulinum toxin type B (rimabotulinumtoxinB).

The formulations described herein are not limited to the specific biologic molecules enumerated above. The foregoing examples are provided to illustrate the breadth and diversity of biologic molecules that may benefit from the present formulation platform and are not intended to be exhaustive.

In some embodiments, the biologic molecule is any protein, peptide, polypeptide, glycoprotein, lipoprotein, nucleoprotein, PEGylated protein, albumin-conjugated protein, lipidated peptide, polyclonal antibody, polyclonal antibody fragment, or other biologic macromolecule that (i) is intended for subcutaneous, intramuscular, intravenous, intravitreal, intrathecal, intraarticular, intradermal, or other parenteral administration, and (ii) may benefit from a formulation that provides one or more of: enhanced stability, improved syringeability, improved injectability, reduced immunogenicity, controlled release, sustained release, or improved bioavailability relative to a reference formulation.

In some embodiments, the biologic molecule has a concentration in the formulation of from about 1 mg/mL to about 700 mg/mL, such as from about 1 mg/mL to about 400 mg/mL, such as from about 10 mg/mL to about 300 mg/mL, such as from about 50 mg/mL to about 250 mg/mL, such as from about 100 mg/mL to about 200 mg/mL. In some embodiments, the biologic molecule has a concentration in the formulation greater than about 700 mg/mL.

In some embodiments, the biologic molecule is a biosimilar of any of the foregoing biologic molecules. In some embodiments, the biologic molecule is an interchangeable biosimilar. In some embodiments, the biologic molecule is a biobetter or next-generation variant of any of the foregoing biologic molecules, including variants having one or more amino acid substitutions, deletions, or insertions relative to the reference biologic molecule; variants having altered glycosylation, PEGylation, or other post-translational modifications; variants having an Fc mutation to modulate effector function, half-life, or FcRn binding; variants comprising a different antibody isotype or subclass; and variants employing novel formulation, conjugation, or delivery technologies.

In some embodiments, the biologic molecule is any molecule that falls within the scope of the classes described above but that is not specifically named herein, including molecules that are developed after the filing date of the present application and that would have been recognized by a person of ordinary skill in the art as belonging to one or more of the foregoing classes.

In some embodiments, the biologic molecule is an antibody-drug conjugate (ADC) or a radioimmunoconjugate. An ADC comprises a monoclonal antibody covalently linked to a cytotoxic payload via a chemical linker. The cytotoxic payload may be a tubulin inhibitor, a DNA-damaging agent, a topoisomerase I inhibitor, an immune agonist, or another cytotoxic, cytostatic, or immunomodulatory agent. The linker may be cleavable or non-cleavable. A radioimmunoconjugate comprises a monoclonal antibody conjugated to a radioactive isotope.

Non-limiting examples include: trastuzumab emtansine (anti-HER2-DM1); trastuzumab deruxtecan (anti-HER2-DXd); sacituzumab govitecan (anti-TROP2-SN-38); enfortumab vedotin (anti-Nectin-4-MMAE); brentuximab vedotin (anti-CD30-MMAE); polatuzumab vedotin (anti-CD79b-MMAE); gemtuzumab ozogamicin (anti-CD33-calicheamicin); inotuzumab ozogamicin (anti-CD22-calicheamicin); tisotumab vedotin (anti-tissue factor-MMAE); loncastuximab tesirine (anti-CD19-PBD dimer); mirvetuximab soravtansine (anti-folate receptor alpha-DM4); belantamab mafodotin (anti-BCMA-MMAF); datopotamab deruxtecan (anti-TROP2-DXd); patritumab deruxtecan (anti-HER3-DXd); telisotuzumab vedotin (anti-c-MET-MMAE); disitamab vedotin (anti-HER2-MMAE); ibritumomab tiuxetan (anti-CD20-yttrium-90); and other ADCs and radioimmunoconjugates, and biosimilars thereof.

Other active pharmaceutical ingredients suitably used in producing the compositions of the present invention by the methods of the present invention are small molecule therapeutic and/or diagnostic agents and salts thereof. Such agents are typically low molecular weight (e.g., less than about 1000 daltons) organic or inorganic compounds that have a desired bioactivity making them useful in treating, ameliorating, preventing and/or diagnosing a disease or disorder once the agent is introduced into the body of an animal (e.g., a human or a veterinary animal). Examples of such small molecule active pharmaceutical ingredients (and salts thereof) suitable for use in accordance with the present invention include, but are not limited to, epinephrine, benzodiazepines, catecholemines, “triptans,” sumatriptan, novantrone, chemotherapy small molecules (e.g., mitoxantrone), corticosteroid small molecules (e.g., methylprednisolone, beclomethasone dipropionate), immunosuppressive small molecules (e.g., azathioprine, cladribine, cyclophosphamide monohydrate, methotrexate), anti-inflammatory small molecules (e.g., salicylic acid, acetylsalicylic acid, lisofylline, diflunisal, choline magnesium trisalicylate, salicylate, benorylate, flufenamic acid, mefenamic acid, meclofenamic acid, triflumic acid, diclofenac, fenclofenac, alclofenac, fentiazac, ketorolac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin, sulindac, phenylbutazone, oxyphenbutazone, azapropazone, feprazone, piroxicam, isoxicam), small molecules used to treat neurological disorders (e.g., cimetidine, ranitidine, famotidine, nizatidine, tacrine, metrifonate, rivastigmine, selegilene, imipramine, fluoxetine, olanzapine, sertindole, risperidone, valproate semisodium, gabapentin, carbamazepine, topiramate, phenytoin, dichlorphenamate), small molecules used to treat cancer (e.g., vincristine, vinblastine, paclitaxel, docetaxel, cisplatin, irinotecan, topotecan, gemcitabine, temozolomide, imatinib, bortezomib), statins (e.g., atorvastatin, amlodipine, rosuvastatin, sitagliptin, simvastatin, fluvastatin, pitavastatin, lovastatin, pravastatin, simvastatin), and other taxane derivatives, small molecules used to treat tuberculosis (e.g., rifampicin), small molecule anti-fungal agents (e.g., fluconazole, ketoconazole), small molecule anti-anxiety agents and small molecule anti-convulsant agents (e.g., lorazepam), small molecule anti-cholinergic agents (e.g., atropine), small molecule β-agonist drugs (e.g., albuterol sulfate), small molecule mast cell stabilizers and small molecule agents used to treat allergies (e.g., cromolyn sodium), small molecule anesthetic agents and small molecule anti-arrhythmic agents (e.g., lidocaine), small molecule antibiotic agents (e.g., tobramycin, ciprofloxacin), small molecule anti-migraine agents (e.g., sumatriptan), and small molecule anti-histamine drugs (e.g., diphenhydramine), and small molecule agents useful in treating endocrine disorders such as Cushing's disease and Cushing's syndrome (e.g., levoketoconazole). Other small molecule therapeutics and diagnostics, and salts thereof, that are suitable for use in the compositions and methods of the present invention will be familiar to those of ordinary skill in the art. Additional formulations comprise combinations of such agents, comprising at least two of the small molecule therapeutics and diagnostics described herein and others that are familiar to those of ordinary skill in the art. Small molecules and salts thereof that can be advantageously used in accordance with the present invention may be obtained commercially from a wide range of sources (e.g., ThermoFisher, Aldrich Chemical and the like), or may be synthesized using methods of chemical and biochemical synthesis that are well-known in the art.

### Methods of Use

The compositions of the present invention can be used to treat, ameliorate, prevent or diagnose a variety of diseases and physical disorders in animals, including veterinary animals or humans, in need of such treatment, amelioration, prevention and diagnosis. Suitable such methods involve the administration of one or more of the paste compositions of the present invention by injection, suitably intracutaneously, subcutaneously, intradermally, or intramuscularly, in relatively low volumes (e.g., 1 μL to 10000 μL or less), resulting in the delivery of a bolus of therapeutic compound in potentially lower volumes and/or more rapidly than can be achieved using other methods of administration of aqueous therapeutic formulations having lower concentrations of active ingredient, e.g., via intravenous infusion. These methods of use may result in less discomfort to the animal post-injection, and also demonstrate certain pharmacokinetic and pharmacodynamic advantages as detailed in the Examples hereinbelow. Diseases and physical disorders suitably treated, prevented, ameliorated or diagnosed using the paste compositions of the present invention will be readily apparent to those of ordinary skill in the art, and the choice of active ingredient to be used as a starting material in producing the pastes of the invention will also be familiar to the ordinarily skilled practitioner based on the disease, physical disorder or condition to be treated, prevented, ameliorated or diagnosed using the paste compositions of the present invention.

In some embodiments, an exemplary such method of the present invention comprises treating or preventing hypoglycemia by administering to a subject having hypoglycemia or at risk for experiencing hypoglycemia a paste formulation or composition as described herein in an amount effective to treat or prevent the hypoglycemia. In some embodiments, the subject is administered a paste formulation comprising glucagon. In certain aspects hypoglycemia can be caused by, or the patient can be at higher risk for experiencing hypoglycemia because of, diabetes or non-diabetes related diseases, conditions, and disorders.

As described by the Workgroup of the American Diabetes Association and the Endocrine Society, (Seaquist, et al., (2013), Diabetes Care, Vol 36, pages 1384-1395) with respect to hypoglycemia a single threshold value for plasma glucose concentration that defines hypoglycemia in diabetes is not typically assigned because glycemic thresholds for symptoms of hypoglycemia (among other responses) shift to lower plasma glucose concentrations after recent antecedent hypoglycemia and to higher plasma glucose concentrations in patients with poorly controlled diabetes and infrequent hypoglycemia.

Nonetheless, an alert value can be defined that draws the attention of both patients and caregivers to the potential harm associated with hypoglycemia. Patients at risk for hypoglycemia (i.e., those treated with a sulfonylurea, glinide, or insulin) should be alert to the possibility of developing hypoglycemia at a self-monitored plasma glucose—or continuous glucose monitoring subcutaneous glucose—concentration of ≤70 mg/dL (≤3.9 mmol/L). Because it is higher than the glycemic threshold for symptoms in both nondiabetic individuals and those with well-controlled diabetes, it generally allows time to prevent a clinical hypoglycemic episode and provides some margin for the limited accuracy of monitoring device at low-glucose levels.

The condition of severe hypoglycemia is an event requiring assistance of another person to actively administer carbohydrates, glucagon, or take other corrective actions. Plasma glucose concentrations may not be available during an event, but neurological recovery following the return of plasma glucose to normal is considered sufficient evidence that the event was induced by a low plasma glucose concentration. Typically, these events begin occurring at plasma glucose concentrations of ≤50 mg/dL (2.8 mmol/L). Documented symptomatic hypoglycemia is an event during which typical symptoms of hypoglycemia are accompanied by a measured plasma glucose concentration≤70 mg/dL (≤3.9 mmol/L). Asymptomatic hypoglycemia is an event not accompanied by typical symptoms of hypoglycemia but with a measured plasma glucose concentration≤70 mg/dL (≤3.9 mmol/L). Probable symptomatic hypoglycemia is an event during which symptoms typical of hypoglycemia are not accompanied by a plasma glucose determination but that was presumably caused by a plasma glucose concentration ≤70 mg/dL (≤3.9 mmol/L). Pseudo-hypoglycemia is an event during which the person with diabetes reports any of the typical symptoms of hypoglycemia with a measured plasma glucose concentration >70 mg/dL (>3.9 mmol/L) but approaching that level.

Further included in the indications which may be treated by the disclosed invention are hypoglycemia-associated autonomic failure (HAAF). As described by Philip E. Cryer, Perspectives in Diabetes, Mechanisms of Hypoglycemia-Associated Autonomic Failure and Its Component Syndromes in Diabetes, Diabetes, Vol. 54, pp. 3592-3601 (2005), “recent antecedent iatrogenic hypoglycemia causes both defective glucose counter-regulation (by reducing epinephrine responses to a given level of subsequent hypoglycemia in the setting of absent decrements in insulin and absent increments in glucagon) and hypoglycemia unawareness (by reducing sympathoadrenal and the resulting neurogenic symptom responses to a given level of subsequent hypoglycemia) and thus a vicious cycle of hypoglycemia.” HAAF affects those with type 1 and advanced type 2 diabetes. Additionally, the invention of the present disclosure may also treat hypoglycemia in patients following islet cell transplantation.

The compositions of the present invention can also be used for the treatment or prevention of hyperinsulinemic hypoglycemia, which broadly refers to the condition and effects of low blood glucose levels that are caused by excessive insulin. The most common type of severe, but typically transient, hyperinsulinemic hypoglycemia arises from the administration of exogenous insulin in patients with Type 1 diabetes. This type of hypoglycemia can be defined as iatrogenic hypoglycemia and is a limiting factor in the glycemic management of type 1 and type 2 diabetes. Nocturnal hypoglycemia (night-time hypo) is a common type of iatrogenic hypoglycemia arising in patients taking exogenous insulin. However, hyperinsulinemic hypoglycemia can also arise due to endogenous insulin, for example in congenital hyperinsulinism, insulinomas (insulin-secreting tumors), exercise-induced hypoglycemia and reactive hypoglycemia. Reactive hypoglycemia is a non-diabetic hypoglycemia and is due to low blood sugar that occurs following a meal-typically within four hours after eating. Reactive hypoglycemia may also be referred to as postprandial hypoglycemia. Symptoms and signs of reactive hypoglycemia can include hunger, weakness, shakiness, sleepiness, sweating, confusion and anxiety. Stomach surgery (e.g. bariatric surgery) is one possible cause, as following surgery food may pass too quickly into the small intestine (e.g. post-bariatric hypoglycemia (PBH)). Additional causes include enzyme deficiencies that make it difficult for the body to breakdown food, or increased sensitivity to the hormone epinephrine.

In some embodiments, the disease, condition, or disorder to be treated or prevented with a paste composition of the present invention is a diabetic condition. Examples of diabetic conditions include, but are not limited to, type 1 diabetes, type 2 diabetes, gestational diabetes, pre-diabetes, hyperglycemia, hypoglycemia, and metabolic syndrome. In some embodiments, the disease, condition, or disorder is hypoglycemia, including but not limited to diabetes-related hypoglycemia, exercise-induced hypoglycemia, and post-bariatric surgery hypoglycemia, or other types of hypoglycemia described herein and known to those of ordinary skill in the art. In some embodiments, the disease, condition, or disorder is diabetes.

In some embodiments, a method of the present invention comprises treating diabetes by administering to a subject having diabetes a therapeutic agent in a paste formulation as described herein in an amount effective to treat the diabetes. In some embodiments, the subject is administered a paste formulation comprising insulin. In some embodiments, the subject is administered a paste formulation comprising pramlintide. In some embodiments, the subject is administered a paste formulation comprising insulin and pramlintide. In some embodiments, the subject is administered a paste formulation comprising exenatide. In some embodiments, the subject is administered a paste formulation comprising glucagon and exenatide.

In certain aspects a paste formulation of the invention comprising epinephrine can be administered to a subject at risk of or suspected of anaphylaxis. Epinephrine is indicated as an emergency treatment of Type I allergic reactions which can arise from multiple sources, including, but not limited to, foods, drugs and/or other allergens, allergen immunotherapy, diagnostic testing substances, insect stings and bites, and idiopathic or exercise-induced anaphylaxis.

In certain embodiments, at least one rheology modifier is added to reduce the loss of yield stress, allowing for consistently lower injection forces. In certain aspects, an example of a rheology modifier can be PS20 or PS80.

Other diseases, disorders, and conditions suitably treated, prevented, ameliorated, or diagnosed using the compositions and methods of the present invention will be readily familiar to the ordinarily skilled artisan and include, without limitation, cancers, infectious diseases, bacterial diseases, endocrine diseases and disorders (including diabetes, hypoglycemia, hypothyroidism, hypercortisolism, and the like), fungal diseases, viral diseases, and other diseases, disorders and conditions involving inflammatory, neurological, osteological, gastrointestinal, circulatory, cardiovascular, skin, muscular, developmental and other symptoms, signs or dysfunctions.

Concentrated suspensions, such as VES formulations, with elevated solids content, while providing significant advantages in terms of drug concentration and reduced injection volumes, may present unique delivery challenges that are not adequately addressed by conventional formulation approaches. Due to the relatively high solids content of VES formulations compared to traditional (i.e., more dilute) suspensions of the same powder(s) and diluent(s), such formulations may be more prone to partial or complete clogging or occlusion of delivery devices, particularly fine-gauge needles suitable for parenteral administration, from the presence of agglomerates. Importantly, this occlusion may occur even when individual agglomerates are smaller than the internal diameter of the needle lumen, as the elevated solids content and potential for particle bridging and/or accumulation within the delivery path may create flow restrictions that compromise reliable dose delivery. Traditional suspension formulations, containing lower relative solids content and therefore less prone to occlusion, typically cannot achieve the high drug concentrations possible with VES formulations and thus may require impractically large injection volumes for many therapeutic applications.

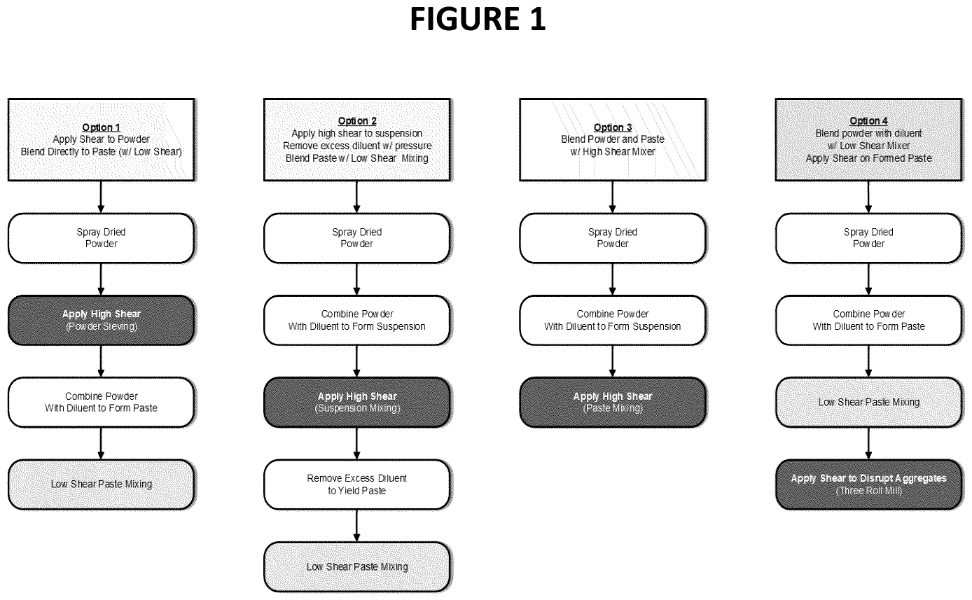

The present invention addresses fundamental limitations inherent in conventional approaches to agglomerate reduction in pharmaceutical suspensions and viscoelastic suspensions. Traditional methods, such as applying shear forces to powders to disrupt agglomerates through screening/sieving prior to combining with diluent (as depicted in Option 1 of FIG. 1), suffer from significant scalability and manufacturing constraints. Powder screening/sieving for agglomerate disruption is inherently limited by the fixed aperture size of the screening/sieving medium, which may be ineffective against agglomerates that are smaller than the mesh openings but still sufficiently large to impact syringeability. Moreover, agglomerates frequently form or reform during post-processing collection, storage, and handling operations prior to mixing, rendering pre-screening/sieving approaches of limited utility for sustained agglomerate control. From a manufacturing perspective, the incorporation of screening/sieving steps into aseptic processing environments presents substantial challenges, including the difficulty of sterilizing complex mesh geometries, the potential for mesh clogging or fouling during extended processing runs, and the inherent limitation that such approaches cannot be readily scaled to the high-throughput volumes required for commercial pharmaceutical manufacturing without prohibitive increases in processing time and equipment/process complexity.

Approaches that attempt to combine powders and diluents directly into concentrated suspensions followed by high-shear mixing to disrupt agglomerates (Option 2 of FIG. 1) or that apply agglomerate disruption processing to already-formed concentrated suspensions through specialized equipment such as three-roll mills (Option 3 of FIG. 1) present distinct disadvantages relative to the present invention. When high-shear forces are applied to disrupt agglomerates in concentrated suspensions, the relatively high viscosity characteristic of such compositions prevents effective energy transmission throughout the bulk of the material. The mixing energy imparted by high-shear equipment (e.g., rotor-stator mixing head) becomes localized in areas adjacent to or in direct contact with the mixing head (impeller, etc.), generating excessive heat in these regions while leaving the majority of the composition volume inadequately processed for effective agglomerate disruption. Due to the viscoelastic properties and relatively high solids content of concentrated suspensions (compared to more dilute suspensions of the same powders and diluents), the mechanical energy required for effective agglomerate disruption may not be propagated uniformly throughout the composition, resulting in heterogeneous processing that is far less effective than the same agglomerate disruption forces applied to more dilute suspensions of identical powders and diluents. Post-formation agglomerate disruption approaches (Option 3 of FIG. 1), while potentially effective at disrupting certain agglomerates, require additional unit operations that may increase manufacturing complexity, extend processing times, and may increase risk for contamination in aseptic environments.

In contrast, the present invention (Option 4 of FIG. 1) achieves superior agglomerate disruption by first preparing a relatively dilute suspension in which the lower viscosity generally promotes more uniform transmission and effective utilization of high-shear mixing forces for agglomerate disruption throughout the composition, followed by controlled removal of excess liquid (diluent) phase to concentrate the solids phase through the separation process described herein. As used in this context, the terms “dilute” and “concentrated” are relative terms referring to different solids content levels of the same powder(s) and diluent(s), wherein the dilute suspension contains the same solid phase (powder(s)) and liquid phase (diluent(s)) components at a lower solids content relative to the final concentrated composition, recognizing that the absolute solids content at which any given powder-diluent combination transitions from more dilute suspension behavior to more concentrated viscoelastic behavior is specific to the physicochemical properties of that particular system as described elsewhere herein. This approach maximizes the disruption of agglomerates under optimal rheological conditions while achieving the desired relatively concentrated solids phase, thereby providing a more efficient, scalable, and aseptically compatible solution that addresses both agglomerate reduction and composition concentration. The apparatus configurations, process parameters, and examples that demonstrate the effectiveness of this approach in producing concentrated suspensions (e.g., VES) with improved syringeability characteristics are described in detail in the sections that follow.

The agglomerate disruption step of the present approach may be accomplished using any suitable high-shear mixing equipment capable of generating sufficient mechanical energy to disrupt powder agglomerates in the dilute suspension. Many of these mixing technologies are commonly used and well-established in pharmaceutical manufacturing operations, providing a significant advantage in terms of regulatory acceptance, process validation, and manufacturing implementation. Non-limiting examples of suitable high-shear mixers include, but are not limited to, rotor-stator mixers (such as those available from Silverson Machines, IKA, or equivalent manufacturers), high-pressure homogenizers, microfluidizers, ultrasonic mixers or sonicators, colloid mills, media mills or bead mills, twin-blade planetary mixers (such as those available from Charles Ross & Son Company, Silverson Machines, or equivalent manufacturers), acoustic or resonant acoustic mixers, vibratory mixers, static or inline mixers, or any other mixer or combination of mixers capable of generating sufficient shear to disrupt agglomerates within the suspension. By applying these conventional mixing technologies to dilute suspensions rather than concentrated compositions, the present invention achieves more effective and uniform agglomerate disruption compared to the approaches described above, while leveraging proven pharmaceutical processing equipment. The mixing equipment and associated apparatus components that contact the suspension may be constructed from materials suitable for pharmaceutical manufacturing and processing, including but not limited to stainless steel (such as 316L stainless steel), nickel-titanium alloys, pharmaceutical-grade polymeric materials, ceramic materials, or combinations thereof. The selection of specific mixer types and materials may be based on factors including the nature of the powder and diluent, the required processing scale, compatibility with aseptic manufacturing requirements, cleaning and sterilization considerations, and the physicochemical properties of the active pharmaceutical ingredients present in the composition.

Utilizing this established mixing equipment in the novel processing approach described herein, the present invention addresses these challenges through a novel processing approach comprising high-shear mixing applied to a diluted suspension followed by controlled separation to yield a more concentrated composition (e.g., VES) with improved particle size distribution through reduced agglomerate content. This approach may provide several advantages over conventional methods: the agglomerate disruption occurs in a diluted, fluid system that may be more amenable to efficient mixing and processing; the process may be more readily scalable and compatible with aseptic manufacturing requirements; and the controlled separation step may allow precise adjustment of the final solids content while maintaining the benefits of agglomerate disruption achieved during the mixing phase. This approach may provide concentrated compositions that maintain relatively elevated drug concentrations while exhibiting improved syringeability and reduced propensity for delivery device occlusion, thereby enabling reliable parenteral delivery through fine-gauge needle-syringe combinations and compatibility with a broad range of injection devices, including autoinjectors, pen injectors, and on-body delivery systems as described herein.

The present invention has been described herein by reference to illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. It will be readily apparent to one of ordinary skill in the relevant arts that other suitable modifications and adaptations to the methods and applications described herein may be made without departing from the scope of the invention or any embodiment thereof. Similarly, it will be understood that the benefits and advantages described above may relate to one embodiment or may relate to several embodiments. Having now described the present invention in detail, the same will be more clearly understood by reference to the following examples, which are included for purposes of illustration only and are not intended to be limiting of the invention.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that changes can be made to the specific embodiments which are disclosed herein without departing from the spirit and scope of the invention.

### Example 1: Manufacturing Process Effects on Stability of Spray-Dried Powders Containing Proteins

Earlier work by some of the present inventors established initial processes for preparing injectable paste formulations containing relatively high concentrations of certain proteins and peptides (see, e.g., U.S. Pat. Nos. 8,790,679; 8,110,209; and 9,314,424; and U.S. Patent Publication Nos. US 2017/0007675 and US 2017/0216529, the disclosures of all of which are incorporated herein by reference in their entireties). In the present study, the effects of various manufacturing parameters on the preparation and stability of spray-dried powders and pastes containing high concentrations of antibodies was examined, with the goal to optimize the manufacturing process for use in preparation of storage-stable commercial formulations comprising high concentrations of therapeutic active ingredients, particularly high molecular weight proteins such as antibodies.

Different paste formulations containing spray dried powders containing an IgG antibody were initially prepared by spray-drying aqueous IgG solutions (e.g., aqueous ‘feed solutions’ containing 20 mg/mL of IgG) that contained various carriers and excipients and at differing pH values (as measured prior to spray drying). Details are shown in Table 2 below.

TABLE 2
Representative Powder Compositions

| Formulation 1 | | Formulation 2 | |
|---|---|---|---|
| Component | Concentration | Component | Concentration |
| IgG | 20 mg/mL | IgG | 20 mg/mL |
| Trehalose | 2.5 mg/mL | Trehalose | 5.15 mg/mL |
| Na⁺/K⁺, Ca⁺⁺, Cl⁻ | Total <0.8 mg/mL | Polysorbate 20 | 0.05 mg/mL |
| | | Methionine | 0.412 mg/mL |
| | | Citrate | 0.192 mg/mL |
| pH | 6.3 | pH | 4.0 |

The two formulations were then spray dried using the following settings and under the conditions shown in Table 3.

TABLE 3
Spray Drying Conditions for two Exemplary Formulations

| | Formulation 1 | Formulation 2 |
|---|---|---|
| Inlet temperature | 90° C. | 70° C. |
| Aspirator settings | 65% (27 m³/hr) | 85% (34 m³/hr) |
| Nozzle gas rate | 40 mm (473 L/hr, 667 L/hr, STP | 40 mm (473 L/hr, 667 L/hr, STP |
| Feed solution pump | 3% (1 mL/min) | 10% (~3 mL/min) |
| rate | | |
| Comments | No secondary drying or sieving | Secondary drying and sieving |
| | after spray drying | after spray drying (150 mTorr, |
| | | 5° C., 2 days) |

Unless otherwise specified, all spray drying studies discussed in this application were performed using the B-290 mini-spray dryer manufactured by BUCHI Corporation equipped with the standard two-fluid nozzle. This spray dryer includes a built-in floating ball flowmeter for the nozzle (atomizing) gas flow rate on a scale of 0-60 mm. Aspirator air flow rates and liquid feed pump settings are input on a scale of 0-100% with the B-290 instrument, with the conversion to standard units of air flow (L/hr) and liquid flow (mL/min) provided in the B-290 operating manual.

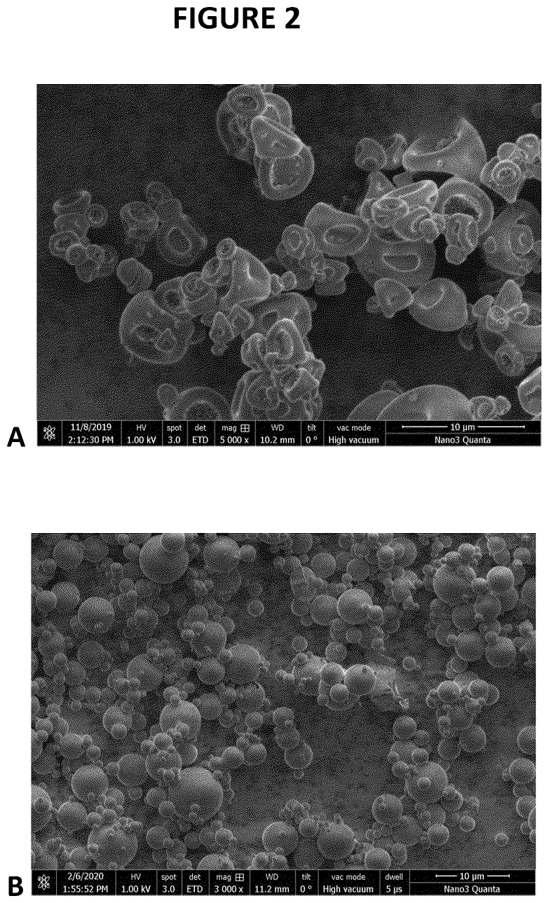

Following preparation of these two spray dried powder formulations, samples were subjected to scanning electron microscopy (SEM) to examine the morphology of the particles of the two formulations. As seen in FIG. 2, SEM micrographs of the two formulations showed distinct differences in the particle morphologies. The particles of Formulation 1 were extensively pitted and showed an irregular shape, while those of Formulation 2 demonstrated much more regular, spherical shape with no apparent pitting. In addition, on average the particles in Formulation 1 were somewhat larger than those in Formulation 2. These physical characteristics together indicate that the particles of Formulation 1 had a higher surface area than those of Formulation 2.

Pastes were then prepared from these two formulations by adding Miglyol® 812 N to them to the point where a paste composition was obtained but prior to conversion of the paste to a suspension, where the relative excess of liquid to solid phase would enable particle settling over time. For Formulation 1 the solids content range where a paste was formed was considerably lower than the range where a paste formed for Formulation 2. Formulation 1 formed a paste in range of approximately 38%-42% w/w solids content, representing a solids concentration of 475 mg/mL solids and an IgG content of approximately 400 mg/mL. In contrast, for formulation 2, a paste containing 65% w/w solids content was prepared, representing approximately 820 mg/mL solids concentration and an IgG concentration of approximately 630 mg/mL. These results suggest that the physical properties of the spray dried powder of Formulation 1 (e.g., larger surface area due to surface rugosity) yielded a lower-concentration paste (both in terms of solids concentration and IgG concentration) for Formulation 1 relative to Formulation 2.

To evaluate the influence of solids content on the injection force needed to deliver the paste through a hypodermic needle (modeling injection of therapeutic paste formulations into an animal), 1 mL of these pastes were loaded into glass syringes (internal diameter ~4.6 mm) and delivered through 27G ultrathin wall ¼ inch (~6 mm exposed length) needle affixed to the syringes via a Luer-lock fitting. The force required to deliver the 1 mL paste in 30 seconds (e.g., 33.3 μL/sec volumetric flow rate) from the syringes was measured using a texture analyzer (force is plotted against the plunger distance traveled). Formulation 1 (42% w/w solids, ~400 mg/mL IgG) required an injection force of about 36N to expel 1 mL of paste in 30 seconds, while an injection force of approximately 60N was required to expel Formulation 2 (65% w/w solids, 630 mg/mL IgG) reflecting in part their differences in overall solids content

A lower injection force may facilitate delivery and improve overall ease-of-administration. Therefore, it would be desirable to produce high active ingredient (e.g., mAb) concentration paste formulations that can be delivered using commercially available syringe/needle combinations at relatively low injection forces. To evaluate the impact of other spray-drying process parameters on the ability to produce stable syringeable therapeutic protein paste formulations, the inventors evaluated the level of protein aggregation in a non-specific IgG formulation pre- and post-spray-drying at different pHs of the initial solution. Aqueous feed solutions of IgG at 20.0 mg/mL were prepared in the “Formulation 2” solution described above in Table 3, but at either pH 4 (using a citrate buffer) or pH 6 (using a histidine buffer). The relative percentage of mAb aggregates post-spray-drying was then determined using size exclusion chromatography. In both formulations, the aggregation level after spray-drying was approximately 2.6% for the pH 6 formulation and 1.9% for the pH 4 formulation, indicating the pH of the starting feed solution may promote a measurable difference in % protein aggregation of the finished spray-dried powder. For certain IgG formulations, the preferred pH for minimizing aggregate formation may be in the range of 3.5-4.0.

To further evaluate the impact of the spray-drying process parameters themselves, a series of experiments were conducted to examine the effects of inlet temperature, aspirator flow rate, and nozzle pressure gas flow rate during the spray-drying process of IgG solutions on the resulting morphology (sphericity), size and size distribution of IgG particles in the resulting spray-dried powder. Eight sets of process parameters were evaluated (Table 4), using a non-specific IgG starting solution at 20 mg/mL in the Formulation 2 solution noted above with a pH of 6.0. All samples were filtered prior to spray-drying, and then subjected to secondary drying under vacuum.

TABLE 4
Spray-Drying Process Parameters

| Formulation | Inlet Temp | Aspirator | Gas Flow | Feed Flow |
|---|---|---|---|---|
| No. | (° C.) | (%) | Rate (mm) | Rate (%) |
| 1 | 90 | 100 | 40 | 10 |
| 2 | 90 | 85 | 60 | 10 |
| 3 | 90 | 85 | 40 | 10 |
| 4 | 90 | 100 | 60 | 10 |
| 5 | 70 | 100 | 40 | 10 |
| 6 | 70 | 85 | 40 | 10 |
| 7 | 70 | 100 | 60 | 10 |
| 8 | 70 | 85 | 60 | 10 |

Following spray-drying, the resultant powders were evaluated visually for appearance, and were then solubilized (dissolved) at 1 mg/mL in water-for-injection (WFI) and evaluated for dissolution time, as well as percentage of aggregation via size exclusion chromatography (SEC). Results are shown in Table 5.

TABLE 5
Effects of Spray-Drying Parameters on Appearance of IgG Powders

| | | | | | IgG | |
|---|---|---|---|---|---|---|
| | | | Gas | Feed | Powder | |
| | Inlet | | Flow | Flow | Dissolution | |
| Formulation | Temp | Aspirator | Rate | Rate | Time | SEC % |
| No. | (C.) | (%) | (mm) | (%) | (1 mg/mL) | Aggregate |
| 1 | 90 | 100 | 40 | 10 | 22-27 | 6.42 |
| 2 | 90 | 85 | 60 | 10 | seconds | 6.35 |
| 3 | 90 | 85 | 40 | 10 | | 6.64 |
| 4 | 90 | 100 | 60 | 10 | | 6.42 |
| 5 | 70 | 100 | 40 | 10 | | 6.28 |
| 6 | 70 | 85 | 40 | 10 | | 6.31 |
| 7 | 70 | 100 | 60 | 10 | | — |
| 8 | 70 | 85 | 60 | 10 | | 6.3 |

As seen in Table 5, powders prepared from the different feed solutions evaluated in this study contained a similar percentage of measured protein aggregation upon dissolution, with each of the powders also demonstrating approximately similar dissolution times in the close range of 22-27 seconds.

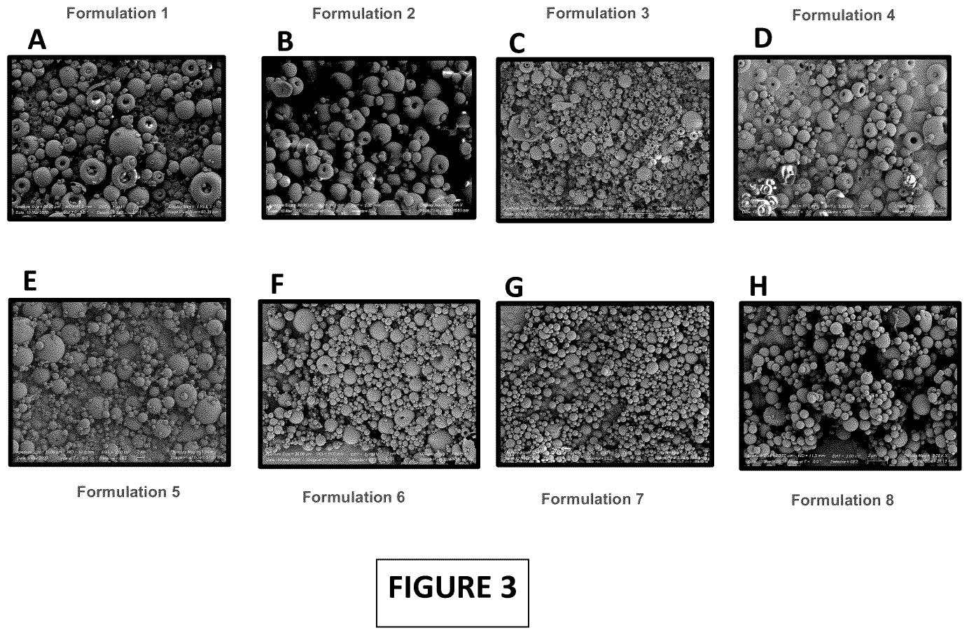

To more closely examine these powders at the particle level, a sample of powder from each of the eight formulations described in Table 5 was examined using scanning electron microscopy (SEM), to evaluate particle size, particle shape, and the distribution of each in a given sample. Results are shown in FIG. 3.

As seen in FIGS. 3A-3D, formulations 5-8, all of which used a spray-dryer inlet temperature of 90° C., exhibited a variety of particle sizes and particle morphologies, with a mixture of small and large particles demonstrating a mixture of spherical and toroidal shapes. In contrast, as seen in FIGS. 3E-3H, Formulations 5-8, all of which used a spray-dryer inlet temperature of 70° C., showed more uniform particles in terms of size and shape, with most particles appearing to be spherical and only a very small number of toroid particles observed. Of these formulations, Formulations 7 and 8 appeared to be the most uniform in size, demonstrating a preponderance of small spherical particles.

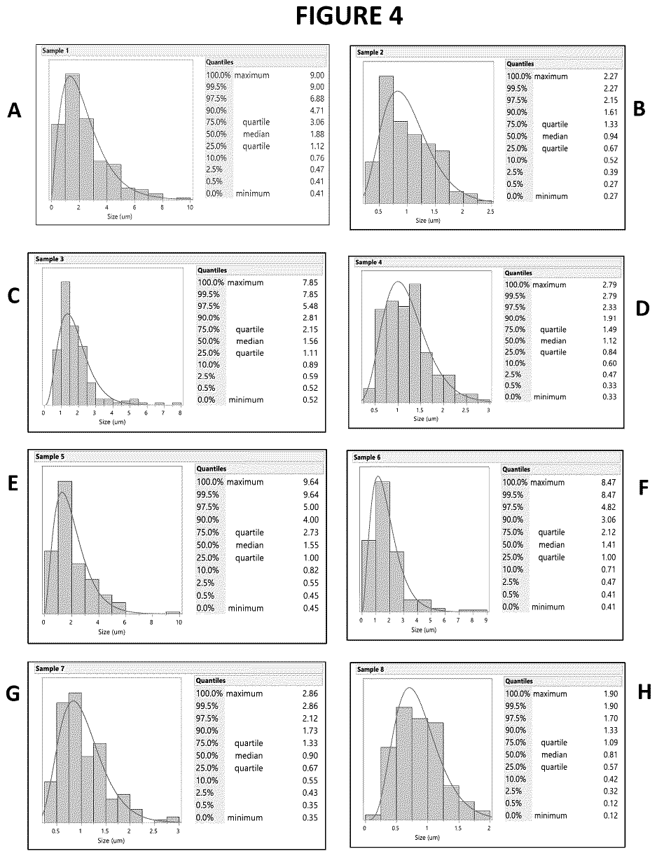

These results were confirmed when particle size distribution was measured, by individually measuring the diameter (on representative SEM photomicrographs) of about 200 IgG particles per formulation; results of these measurements are shown in FIG. 4. As seen in FIG. 4, Formulations 1-4 which used a spray-dryer inlet temperature of 90° C. exhibited a larger particle size distribution (FIGS. 4A-4D) compared to Formulations 5-8 which used a spray-dryer inlet temperature of 70° C. (FIGS. 4E-4H). Interestingly, at a given spray-dryer inlet temperature, a higher gas flow rate tended to favor the production of smaller and more uniform particles; compare, for example, those formulations dried at a 40 mm gas flow rate (FIGS. 4A, 4C, 4E and 4F) to their corresponding formulations dried at a 60 mm gas flow rate (FIGS. 4B, 4D, 4G and 4H, respectively).

Taken together, the results of these studies indicate that the spray-drying settings can influence the production of spherical particles with the appropriate size distribution. In particular, an inlet temperature of about 70-90° C., and more particularly about 70-80° C., and a gas flow rate of about 40-60 mm, and more particularly about 60 mm, appeared to produce smaller and more spherical particles in the powder. In fact, it was determined that on the B-290 spray dryer an inlet temperature of about 70° C. is the lowest temperature that can be used to obtain spherical particles; using a lower temperature with the feed solution and process parameters described in this Example, with the expectation that the protein or peptide might be less prone to temperature-induced denaturation, actually had the effect of producing more toroidal particles of nonuniform size distribution (data not shown). The ability to produce smaller spherical particles with an appropriate size distribution is important for the ultimate production of high solids content, high protein concentration pastes that may be suitably injected in relatively small volumes into animals, particularly humans, for therapeutic and diagnostic purposes.

### Example 2: Effects of Formulation Excipients on Production and Injectability of Spray-Dried Proteins

Beyond the spray-dryer settings, results from the present inventors indicated that the components of the formulation that is spray-dried can have an impact on the physical and performance properties of the spray-dried powder prepared from a given formulation. To further examine and optimize these formulation component effects, a representative monoclonal antibody (trastuzumab or “TmAb”, the API in the commercial drug product Herceptin®) was formulated in the presence of a variety of excipients and buffering agents, pharmaceutically acceptable carriers or bulking agents, surfactants, etc., and the impact of each excipient on the level of aggregation of the formulation both at time 0 after powder manufacture by spray-drying and upon storage of the powder was evaluated. TmAb is a protein that in monomeric form is 148 kDa in size, but upon aggregation it forms dimers and other multimers of larger molecular weight which are themselves not only immunogenic but also serve as nucleation centers for the formation of even larger aggregates in solution; such aggregates can be immunogenic and/or be cleared by the immune system before the antibody has a chance to exert it therapeutic effect. Therefore, it would be ideal from a therapeutic standpoint to be able to prepare powders (e.g., via spray drying) containing TmAb and having low levels of aggregation upon storage, which can then be suitably used in high solids content/high concentration pastes for injection into animals including humans.

For preparation, a commercial TmAb solution was dialyzed (50 kDa molecular weight cutoff) against the desired formulation buffer (see table below) overnight at 4° C. with constant stirring. The dialyzed TmAb solution was then spray-dried using an inlet temperature of 70° C., a nozzle flow rate of 40 mm, 85% aspirator setting and 10% feed pump (about 3 mL/min). Once the powder had been produced, to lower the moisture content to a target level of <1% (w/w) the powder was secondarily dried (e.g., under vacuum) at 150 mT, 5° C. for one day, then at 30° C. for three hours, and the powder was then stored in glass vials backfilled with nitrogen and stoppered to produce a closed system. Samples were stored at 40° C. for four days or at 50° C. for four hours and compared to t=0 samples (immediately after lyophilization) for degree of aggregation and other stability parameters by solubilizing the powder sample to a concentration of 1 mg/ml TmAb in water and then analyzing the solutions by size exclusion, ion exchange and reverse phase chromatography.

In a first round of experiments, five formulations were prepared each containing 20 mg/mL TmAb, trehalose at 5.15 mg/mL, polysorbate 20 at 0.05 mg/mL, methionine at 0.412 mg/mL, as well as the remaining excipients and at the pH adjusted to the values shown in Table 6.

TABLE 6
pH and Buffer Species Formulations (Round 1)

| | Feed | | Feed | | Feed | | Feed | | Feed | |
|---|---|---|---|---|---|---|---|---|---|---|
| | Soln. | Powder | Soln. | Powder | Soln. | Powder | Soln. | Powder | Soln. | Powder |
| | Batch | Batch | Batch | Batch | Batch | Batch | Batch | Batch | Batch | Batch |
| | TF2 | TF2 | TF3 | TF3 | TF4 | TF4 | TF5 | TF5 | TF6 | TF6 |
| TmAb | 20.0 | 77.51% | 20.0 | 77.65% | 20.0 | 77.65% | 20.0 | 77.81% | 20.0 | 77.81% |
| | mg/mL | | mg/mL | | mg/mL | | mg/mL | | mg/mL | |
| TmAb | — | 565.4 | — | 572.9 | — | 572.9 | — | 574.1 | — | 574.1 |
| mg/mL | | mg/mL | | mg/mL | | mg/mL | | mg/mL | | mg/mL |
| in paste | | | | | | | | | | |
| (assume | | | | | | | | | | |
| 60% | | | | | | | | | | |
| w/w | | | | | | | | | | |
| load) | | | | | | | | | | |
| Citrate | 0.192 | 0.74% | — | — | — | — | — | — | — | — |
| | mg/mL | | | | | | | | | |
| Adipate | — | — | 0.146 | 0.57% | 0.146 | 0.57% | — | — | — | — |
| | | | mg/mL | | mg/mL | | | | | |
| Lactate | — | — | — | — | — | — | 0.090 | 0.35% | 0.090 | 0.35% |
| | | | | | | | mg/mL | | mg/mL | |
| pH | 4.0 | — | 4.0 | — | 5.0 | — | 4.0 | — | 5.0 | — |
| Total | 25.80 | 100% | 25.76 | 100% | 25.76 | 100% | 25.70 | 100% | 25.70 | 100% |
| | mg/mL | | mg/mL | | mg/mL | | mg/mL | | mg/mL | |

These formulations were then evaluated for level of protein aggregation in the pre-spray-dried formulation and in the solubilized spray-dried powder, via size exclusion chromatography. Results are shown in Table 7.

TABLE 7
Effect of pH and Buffer Species on TmAb Aggregation (Round 1)

| | % Aggregation, | % Aggregation, |
|---|---|---|
| | Pre-spray-drying | Post-spray-drying |
| pH 4.0, Citrate (TF2) | 0.48 | 0.61 |
| pH 4.0, Adipate (TF3) | 0.46 | 0.62 |
| pH 5.0, Adipate (TF4) | 0.46 | 0.63 |
| pH 4.0, Lactate (TF5) | 0.49 | 0.67 |
| pH 5.0, Lactate (TF6) | 0.54 | 0.66 |

To expand upon these studies, a second round of formulations were prepared containing the same levels of TmAb, trehalose and polysorbate 20 as described for Round 1 above, but containing either succinate or lactate buffer and at a pH of 4-6, as shown in Table 8:

TABLE 8
pH and Buffer Species Formulations (Round 2).

| | Feed | | Feed | | Feed | | Feed | |
|---|---|---|---|---|---|---|---|---|
| | Soln. | Powder | Soln. | Powder | Soln. | Powder | Soln. | Powder |
| | Batch | Batch | Batch | Batch | Batch | Batch | Batch | Batch |
| | TF26 | TF26 | TF27 | TF27 | TF28 | TF28 | TF29 | TF29 |
| TmAb | 20.0 | 77.51% | 20.0 | 77.65% | 20.0 | 77.65% | 20.0 | 77.81% |
| | mg/mL | | mg/mL | | mg/mL | | mg/mL | |
| TmAb | — | 562.4 | — | 562.4 | — | 562.4 | — | 565.4 |
| mg/mL | | mg/mL | | mg/mL | | mg/mL | | mg/mL |
| in paste | | | | | | | | |
| (assume | | | | | | | | |
| 60% | | | | | | | | |
| w/w | | | | | | | | |
| load) | | | | | | | | |
| Sucinate | 0.590 | 2.25% | 0.590 | 2.25% | 0.590 | 2.25% | — | — |
| | mg/mL | | mg/mL | | mg/mL | | | |
| Lactate | — | — | — | — | — | — | 0.450 | 1.72% |
| | | | | | | | mg/mL | |
| pH | 4.0 | — | 5.0 | — | 6.0 | — | 4.0 | — |
| Total | 26.24 | 100% | 26.24 | 100% | 26.24 | 100% | 26.10 | 100% |
| | mg/mL | | mg/mL | | mg/mL | | mg/mL | |

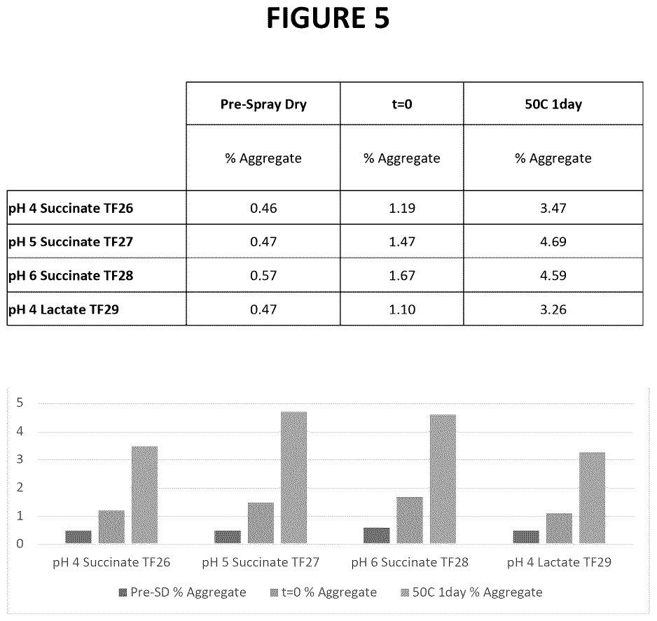

These formulations were then evaluated for level of protein aggregation in the pre-spray-dried formulation and in the solubilized spray-dried powder, via size exclusion chromatography, comparing samples tested pre-spray-drying and those pulled at t=0 after spray-drying and others stored at 50° C. for one day. Results are shown in FIG. 5. Together with those from the Round 1 formulations, these results indicate that the optimal buffer and pH conditions for the pre-spray-dry solution, in order to minimize the amount of aggregation in both the pre-sprayed solution and in the post-sprayed and dried powder, is the use of a lactate buffer and a pH of from about 3.5 to about 4.5, e.g., about 4.0, particularly for IgG-containing formulations.

Next, the impact of trehalose amounts in the pre-spray-dry formulation on the aggregation in solution and in the post-spray-dried powder was evaluated. TmAb formulations (20 mg/mL) in a 5 mM lactate buffer, pH 4.0 or 6.0, were prepared, containing the amounts of trehalose and other excipients shown in Table 9:

TABLE 9
Formulations with varying sugar content

| | Feed | | Feed | | Feed | |
|---|---|---|---|---|---|---|
| | Soln. | Powder | Soln. | Powder | Soln. | Powder |
| | Batch | Batch | Batch | Batch | Batch | Batch |
| | TF30 | TF30 | TF31 | TF31 | TF32 | TF32 |
| TmAb | 20.0 | 76.63% | 20.0 | 64.62% | 20.0 | 55.63% |
| | mg/mL | | mg/mL | | mg/mL | |
| TmAb | — | 565.4 | — | 476.8 | — | 410.4 |
| mg/mL in | | mg/mL | | mg/mL | | mg/mL |
| paste | | | | | | |
| (assume | | | | | | |
| 60% w/w | | | | | | |
| load) | | | | | | |
| Trehalose | 5.15 | 19.73% | 10.0 | 32.31% | 15 | 41.72% |
| | mg/mL | | mg/mL | | mg/mL | |
| Polysorbate | 0.5 | 1.92% | 0.5 | 1.62% | 0.5 | 1.39% |
| 20 | mg/mL | | mg/mL | | mg/mL | |
| Lactate | 0.45 | 1.72% | 0.45 | 1.45% | 0.45 | 1.25% |
| | mg/mL | | mg/mL | | mg/mL | |
| pH | 4.0 | — | 4.0 | — | 4.0 | — |
| Total | 26.10 | 100% | 30.95 | 100% | 35.95 | 100% |
| | mg/mL | | mg/mL | | mg/mL | |
| | | | | | Feed Soln. | Powder |
| | Feed | | Feed | | Batch TF25 | Batch TF25 |
| | Soln. | Powder | Soln. | Powder | (Batch TF7, | (Batch TF7, |
| | Batch | Batch | Batch | Batch | post-SD | post-SD |
| | TF7 | TF7 | TF13 | TF13 | lyophilized) | lyophilized) |
| TmAb | 20.0 | 51.28% | 20.0 | 32.88% | 20.0 | 51.28% |
| | mg/mL | | mg/mL | | mg/mL | |
| TmAb | — | 378.4 | — | 242.6 | — | 378.4 |
| mg/mL in | | mg/mL | | mg/mL | | mg/mL |
| paste | | | | | | |
| (assume | | | | | | |
| 60% w/w | | | | | | |
| load) | | | | | | |
| Trehalose | 18.18 | 46.61% | 40 | 65.76% | 18.18 | 46.61% |
| | mg/mL | | mg/mL | | mg/mL | |
| Polysorbate | 0.081 | 0.21% | 0.081 | 0.13% | 0.081 | 0.21% |
| 20 | mg/mL | | mg/mL | | mg/mL | |
| Histidine | 0.743 | 1.90% | 0.743 | 1.22% | 0.743 | 1.90% |
| | mg/mL | | mg/mL | | mg/mL | |
| pH | 6.0 | — | 6.0 | — | 6.0 | — |
| Total | 39.00 | 100% | 60.82 | 100% | 39.00 | 100% |
| | mg/mL | | mg/mL | | mg/mL | |

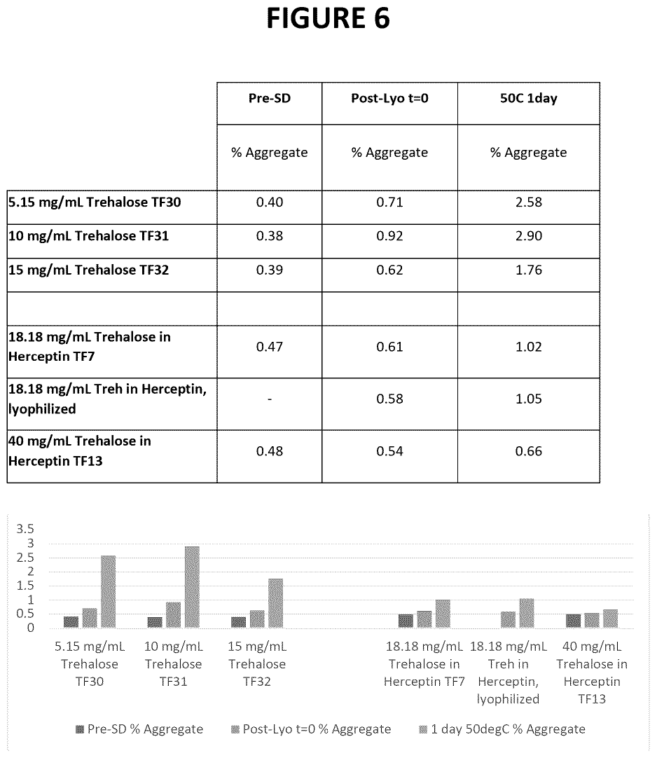

These formulations were then evaluated for level of protein aggregation in the pre-spray-dried aqueous formulation (feed solution) and in the powder post-spray drying, via size exclusion chromatography, comparing samples tested pre-spray-drying and those pulled at t=0 after spray-drying and lyophilization, and others stored at 50° C. for one day. Results are shown in FIG. 6. These results indicate that the trehalose has a stabilizing effect upon spray-drying and post-spray-drying storage, in terms of reducing the aggregation of the powder. However, relatively high amounts of trehalose are required in the formulation to achieve such stability, which thereby reduces the active content (in this case, TmAb) in the paste formulation. Thus, the amount of trehalose to be included must be optimized along with other excipients and formulation components in order to enhance stability while also permitting a relatively high active ingredient content to be included in the paste formulations. The present inventors also have results suggesting that for the formulations prepared under the conditions of this Example, using sucrose in place of trehalose, at similar concentrations, may provide a greater stabilization effect on the powdered formulations. Other excipients that can be advantageously used in a similar way include amino acids, advantageously one or more naturally occurring amino acids, such as hydrophobic amino acids (which may help prevent the hydrophobic core of one TmAb molecule from binding to a second TmAb, thus reducing aggregation), acidic/basic amino acids such as arginine which has a reported ability to stabilize protein and peptide formulations, even in the dry state, and a combination of sugars such as dextran/trehalose coformulations.

Continuing with these studies, the effect of polysorbate 20 content in the pre-spray-dry formulation on the aggregation in the feed solution and in the post-spray-dried powder was evaluated. In the initial round of these studies, TmAb formulations (20 mg/mL) in a 4.8 mM histidine buffer at pH 6.0 were prepared, containing the polysorbate 20 and other excipient contents shown in Table 10:

TABLE 10
Evaluation of Polysorbate 20 content

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF20 | TF20 | TF22 | TF22 | TF23 | TF23 | TF24 | TF24 |
| TmAb | 20.0 | 75.78% | 20.0 | 76.94% | 20.0 | 71.70% | 20.0 | 66.90% |
| | mg/mL | | mg/mL | | mg/mL | | mg/mL | |
| TmAb | — | 559.1 | — | 567.7 | — | 529.0 | — | 493.6 |
| mg/mL in | | mg/mL | | mg/mL | | mg/mL | | mg/mL |
| paste | | | | | | | | |
| (assume | | | | | | | | |
| 60% w/w | | | | | | | | |
| load) | | | | | | | | |
| Trehalose | 5.15 | 19.51% | 5.15 | 19.81% | 5.15 | 18.46% | 5.15 | 17.23% |
| | mg/mL | | mg/mL | | mg/mL | | mg/mL) | |
| Polysorbate | 0.5 | 1.89% | 0.1 | 0.38% | 2 | 7.17% | 4 | 13.38% |
| 20 | mg/mL | | mg/mL | | mg/mL | | mg/mL | |
| Histidine | 0.743 | 2.82% | 0.743 | 2.86% | 0.743 | 2.66% | 0.743 | 2.49% |
| | mg/mL | | mg/mL | | mg/mL | | mg/mL | |
| pH | 6.0 | — | 6.0 | — | 6.0 | — | 6.0 | — |
| Total | 26.39 | 100% | 25.99 | 100% | 27.89 | 100% | 29.89 | 100% |
| | mg/mL | | mg/mL | | mg/mL | | mg/mL | |

Other formulations containing either Polysorbate 20 or Polysorbate 80 were also prepared as shown in Table 11:

TABLE 11
Polysorbate Formulations (continued)

| | Feed Solution | Powder Batch | Feed Solution | Powder Batch |
|---|---|---|---|---|
| | Batch TF13 | TF13 | Batch TF14 | TF14 |
| TmAb | 20.0 mg/mL | 32.88% | 20.0 mg/mL | 32.88% |
| TmAb mg/mL | — | 242.6 mg/mL | — | 242.6 mg/mL |
| in paste | | | | |
| (assume | | | | |
| 60% w/w load) | | | | |
| Trehalose | 40 mg/mL | 65.76% | 40 mg/mL | 65.76% |
| Polysorbate 20 | 0.081 mg/mL | 0.13% | — | — |
| Polysorbate 80 | — | — | 0.081 mg/mL | 0.13% |
| Histidine | 0.743 mg/mL | 1.22% | 0.743 mg/mL | 1.22% |
| pH | 6.0 | — | 6.0 | — |
| Total | 60.82 mg/mL | 100% | 60.82 mg/mL | 100% |

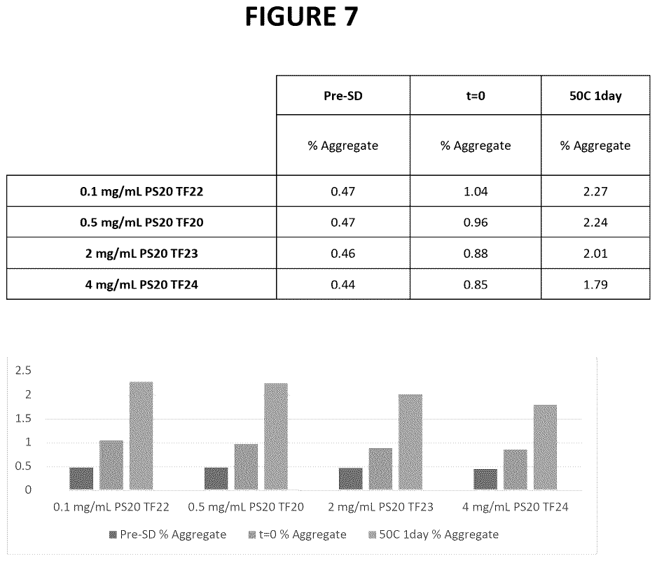

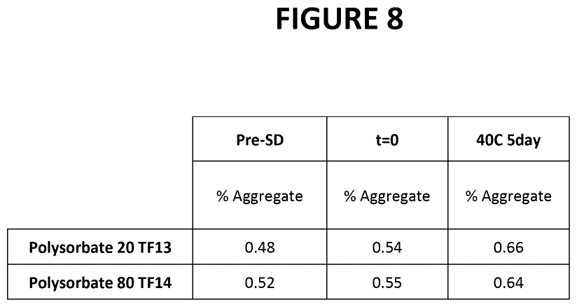

These formulations were then evaluated for level of protein aggregation in the pre-spray-dried aqueous formulation and in the solubilized spray-dried powder, via size exclusion chromatography, comparing samples tested pre-spray-drying and those pulled at Time 0 (e.g., within 1 day) after spray-drying and lyophilization, and others stored at 50° C. for one day. Results are shown in FIGS. 7-8. These results indicate that the polysorbate has a stabilizing effect upon spray-drying and post-spray-drying storage stability (i.e., reducing the aggregation of the powder). Under the conditions evaluated in this study, there did not appear to be a significant difference between polysorbate 20 and polysorbate 80 in stabilization of the powders. Moreover, only minor incremental gains in stability are seen with increasing amounts of polysorbate added to the pre-spray dry formulations; about 0.5 mg/mL of polysorbate 20 appears to provide a suitable improvement in stabilization without adding too much solids mass to the formulation (which would, as noted above for trehalose, negatively impact (dilute) the amount of active, in this case TmAb, that could be included in the paste formulation)

Next, the inclusion of various amino acids as excipients was evaluated for their effects upon aggregation and storage stability. First, various methionine-containing formulations were prepared according to the component amounts shown in Table 12:

TABLE 12
Methionine-containing Formulations

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF30 | TF30 | TF33 | TF33 | TF34 | TF34 | TF35 | TF35 |
| TmAb | 20.0 | 76.63% | 20.0 | 72.46% | 20.0 | 68.73% | 20.0 | 62.31% |
| | mg/mL | | mg/mL | | mg/mL | | mg/mL | |
| TmAb | — | 565.4 | — | 534.6 | — | 507.1 | — | 459.7 |
| mg/mL in | | mg/mL | | mg/mL | | mg/mL | | mg/mL |
| paste | | | | | | | | |
| (assume | | | | | | | | |
| 60% w/w | | | | | | | | |
| load) | | | | | | | | |
| Trehalose | 5.15 | 19.73% | 5.15 | 18.66% | 5.15 | 17.70% | 5.15 | 16.04% |
| | mg/mL | | mg/mL | | mg/mL | | mg/mL | |
| Polysorbate | 0.5 | 1.92% | 0.5 | 1.81% | 0.5 | 1.72% | 0.5 | 1.56% |
| 20 | mg/mL | | mg/mL | | mg/mL | | mg/mL | |
| Methionine | — | — | 1.5 | 5.43% | 3 | 10.31% | 6 mg/mL | 18.69% |
| | | | mg/mL | | mg/mL | | | |
| Lactate | 0.450 | 1.72% | 0.450 | 1.63% | 0.450 | 1.55% | 0.450 | 1.40% |
| | mg/mL | | mg/mL | | mg/mL | | mg/mL | |
| | (5 mM) | | | | | | | |
| pH | 4.0 | — | 4.0 | — | 4.0 | — | 4.0 | — |
| Total | 26.10 | 100% | 27.60 | 100% | 29.10 | 100% | 32.10 | 100% |
| | mg/mL | | mg/mL | | mg/mL | | mg/mL | |

In a similar fashion, the inclusion of proline or glycine was evaluated in formulations prepared as shown in Tables 13 and 14:

TABLE 13
Proline/Glycine-containing Formulations

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF30 | TF30 | TF40 | TF40 | TF41 | TF41 | TF42 | TF42 |
| TmAb | 20.0 | 76.63% | 20.0 | 55.40% | 20.0 | 55.40% | 20.0 | 43.38% |
| | mg/mL | | mg/mL | | mg/mL | | mg/mL | |
| TmAb | — | 565.4 | — | 408.8 | — | 408.8 | — | 320.1 |
| mg/mL in | | mg/mL | | mg/mL | | mg/mL | | mg/mL |
| paste | | | | | | | | |
| (assume | | | | | | | | |
| 60% w/w | | | | | | | | |
| load) | | | | | | | | |
| Trehalose | 5.15 | 19.73% | 5.15 | 14.27% | 5.15 | 14.27% | 5.15 | 11.17% |
| | mg/mL | | mg/mL | | mg/mL | | mg/mL | |
| Polysorbate | 0.5 | 1.92% | 0.5 | 1.39% | 0.5 | 1.39% | 0.5 | 1.08% |
| 20 | mg/mL | | mg/mL | | mg/mL | | mg/mL | |
| Proline | — | — | 10 | 27.70% | — | — | 10 | 21.69% |
| | | | mg/mL | | | | mg/mL | |
| Glycine | — | — | — | — | 10 mg/mL | 27.70% | 10 | 21.69% |
| | | | | | | | mg/mL | |
| Lactate | 0.450 | 1.72% | 0.450 | 1.25% | 0.450 | 1.25% | 0.450 | 0.98% |
| | mg/mL | | mg/mL | | mg/mL | | mg/mL | |
| pH | 4.0 | — | 4.0 | — | 4.0 | — | 4.0 | — |
| Total | 26.10 | 100% | 36.10 | 100% | 36.10 | 100% | 46.10 | 100% |
| | mg/mL | | mg/mL | | mg/mL | | mg/mL | |

TABLE 14
Proline-containing Formulations (continued)

| | Feed | | Feed | | Feed | |
|---|---|---|---|---|---|---|
| | Solution | Powder | Solution | Powder | Solution | Powder |
| | Batch | Batch | Batch | Batch | Batch | Batch |
| | TF30 | TF30 | TF49 | TF49 | TF50 | TF50 |
| TmAb | 20.0 | 76.63% | 20.0 | 62.31% | 20.0 | 55.40% |
| | mg/mL) | | mg/mL | | mg/mL | |
| TmAb | — | 565.4 | — | 459.7 | — | 408.8 |
| mg/mL in | | mg/mL | | mg/mL | | mg/mL |
| paste | | | | | | |
| (assume | | | | | | |
| 60% w/w | | | | | | |
| load) | | | | | | |
| Trehalose | 5.15 | 19.73% | 5.15 | 16.04% | 5.15 | 14.27% |
| | mg/mL | | mg/mL | | mg/mL | |
| Polysorbate | 0.5 | 1.92% | 0.5 | 1.56% | 0.5 | 1.39% |
| 20 | mg/mL | | mg/mL | | mg/mL | |
| Proline | — | — | 6 | 18.69% | 10 | 27.70% |
| | | | mg/mL | | mg/mL | |
| Lactate | 0.450 | 1.72% | 0.450 | 1.40% | 0.450 | 1.25% |
| | mg/mL | | mg/mL | | mg/mL | |
| pH | 4.0 | — | 4.0 | — | 4.0 | — |
| Total | 26.10 | 100% | 32.1 | 100% | 36.1 | 100% |
| | mg/mL | | mg/mL | | mg/mL | |

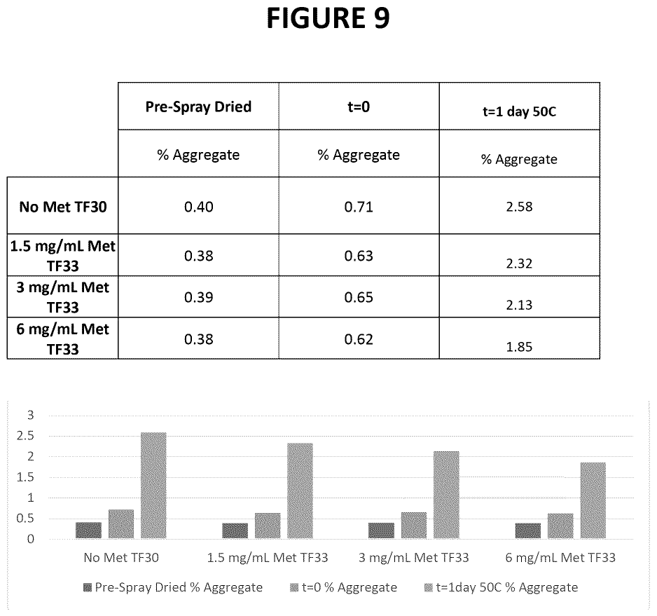

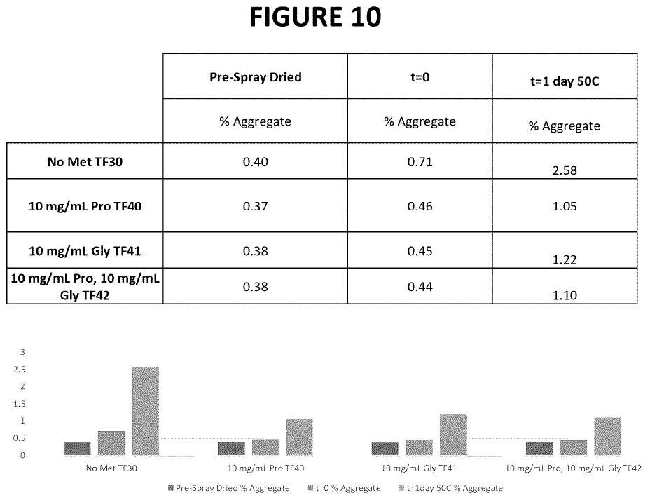

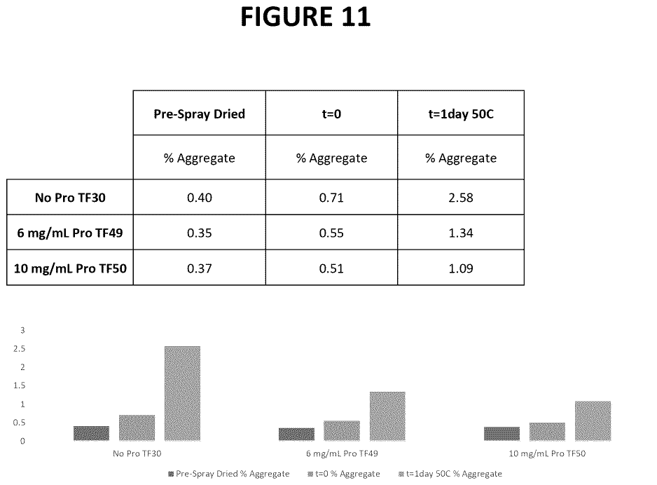

These formulations were then evaluated for level of aggregation in the pre-spray-dried formulation and in the solubilized spray-dried powder, via size exclusion chromatography, comparing samples tested pre-spray-drying and those samples after spray-drying and lyophilization (Time 0 (T0) samples), and others stored at 50° C. for one day. Results are shown in FIG. 9 (for methionine), FIG. 10 (for proline/glycine Round 1), and FIG. 11 (for proline Round 2). Taken together, these results indicate that while methionine (FIG. 9), proline (FIGS. 10 and 11) and glycine (FIG. 10) all act as stabilizing excipients, under the conditions evaluated in this study, relatively high amounts of these amino acids may be required to see more than just marginal impact upon stability. Since increased content of these excipients in the feed solution (while holding mAb content relatively constant) would dilute the active ingredient in the powder and resulting paste formulations, these amino acids may not be favored excipients for these formulations produced and evaluated under the conditions of this Example, as the reduction in active content in these formulations in exchange for only a minor increase in stability is not worth the trade-off.

Finally, formulations comprising cysteine at various concentrations were prepared according to Tables 15, 16, and 17:

TABLE 15
Cysteine-containing Formulations

| | Feed | | Feed | | Feed | |
|---|---|---|---|---|---|---|
| | Solution | Powder | Solution | Powder | Solution | Powder |
| | Batch | Batch | Batch | Batch | Batch | Batch |
| | TF36 | TF36 | TF37 | TF37 | TF38 | TF38 |
| TmAb | 20.0 | 72.46% | 20.0 | 55.40% | 20.0 | 48.66% |
| | mg/mL | | mg/mL | | mg/mL | |
| | (0.135 mM) | | (0.135 mM) | | (0.135 mM) | |
| TmAb | — | 534.6 | — | 408.8 | — | 359.0 |
| mg/mL in | | mg/mL | | mg/mL | | mg/mL |
| paste | | | | | | |
| (assume | | | | | | |
| 60% w/w | | | | | | |
| load) | | | | | | |
| Trehalose | 5.15 | 18.66% | 5.15 | 14.27% | 5.15 | 12.53% |
| | mg/mL | | mg/mL | | mg/mL | |
| | (15.0 mM) | | (15.0 mM) | | (15.0 mM) | |
| Polysorbate | 0.5 | 1.81% | 0.5 | 1.39% | 0.5 | 1.22% |
| 20 | mg/mL | | mg/mL | | mg/mL | |
| Cysteine | 1.5 | 5.43% | 10 | 27.70% | 15 | 36.50% |
| | mg/mL | | mg/mL | | mg/mL | |
| Lactate | 0.450 | 1.63% | 0.450 | 1.25% | 0.450 | 1.09% |
| | mg/mL | | mg/mL | | mg/mL | |
| | (5 mM) | | (5 mM) | | (5 mM) | |
| pH | 4.0 | — | 4.0 | — | 4.0 | — |
| Total | 27.60 | 100% | 36.10 | 100% | 41.10 | 100% |
| | mg/mL | | mg/mL | | mg/mL | |

TABLE 16
Cysteine-containing Formulations (continued)

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF43 | TF43 | TF44 | TF44 | TF45 | TF45 | TF46 | TF46 |
| TmAb | 20.0 | 72.46% | 20.0 | 68.73% | 20.0 | 62.31% | 20.0 | 55.40% |
| | mg/mL | | mg/mL | | mg/mL | | mg/mL | |
| TmAb | — | 534.6 | — | 507.1 | — | 459.7 | — | 408.8 |
| mg/mL in | | mg/mL | | mg/mL | | mg/mL | | mg/mL |
| paste | | | | | | | | |
| (assume | | | | | | | | |
| 60% w/w | | | | | | | | |
| load) | | | | | | | | |
| Trehalose | 5.15 | 18.66% | 5.15 | 17.70% | 5.15 | 16.04% | 5.15 | 14.27% |
| | mg/mL | | mg/mL | | mg/mL | | mg/mL | |
| Polysorbate | 0.5 | 1.81% | 0.5 | 1.72% | 0.5 | 1.56% | 0.5 | 1.39% |
| 20 | mg/mL | | mg/mL | | mg/mL | | mg/mL | |
| Cysteine | 1.5 | 5.43% | 3 | 10.31% | 6 | 18.69% | 10 | 27.70% |
| | mg/mL | | mg/mL | | mg/mL | | mg/mL | |
| Lactate | 0.450 | 1.63% | 0.450 | 1.55% | 0.450 | 1.40% | 0.450 | 1.25% |
| | mg/mL | | mg/mL | | mg/mL | | mg/mL | |
| pH | 4.0 | — | 4.0 | — | 4.0 | — | 4.0 | — |
| Total | 27.6 | 100% | 29.1 | 100% | 32.1 | 100% | 36.1 | 100% |
| | mg/mL | | mg/mL | | mg/mL | | mg/mL | |

TABLE 17
Cysteine-containing Formulations (continued)

| | Feed | Powder | Feed Solution | Powder |
|---|---|---|---|---|
| | Solution | Batch | Batch | Batch |
| | Batch TF47 | TF47 | TF48 | TF48 |
| TmAb | 20.0 mg/mL | 61.68% | 20.0 mg/mL | 54.91% |
| | (0.135 mM) | | (0.135 mM) | |
| TmAb mg/mL in | — | 455.1 | — | 405.1 |
| paste (assume | | mg/mL | | mg/mL |
| 60% w/w load) | | | | |
| Trehalose | 5.15 mg/mL | 15.88% | 5.15 mg/mL | 14.14% |
| | (15.0 mM) | | (15.0 mM) | |
| Polysorbate 20 | 0.5 mg/mL | 1.54% | 0.5 mg/mL | 1.37% |
| Cysteine | 6 mg/mL | 18.50% | 10 mg/mL | 27.45% |
| Proline | — | — | — | — |
| Lactate | — | — | — | — |
| Histidine | 0.774 mg/mL | 2.39% | 0.774 mg/mL | 2.12% |
| | (5 mM) | | (5 mM) | |
| pH | 6.0 | — | 6.0 | — |
| Total | 32.42 mg/mL | 100% | 36.42 mg/mL | 100% |

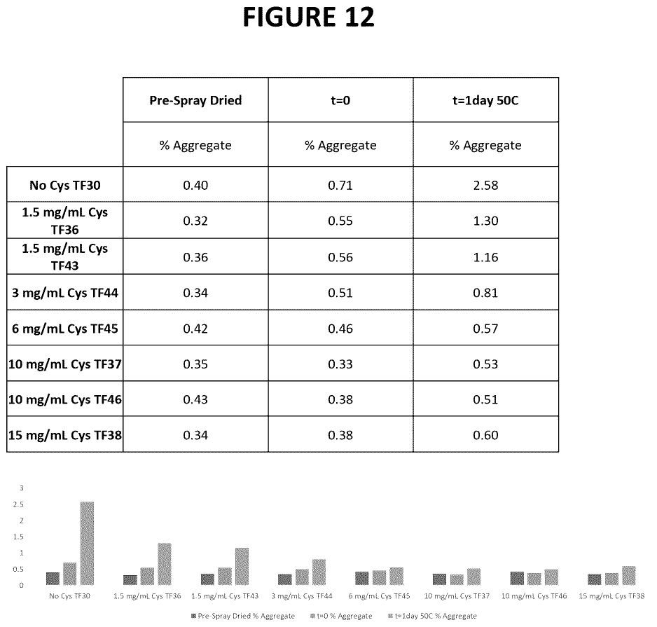

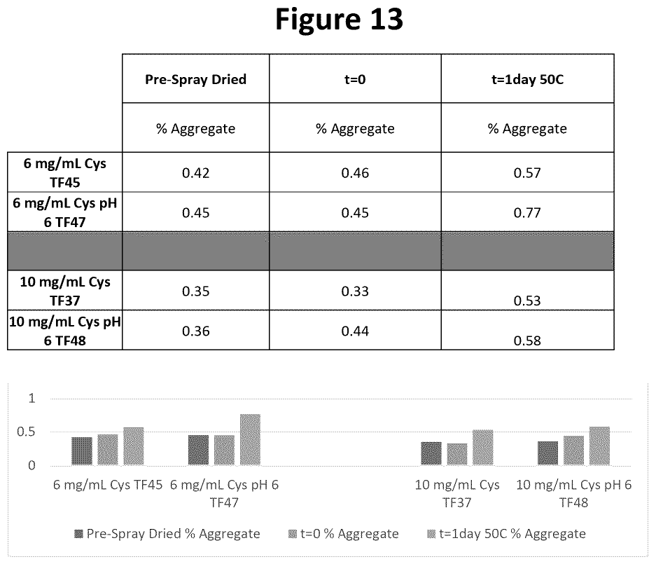

These formulations were then evaluated for mAb aggregation in the pre-spray-dried formulation and in the solubilized spray-dried powder, via size exclusion chromatography, comparing samples tested pre-spray-drying and those pulled at t=0 after spray-drying and lyophilization, and others stored at 50° C. for one day. Results are shown in FIGS. 12-13, and suggest that including cysteine even at low amounts in the pre-spray-dried formulation may have a strongly positive effect on storage stability of the spray-dried powders prepared under the conditions described in this example.

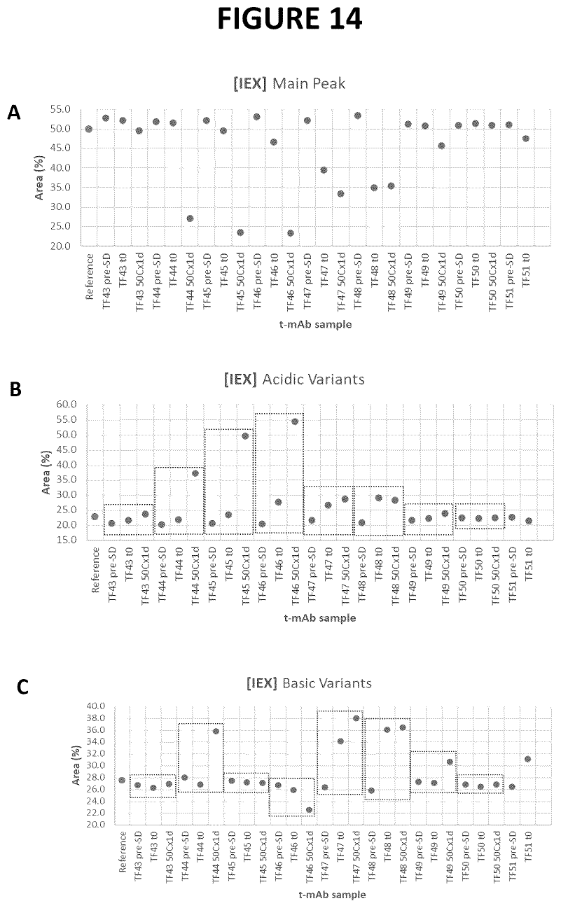

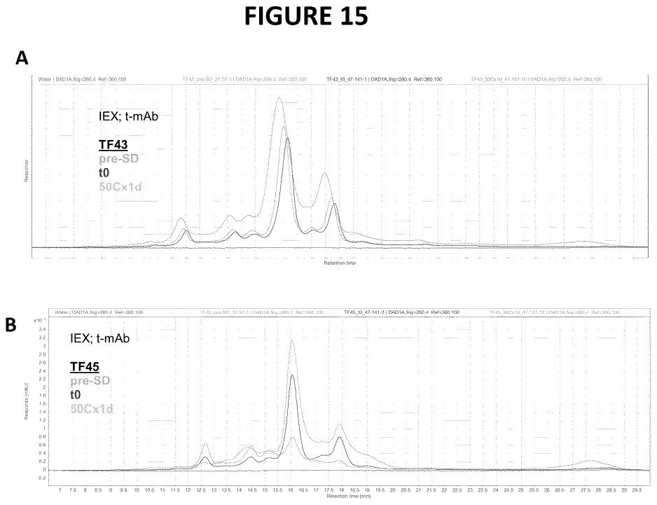

To further evaluate the effects of cysteine, powders prepared from various cysteine-containing formulations were dissolved and then analyzed by ion exchange chromatography. These results are shown in FIGS. 14 and 15; FIG. 14 shows the levels (in terms of percentage of total, indicated by AUC measurement) of the main peak (FIG. 14A), acidic variants (FIG. 14B) and basic variants (FIG. 14C) present in cysteine-containing formulations of TmAb, while FIG. 15 provides representative trace of the stability profiles of the two formulations, one containing 1.5 mg/mL cysteine (FIG. 15A) and the other containing 6 mg/mL cysteine (FIG. 15B). Taken together, these results indicate that high levels of cysteine may disrupt the internal Cys-Cys bonds that are present in the TmAb molecule, possibly leading to inactivation of the antibody and therefore loss of its therapeutic effects in addition to a loss in storage stability. Thus, inclusion of a lower cysteine content (e.g., 1.5 mg/mL) in the pre-spray-drying formulation may enhance stability while avoiding the loss of storage stability and potential loss of bioactivity brought on by inclusion of higher amounts of cysteine in the formulations.

Finally, dialyzed solutions of TmAb were prepared in an optimized excipient formulation and used to optimize certain spray-drying parameters, in particular the optimum feed solution concentration and inlet temperature settings for maximizing protein concentration and storage stability while minimizing aggregation. Formulations were prepared as shown in Table 18:

TABLE 18
Formulations for Optimizing Spray-Drying Parameters

| | Feed | | Feed | | Feed | |
|---|---|---|---|---|---|---|
| | Solution | | Solution | | Solution | |
| | Batch | Powder | Batch | Powder | Batch | Powder |
| | TF29a | Batch | TF29b | Batch | TF29c | Batch |
| | (70C inlet) | TF29a | (80C inlet) | TF29b | (90C inlet) | TF29c |
| TmAb | 20.0 mg/mL | 76.63% | 20.0 mg/mL | 76.63% | 20.0 mg/mL | 76.63% |
| | (0.135 mM) | | (0.135 mM) | | (0.135 mM) | |
| TmAb | — | 565.4 mg/mL | — | 565.4 mg/mL | — | 565.4 mg/mL |
| mg/mL in | | | | | | |
| paste | | | | | | |
| (assume | | | | | | |
| 60% w/w | | | | | | |
| load) | | | | | | |
| Trehalose | 5.15 mg/mL | 19.73% | 5.15 mg/mL | 19.73% | 5.15 mg/mL | 19.73% |
| | (15.0 mM) | | (15.0 mM) | | (15.0 mM) | |
| Polysorbate | 0.5 mg/mL | 1.92% | 0.5 mg/mL | 1.92% | 0.5 mg/mL | 1.92% |
| 20 | | | | | | |
| Lactate | 0.450 mg/mL | 1.72% | 0.450 mg/mL | 1.72% | 0.450 mg/mL | 1.72% |
| | (5 mM) | | (5 mM) | | (5 mM) | |
| pH | 4.0 | — | 4.0 | — | 4.0 | — |
| Total | 25.1 mg/mL | 100% | 25.1 mg/mL | 100% | 25.1 mg/mL | 100% |
| | Feed | | Feed | | Feed | |
| | Solution | | Solution | | Solution | |
| | Batch | Powder | Batch | Powder | Batch | Powder |
| | TF52a | Batch | TF52b | Batch | TF52c | Batch |
| | (70C inlet) | TF52a | (80C inlet) | TF52b | (90° C. inlet) | TF52c |
| TmAb | 30.0 mg/mL | 76.63% | 30.0 mg/mL | 76.63% | 30.0 mg/mL | 76.63% |
| | (0.202 mM) | | (0.202 mM) | | (0.202 mM) | |
| TmAb | — | 565.4 mg/mL | — | 565.4 mg/mL | — | 565.4 mg/mL |
| mg/mL in | | | | | | |
| paste | | | | | | |
| (assume | | | | | | |
| 60% w/w | | | | | | |
| load) | | | | | | |
| Trehalose | 7.72 mg/mL | 19.73% | 7.72 mg/mL | 19.73% | 7.72 mg/mL | 19.73% |
| | (22.5 mM) | | (22.5 mM) | | (22.5 mM) | |
| Polysorbate | 0.75 mg/mL | 1.92% | 0.75 mg/mL | 1.92% | 0.75 mg/mL | 1.92% |
| 20 | | | | | | |
| Lactate | 0.675 mg/mL | 1.72% | 0.675 mg/mL | 1.72% | 0.675 mg/mL | 1.72% |
| | (7.5 mM) | | (7.5 mM) | | (7.5 mM) | |
| pH | 4.0 | — | 4.0 | — | 4.0 | — |
| Total | 37.65 mg/mL | 100% | 37.65 mg/mL | 100% | 37.65 mg/mL | 100% |

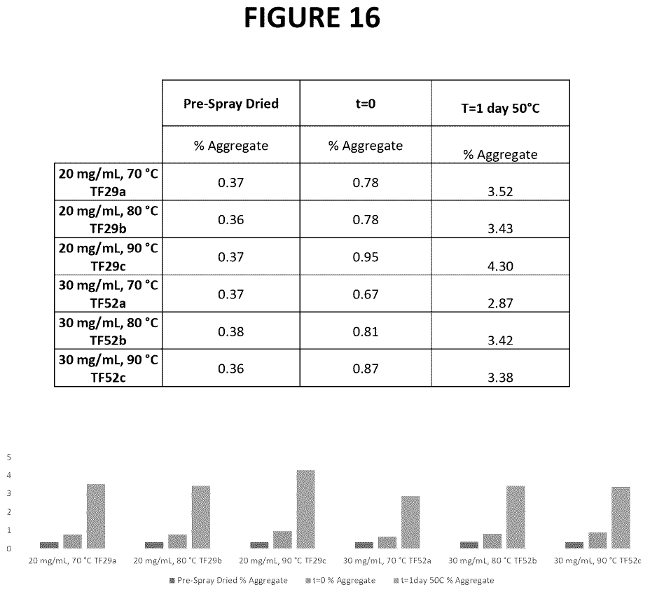

Additional formulations were evaluated by ion exchange chromatography for aggregation both pre-spray drying and at t=0 and t=1 day at 50° C. post-spray-drying. Results are shown in FIG. 16. These results demonstrate that use of a higher feed solution concentration (e.g., 30 mg/mL TmAb vs. 20 mg/mL TmAb) results in the production of less aggregated starting solutions, less aggregated spray-dried powders and more storage-stable powders at a given spray dryer inlet temperature (compare, e.g., results in FIG. 16 for Batch 52a vs. Batch 29a; Batch 52b vs. Batch 29b; and Batch 52c vs. Batch 29c). In addition, at a given feed solution concentration, the 70° C. inlet temperature appeared to result in the production of less aggregated (at t=0) and more storage stable (at t=1 day at 50° C.) powders, vs. those prepared at higher inlet temperatures (compare, e.g., results in FIG. 16 for Batch 29a vs. Batches 29b and 29c; and those for Batch 52a vs. Batches 52b and 52c); this result confirms those reported above in Example 1 with respect to the optimum inlet temperature.

Based on the studies described above, the composition detailed in Table 19 represents one example formulation exhibiting good antibody stability and while also providing for high antibody drug concentration (>400 mg/mL) in the resulting paste formulations:

TABLE 19
XeriJect ® Paste Formulation for a High Concentration TmAb Paste

| | Feed Solution | Powder Batch |
|---|---|---|
| Antibody (e.g., TmAb) conc. | 20.0 mg/mL | 62.31% |
| | (0.135 mM) | |
| Antibody mg/mL in paste | — | 459.7 mg/mL |
| (assume 60%w/w load) | | |
| Trehalose | 5.15 mg/mL | 16.04% |
| | (15.0 mM) | |
| Polysorbate 20 | 0.5 mg/mL | 1.56% |
| Proline | 6 mg/mL | 18.69% |
| Lactate | 0.450 mg/mL | 1.40% |
| | (5 mM) | |
| pH | 4.0 | — |
| Total | 32.1 mg/mL | 100% |

Altogether, these studies provide examples of representative aqueous feed solution formulations and spray-drying and process conditions to produce highly-concentrated, high solids content, and storage-stable dry powder preparations of therapeutic proteins such as monoclonal antibodies, which are suitable for use in preparing the high concentration injectable paste formulations that permit the intracutaneous, subcutaneous and/or intramuscular injection of therapeutic peptides and proteins that previously could only be administered intravenously over considerably longer time periods. In fact, using the approach described in this Example, it is possible to prepare a matrix of formulation and apparatus parameters that would facilitate excipient and process parameter screening to promote the development of suitable formulations for the preparation of high solids concentration formulations that may be advantageously spray-dried into flowable, storage-stable paste formulations for therapeutic uses. This approach-preparing high solids concentrations injectable paste therapeutic formulations, a technology developed by Xeris Pharmaceuticals, Inc. under its XeriJect® technology platform that is described herein—therefore provides a number of patient benefits, including convenience of dosing, avoidance of discomfort and possibly efficacy of the therapeutic peptide/protein formulation.

### Example 3: Production of High Concentration Pastes Comprising Therapeutic Monoclonal Antibodies

XeriJect® (XJ) is a proprietary formulation technology that can significantly increase the concentration and/or the thermostability of an active pharmaceutical ingredient (API) in a dose. With the XeriJect® technology, dry particles of an active pharmaceutical ingredient (API), preferably prepared by spray-drying according to the methods described in Examples 1 and 2 above, are blended with a non-solvent liquid and mixed to form a paste. As described previously, a paste is a two-phase composition residing on the spectrum between suspension and wetted solid, wherein the solids concentration in the powder. With this approach, drug concentrations of 30% w/w or higher can be achieved (250 mg/mL or higher). This technology represents a significant improvement over current therapies, which often must be administered as a long-duration IV infusion of a low-concentration solution in a clinic. This technology can be used to deliver high doses of proteins, such as antibodies, or small molecules to a patient with a bolus subcutaneous dose. In addition, in certain formulations the XeriJect® technology can provide increased thermostability of the formulation (particularly the active pharmaceutical ingredient(s) in the formulation) even at standard lower-dose concentrations.

In these studies, the XeriJect® technology was evaluated as a platform for the small volume subcutaneous delivery of therapeutics that previously were only capable of being administered intravenously. Specifically, several monoclonal antibody products that are commercially available were formulated into XeriJect® paste formulations:

TABLE 20
Commercial Drug Products Prepared in Paste Form Using XeriJect ®
Technology

| | | | | | 400 mg/mL |
|---|---|---|---|---|---|
| | | Delivered | Delivery | Current | Paste |
| Product | Indication | Dose | Route | Volume | Formulation |
| OPDIVO ® | Non-small Cell Lung | 240 mg | I.V. | 250 mL | 0.60 mL |
| | Cancer | | | | |
| HUMIRA ® | Crohn's Disease | 160 mg | S.C. | 3.2 mL | 0.40 mL |
| HUMIRA ® | Rheumatoid Arthritis | 40 mg | S.C. | 0.8 mL | 0.10 mL |
| XGEVA ® | Skeletal Events (from bone | 120 mg | S.C. | 1.7 mL | 0.30 mL |
| | metastases) | | | | |
| ENTYVIO ® | Inflammatory Bowel | 300 mg | I.V. | 250 mL | 0.75 mL |
| | Disease | | | | |
| REMICADE ® | Crohn's Disease | 350 mg¹ | I.V. | 250 mL | 0.88 mL |
| REMICADE ® | Rheumatoid Arthritis | 210 mg² | I.V. | 250 mL | 0.53 mL |
| TYSABRI ® | Multiple Sclerosis | 300 mg | I.V. | 115 mL | 0.75 mL |
| KADCYLA ® | Metastatic Breast Cancer | 252 mg³ | I.V. | 250 mL | 0.63 mL |
| YERVOY ® | Metastatic Melanoma | 210 mg⁴ | I.V. | 100 mL | 0.53 mL |

¹5 mg/kg, 70 kg patient
²3 mg/kg, 70 kg patient
³3.6 mg/kg, 70 kg patient
⁴3 mg/kg, 70 kg patient

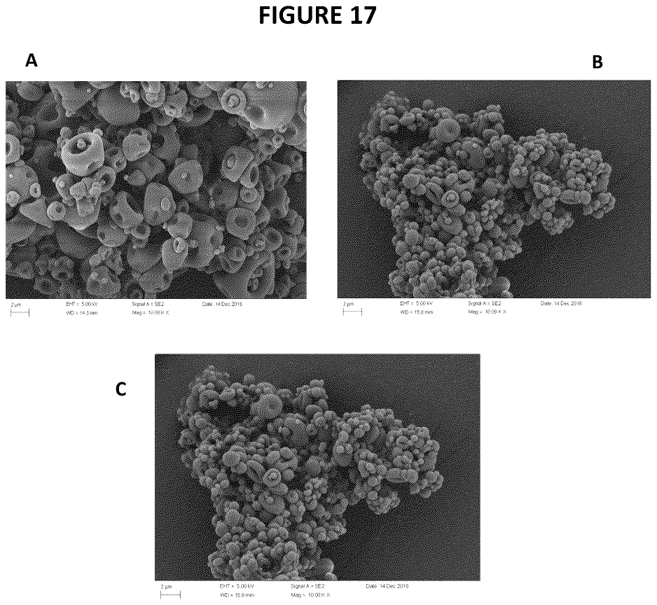

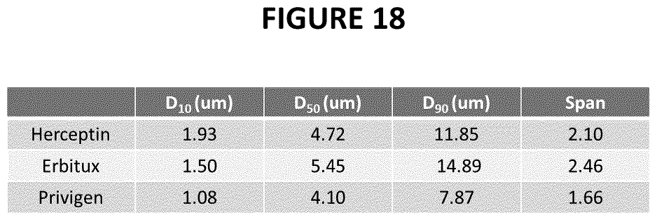

To set a baseline, samples of three other commercial products—Herceptin® (TmAb; see Examples 1-2), Erbitux® (cetuximab; Eli Lilly and Company), and Privigen® (immune globulin; CSL Behring AG)—were spray-dried from the commercial formulations with minimal modification, and then examined by scanning electron microscopy to observe the morphology and size of the antibody particles in the powdered products. Representative photomicrographs are shown in FIG. 17, which shows particles observed in spray-dried formulations of TmAb (FIG. 17A), cetuximab (FIG. 17B) and immune globulin (FIG. 17C) which are reminiscent of those observed for other therapeutic proteins in Example 1. Most of these commercial products demonstrated a broad range of particle sizes, and the TmAb commercial formulation also showed a high degree of toroidal particles which, based on the results presented in Example 1, are known to result in less than optimal powder materials for use in preparing therapeutic pastes using the XeriJect® formulation technology. In fact, when the particle size distribution for these three formulations was obtained by laser diffraction, there was a polydisperse character as characterized by the range observed for the 10th percentile to the 90th percentile of particle diameters in all of the formulations, as shown in FIG. 18.

To prepare XeriJect® paste formulations of these commercial products, small batches of each were spray-dried and pastes prepared according to the methods described above in Examples 1 and 2. API instead of trehalose was added to the commercial cetuximab and immune globulin formulations, and the cetuximab salt content was reduced by dialysis. The TmAb formulation was spray-dried from the commercial formulation. Pastes were prepared from the spray-dried powders and triacetin (density=1.16 g/mL) was included such that the final estimated paste density was about 1.24 g/mL. The total solids and active ingredient contents in each paste formulation were then calculated to be at the values shown in Table 21:

TABLE 21
Solids and Active Ingredient Content of XeriJect ® mAb Pastes

| | | | mg Solids | | |
|---|---|---|---|---|---|
| | Residual | Percent | Per mL | % mAb in | mg mAb Per |
| | Moisture | Solids | Paste | Formulation | mL Paste |
| Herceptin | 0.88% | 45.2% | 560.5 mg | 51.3% | 287.5 mg |
| Erbitux | 2.29% | 42.0% | 520.8 mg | 66.5% | 346.3 mg |
| Privigen | 1.38% | 42.0% | 520.8 mg | 60.0% | 312.5 mg |

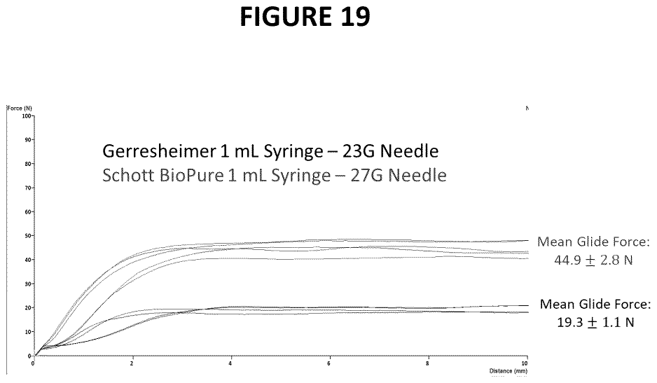

Following paste preparation, the formulations were evaluated for the injection force necessary to deliver 1 mL of a given paste in two different syringe/needle combinations: a 1 mL syringe with a staked 23 gauge ½ inch regular wall (RW) needle (Gerresheimer AG; Bunde, Germany) and a syriQ BioPure® ImL long syringe with a 27 gauge ½ inch regular wall (RW) needle (Schott AG; Mainz, Germany). For each configuration, a plunger velocity of 3.0 mm/sec (corresponding to a volumetric flow rate of approximately 98 μL/sec) was used to generate the injection force profile. Representative results with a preparation of Privigen® (immune globulin) prepared with triacetin as the diluent (continuous phase) at a 42% solids content are shown in FIG. 19. As expected, a significantly lower injection force was necessary to deliver the 1 mL paste in the larger (lower gauge) needle; this result confirms prior results using lower concentration paste formulations. Importantly, however, the full 1 mL of the paste product was delivered from even the 27G needle without the need for overly excessive injection force. The ability to intracutaneously inject paste formulations of therapeutic antibodies in low volumes using relatively small gauge needles appropriate for intracutaneous and/or intramuscular injection and having relatively low injection forces significantly enhances the patient benefits of such injections, in terms of decreasing discomfort and the time required to receive a therapeutic antibody treatment, compared to standard IV administration of such antibodies.

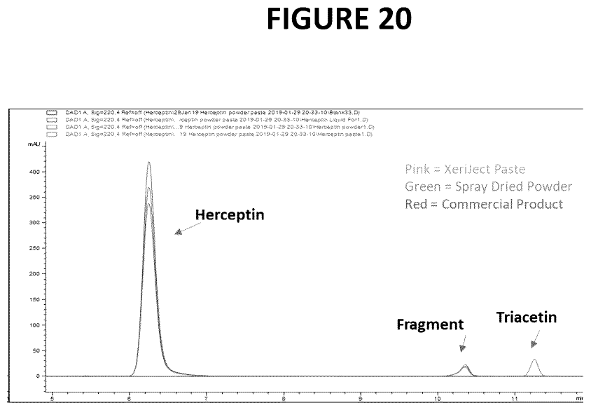

Next, the ability to formulate TmAb into a usable high-concentration paste was evaluated. The commercial product was reconstituted in water, and samples were held in solution, spray-dried into a powder, or formulated from the spray-dried powder into a paste (45.2% solids in triacetin) using the XeriJect® technology described above. Evaluation of these samples by size exclusion chromatography demonstrated very little difference between them (FIG. 20)—all of the samples contained both a main peak and a fragment with identical retention profiles, while the XeriJect® paste sample contained an additional peak corresponding to the triacetin in the paste formulation. Thus, the spray-drying and paste formation processes did not result in the aggregation of the TmAb protein when compared to reconstituted commercial product in solution.

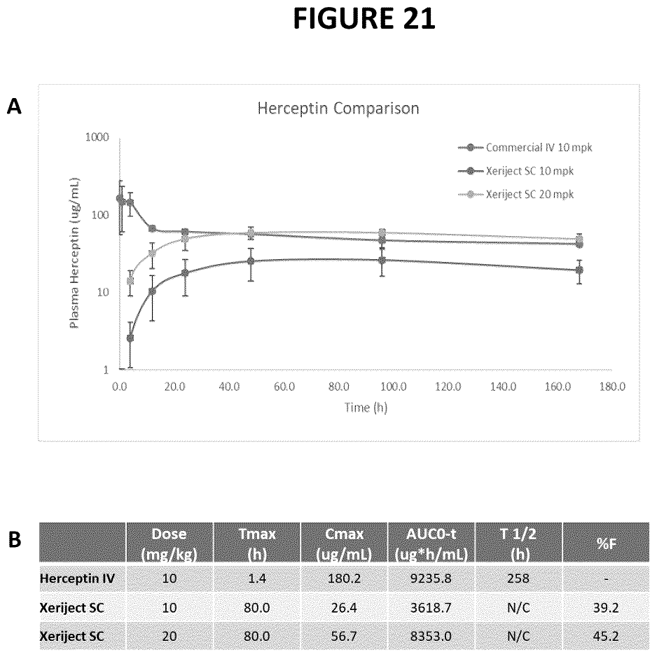

To evaluate the pharmacokinetics of these various TmAb formulations, and specifically those of the XeriJect® paste formulations, the commercial antibody solution was spray-dried as described in the preceding examples, producing a spray-dried powder that was used to prepare a high solids content paste as described above. The API loading in this formulation was 259 mg/mL, and it was dosed by subcutaneous injection to male Sprague Dawley rats at two different dose volumes, using commercially available syringe/needle combinations. A commercial formulation of trastuzumab was also dosed by IV into control animals for comparison. As seen in FIG. 21, the IV dose of trastuzumab showed an early spike in drug level, where the XeriJect® formulations did not have a spike and rose to a plateau drug level after ~24 hours (FIG. 21A). Over the next six days of sampling, the XeriJect® formulation and the IV dose maintained their plateau drug levels. As depicted in FIG. 21B, the pharmacokinetics for the XeriJect® formulation were dose-dependent, with the 20 mg/kg XeriJect® plateau drug level being similar to that of the 10 mg/kg IV dose. The Cmax was blunted by about 15% for both the 10 mg/kg and 20 mg/kg doses of XeriJect® TmAb compared with IV TmAb, while the bioavailability (AUCo-t) of the 10 mg/kg and 20 mg/kg doses of XeriJect® TmAb compared with 10 mg/kg IV TmAb was 39 and 45%, respectively (dose normalized). Finally, while the T_{1/2} of the IV TmAb was about 10 days, the T_{1/2} for the XeriJect® formulations was longer than the time allotted for the study and was not determined here. As those of ordinary skill in the pharmaceutical and medical arts will readily understand, a blunted Cmax and sustained exposure (i.e., longer T_{1/2}) can be favorable for compounds that have Cmax-driven toxicity profiles and AUC-driven efficacy, thereby providing another benefit of the pastes prepared using the XeriJect® technology of the present invention. These results indicate that a high amount of drug can be administered in a relatively low volume in a bolus injection, while achieving therapeutically beneficial circulating drug levels with similar kinetics (albeit longer-lasting) as are achieved with IV administration. The XeriJect® approach thus significantly improves this particular drug treatment/delivery and may be similarly useful for other treatments using high molecular weight peptides and proteins such as therapeutic antibodies and enzymes.

### Example 4: Production of High Solids Pastes Comprising Therapeutic Enzymes

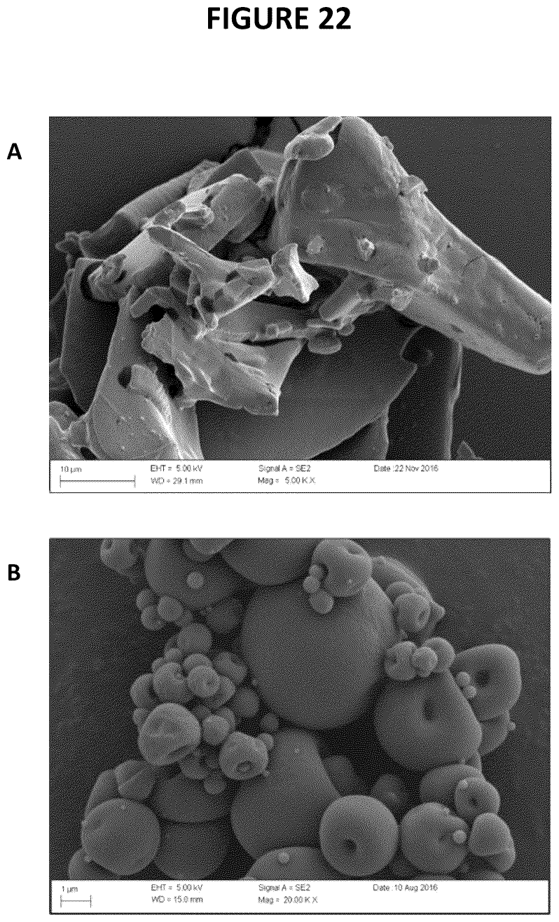

To further examine the utility of the XeriJect® technology provided by the present invention, high solid concentration pastes comprising therapeutic enzymes were prepared. In these exemplary studies, a multimeric PEGylated therapeutic enzyme was used as the active ingredient. In a first step, solutions of the PEGylated enzyme were converted into dry powders by lyophilization and by the spray-drying processes described in the preceding examples. Samples of each powder preparation were then examined by scanning electron microscopy for morphology and size distribution. Representative photomicrographs are shown in FIG. 22. As seen in FIG. 22A, the PEGylated enzyme powder prepared by lyophilization of the feed solution demonstrated large, irregular particles with a relatively high specific surface area. In contrast, as seen in FIG. 22B, the powder prepared by the spray-drying processes described herein demonstrated small, spherical particles with a comparatively lower specific surface area. Moreover, when pastes were prepared from both of these powders, the pastes prepared from spray-dried powders exhibited a higher solids concentration, and thus a higher enzyme concentration, than pastes prepared from the lyophilized powders, as shown in Table 22:

TABLE 22
Characteristics of Enzyme Pastes

| | | | | Enzyme | |
|---|---|---|---|---|---|
| | % Solids | Paste | Solids | (% w/w) | Approx. Enzyme |
| Powder | Content | Density | Concentration | In Powder | Concentration |
| Lyophilized | 36.9% | 1.24 g/mL | 457.6 mg/mL | 16.8% | 77.0 mg/mL |
| Spray-Dried | 43.0% | 1.26 g/mL | 544.9 mg/mL | 16.8% | 91.7 mg/mL |

These results correspond with the discussion in the foregoing examples herein, that powders that comprise more regular, spherical particles of smaller size and surface area distribution are more suitable for the production of flowable pastes of active ingredients, and ultimately are more suitable for therapeutic subcutaneous injection into animals. Therefore, the remainder of these studies were conducted using spray-dried powders as starting materials for the production of the therapeutic XeriJect® pastes.

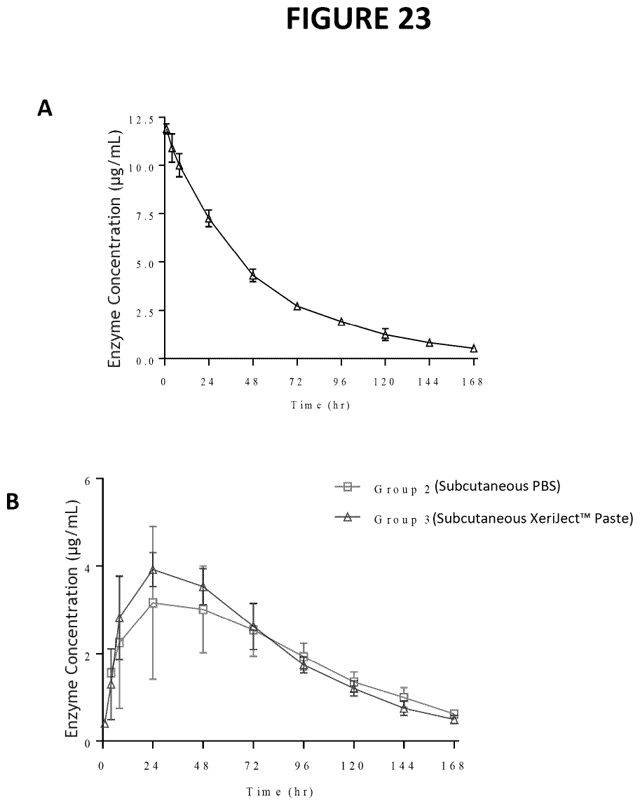

To study the pharmacokinetic parameters of the XeriJect® enzyme paste formulations, the spray-dried paste enzyme preparations described above were dosed by subcutaneous injection to male Sprague Dawley rats, using commercially available syringe/needle combinations. An aqueous formulation of the enzyme (in PBS) was also dosed by IV and subcutaneously into control animals for comparison. Plasma enzyme concentrations in each group of animals were then determined over time, with results shown in FIG. 23. The aqueous (PBS) formulation administered intravenously demonstrated an initial spike of enzyme in the plasma followed by a rapid decline over the next 48-72 hours (FIG. 23A). In contrast, the subcutaneous aqueous (PBS) and XeriJect® paste formulations demonstrated similar pharmacokinetic profiles with a slower rise to peak plasma concentration followed by a more prolonged and gradual decrease (FIG. 23B). As shown in Table 23, the T½, Tmax, Cmax and AUC(0-t) values of the subcutaneously administered formulations were also similar and significantly different from those for the IV-administered enzyme, reminiscent of results obtained with IV aqueous vs. subcutaneous XeriJect® paste formulations of TmAb described in Example 2 herein:

TABLE 23
Pharmacokinetic Profiles of IV and Subcutaneous Enzyme Formulations

| | T1/2 | Tmax | Cmax | AUC(0-t) |
|---|---|---|---|---|
| Group | (hr) | (hr) | (μg/mL) | (hr * μg/mL) |
| Intravenous | 37.0 ± 1.8 | 1.0 ± 1.0 | 11.9 ± 0.3 | 576.0 ± 19.1 |
| PBS | | | | |
| Subcutaneous | 46.7 ± 3.1 | 24.0 ± 24.0 | 3.38 ± 1.4 | 331.0 ± 106 |
| PBS | | | | |
| Subcutaneous | 38.1 ± 1.6 | 24.0 ± 24.0 | 3.92 ± 0.4 | 351.0 ± 53.4 |
| XeriJect ® | | | | |

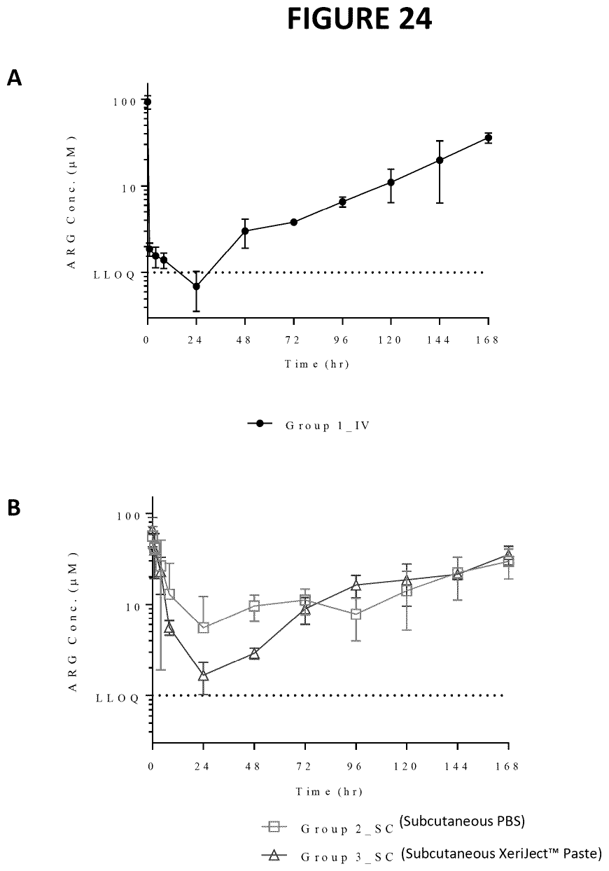

Pharmacodynamic analysis of plasma samples from the treated animals demonstrated a difference not only between IV-administered (FIG. 24A) and subcutaneous-administered (FIG. 24B) enzyme formulations, but also between subcutaneously administered aqueous (PBS) and XeriJect® paste formulations (FIG. 23B). Specifically, while the aqueous and paste formulations exhibited similar pharmacokinetic profiles as described above, the XeriJect® paste formulation showed a greater peak target depletion in pharmacodynamic studies (6 μM vs. 2 μM). Moreover, the results of a post-sacrifice pathology report (not shown) indicated that there were no significant injection site reactions (and thus no post-injection discomfort) observed in animals that had been injected subcutaneously with the XeriJect® paste formulations.

Taken together, these results indicate that the paste formulations of high concentration high molecular weight protein therapeutics that are provided by the present invention are useful in delivering controlled- or sustained-release depots of a therapeutic protein in small volumes subcutaneously in a way that improves the patient experience from that obtained with traditional intravenous administration of such therapeutic proteins.

### Example 5: Production of High Solids Pastes Comprising High Concentration Glucagon

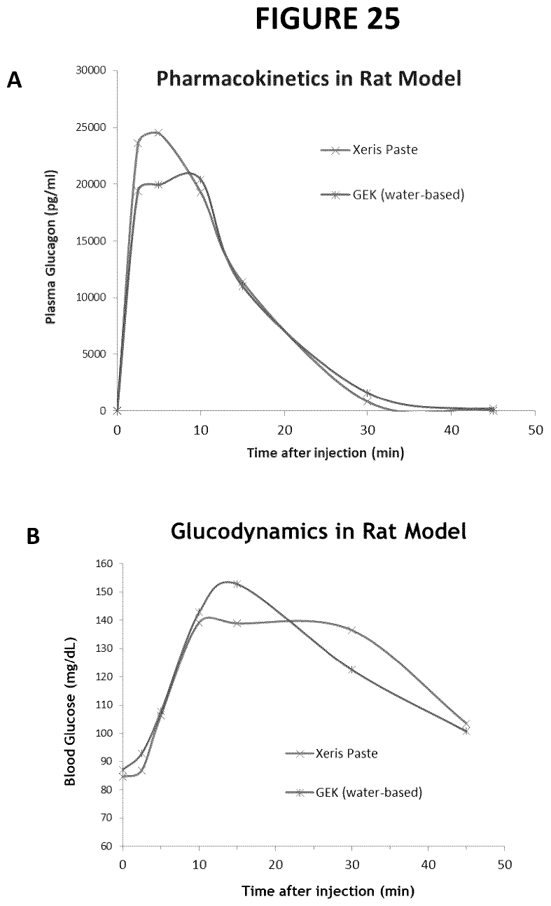

In additional studies, XeriJect® paste formulations containing high-concentration glucagon were prepared by thin-film-freezing (a particle engineering technology producing powders having relatively high surface area) from an aqueous solution containing glucagon, trehalose, and a buffering agent (glycine) and adjusted to pH 3.0. The thin-film freeze-dried powder was formulated into a paste by mildly grinding and blending with sufficient triacetin create a paste. 1 mg of glucagon was administered as 5 ul of the paste subcutaneously to rats at the same dose as a larger volume (1 ml) of commercially available aqueous glucagon formulation (glucagon emergency kit or “GEK”; Eli Lilly). This allowed for a direct comparison of the pharmacokinetics and pharmacodynamics of the XeriJect® paste formulation to a commercial aqueous solution. Results are shown in FIG. 25.

Examination of the pharmacokinetic profiles of the two formulations (FIG. 25A) indicated that despite being injected in a 200× lower volume, the XeriJect® (Xeris) paste formulations exhibited comparable pharmacokinetics as the aqueous formulations of glucagon injected in a higher volume. The same was true of the pharmacodynamic (glucodynamic) profiles (FIG. 25B), with the XeriJect® (Xeris) paste formulations showing similar pharmacodynamics as observed for the aqueous GEK formulations, albeit a slower decay over time as has been seen with other therapeutic peptides (see preceding Examples). These results indicate that the paste formulations of a higher concentration peptide (glucagon) that are provided by the present invention are useful in delivering of glucagon in small volumes subcutaneously in a way that improves the patient experience from that obtained with traditional intramuscular administration of aqueous formulations of glucagon that require relatively larger volume injections.

### Example 6: Production of High Solids Pastes Comprising Insulin

In addition to providing injectable formulations having very high concentrations of therapeutic agents, injectable pastes described herein may also be utilized to significantly enhance the thermostability of a therapeutic agent, including those administered at relatively lower concentrations. One such example is human insulin, which is commercially available in concentrations ranging from u100 (~3.5 mg/mL) to u500 (~17.4 mg/mL). Insulin dose volumes are patient-dependent, but generally range from 30-100 μL of a u100 formulation. Therefore, significantly increasing the drug concentration to reduce the dose volume is not necessary for this therapeutic protein. However, commercial insulin drug products are formulated as aqueous solutions requiring refrigerated (2-8° C.) shipping and long-term storage conditions. This cold-chain requirement can limit the availability, as well as compromise the quality, of commercial insulins in third-world countries. Accordingly, there is a need for injectable insulin formulations with significantly improved thermostability which may exhibit long-term stability (i.e., storage stable for at least one year and more preferably for at least 18, 24, 30, or 36 months) at temperatures of at least 25° C., 30° C., or 35° C.

An example of the enhanced thermostability of spray-dried insulin powder relative to commercial aqueous insulin drug products is provided herein. Insulin powders were spray dried from an aqueous feed solution containing a buffer selected from glycine or histidine (ranging from ~5-20 mM), a surfactant selected from PS20 or PS80 (ranging from ~0.001-0.1% (w/v) and the sugar/disaccharide trehalose (from dihydrate) at pH 8.5. Due to the high solids content of the pastes, coupled with the relatively low concentration of insulin required in the final formulation (e.g. u100=3.5 mg/mL), the excipient concentration in the feed solution exceeded 99% of the total (by weight). This high ratio of excipients-to-insulin allowed the paste compositions (having ~55-65% (w/w) solids content (powder concentration of approximately 600-780 mg/mL) and measured densities from ~1.1-1.2 g/mL) to contain a final insulin content of approximately 3.5 mg/mL, corresponding to u100.

The Buchi B-290 mini-spray dryer used to prepare the powders had an inlet temperature of 140° C., an atomizing nozzle pressure of 60 (measured from the floating ball flow meter incorporated in the instrument), a liquid feed rate of 10% (~3 mL/minute) and an aspirator setting of 90%. The spray dried powders were further dried (i.e., secondary dried) under vacuum to reduce the measured moisture content below approximately 1% (w/w), and then stored in glass vials and placed in a 40° C./75% RH stability chamber. The chemical stability of the insulin powders were evaluated following 123 days (~4 months) and revealed an insulin peak purity loss of less than 2% over that storage period.

As a comparison, commercial Humulin® R insulin (aqueous solution) was stored at 40° C. in glass vials and revealed a decrease in purity of approximately 7% over 1 month as measured using the same UHPLC method used for the powders. The USP monograph for insulin injection drug products is 95-105% of label claim, indicating that the commercial drug products fall below their stability specification within one month at accelerated conditions. Therefore, the ability to prepare thermostable spray dried insulin powders for use in a therapeutic paste can improve the thermal stability of currently available commercial formulations, while still allowing for a comparable injection volume.

### Example 7: Production of High Solids Pastes Comprising High Dose Human Proteins or Peptides

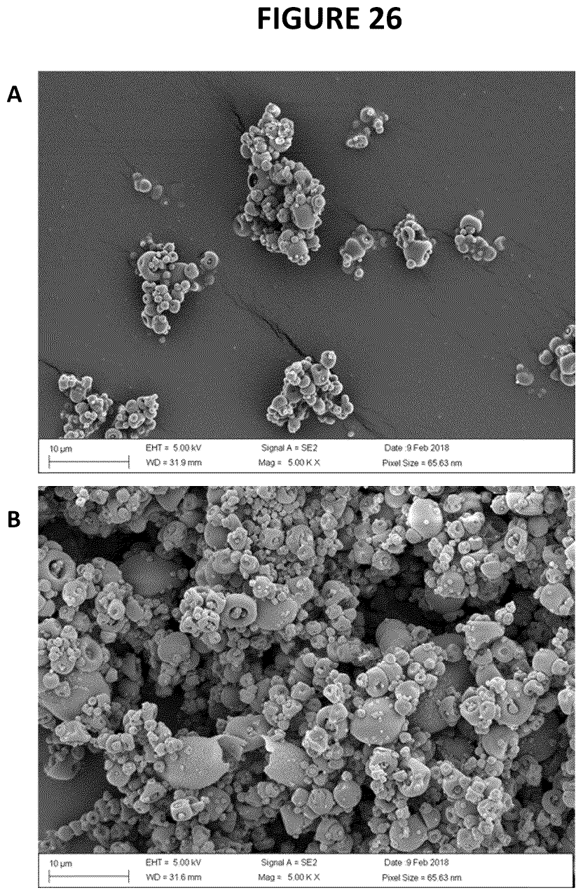

In additional studies, XeriJect® paste formulations of high-concentration human recombinant proteins were prepared according to the spray-drying methods described in the preceding examples. In a first step, solutions of a human recombinant protein prepared and optimized according to the methods described herein (see Examples 1 and 2) were converted into dry powders by the spray-drying processes described in the preceding examples. Samples of the powder preparation were then examined by scanning electron microscopy for morphology and size distribution. Representative photomicrographs are shown in FIG. 26.

As discussed in the preceding examples, the ability to inject high concentrations and amounts of protein in a relatively low-volume subcutaneous injection of a paste formulation requires the formation of appropriately sized small and generally spherical particles in the powder used to prepare the therapeutic paste. As seen in FIGS. 26A and 26B, the protein powder prepared by the spray-drying processes described herein produced small, spherical particles with a relatively low per-particle specific surface area. Population statistics across the powder preparations (not shown) determined that the median particle size was optimal for paste formation, based on the studies described elsewhere herein (see, e.g., the preceding Examples).

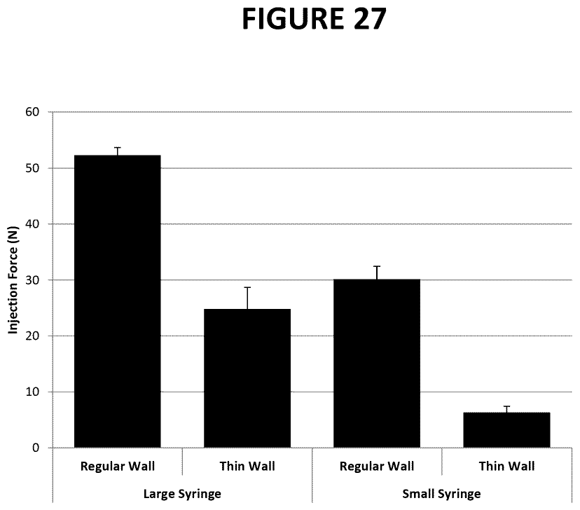

These powders were then used to prepare XeriJect® paste formulations using the methods described above, resulting in pastes with about 45% solids content and having in excess of 300 mg/mL of protein in the pastes. A therapeutic dose of pastes having this concentration of active was calculated to be only about 150 μL. The injection force required to deliver this volume of the pastes prepared in this example were then measured as described above, in commercially available large and small syringes affixed with either regular wall or thin wall 27G needles, at a volumetric flow rate of 30 μL per second. Results of these studies are shown in FIG. 27 and demonstrated that injection forces as low as 6N are capable with the optimal syringe/needle configuration (i.e., a small syringe with a thin wall needle).

### Example 8: Production of High Concentration Pastes Comprising Bevacizumab

In these studies, the XeriJect® technology was evaluated as a platform for the small volume subcutaneous delivery of a commonly used therapeutic monoclonal antibody formulation, bevacizumab (commercialized by Genentech under the AVASTIN® brand) that previously has only been capable of being administered intravenously. Bevacizumab is a human monoclonal antibody that binds Vascular Endothelial Growth Factor (VEGF), preventing interaction of VEGF with its receptor and retarding or preventing neovascularization particularly in cancerous tissues. As such, it has been approved for a number of indications in humans, including the treatment of metastatic colorectal cancer, non-squamous non-small cell lung cancers, glioblastomas and metastatic renal cell carcinomas. It is typically administered intravenously (IV) in a dosage of 10 mg/kg at an aqueous concentration of 25 mg/mL via a 30-90 minute infusion about every two weeks. The half-life of this product is about 20 days with an average clearance rate of about 0.262 L/day, depending on patient-specific parameters such as body mass, gender and tumor burden. Thus, the present inventors evaluated whether the XeriJect® technology could be used to create formulations of bevacizumab that could be administered in a lower volume/higher dose and that would have at least similar if not more advantageous pharmacokinetics, both compared to the IV product. As described elsewhere herein, such formulations would provide significant benefits to patients and caregivers including the use of a very small volume injection which is easier to administer as a subcutaneous dose rather than via an IV route, both resulting in less discomfort to the patient.

To conduct these studies, commercially available bevacizumab drug substance was formulated into two different therapeutic paste formulations, named XJ-1 and XJ-2. The composition of the pre-spray dried aqueous solutions (mg/mL) and the approximate % wt. of the spray-dried powders are provided in Table 24.

TABLE 24
Bevacizumab Formulations Prepared Using XeriJect ® Technology

| | XJ-1 Formulation | | XJ-2 Formulation | |
|---|---|---|---|---|
| Component | Concentration | % (w/w) | Concentration | % (w/w) |
| Bevacizumab | 35 mg/mL | 62.67 | 35 mg/mL | 62.67 |
| Trehalose | 10.3 mg/mL | 18.44 | 10.3 mg/mL | 18.44 |
| Dihydrate | | | | |
| Polysorbate 80 | 0.1 mg/mL | 0.18 | 0.1 mg/mL | 0.18 |
| Malic Acid | 0.45 mg/mL | 0.81 | 0.45 mg/mL | 0.81 |
| Proline | 10 mg/mL | 17.91 | 0 | 0 |
| Histidine | 0 | 0 | 10 mg/mL | 17.91 |
| pH | 3.8 | — | 6.0 | — |
| Total | 55.85 mg/mL | 100% | 55.85 mg/mL | 100% |

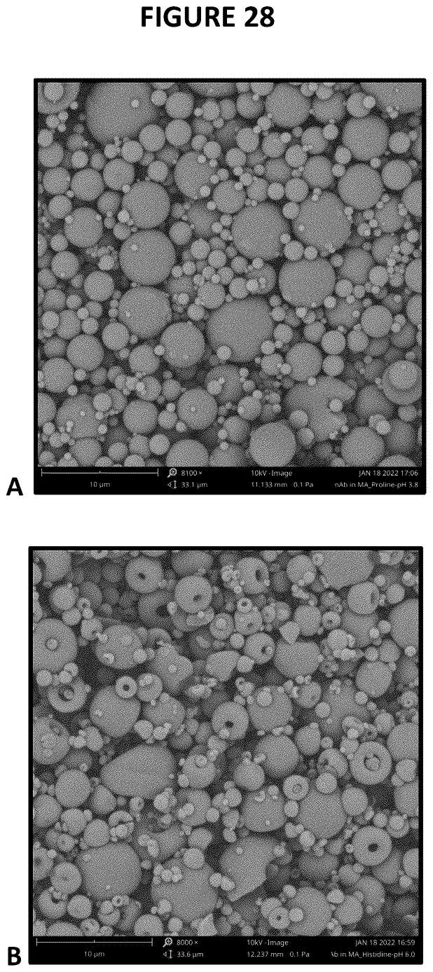

To more closely examine these powders at the particle level, a sample of powder from each of these two formulations was examined using SEM to evaluate particle size, size distribution and shape (morphology) as discussed in Examples 1-3 above. Representative photomicrographs are shown in FIG. 28, which shows that particles observed in spray-dried formulations of XJ-1 (FIG. 28A) and XJ-2 (FIG. 28B) demonstrated a range of spherical particles which had a mean size of about 3.3 μm (for XJ-1) or about 3.0 μm (for XJ-2). The XJ-2 formulation also showed a somewhat higher degree of toroidal particles compared to the XJ-1 formulation.

Based on these results, these formulations were used to prepare XeriJect® formulations of bevacizumab for use in animal pharmacokinetic studies. For each paste, XJ-1 and XJ-2 powder was separately blended with Miglyol 812 in an HDPE container using a planetary-orbital mixer. The solids content of each of the two pastes formulation were 62% for XJ-1 and 55% for XJ-2, respectively. The measured (absorbance at 280 nm) mAb content for the two formulations were for 429 mg/mL for XJ-1 and 328 mg/mL for XJ-2.

In these studies, four groups of female Göttingen minipigs were dosed with a single 100 mg dose of one of four different bevacizumab (BmAb) formulations, either intravenously (IV) or subcutaneously (SC), as provided in Table 25:

TABLE 25
Bevacizumab PK Study Design

| | | | | | Dose | |
|---|---|---|---|---|---|---|
| | | | No. of | Dose | volume/ | |
| Group | Formulation | Route | Animals | Concentration | animal | Dose |
| 1 | Avastin ® | IV | 4 | 25.87 mg/mL | 4.1 mL | 106 mg |
| 2 | Avastin | SC | 4 | 25.87 mg/mL | 4.1 mL | 106 mg |
| 3 | XJ-1BmAb | SC | 4 | 438.67 mg/mL | 0.21 mL | 94 mg |
| 4 | XJ-2 BmAb | SC | 4 | 327.56 mg/mL | 0.22 mL | 82 mg |

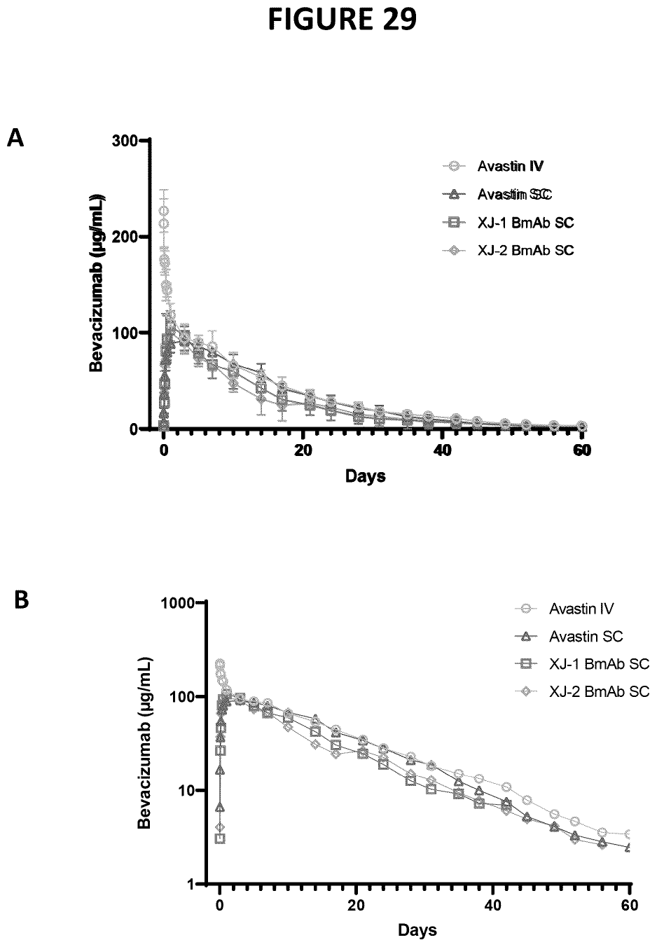

After injection of each formulation, plasma was sampled from the animals at post-injection times of 5 and 30 minutes, 2, 4, 8, 12 and 24 hours, and 3, 5, 7, 10, 14, 17, 24, 28, 31, 35, 38, 42, 45, 49, 56 and 60 days. Plasma samples were then measured for concentration of circulating bevacizumab, to allow an assessment of time to maximum plasma concentration (Tₘₐₓ), maximum concentration absorbed (Cₘₐₓ), plasma half-life (T_{1/2}), dose corrected exposure (AUC), and partial exposure (AUC). Results of these studies are shown in FIGS. 29-34.

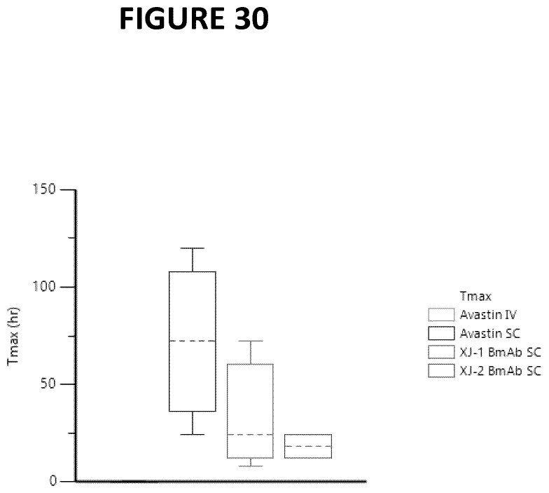

As seen in FIG. 29, the two XeriJect® bevacizumab formulations, XJ-1 and XJ2, showed very similar plasma concentration kinetics as the Avastin® formulation delivered subcutaneously, whether these results were plotted on a linear scale (FIG. 29A) or a semilogarithmic scale (FIG. 29B). Avastin administered intravenously showed the typical bolus effect at early time points that is commonly seen with IV-administered therapeutics. The time required to maximum plasma concentration (Tₘₐₓ) was then evaluated for each formulation, with results shown in FIG. 30. Tₘₐₓ for both Xeriject formulations was found to be shorter than the Avastin formulation administered subcutaneously (18 hours for XJ- 1 and 24 hours for XJ-2, vs. 72 hours for SC Avastin), although as expected it was longer than that seen with IV-administered Avastin (0.08 hours).

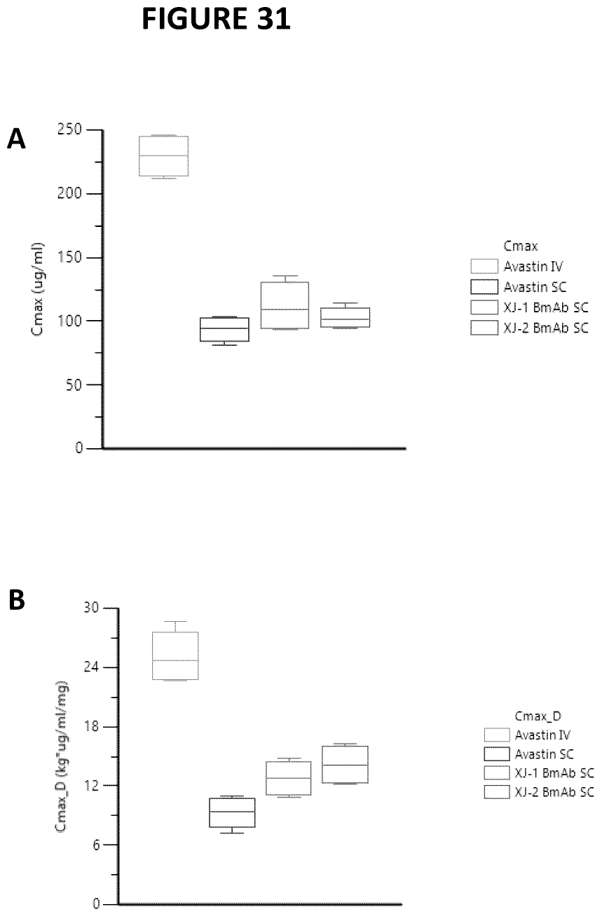

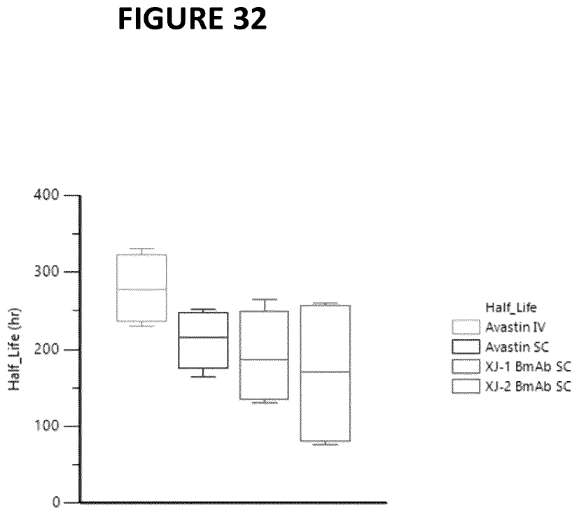

Next, the maximum absorption (Cₘₐₓ) of these various formulations was evaluated, with results shown in FIG. 31 for uncorrected Cₘₐₓ values (FIG. 31A) or Cₘₐₓ values corrected for dose and body mass of the animals (FIG. 31B). These results, together with the Tₘₐₓ results shown in FIG. 29, indicate that the Xeriject formulations of bevacizumab have more rapid absorption than Avastin administered subcutaneously, as indicated by shorter Tₘₐₓ (FIG. 30) and higher dose-corrected Cₘₐₓ (FIG. 31B) values. An assessment of the half-life for each of these formulations showed that subcutaneously administered bevacizumab formulations (both Xeriject and Avastin) demonstrated shorter half-lives than was observed for IV-administered Avastin (FIG. 32).

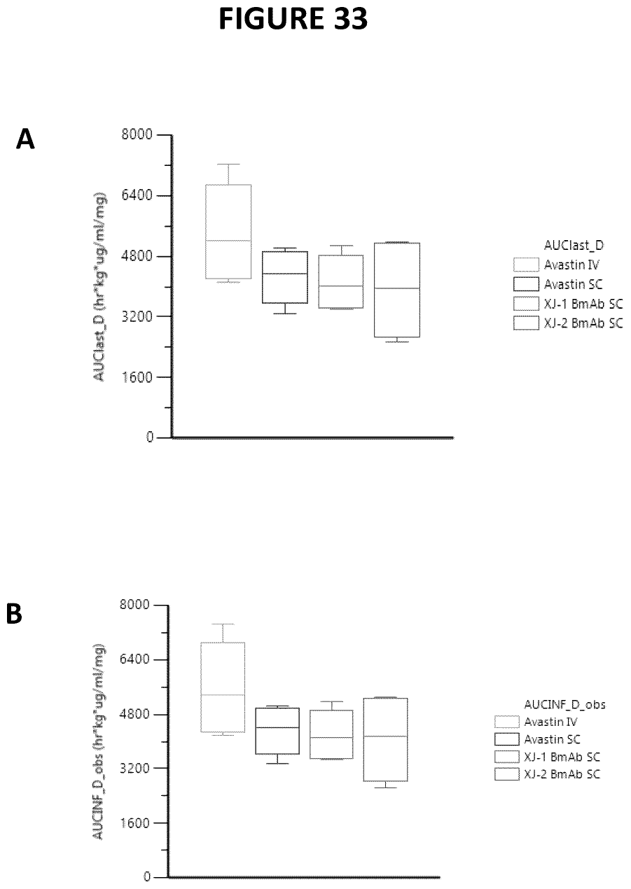

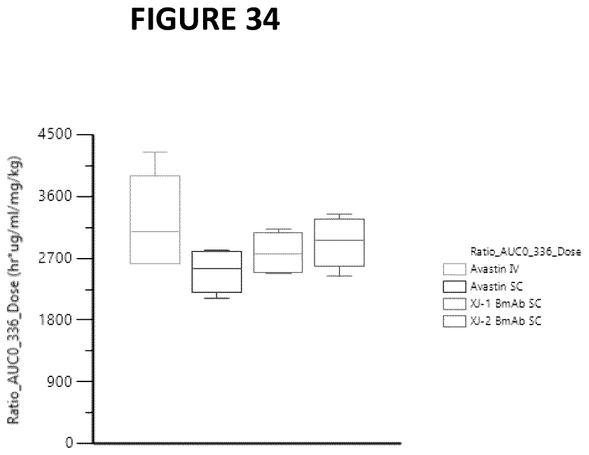

To assess total exposure of animals to the antibody, dose-corrected exposure (both AUCₗₐₛₜ and AUCo) were evaluated. Results are shown in FIG. 33 and demonstrated that dose-corrected exposure (both AUCₗₐₛₜ (FIG. 33A) and AUCo. (FIG. 33B)) were similar between the Xeriject formulations and the subcutaneously administered Avastin formulation, with both assessments showing a somewhat higher exposure value in animals receiving intravenously administered Avastin. Similar results were observed when assessing dose-corrected 14-day partial exposure (AUC_{0-336 hr}; FIG. 34), although one of the Xeriject formulations (XJ-2) showed a somewhat higher partial exposure value compared to Avastin that was subcutaneously administered.

Collective results for these studies, uncorrected for dose or corrected for dose, are presented in Tables 26 and 27:

TABLE 26
Collective Pharmacokinetic Results, Xeriject vs. Avastin (Uncorrected)

| | Mean | | | | | | |
|---|---|---|---|---|---|---|---|
| | Dose | Tₘₐₓ | Cₘₐₓ | AUC₃₃₆ | AUCₗₐₛₜ | AUC_{∞} | T_{1/2} |
| Formulation | (mg) | (hr)* | (μg/mL) | (μg*hr/mL) | (μg*hr/mL) | (μg*hr/mL) | (hr) |
| Avastin ®, | 106 | 0.08 | 230 | 28380 | 47990 | 49271 | 227 |
| IV | | | | | | | |
| Avastin, SC | 106 | 72 | 94 | 25574 | 43336 | 44098 | 215 |
| XJ-1 BmAb | 94 | 24 | 110 | 23780 | 35001 | 35915 | 187 |
| XJ-2 BmAb | 82 | 18 | 99 | 21413 | 28603 | 29928 | 170 |

*Tₘₐₓ values are median; all other values show mean.

TABLE 27
Collective Pharmacokinetic Results, Xeriject vs. Avastin (Dose-corrected)

| | Mean | Cₘₐₓ/D | AUC₃₃₆/D | AUCₗₐₛₜ/D | AUC_{∞}/D | Bio- | Bio- |
|---|---|---|---|---|---|---|---|
| | Dose | (kg* | (hr*kg* | (hr*kg* | (hr*kg* | availability | availability |
| Formulation | (mg) | μg/mL/mg) | μg/mL/mg) | μg/mL/mg) | μg/mL/mg) | (AUCₗₐₛₜ/D) | (AUC_{∞}/D) |
| Avastin ® | 106 | 24.7 | 3084 | 5215 | 5357 | N/A | N/A |
| IV | | | | | | | |
| Avastin | 106 | 9.4 | 2553 | 4334 | 4409 | 83% | 82% |
| SC | | | | | | | |
| XJ-1 | 94 | 12.7 | 2758 | 4007 | 4112 | 77% | 77% |
| BmAb | | | | | | | |
| XJ-2 | 82 | 14.1 | 2968 | 3953 | 4146 | 76% | 77% |
| BmAb | | | | | | | |

All values show mean.

Taken together, the results presented in this example demonstrate that bevacizumab plasma concentration-time curves in minipigs are similar for two different XeriJect® bevacizumab formulations administered subcutaneously and Avastin® administered subcutaneously, while Avastin administered intravenously has a higher peak exposure. Both bevacizumab paste formulations demonstrated more rapid absorption than Avastin administered subcutaneously, as indicated by shorter Tₘₐₓ and significantly higher dose-corrected Cₘₐₓ values for both of the Xeriject formulations. In addition, the dose-corrected exposure (AUCₗₐₛₜ and AUCo) was similar between Xeriject formulations and Avastin administered subcutaneously. The dose-corrected partial 2-week exposure (AUC₃₃₆) trended higher for the XJ-2 bevacizumab formulation compared to Avastin administered subcutaneously. Finally, the total bioavailability of Avastin administered subcutaneously and the Xeriject bevacizumab formulations was lower than observed for Avastin administered intravenously, although both were likely to still be within therapeutic dose levels. Thus, the Xeriject technology can be used to prepare high concentration formulations of bevacizumab that can be injected subcutaneously at lower volumes and less frequently than those currently used to deliver Avastin intravenously, while still delivering therapeutic levels of antibody that appears to be well-tolerated and rapidly absorbed in subject animals.

### Example 9: Nonclinical Evaluation of High Solids Pastes Comprising Insulin

To evaluate the pharmacokinetic (PK) and pharmacodynamic (PD) profiles of insulin pastes, two paste formulations (XJ-6 and XJ-8) were prepared as described in Example 6 and evaluated in Yucatan minipigs. Both XJ-6 and XJ-8 were formulated to have an insulin content corresponding to u200 (7.5 mg/mL). Insulin powders were prepared by spray drying, with a feed solution containing a total solids-loading (dissolved material) of 50 mg/mL and comprising recombinant human insulin (0.52 mg/mL), trehalose (from dihydrate; 49.0 mg/mL), histidine (0.3 mg/mL) PS80 (0.01 mg/mL), EDTA (0.1 mg/mL) and pH adjusted to 9.0 (±0.1) using NaOH and/or HCl.

This solution was filtered (0.2 μm PVDF membrane) and spray dried with a BUCHI B-290 instrument at the following settings: inlet temp=140° C./liquid feed rate=8%/nozzle gas pressure ball meter reading=60 mm/aspirator setting=90%. The powder was secondary dried under vacuum to reduce the moisture content of the powder to <2% (w/w). The powder was blended with either pure Miglyol 812 (XJ-6) or Miglyol 812 containing 1% (v/v) PS80 (XJ-8). The solids contents of XJ-6 and XJ-8 were 57% and 56%, corresponding to solids concentrations of approximately 680 mg/mL and 670 mg/mL, respectively.

These test articles (XJ-6 (XeriJect-6) and XJ-8 (XeriJect-8)) were evaluated for pharmacokinetics (PK) and pharmacodynamics (PD) (changes in blood glucose) in the Yucatan minipig and compared to the commercial product Humulin R (u100). Each formulation was administered by subcutaneous injection to 6 male Yucatan minipigs at a dose of 0.5 U/kg insulin (0.017 mg/kg insulin).

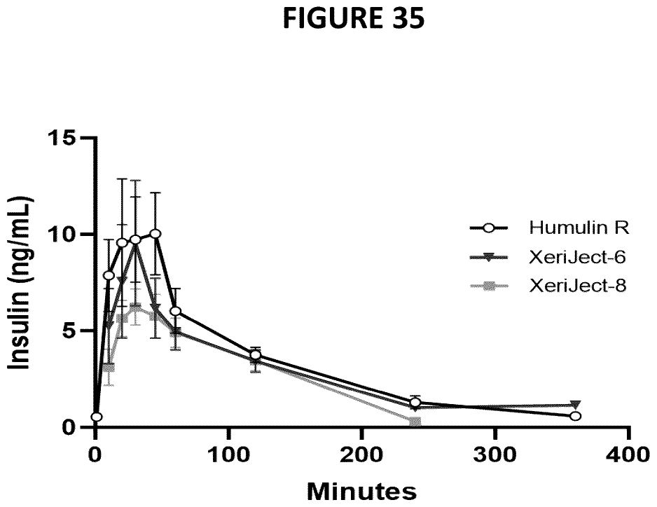

The insulin pharmacokinetic profiles of all three formulations were similar, although insulin exposure was lower with XJ-6 as shown in FIG. 35. Administration of 0.5 U/kg Humulin R (3.5 mg/mL insulin) SC to minipigs produced plasma insulin exposure with a mean (±SD) Cₘₐₓ of 13±3 ng/mL and mean (±SD) AUCₗₐₛₜ of 1175±144 ng*min/mL. The median Tₘₐₓ was 30 min (range: 10 min to 45 min) and mean (±SD) half-life was 79±12 min. Administration of 0.5 U/kg XJ-6 (17.4 mg/mL insulin) SC to minipigs produced similar, though slightly lower insulin exposure compared to Humulin R with a mean (±SD) Cₘₐₓ of 10±3 ng/ml and a mean (±SD) AUCₗₐₛₜ of 902±139 ng*min/mL. The median tₘₐₓ (30 min, range: 30 min to 60 min) was similar to Humulin R and the half-life (129 min) was 63% longer. XJ-8 (17.4 mg/mL insulin) administration of 0.5 U/kg SC to minipigs produced lower insulin exposure compared to Humulin R with a mean (±SD) Cₘₐₓ of 7.0±1.1 ng/ml and a mean (±SD) AUCₗₐₛₜ of 643±92 ng*min/mL. The median Tₘₐₓ (30 min, range: 20 min to 45 min) and half-life (67 min) were similar to Humulin R.

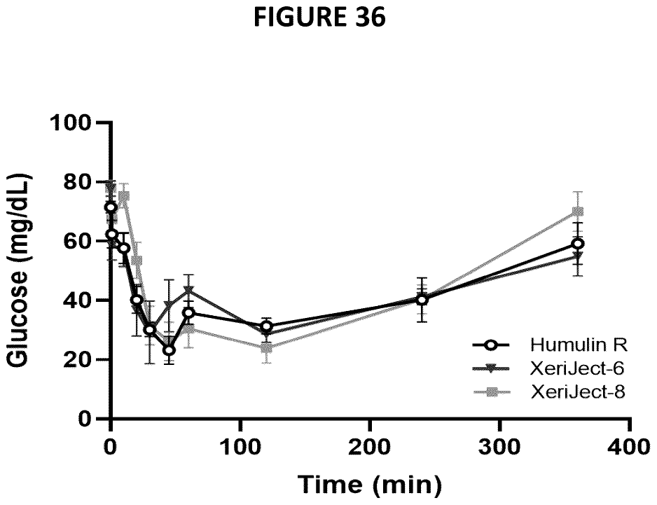

Administration of Humulin R, XJ-6 and XJ-8 produced similar PD responses by rapidly decreasing blood glucose levels as demonstrated in FIG. 36. Humulin R decreased blood glucose to a mean (±SD) of 21±4 mg/dL from a mean (±SD) baseline of 72±2 mg/dL by a median time of 38 min (range: 30 min to 45 min). XeriJect-6 decreased blood glucose levels to a mean (±SD) of 14±6 mg/dL from a mean (±SD) baseline of 78±3 mg/dL by a median time of 75 min (range: 20 min to 360 min). XeriJect-8 decreased blood glucose levels to a mean (±SD) of 17±6 mg/dL from a mean (±SD) baseline of 78±3 mg/dL by a median time of 83 min (range: 30 min to 120 min). Some instances of hypoglycemia occurred following insulin administration and animals were treated with oral administration of 50% dextrose. Pharmacodynamic effects of test articles on glucose levels shown here are only approximate for minipigs provided dextrose for treatment of hypoglycemia.

### Example 10: Production, Characterization and Preparation of an Injectable Paste Comprising Immune Globulin G (Igg)

The following example describes the preparation of injectable paste formulations containing high concentrations of the polyclonal antibody (pAb) immune globulin G (IgG). The solids phase in this example comprised an IgG powder prepared by spray drying an aqueous feed solution having a solids-loading of approximately 51 mg/mL, of which 40 mg/mL is the IgG protein (corresponding to over 78% (wt.) of the total solids-loading). Prior to the final feed solution preparation, IgG was buffer-exchanged against an aqueous solution to yield a final feed solution composition listed in Table 28.

TABLE 28
Formulation Feed Solution Composition for IgG Powder Preparation

| Composition | Concentration | % wt. |
|---|---|---|
| IgG | 40 mg/mL | 78.66 |
| Trehalose (from Dihydrate) | 10.3 mg/mL | 20.26 |
| Polysorbate 20 | 0.1 mg/mL | 0.20 |
| Lactic Acid | 0.45 mg/mL | 0.88 |
| pH | 3.8 | — |
| Total | 50.85 | 100 |

This formulation feed solution was spray dried using BUCHI B-290 spray dryer parameters and conditions shown in Table 29.

TABLE 29
Spray Dryer Parameters and Conditions for IgG Powder Preparation

| Parameter | Values |
|---|---|
| Inlet temperature | 80° C. |
| Aspirator | 70% (28 m³/h) |
| Feed flow rate | 10% (3 mL/min) |
| Nozzle flow rate | 60 mm (742 L/h) |

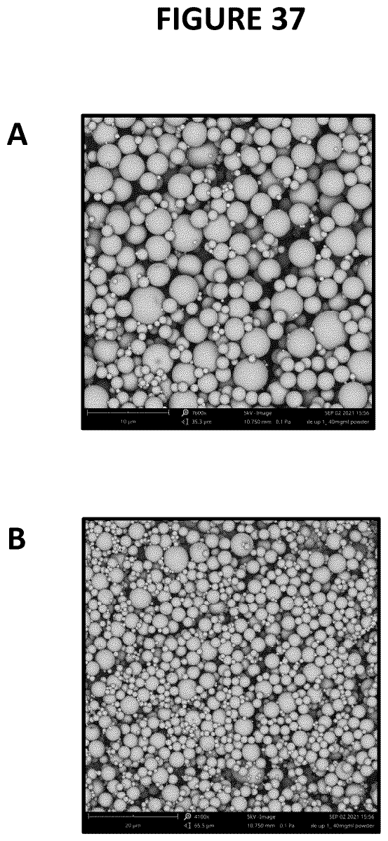

The spray dried powder was secondary dried under reduced pressure to lower moisture content (<1% (w/w)) and then evaluated by scanning electron microscopy (SEM) to examine the particle morphology. As seen in FIGS. 37A and 37B, the particles exhibited a generally spherical shape with a relatively smooth surface, minimal-to-no observed surface pitting (dimpling), and a moderately polydisperse size distribution (span ~2.0).

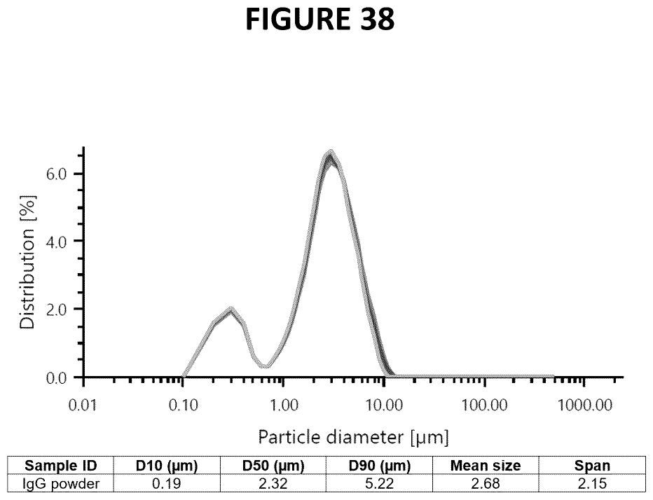

Next, the particle size and particle size distribution of the IgG powder was determined by laser diffraction (where the sample was dispersed in a non-solvent (e.g., propyl alcohol) with continuous sample sonication to disrupt powder agglomerates). As shown in FIG. 38, a small particle population having a D90<10 μm with a moderate polydisperse (span ~2) and bi-modal distribution where there is a defined population of fine particles (<1 μm) and a population of larger (1-10 μm) particles. It is noted that under the conditions described in this example a relatively bi-modal particle size distribution was observed. However, changes to the formulation and/or process parameters and/or equipment (e.g., larger scale spray dryer, etc.) and/or characterization method can shift the observed particle size distribution to generally unimodal or multi-modal (e.g., bimodal, trimodal, etc) profiles that remain suitable for preparing high solids concentration pastes.

Following characterization by SEM, laser diffraction, and moisture content analysis (data not shown), a paste was prepared by blending the powder with Miglyol 812 N (using a planetary-orbital mixer) to a solids content of 65%. The density of the powder measured by helium pycnometry was approximately 1.2 g/mL, corresponding to a solids concentration of approximately 780 mg/mL. The solids-loading of the feed solution, particularly the weight-percent of the protein in the feed solution as a percentage of total solids-loading (~78% w/w), may be translated to the approximate weight-percent of IgG in the spray dried powder (~78% (w/w)), which may then be used to determine the approximate protein concentration in the paste (~600 mg/mL).

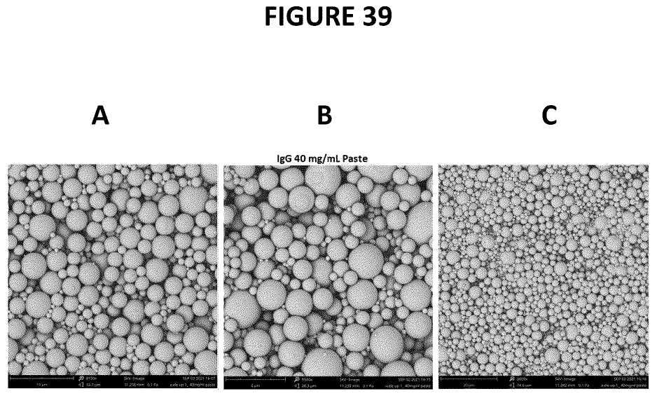

The IgG paste was also imaged by SEM to examine any changes in the morphology and/or size distribution of the particles (post-blending with Miglyol) and to observe the particle packing in the paste. As shown in FIGS. 39A, 39B, and 39C, after the preparation of the IgG paste, the morphology and size distribution of the particles comprising the solids phase of the paste remained relatively unchanged. The SEM analysis of the IgG paste indicated good particle packing/arrangement arising from the highly concentrated solids phase of the paste, which imparts the semi-solid and viscoelastic properties of the paste and may sterically inhibit the particles from settling over time under storage conditions and periods relevant to a pharmaceutical drug product.

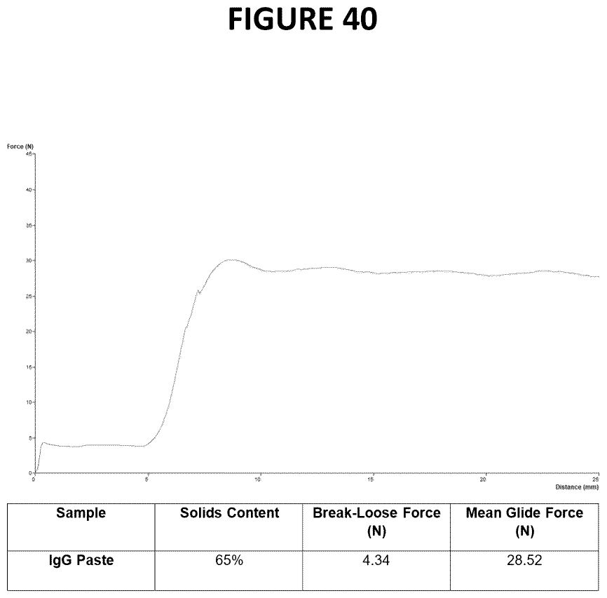

Finally, to evaluate the injectability of the high solids concentration IgG paste, 1 mL of the paste was loaded into cyclic olefin copolymer (COC) syringes (Schott) having a barrel internal diameter of 5.0 mm and delivered through a 25-gauge, ultra-thin wall, 6 mm needles. FIG. 40 provides the injection force profile (plotted as force in Newton (N; Y-axis) versus plunger distance traveled in millimeters (mm, X-axis)) of the 65% solids content IgG paste measured using a TA.XT Plus model texture analyzer (Stable Micro Systems). The measured injection force (mean glide force) was below 30 N at a volumetric flow rate of 31 μL/sec, as measured by a texture analyzer.

Thus, this example demonstrates that paste compositions having both a high solids concentration (>700 mg/mL) and a high protein concentration (>500 mg/mL) can be prepared and delivered through syringes and needles relevant to intracutaneous injection with moderate injection forces.

Together with those of the preceding examples, these results indicate that the paste formulations of high concentration therapeutic proteins that are provided by the present invention are useful in delivering controlled- or sustained-release depots of therapeutic proteins, including those of relatively high molecular weights, in small volumes subcutaneously in a way that improves the patient experience from that obtained with higher volume administration of aqueous formulations of recombinant human proteins.

### Example 11: High Concentration (>70% (w/w)) Monoclonal Antibody Powders

As the indications treated by therapeutic monoclonal antibody (mAb) drug products continue to expand, there is continuing need for higher doses and corresponding higher concentration formulations to minimize injection volume, reduce injection time, and improve patient compliance. An additional benefit of higher concentration mAb formulations is the potential to change the administration route (e.g., intravenous to subcutaneous administration).

One approach to increasing mAb content in a formulation is by increasing the mAb content in the powder that constitutes the dispersed phase of a suspension, slurry, or paste. As a non-limiting example, for a formulation that contains 60% (w/w) powder dispersed in a non-solvent carrier (e.g., the continuous phase), and having a formulation density of 1.1 g/mL, the powder content in the formulation is approximately 660 mg/mL. If the mAb content in the powder is 50% (w/w), the mAb content in the formulation is approximately 330 mg/mL. Alternatively, if the mAb content in the powder is 90% (w/w), with all other factors remaining constant, the mAb content in the formulation is approximately 590 mg/mL. This increase in mAb content would reduce the injection volume for an example 1250 mg dose from ~3.80 mL for the 330 mg/mL formulation down to 2.10 mL for the higher 590 mg/mL formulation. This latter volume (2.10 mL) could potentially be administered as a single injection (e.g., via a standard 2.25 mL syringe), while the latter would require multiple injections (potentially at multiple sites) or a longer infusion-based (either IV or SC) delivery.

However, increasing the protein content in a spray dried powder is accompanied by a corresponding decrease in the concentration of the stabilizing excipient(s) in the formulation (e.g., amino acids, sugars/disaccharides, surfactants, etc.). While there are many factors that influence the overall stability of a biologic/protein molecule (e.g., intrinsic stability of the molecule, the processing conditions (which influences the type, magnitude, and duration of the stresses to which the molecule is exposed), etc.), in general the lower the concentration of the stabilizing excipient(s) in a formulation, the greater the potential instability of the biologic/protein in the formulation, either immediately following processing (e.g., spray drying an aqueous feed solution to a powder) or over the proposed shelf-life. Accordingly, mAbs are generally processed to powders (e.g., via spray drying, freeze drying, etc.) using an excipient concentration of 30% (w/w) or greater in the resulting powder.

Increasing protein content in spray-dried powders may thus adversely impact the storage stability of those powders and reduce their usefulness in therapeutic applications. The present inventors reasoned, however, that the methods and formulations of the present invention could overcome such adverse effects on storage stability. To examine this possibility more closely, a study was performed to evaluate how overall protein content in a spray dried powder prepared by the methods of the present invention can influence stability over a short-term three-month storage period. Sample formulations were prepared by spray drying an aqueous feed solution containing the monoclonal antibody (mAb) bevacizumab (referred to hereafter as “BmAb”), which is the active pharmaceutical ingredient in the commercial drug product, Avastin®. The feed solutions were spray dried to powders using a Buchi B-290 bench top spray dryer, having the following process parameters: Inlet Temperature: 80° C.; Liquid Feed Rate=10% (based on B-290 instrument setting, and corresponding to ~3 mL/min); Nozzle (Atomizing) Pressure: 60 (based on B-290 internal flow meter and corresponding to ~750 L/hr); Aspirator: 70% (based on B-290 instrument setting and corresponding to a flow of ~28 m³/hr).

The composition of the spray dried feed solutions that were evaluated in this study are provided below (Table 30) and listed according to their target mAb content in the aqueous feed solution upstream of spray drying.

TABLE 30
Target Feed Solution Compositions (~40 mg/mL total solids loading)

| mAb | | | | |
|---|---|---|---|---|
| Content | Trehalose | PS80 | Proline | Malate |
| (% w/w) | (% w/w) | (% w/w) | (% w/w) | (% w/w) |
| 62.66% | 18.44% | 0.18% | 17.91% | 0.81% |
| 70.00% | 14.82% | 0.14% | 14.39% | 0.65% |
| 80.00% | 9.88% | 0.10% | 9.59% | 0.43% |
| 90.00% | 4.94% | 0.05% | 4.80% | 0.22% |
| 100.00% | 0.00% | 0.00% | 0.00% | 0.00% |

The mAb content is provided in Table 30 as the weight-percent (% wt) of total solids loading, where total solids loading consists of the solubilized components that are not volatile during the powder preparation process and that will therefore be incorporated into the resulting powder. For example, an aqueous feed solution that contains a mAb content of 25 mg/mL and a combined excipient content of 25 mg/mL (where excipients include amino acid(s), sugar(s), disaccharide(s), surfactant(s), and/or other common pharmaceutical excipients that are non-volatile) will have a total solids-loading of approximately 50 mg/mL. In this example, the mAb content of the feed solution was 50% (w/w) of total solids loading, and the resulting spray dried powder had a corresponding mAb content of approximately 50% (w/w). It is to be noted that due to the residual moisture content in the powder (even post-secondary drying (e.g., under vacuum)), as well as different salts, ions, etc., that are used during preparation of the feed solution (e.g., pH adjustment), the mAb content in the powder is typically less than targeted based on the theoretical/calculated solids loading, but is generally within ~10% of the target weight-percent.

All feed solutions were prepared by initially washing (via tangential flow filtration (TFF)) a stock BmAb formulation purchased from a commercial drug substance supplier (Dr. Reddy's Laboratories SA; Basel, Switzerland). The stock drug substance was washed with approximately five diavolumes of water (removing most non-mAb components) to a final mAb concentration of ~43 mg/mL (measured by A₂₈₀). All formulations evaluated in this Example were prepared from this washed BmAb solution. Feed solution excipients were then spiked in from concentrated aqueous stock solutions of the individual excipients to achieve the final target composition for each feed solution. All feed solutions were prepared to a total solids-loading of approximately 40 mg/mL and pH adjusted to approximately 3.8 (with HCl and/or NaOH) immediately prior to spray drying. As will be known and understood by the ordinarily skilled artisan, while the TFF process may remove the majority of the non-protein formulation excipients/components, it may not completely remove the non-mAb components, and these may be carried over into subsequent processing steps.

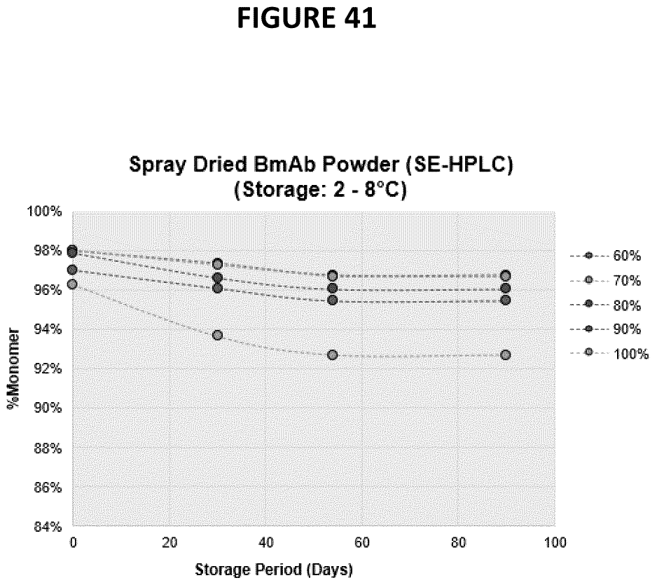

Spray dried powder samples were secondary dried under vacuum (at 5° C. overnight) to lower residual moisture content to <1% (w/w). The BmAb powders were then stored in 2R ISO glass vials (stoppered and crimp-sealed) and placed in a 2-8° C. stability chamber to simulate refrigerated storage conditions. At approximately one-month intervals (days 30, 54, and 90), samples were removed from the stability chamber and evaluated by size-exclusion ultra-high-performance liquid chromatography (SE-UHPLC) to assess the level of mAb aggregates (e.g., high molecular weight (HMW) products) forming over time in the spray dried powder. Samples were also analyzed immediately after processing (day 0) to obtain a baseline comparison to evaluate degradation over time. Data from the stability studies are shown in FIG. 41.

The data provided in FIG. 41 indicate that increasing the mAb content (as a percentage of total solids loading) in the feed solution leads to more aggregates forming during the spray drying process, where the protein is subjected to different levels of stresses (including shear stress as well as thermal and interfacial stresses), even upon storage at 2-8° C. The 60 and 70% (w/w) mAb content formulations exhibited comparable stability across the studied timepoints. Increasing the mAb content from 80 to 90% promoted increased degradation, however this instability was relatively minor compared to the 100% formulation, which contained no added excipients. All spray dried powders exhibited the non-linear degradation profile typically observed with solid-state formulations, where initial degradation appeared to significantly slow down, essentially plateauing over longer-term storage periods. Therefore, while the 80 and 90% mAb content powders did exhibit increased aggregate formation compared to their 60 and 70% counterparts, in this short-term study they exhibited the potential to achieve long-term (>1 year, 2 years, etc.) storage stability when using the standard ≥95% monomer stability specification used for many mAb formulations.

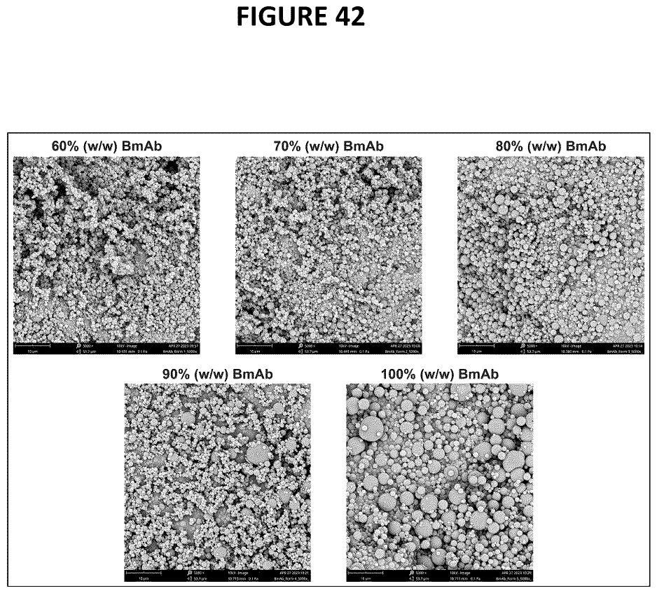

In addition to BmAb stability, the spray dried powders were also characterized by scanning electron microscopy (SEM) to qualitatively assess powder particle size and morphology. As shown in FIG. 42, all samples appeared to exhibit an overall spherical morphology, which may be the preferred morphology for a particular formulation. There were some size differences observed, particularly for the 100% mAb sample, however particle size may be controlled by adjusting/optimizing the spray dryer process parameters (e.g., inlet temperature, atomization pressure, feed rate). As discussed above, all samples in this study were prepared using the same spray dryer process parameters.

Based on the observations from this study, high mAb concentration formulations (>350 mg/mL) may be prepared by greatly increasing the mAb content of the powder/dispersed phase of a formulation. As a non-limiting example, Table 31 provides the potential mAb contents (by % wt mAb content in the powder) that could be achieved in a viscoelastic suspension formulation having a solids content of 60% and a density of 1.1 g/mL (for a total powder content of approximately 660 mg/mL).

TABLE 31
Potential mAb contents of a viscoelastic suspension formulation

| | | Formulation | Powder | |
|---|---|---|---|---|
| Formulation | Formulation | Powder | mAb | Formulation |
| Density | Solids Content | Content | Content | mAb Content |
| 1100 | 60% | 660 | 60% | 396 |
| 1100 | 60% | 660 | 70% | 462 |
| 1100 | 60% | 660 | 80% | 528 |
| 1100 | 60% | 660 | 90% | 594 |
| 1100 | 60% | 660 | 100% | 660 |

It is to be noted that additional formulation development will likely improve the composition of the feed solution and suppress process-related and/or storage stability degradation, particularly as differing concentrations of mAb could require unique formulations (e.g., excipient types/concentrations, pH, etc.) to further maximize stability. However, in this initial feasibility study all feed solutions were derived from a single formulation developed for a 60-70% (w/w) mAb content powder, and the mAb content of the formulations in the present study was increased by diluting all excipients out equally. The present inventors believe that further optimizing the feed solution formulation could further inhibit mAb degradation, particularly the instability promoted during the powder preparation step (e.g., spray drying), which should extend the shelf-life of a high-concentration mAb formulation. Thus, by adjusting the mAb content in the powder/dispersed phase for a formulation, drug concentrations approaching, and even exceeding, 600-900 mg/mL, can be achieved.

### Example 12: Toxicology and Toxicokinetic Examinations of High Concentration Monoclonal Antibody Powders

As a part of the development of the present formulation technology, the inventors examined the potential toxicity and toxicokinetic characteristics of an exemplary high-concentration monoclonal antibody (mAb) formulation in non-human primates. In this example, the potential toxicity of a formulation of Trastuzumab prepared by the methods described herein and administered subcutaneously to cynomolgus monkeys was determined, in comparison to a US-marketed product comprising the same mAb (HERCEPTIN®) as active pharmaceutical ingredient (API), when administered to monkeys via intravenous infusion as directed by current HERCEPTIN labeling.

Male and female cynomolgus monkeys (three each per test group) were dosed with Trastuzumab formulations of the present invention (“XeriJect® Trastuzumab”), with vehicle control, or with commercial (US formulation) HERCEPTIN, once weekly for five weeks (dosages administered on days 1, 8, 15, 22 and 29). Dose levels and concentrations were as shown in Table 32.

TABLE 32
Toxicity and Toxicokinetic Testing - Experimental Design

| | | | Dose | Dose |
|---|---|---|---|---|
| Group | | Dose Level | Volume | Concentration |
| No. | Test Sample | (mg/kg/day) | (mL/kg) | (mg/mL) |
| 1 | Vehicle control | 0 | 0.12 | 0 |
| 2 | XeriJect ® | 50 | 0.12 | 417 |
| | Trastuzumab | | | |
| 3 | HERCEPTIN ® | 50 | 2.38 | 21 |

Toxicokinetic sampling of each monkey was performed by blood draws on the days of dose 1 and dose 4 (days 1 and 22), and anti-drug antibody samples were taken on day 0 and on day 30. After day 30 sampling, animals were humanely sacrificed, and necropsy was then performed.

### Results

1. Clinical Observations and Body Weights. All animals in each group survived to the scheduled necropsy. There were no clinical observations noted in any group for any test sample. All clinical parameters were within the expected levels based on the species, age and sex. Similarly, there were no body weight changes noted in any group for any test sample.
2. Injection Site Observations. There were no definitive injection site observations related to any test sample administered to any animal. A slight edema was noted following dosing, but the incidence of edema was similar between the active test sample groups and the vehicle groups. Similarly, there was no erythema noted in any animal following dosing, either active or control.
3. Ophthalmology Observations. There were no drug-related ophthalmology observations in any of the three groups of animals.
4. Electrocardiology Observations. Transient sinus tachycardia was observed in each animal upon dose administration, but the cardiac rate returned to normal sinus quickly thereafter, and all electrocardiograms in all animals were otherwise qualitatively and quantitatively within normal limits. There appeared to be no effect on qualitative or quantitative ECG parameters from the subcutaneous injection of XeriJect Trastuzumab or intravenous infusion of HERCEPTIN.
5. Hematology, Coagulation, Clinical Chemistry and Urinalysis Observations. There were no drug-related observations for any of these clinical parameters in any of the three groups of animals.
6. Pathology Observations. At terminal euthanasia, there were no organ weight differences observed in any of the animal groups, and no drug-related gross anatomical differences were observed upon necropsy. Macroscopically, the only gross findings observed were considered incidental, of the nature occasionally observed in cynomolgus monkeys, and/or were of similar incidence in control and dosed animals and, therefore, were considered unrelated to administration of XeriJect® Trastuzumab or HERCEPTIN®. Microscopic necropsy observations were similar—no XeriJect Trastuzumab- or HERCEPTIN-related microscopic findings were observed. All microscopic findings were considered incidental because they were of the nature occasionally observed in cynomolgus monkeys and/or were of similar incidence and severity in control and dosed animals and, therefore, were attributed to normal biologic variation, study procedures (e.g., those microscopic findings observed at the subcutaneous and intravenous administrations sites), and/or variation in sexual maturity and unrelated to the administration of the test materials.

Conclusion. Taken together, these results demonstration that were no XeriJect Trastuzumab-related mortality, clinical signs, effects on body weights, injection site observations, ophthalmology, electrocardiograminations, clinical pathology parameters, organ weights, and macroscopic and microscopic examinations during and following a four-week drug administration protocol. Thus, weekly subcutaneous administration of XeriJect Trastuzumab at a weekly dose of 50 mg/kg/day was well tolerated and showed no adverse clinical, pathology, or toxicological events when compared to the intravenous administration of HERCEPTIN.

Together with the pharmacokinetic results observed in minipigs as described in Examples 8 and 9 herein, these results support future studies of high-solids/high-dose mAb formulations, such as those of the present invention, as potential drug products designed to treat, prevent and/or diagnose a variety of diseases and disorders in humans and veterinary animals.

### Example 13: Methods for Preparing Concentrated Suspensions with Improved Syringeability

As described in detail in the preceding sections, the presence of powder agglomerates in concentrated suspensions (e.g., VES) may impede delivery through fine-gauge needles suitable for parenteral injection, including but not limited to subcutaneous and intramuscular injection. Individual powder agglomerates need not be larger than the lumen diameter of the intended delivery needle to contribute to occlusion; the convergence of multiple sub-lumen agglomerates at a constriction point in the delivery flow path may impede or arrest the flow of the composition, as described elsewhere herein. The following examples demonstrate various approaches to agglomerate disruption and illustrate the effectiveness of the high-shear mixing and separation approach described herein.

Various approaches exist for directly preparing concentrated suspension (e.g., VES) at or near the target solids content. In one approach, mixers that provide relatively low shear and that are primarily effective for dispersion rather than agglomerate disruption (referred to herein as “dispersion mixers”), such as planetary-centrifugal mixers (e.g., those available from THINKY USA, Inc., Laguna Hills, CA, or equivalent), may be used to disperse the powders in a diluent. The present inventors have found, however, that the use of such dispersion mixers, while effective for dispersion (i.e., distributing the powder relatively homogeneously throughout a diluent), may not sufficiently disrupt agglomerates present in the powder. That is, powder agglomerates that are present at the start of mixing with a dispersion mixer may remain substantially intact upon completion of mixing, particularly where such agglomerates are robust (i.e., held together by strong interparticle forces). The distinction between dispersion and agglomerate disruption is significant, as a composition may appear well-dispersed upon visual inspection or by macroscopic assessment, yet may still contain a substantial population of agglomerates that could impede delivery through the intended flow path as described above.

In another approach, high-shear mixers may be used to mix the composition at or near the target solids content. However, as described in detail in the preceding sections, concentrated suspensions may exhibit high viscosity and non-Newtonian rheological behavior that may impede effective energy transmission throughout the bulk of the material, resulting in localized shear and heat generation that may be detrimental to thermally sensitive active pharmaceutical ingredients.

The present inventors have explored various approaches to disrupting or reducing powder agglomerates in concentrated suspensions described herein. In one approach, the powder (e.g., micronized powder) may be processed through a fine-mesh sieve or screen prior to being combined with the liquid phase (e.g., diluent) to form the concentrated suspension. As used herein, this processing step encompasses both conventional sieving (e.g., passing powder through a vibratory sieve or other gravity- or vibration-assisted sieving device) and screening (e.g., applying mechanical force to press the powder through a mesh screen), and may be referred to interchangeably as sieving or screening. It is noted that micronized powders, which are characterized by a high surface area-to-volume ratio, may not be effectively processed by conventional vibratory sieving alone, as such powders may agglomerate on the sieve surface rather than passing through the mesh openings. Micronized powders may also acquire electrostatic charge during processing steps such as air jet sieving, which may further promote agglomeration and impede effective sieving. In such cases, the application of mechanical force to press the powder through the mesh openings of a mesh-screen may be more effective at disrupting agglomerates and passing the powder through the screen than passive or vibration-assisted sieving. By passing the powder through a screen or sieve having a mesh opening size larger than the primary particle size of the powder (e.g., 20, 32, 45, 63, 75, 90, 106, 125 microns, or other suitable mesh sizes), agglomerates of primary particles that are larger than the mesh opening size may be disrupted as they are forced through the mesh openings, while the mechanical forces generated during passage of the powder through the mesh may additionally disrupt agglomerates that are smaller than the nominal mesh opening size. The mesh may further exclude any material that cannot be disrupted to a size below the mesh opening. Accordingly, the powder entering the subsequent blending step may contain fewer and/or smaller agglomerates. The sieved powder may then be combined with the diluent using a dispersion mixer (e.g., a planetary-centrifugal mixer) to form the concentrated suspension (e.g., VES). The present inventors have observed that concentrated compositions (e.g., viscoelastic suspensions), prepared from powders processed through a fine-mesh sieve prior to blending with the diluent may exhibit improved syringeability when delivered through fine-gauge needles, as compared to compositions prepared from the same powder that was not sieved prior to blending. Such improvements may include lower injection forces, more consistent injection force profiles, and a reduction in the frequency and/or magnitude of transient force peaks or spikes indicative of partial or complete occlusion of the delivery path by powder agglomerates.

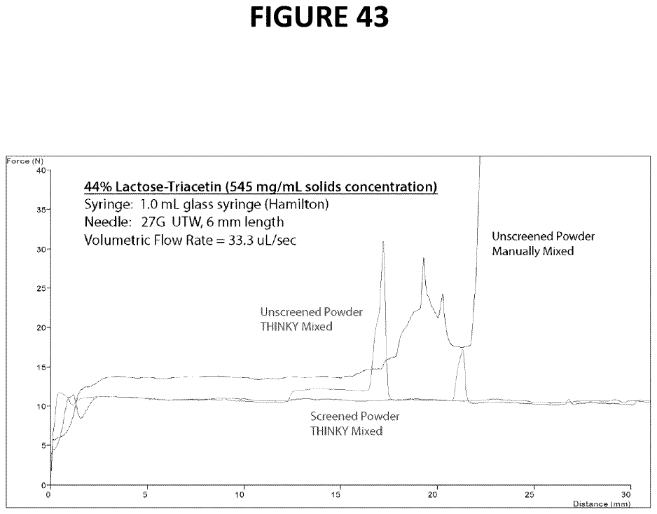

In one non-limiting example, concentrated suspensions were prepared by blending a micronized, crystalline powder with a hydrophobic diluent that was a nonsolvent with respect to the powder. In this example, the powder was micronized lactose monohydrate (Lactohale®, DFE Pharma) and the diluent was triacetin. The powder content in the composition was approximately 525 mg/mL (corresponding to approximately 44% (w/w) solids content). In a first preparation, the powder was combined with the diluent without any pre-processing of the powder, and the resulting composition was blended using a planetary-centrifugal mixer. In a second preparation, the powder was passed through a fine-mesh screen having a pore size of approximately 75 microns prior to being combined with the diluent, and the resulting composition was blended using the same planetary-centrifugal mixer under comparable conditions. The powder was passed through the fine-mesh screen by applying mechanical force to press the powder through the mesh openings, thereby disrupting powder agglomerates that were larger than the mesh opening size and reducing the overall agglomerate population in the powder entering the blending step. The resulting viscoelastic compositions from both preparations were evaluated for syringeability by measuring the injection force required to deliver the compositions through a 27-gauge needle. The results, shown in FIG. 43, demonstrate that the composition prepared from the powder processed through the fine-mesh screen (“Screened”) exhibited a more consistent injection force profile, with fewer and lower-magnitude force peaks, when delivered through the 27-gauge needle, as compared to the composition prepared from the powder not pressed through the fine-mesh screen (“Unscreened”), under otherwise comparable conditions. These results indicate that the disruption of powder agglomerates prior to blending with the diluent may improve the syringeability of the resulting viscoelastic composition, and support the broader finding disclosed herein that agglomerate disruption, by any suitable method, may be beneficial to the delivery performance of the concentrated suspensions (e.g., VES compositions) described herein.

While the approach described above, wherein powder is passed through a fine-mesh screen or sieve, demonstrates that the disruption of powder agglomerates prior to blending may improve the syringeability of the resulting concentrated suspensions, the present inventors have recognized that this approach presents practical limitations that may impede its implementation at commercial manufacturing scales. Specifically, processing micronized powders through fine-mesh screens may be a laborious, operator-dependent process that may be difficult to perform reproducibly at scale, and may be particularly challenging to implement within an aseptic manufacturing environment (e.g., within an isolator or cleanroom) as would be required for the production of injectable therapeutic compositions intended for parenteral administration. The screening or sieving approach is therefore disclosed herein as a potentially suitable method for laboratory-scale or development-scale preparation of concentrated suspensions (e.g., viscoelastic compositions) but may not represent an optimal approach for commercial-scale aseptic manufacturing.

In another approach, the present inventors examined the use of post-manufacture processing to reduce the presence of powder agglomerates in concentrated suspensions (e.g., viscoelastic compositions). As used herein, the term ‘post-manufacture processing’ refers to any processing step that is performed on the concentrated suspension after the powder and diluent have been combined by any suitable process (e.g., mixing, blending, dispersing, or otherwise incorporating the powder into the diluent) to form the concentrated suspension, for the purpose of disrupting, reducing the size of, or dispersing agglomerates that remain in the composition following its preparation. Post-manufacture processing may be performed using a milling device or any other suitable device or method, and is not limited to any particular type of equipment, number of processing passes, or mode of operation.

As used herein, the term ‘milling device’ refers broadly to any device, instrument, or apparatus that is configured to apply shear, compressive, impact, acoustic, hydraulic, cavitational, or other forces or energy to a suspension or concentrated suspension so as to disrupt, reduce the size of, or disperse agglomerates contained therein, without necessarily reducing the primary particle size of the powder. The term encompasses, without limitation, roll mills (including but not limited to three-roll mills, single-roll mills, and five-roll mills), ointment mills, bead mills, media mills, basket mills, immersion mills, colloid mills, attrition mills, high-pressure homogenizers, and any other device capable of applying sufficient force or energy to disrupt powder agglomerates within a suspension or concentrated suspension. In this approach, the powder and diluent may initially be combined by any suitable process to form the concentrated suspension, and the resulting composition is subsequently passed through a milling device to disrupt agglomerates that remain in the composition following its preparation. One example of such a milling device is a three-roll mill, which may be used for breaking down agglomerates in concentrated suspensions and improving material consistency and homogeneity. In a three-roll mill, the composition is passed between a series of rollers operating at different speeds, such that the differential surface speed between adjacent rollers generates shear forces sufficient to disrupt agglomerates within the composition as it passes through the gap between the rollers. The present inventors have observed that processing a concentrated suspension through a three-roll mill following its preparation may reduce the presence of agglomerates in the composition and may improve the syringeability of the composition for delivery through fine-gauge needles.

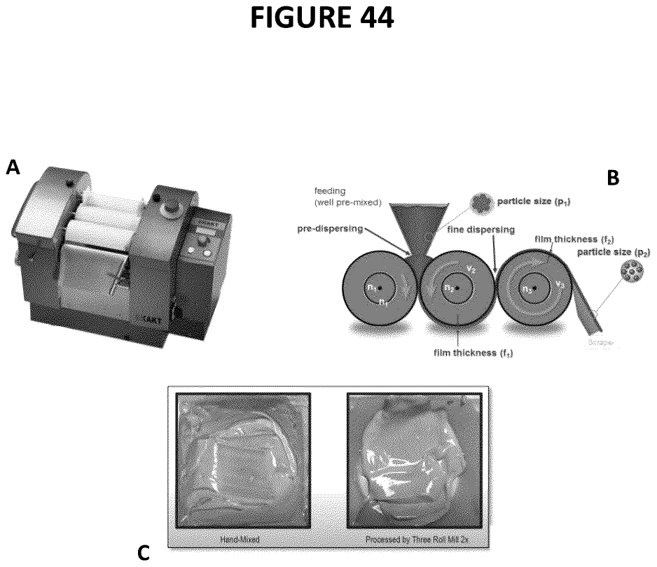

As a further non-limiting illustration, the operation of a three-roll mill (also referred to in the pharmaceutical compounding art as an “ointment mill”) in the context of the present disclosure is described. As shown in FIG. 44, a three-roll mill comprises three horizontally positioned rollers (commonly referred to as the feed roller, the center roller, and the apron roller) that rotate in alternating directions relative to one another and at progressively higher surface speeds from the feed roller to the apron roller. In operation, the concentrated suspension is introduced between the feed roller and the center roller, where it is drawn into the nip between the two rollers. As used herein, the term “nip” refers to the region of minimum clearance between two adjacent rollers, where the composition is compressed and subjected to maximum shear. The nip between the feed roller and the center roller is referred to herein as the “first nip.” As the composition passes through the first nip, the differential surface speed between the feed roller and the center roller generates shear forces that act upon the composition, disrupting agglomerates contained therein. The composition then transfers by adhesion from the feed roller to the faster-moving center roller, and is carried into the nip between the center roller and the apron roller (referred to herein as the “second nip”), where it is subjected to additional and typically higher shear forces due to the greater differential surface speed and, in many configurations, a narrower gap width. Upon exiting the second nip, the processed composition adheres to the apron roller and is removed from the roller surface by a scraper or take-off blade.

The gap width at each nip may be independently controlled to disrupt agglomerates, wherein agglomerates larger than the gap width are disrupted or excluded by the physical constraint of the gap, and wherein shear forces generated in the nip may additionally disrupt agglomerates smaller than the gap width. The gap width may be in the range of micrometers (e.g., approximately 100 microns, 50 microns, 20 microns, or other suitable gap widths). The composition may be passed through the three-roll mill in a single pass or in multiple successive passes to achieve the desired degree of agglomerate disruption. Processing the concentrated suspension through the three-roll mill may result in a more homogeneous composition with fewer and/or smaller powder agglomerates when compared to the same composition that has not been processed through the mill, as assessed by any suitable analytical method, including but not limited to visual inspection, microscopy (e.g., optical microscopy or scanning electron microscopy), particle size analysis, or injection force profiling.

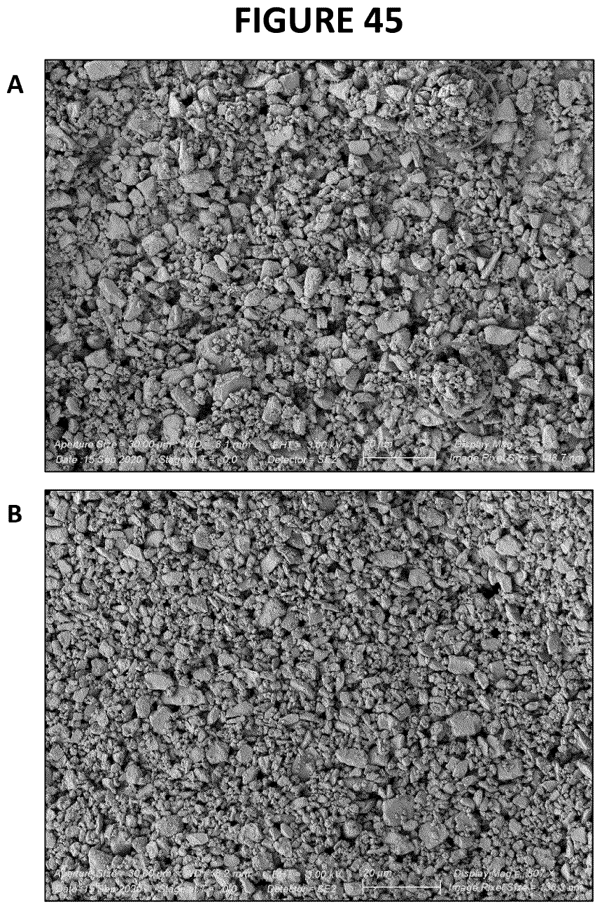

In one non-limiting example of this approach, inhalation-grade micronized lactose monohydrate (e.g., Lactohale®, DFE Pharma) was combined with a diluent (in this example, triacetin) to produce a concentrated suspension having a solids content of approximately 40% (w/w). The powder and diluent were combined using a planetary-centrifugal mixer. The resulting composition was then passed through a three-roll mill, with the first nip (between the feed roller and the center roller) set to a gap width of approximately 80 microns and the second nip (between the center roller and the apron roller) set to a gap width of approximately 30 microns, as measured using feeler gauges (also known as thickness gauges). The second nip gap of approximately 30 microns represented the smallest gap setting available on the mill used in this example. The morphology of the particles in the milled and unmilled compositions was examined by scanning electron microscopy (SEM), and representative micrographs are shown in (FIG. 45). The unmilled composition (FIG. 45A) contained a notable number of agglomerates (circled in the figure), appearing as flocculates of primary micronized particles. In contrast, the composition that had been processed through the three-roll mill (FIG. 45B) exhibited fewer and smaller agglomerates compared to the unmilled composition. It is noted that the three-roll mill process did not appear to reduce the size of the primary particles of the powder (i.e., the process was not used for particle comminution), but rather disrupted agglomerates of primary particles, resulting in a more uniform composition with a reduced agglomerate population as assessed by SEM.

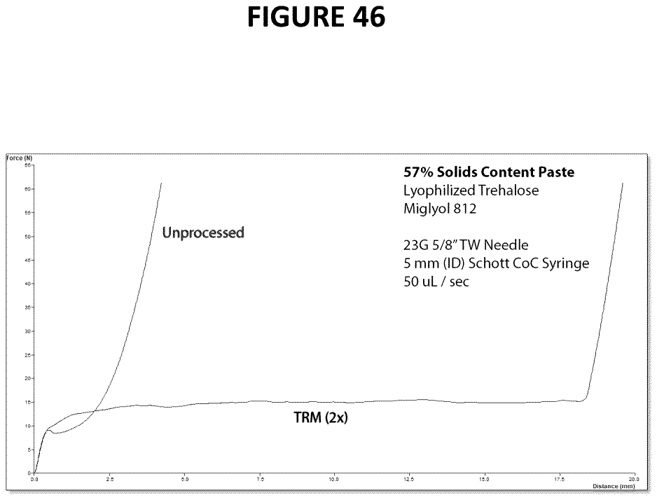

In a second non-limiting example, the effect of three-roll milling on the syringeability of the concentrated suspension was evaluated. Lyophilized trehalose (the powder) was combined with Miglyol® 812 (a medium-chain triglyceride serving as the diluent) using a planetary-centrifugal mixer (THINKY ARE-310, or equivalent) to produce a concentrated suspension having a solids content of approximately 57% (w/w). A first portion of the resulting composition was retained without further processing (the ‘unmilled composition’), and a second portion was passed through a three-roll mill (the ‘milled composition’). Both the unmilled and milled compositions were filled into 1 mL cyclic olefin copolymer (COC) syringes (having an internal barrel diameter of approximately 5 mm) fitted with fine-gauge needles (in this example, 23-gauge, ⅝-inch thin-wall needles), and the injection force profiles were measured during delivery of the compositions at a volumetric flow rate of 50 μL/sec. The unmilled (‘Unprocessed’) composition exhibited poor syringeability, with injection forces rising rapidly after the initiation of delivery and exceeding approximately 60 N, indicating substantial resistance to flow through the needle, consistent with partial or complete occlusion of the delivery path by powder agglomerates (FIG. 46). In contrast, the milled composition (‘TRM (2×)’) demonstrated substantially improved syringeability, with the entire syringe contents expelled with comparatively consistent injection force (FIG. 46). The composition, referred to in FIG. 46 as a ‘paste,’ exhibited the concentrated, non-pourable consistency characteristic of a concentrated suspension (e.g., VES) at this solids content. No phase separation of the powder and diluent was observed during delivery of the milled composition, and the entire volume of the composition was delivered smoothly from the syringe without interruption or evidence of flow path occlusion. These results demonstrate that post-manufacture processing of the concentrated suspension through a three-roll mill may substantially improve the syringeability of the composition for delivery through fine-gauge needles suitable for parenteral injection.

It will be appreciated that the three-roll mill described above is one example of a class of milling and dispersion devices that may be used to disrupt agglomerates in a concentrated suspension (e.g., a viscoelastic composition), whether the composition was prepared by a dispersion mixer, a higher-shear mixer, or any other mixing process or combination of processes. Other suitable devices include, but are not limited to, the milling and dispersion devices described above. The selection of a particular milling or dispersion device may be based on the viscosity of the composition, the nature and strength of the agglomerates to be disrupted, the sensitivity of the active pharmaceutical ingredients to shear and thermal exposure, the required throughput, and the compatibility of the device with aseptic manufacturing requirements, where applicable. It is noted that each of the foregoing devices may present one or more of the practical limitations described herein with respect to the three-roll mill, including but not limited to the labor-intensive nature of the process, the difficulty of implementing the process within an aseptic manufacturing environment, incomplete material recovery, and the potential for thermal or mechanical degradation of the active pharmaceutical ingredients during processing.

However, the approach of processing or treating a concentrated suspension (e.g., VES) after its manufacture to disrupt agglomerates, as exemplified by the three-roll mill and the other milling and dispersion devices described above, presents practical limitations for commercial-scale manufacturing. Such post-manufacture processing adds one or more additional steps downstream of suspension preparation, adding time, complexity, and potential for variability to the manufacturing process. Many of the devices suitable for post-manufacture agglomerate disruption, including but not limited to the three-roll mill, are open processes (i.e., the composition is exposed to the environment during processing), which may make it difficult to maintain the aseptic conditions required for the production of injectable therapeutic compositions. Certain devices may further present contamination risks arising from their mechanical construction. For example, in a three-roll mill, the motor or drive mechanism is mechanically coupled to the rollers, and lubricants (e.g., oil, grease, or other materials used in the drive train, bearings, or gearing) may migrate to the roller surfaces and be introduced into the composition during processing. In addition, lubricants and other materials associated with the drive mechanism may be aerosolized or may form airborne droplets during processing, potentially contaminating the surrounding environment and compromising the aseptic conditions required for the manufacture of injectable compositions. The potential for such contamination may render these devices unsuitable for the manufacture of injectable therapeutic compositions, where the introduction of extraneous materials, including lubricant-derived contaminants, into the composition is unacceptable. Even where closed-process alternatives exist, they may introduce additional complexity in terms of cleaning, sterilization, and validation, and may present analogous contamination risks from moving parts, seals, grinding media, or other components that contact the composition during processing. Material recovery from post-manufacture processing devices may also be incomplete, resulting in product loss that may be particularly undesirable when the active pharmaceutical ingredient is costly or available in limited quantities. The present inventors have therefore recognized the need for an alternative approach that may provide effective agglomerate disruption while being more readily scalable to commercial manufacturing volumes, more readily adaptable to aseptic processing environments, less susceptible to contamination of the composition by extraneous materials, and more readily integrated into a streamlined manufacturing process with fewer discrete processing steps.

The present inventors have recognized that the limitations described above may be addressed by diluting the suspension to a solids content sufficiently low that the composition behaves as a pourable suspension rather than a viscous concentrated suspension (e.g., VES), and applying high-shear mixing to the diluted suspension to disrupt the agglomerates while the viscosity is low enough for the shear to propagate more uniformly through the composition. At a sufficiently low solids content (e.g., below about 15% by weight, though not limited to any particular solids content or range of solids contents), the suspension may be mixed effectively by a wide variety of high-shear mixing equipment that would be inadequate for mixing the composition at its target solids content. Suitable high-shear mixers for disrupting agglomerates in the diluted suspension may include, but are not limited to, overhead mixers (e.g., propeller, paddle, or turbine type), saw tooth or dissolver disc mixers, rotor-stator mixers (e.g., those available from IKA, Silverson Machines, or equivalents), colloid mills, high-pressure homogenizers, microfluidizers, ultrasonic mixers or sonicators, acoustic or resonant acoustic mixers, vibratory mixers, ball mills or media mills, static or inline mixers, or any other mixer or combination of mixers capable of generating sufficient shear to disrupt the agglomerates present in the diluted suspension. The excess diluent in the diluted suspension additionally serves as a heat sink, absorbing and distributing the thermal energy generated during high-shear mixing and thereby mitigating the localized heat buildup that would otherwise occur in a high-viscosity composition. The selection of a particular mixer or combination of mixers, and the mixing conditions (e.g., mixing speed, duration, temperature, and number of passes), may be determined by the operator or prescribed by a predetermined protocol based on the characteristics of the powder, the nature and strength of the agglomerates to be disrupted, the viscosity of the diluted suspension, and the sensitivity of the active pharmaceutical ingredients to shear and thermal exposure. The high-shear mixing step may be performed in a single pass or in multiple passes, and may employ a single mixer or a sequence of two or more different mixers. Following the disruption of agglomerates by high-shear mixing in the diluted suspension, the excess diluent is removed from the suspension to increase the solids content and produce a concentrated suspension. This concentration step may be performed using a separation apparatus of the type described herein.

It will be appreciated that the high-shear mixing and separation approach described herein is not limited to powders that are agglomerated, and may be applied to any powder and diluent combination where preparation of a concentrated suspension from a dilute starting suspension is desired, regardless of the agglomerate status of the starting powder.

It will be appreciated that the viscoelastic character of concentrated suspensions (e.g., VES) described herein is a function of the combined properties of the powder and the diluent, including but not limited to the particle size, particle size distribution, specific surface area, particle morphology, and packing behavior of the powder, the viscosity and surface tension of the diluent, and the interactions between the powder and the diluent, and is not determined solely by the solids content of the composition. Accordingly, a concentrated suspension may exhibit viscoelastic behavior at a wide range of solids contents depending on the particular powder and diluent employed, and the compositions, methods, and apparatus described herein are not limited to any particular solids content or range of solids contents.

As a non-limiting example of the process, a suspension is prepared by mixing the powder with relatively high volumes of diluent to yield a flowable suspension (e.g., at a solids content sufficiently low that the suspension exhibits flow characteristics amenable to high-shear processing). This dilute suspension generally is not characterized by a yield stress, and typically flows readily when poured. High-shear mixing (e.g., using a rotor-stator mixer or other suitable high-shear mixer as described herein) is then applied to the suspension, which, in contrast to a concentrated suspension (e.g., VES), is of sufficiently low viscosity to be processed effectively by high-shear mixing. The excess diluent present in the suspension relative to the target concentrated suspension may also serve to dissipate some of the heat generated during the mixing process relative to a composition at higher solids content. Following mixing, the suspension is transferred to a separation apparatus configured to remove the excess diluent, thereby concentrating the powder relative to the starting suspension. In this example, the separation apparatus comprises a semi-permeable barrier, such as a membrane, that functions to retain the powder while permitting the excess diluent to pass through and be removed upon the application of a force or pressure, such as a mechanical, pneumatic, or gravitational force. The force or pressure may be applied until the target solids content of the concentrated suspension is substantially achieved, as may be determined through any suitable method. In one non-limiting example, the separated diluent is measured gravimetrically, and, based on the known powder and diluent contents of the suspension being processed, the approximate endpoint at which the force or pressure should be removed and the separation process discontinued can be determined. Alternatively, the composition may be over-concentrated, such that the resulting solids content of the processed composition exceeds the targeted solids content. In such instances, the composition may be diluted with additional diluent to substantially achieve the desired solids content of the concentrated suspension. To incorporate the diluent into the over-concentrated composition, the composition may be mixed through a variety of methods, including but not limited to manual mixing, or other mixing suited to viscous compositions, such as planetary-centrifugal mixing. The mixing method employed for diluent incorporation need not be the same as the high-shear mixing used in the initial preparation of the suspension.

The resulting solids content of the processed concentrated suspension may be determined through a variety of means. In one non-limiting example, the solids content is determined by empirically measuring the densities of the powder(s) and diluent(s) used to prepare the concentrated suspension, as well as the density of the resulting concentrated suspension, using techniques known in the art. For instance, the powder density may be measured using helium pycnometry, the diluent density may be measured using a liquid density meter, and the concentrated suspension density may be measured using helium pycnometry or any other suitable method. Using the following formula, where ρV, ρP, and ρD are the measured concentrated suspension (e.g., VES), powder, and diluent densities, respectively, the approximate solids content of the composition may be calculated by:

$$SC = \frac{{ρ}_{p} ( {ρ}_{V} - {ρ}_{D} )}{{ρ}_{V} ( {ρ}_{p} - {ρ}_{D} )}$$

It will be appreciated that the solids content calculated by the above formula represents an approximation based on the measured densities of the components and the resulting concentrated suspension, and may differ from the actual solids content due to variability inherent in density measurements, including without limitation instrument precision, temperature fluctuations, sample heterogeneity, entrapped air or gas within the composition, and the assumptions underlying the density-based model (e.g., ideal mixing and the absence of volume changes upon mixing). Accordingly, the calculated solids content is intended as a practical estimate suitable for process monitoring and corrective adjustment, and the actual solids content of the composition may vary from the calculated value.

This calculation may be used to verify the accuracy of the separation process and to inform any corrective action that may be required. In the case of an over-concentrated composition, wherein the solids content exceeds the targeted solids content, the mass of the composition and the calculated solids content may be used to determine the volume of additional diluent required to achieve the target solids content. Conversely, in the case of an under-concentrated or insufficiently concentrated composition, wherein the solids content is below the targeted solids content, the calculated solids content and the mass of the composition may be used to determine the extent to which the composition should be further concentrated (e.g., by additional separation) to achieve the target solids content. Accordingly, the formula above provides a means for quality control of the separation process, enabling adjustment in either direction to arrive at the desired concentrated suspension.

As described above, concentrated suspensions may be prepared by the high-shear mixing and separation approach, wherein a dilute suspension is first subjected to high-shear mixing to disrupt powder agglomerates, followed by removal of excess diluent using a separation apparatus to concentrate the suspension and yield a concentrated suspension with the desired solids content. The separation and concentration step may be performed using a separation apparatus of the type described herein. The suspension may be subjected to one or more separation cycles to achieve the target solids content, and the composition may be re-diluted and re-processed through the separation apparatus if additional agglomerate disruption, refined concentration control, or other processing objectives are desired. The process described herein may be performed in a batch, semi-batch, or continuous mode, and the high-shear mixing and separation steps may be performed sequentially or may be integrated into a continuous or semi-continuous process. In some embodiments, the high-shear mixing and separation steps may be performed in a single integrated apparatus, or the output of the high-shear mixer may be directly coupled to the inlet of the separation apparatus.

In one non-limiting embodiment of an integrated or semi-continuous process, the dilute suspension may be recirculated through an inline high-shear mixer (e.g., an inline rotor-stator mixer or other suitable inline mixing device) via a recirculation loop comprising a reservoir vessel, transfer lines, and a pump or other fluid-conveying means. The suspension may be recirculated through the inline mixer for a predetermined number of passes, a predetermined duration, or until a desired degree of agglomerate disruption has been achieved, as assessed by any suitable monitoring method, including without limitation inline particle size analysis (e.g., laser diffraction, focused beam reflectance measurement, or other particle characterization techniques), inline optical monitoring, or sampling and offline analysis. Upon achieving the desired degree of agglomerate disruption, the suspension may be directed to the separation apparatus, for example by opening a valve or actuating a flow diversion mechanism in the recirculation loop. Following concentration to the target solids content in the separation apparatus, the concentrated suspension may be transferred directly to downstream processing equipment, such as a syringe filling instrument or other container filling system, with or without intermediate storage or handling. In one such embodiment, the separation medium of the separation apparatus may be replaced with or converted to an extrusion geometry (e.g., a nozzle, orifice, or other suitable flow path) through which the concentrated suspension may be pushed, extruded, or otherwise conveyed by reapplying the same force or pressure (e.g., via the pushing device) used during the separation process. Alternatively, the separation apparatus may comprise interchangeable assemblies, wherein a first assembly comprising the separation medium is used during the concentration step, and a second assembly comprising an extrusion or dispensing geometry is substituted following concentration to facilitate direct transfer of the concentrated suspension to downstream filling or packaging equipment. In a further alternative, a nozzle or valve may be opened in the pushing device or in the main body while simultaneously blocking or converting the separation medium to a barrier to prevent further passage of diluent therethrough, thereby converting the action of the pushing device from concentrating the suspension to expelling or transferring the concentrated suspension through the alternative flow path. In each of the foregoing embodiments, the force or pressure applied during the dispensing or transfer operation may be the same as, or different from, the force or pressure used during the separation process, and may be applied according to any suitable profile as described herein. In some embodiments, the concentrated suspension may be passed through a static mixer positioned between the separation apparatus and the downstream collection, filling and/or processing equipment to improve homogeneity following the separation process.

The static mixer provides gentle mixing to address any non-uniformity that may result from the directional pressing during separation, while maintaining the benefits of a closed, integrated process suitable for aseptic manufacturing. Such an integrated process may reduce handling, may improve manufacturing yield, may minimize exposure of the composition to the environment, may support aseptic manufacturing requirements, and may enable a substantially closed processing pathway from the preparation of the dilute suspension through the filling of the final dosage form, though it will be appreciated that one or more steps of the process may require temporary opening of the apparatus (e.g., for sampling, venting, or operator inspection) without departing from the scope of the present disclosure.

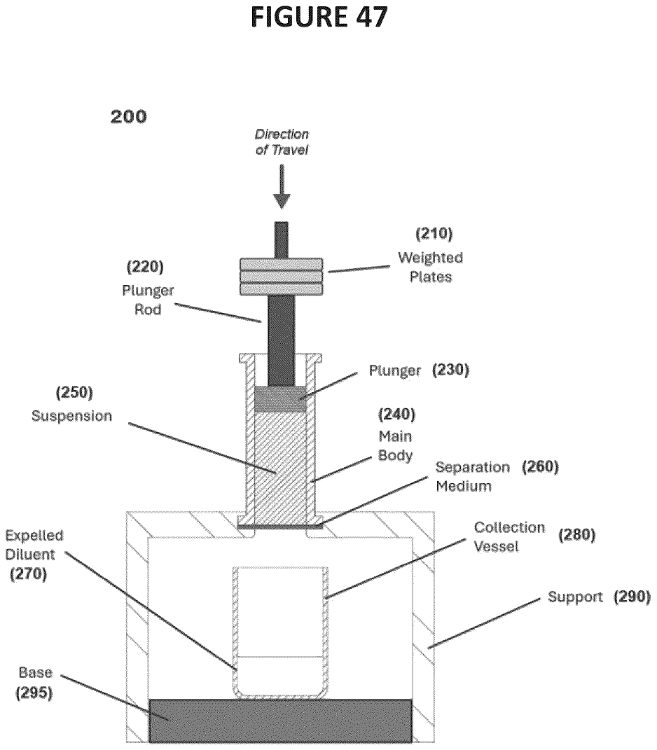

Referring to FIG. 47, the following non-limiting example describes the production of a concentrated suspension (e.g., a viscoelastic suspension) using an apparatus (200) assembled in accordance with the present disclosure. In this embodiment, the main body (240) comprises a substantially hollow, generally cylindrical chamber formed of polypropylene, having an internal bore diameter of approximately 2.5 inches (approximately 63 mm) and a chamber height of approximately 4.75 inches (approximately 121 mm). The main body (240) defines an interior volume configured to receive and retain the suspension (250) during the separation process. The main body (240) comprises an upper opening configured to receive the plunger (230) following the addition of the suspension to the main body, and a lower opening in fluid communication with the interior volume.

The separation medium (260) comprises a 0.45 μm pore size polyvinylidene fluoride (PVDF) membrane filter having a diameter of approximately 63 mm, placed on top of a thin perforated metal disk that provides structural support to the membrane during the application of force. A paper gasket was placed between the metal disk and the PVDF membrane. This assembly was then placed on a silicone gasket having a perforated metal screen and housed within a polypropylene body having a perforation configured to permit the separated diluent (270) to pass therethrough. The polypropylene cap is attached to the lower opening of the main body (240) via a twist-lock mechanism. In this configuration, the separation medium (260) is oriented substantially horizontally at the lower end of the main body (240), spanning the cross-sectional area of the interior volume, and is configured to permit passage of the liquid phase (the diluent) therethrough while retaining substantially all of the solid phase (the powder) within the interior volume above the separation medium (260).

The plunger (230), functioning as a pushing device, comprises a generally cylindrical body formed of food-grade silicone, having an outer cross-sectional profile that substantially corresponds to the internal bore of the main body (240) such that the plunger (230) forms a substantially fluid-tight seal against the inner wall of the main body (240) and is capable of translating axially within the interior volume. The plunger (230) is inserted through the upper opening of the main body (240) using a venting tube (e.g., thin gauge, 2″ length needle), such that its lower face is in close proximity to, or in contact with, the upper surface of the suspension (250). A plunger rod (220), comprising an aluminum bar having a lip or flange at its upper end, is disposed above and operatively coupled to the plunger (230) and extends upwardly through the upper opening of the main body (240). The lip or flange of the plunger rod (220) is configured to support one or more weighted plates (210). In this embodiment, weighted plates (210) totaling approximately 31 pounds are disposed on the lip of the plunger rod (220) above the main body (240). The weighted plates (210) apply a substantially constant gravitational force in a downward direction (as indicated by the directional arrow labeled “Direction of Travel” in FIG. 47), which is transmitted through the plunger rod (220) to the plunger (230) and through the plunger (230) to the suspension (250), thereby urging the liquid phase of the suspension (250) through the separation medium (260).

The separated diluent (270), having passed through the separation medium (260), drains by gravity into a collection vessel (280) disposed below the main body (240). The collection vessel (280) comprises a graduated plastic beaker, the graduated markings of which permit visual monitoring of the volume of separated diluent (270) collected during the separation process. The main body (240), separation medium (260), and collection vessel (280) are supported by a support (290) comprising a frame assembled from stainless steel T-slotted rails, which provides a rigid and stable structure for the apparatus (200). The apparatus (200) may be supported on a base (295) comprising a laboratory bench.

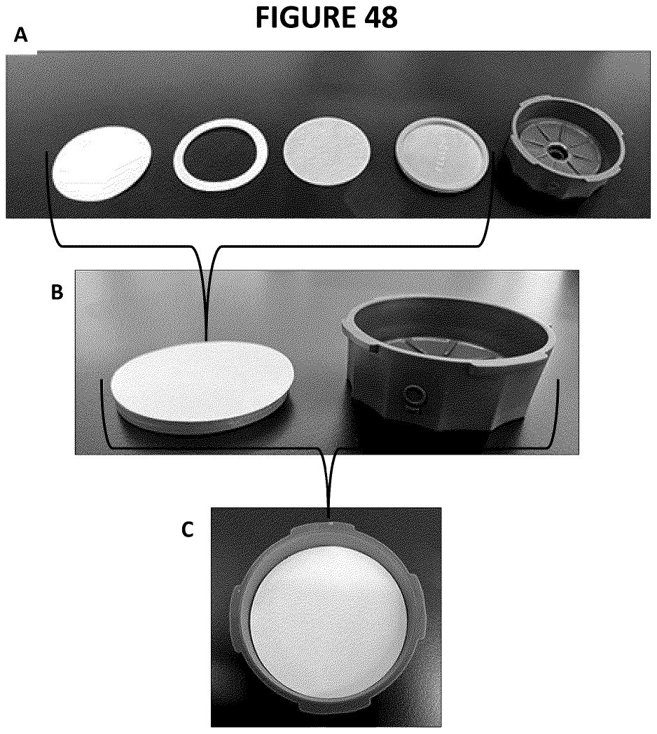

FIG. 48 depicts an example of a separation medium assembly in accordance with the present disclosure. Panel A shows the individual components of the assembly in an unassembled, exploded arrangement. From left-to-right as depicted (and corresponding to the order of assembly from the interior volume toward the exterior of the apparatus): a 0.45 μm pore size PVDF membrane filter; a paper gasket configured to seat and seal the membrane filter; a perforated metal support having apertures of approximately 250 μm, configured to provide structural support to the membrane filter during the application of force or pressure while permitting the separated diluent to pass therethrough; a silicone gasket configured to form a seal between the separation medium components and the housing to substantially prevent loss of the solid phase from the assembly; and a polypropylene housing configured to retain the foregoing components and to attach to the main body via a twist-lock mechanism. Panel B shows the subassembly of the four components alongside the polypropylene housing. Panel C shows the fully assembled separation medium assembly as viewed from the interior volume side, with the membrane filter visible within the polypropylene housing. The separation medium assembly depicted in FIG. 48 represents one non-limiting configuration; other configurations, component materials, pore sizes, gasket types, and attachment mechanisms are contemplated by the present disclosure.

The selected pore size of the separation media will depend on the size range of the powder in the suspension. The separation media may comprise any suitable material capable of retaining the powder while permitting passage of the diluent, including but not limited to paper-based media, polymeric membranes, or composite materials. Non-limiting examples of suitable polymeric membrane materials include polyvinylidene fluoride (PVDF), polyethersulfone (PES), nylon, polypropylene, cellulose acetate, regenerated cellulose, mixed cellulose esters, polytetrafluoroethylene (PTFE), and glass fiber, among others. Commercially available membrane products suitable for pharmaceutical processing include, by way of example and not limitation, Durapore® membranes (PVDF, available from Millipore), Supor® membranes (PES, available from Pall Corporation), Versapor® membranes (acrylic copolymer on nylon support, available from Pall Corporation), and other sterile and non-pyrogenic membrane products designed for pharmaceutical applications. Suitable pore sizes may include, without limitation, 0.2 μm or 0.45 μm, which are commonly used in pharmaceutical processing, though pore sizes ranging from approximately 0.1 μm to several micrometers or larger may be employed depending on the powder characteristics. However, if the suspension contains powder having particles that are larger than these commonly used membrane sizes, then larger separation media pore sizes may also be used. As will be appreciated by one of ordinary skill in the art, a larger separation media pore size may lower the force and/or time required to remove the excess diluent, with the potential benefit of speeding up the overall manufacturing time.

As will also be understood by one of ordinary skill in the art, in addition to pore size, the separation media material and surface chemistry may also be selected to reduce the resistance to separation, minimize extractables and leachables, provide chemical compatibility with the suspension components, and optimize wetting characteristics for the particular diluent employed. The surface chemistry of the membrane may be further tailored through treatments, coatings, or chemical modifications to optimize its interaction with the specific powder and diluent combination, thereby enhancing separation efficiency, reducing processing time, and minimizing potential interactions between the membrane and the suspension components that could affect the quality or purity of the resulting composition.

In this approach, a suspension of powder and diluent is first prepared using high-shear mixing to disrupt agglomerates that may be present in the powder and achieve dispersion in the diluent prior to concentration through the separation apparatus. In this example, the high-shear mixing is performed using a rotor-stator mixer, which generates intense hydraulic shear through the interaction of a high-speed rotor and a precision-machined stator, providing shear rates sufficient for rapid agglomerate disruption. In this example, a laboratory-scale rotor-stator mixer (such as the Silverson L5M or equivalent, featuring 316L stainless steel wetted parts and variable speed control) is used, though any suitable rotor-stator mixer or other high-shear mixer capable of generating sufficient shear to disrupt powder agglomerates may be employed. The suspension is prepared at a sufficiently low solids content (e.g., below about 15% by weight, though not limited to any particular solids content or range) such that it exhibits relatively low viscosity and lacks the yield stress that may be characteristic of concentrated suspensions (e.g., VES), allowing it to flow readily and be subjected to effective high-shear mixing without the heat buildup and localized shear problems that may occur if attempting to mix a more viscous composition characteristic of a concentrated suspension.

Following high-shear mixing, the resulting suspension is transferred (e.g., poured, pumped, or otherwise transferred) into the separation apparatus described above. After the suspension is transferred into the main body, a pushing device may be inserted into the open end of the apparatus above the suspension (FIG. 47). The pushing device may comprise a plunger assembly as described above, or any other suitable device configured to translate within the main body. The preparation of concentrated compositions using this method may be accomplished at laboratory scale or other scales using a separation apparatus configured for liquid extraction from suspensions, equipped with pharmaceutical grade separation media, such as the Durapore® membranes from Millipore. Such an approach is depicted in FIG. 48, showing the exploded view (FIG. 48A) of the separation media assembly, the subassembly of components alongside the housing (FIG. 48B), and the final assembled separation assembly (FIG. 48C) used to prepare the concentrated compositions described below.

The parameters of the separation process may be selected and controlled to achieve the target solids content and rheological properties of the final composition while avoiding over-concentration that could result in a composition having rheological properties unsuitable for the intended application. As described elsewhere herein, over-concentrated compositions may be corrected, but preventing over-concentration through appropriate process control is generally more efficient than corrective measures. The separation process parameters may be optimized based on the specific powder and diluent combination, the target concentrated suspension (e.g., VES) properties, and the intended delivery method (e.g., syringe and needle gauge). Process monitoring and feedback systems, whether manual or automated, may be employed to ensure consistent results and prevent under-concentration or over-concentration of the composition. The controlled separation approach described herein may provide a scalable method for preparing concentrated suspensions (e.g., viscoelastic compositions) with controlled rheological properties, solids content, and syringeability, complementing the high-shear mixing step, which may enable concentration of the agglomerate-reduced suspension to the desired solids content for the intended pharmaceutical application. While the high-shear mixing and separation approach described herein may provide effective agglomerate disruption with the practical advantages of scalability, aseptic compatibility, and reduced contamination risk relative to post-manufacture milling approaches, it will be appreciated that the methods described herein may be used in combination with any of the other agglomerate disruption approaches disclosed herein, including without limitation sieving, post-manufacture milling, or other processing steps, where additional agglomerate reduction is desired.

### Example 14: Preparation of Concentrated Suspensions with Reduced Agglomerates

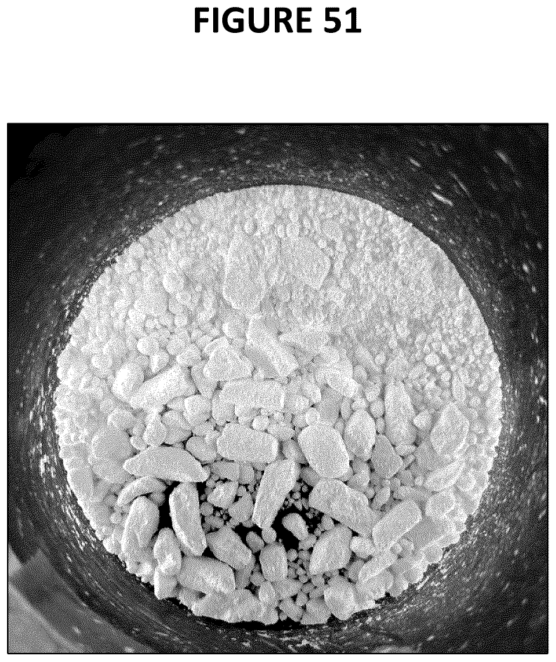

By way of example and not limitation, the effectiveness of the methods described herein at removing powder agglomerates and producing a concentrated suspension (e.g., a viscoelastic composition) suitable for delivery through fine-gauge needles was demonstrated in a comparative study using spray-dried trehalose powder. The spray-dried trehalose powder had a primary particle size distribution generally in the range of 1 to 10 micrometers as characterized by laser diffraction, and may be considered a micronized powder. In this study, the powder was subjected to tumbling in a rotating container continuously at high speed for one hour to promote agglomerate formation through particle interactions and interparticle forces. This tumbling process was intentionally prolonged to create agglomerates that were far more extensive than would typically be encountered in normal powder handling and processing, thereby providing a rigorous test of the agglomerate disruption capabilities of the methods described herein. As shown in FIG. 51, the powder became extensively agglomerated, with discrete and robust powder clusters ranging from tens of microns to several millimeters in size (note that the diameter of the container shown in FIG. 51 is approximately 65 mm). This highly agglomerated powder was used to prepare concentrated suspensions by blending with Miglyol 840 (propylene glycol dicaprylocaprate) using two different approaches for comparison:
(1) the standard approach, in which a concentrated suspension was directly prepared by mixing the agglomerated powder and Miglyol840 in a planetary-centrifugation mixer (e.g., THINKY or equivalent) for approximately 2 minutes at 2000 RPM without additives or additional processing steps; and
(2) the high-shear mixing and separation approach, in which a suspension was initially prepared at approximately 3% (w/w) solids content by adding approximately 250 g of Miglyol 840 to 7 g of the agglomerated powder, followed by the application of high-shear mixing using a rotor-stator mixer (e.g., Silverson L5M-A or equivalent) at maximum speed (approximately 10,200 RPM) for approximately 2 minutes to disrupt agglomerates in the dilute suspension. The mixed suspension was then transferred to a separation apparatus and the excess diluent was removed over approximately 1 hour using approximately 31 lbs of applied weight as described above. The separation apparatus and separation media assembly used in this example are shown in FIG. 47 and FIG. 48. The resulting concentrated suspension was then transferred to a planetary-centrifugation mixer (e.g., THINKY ARE-310 or equivalent) and mixed for approximately 2 minutes (at 2000 RPM) to apply the same treatment as the comparator suspension (standard approach).

The suspension prepared by the high-shear mixing and separation approach was prepared first, and its density was measured by helium pycnometry to determine the solids content, which was calculated to be approximately 58% (w/w). The suspension prepared by the standard approach was then prepared at the same solids content (approximately 58% (w/w)) by directly mixing the same powder and Miglyol 840 in appropriate proportions using a planetary-centrifugation mixer, operated for approximately 2 minutes at 2000 RPM, allowing for direct comparison of injection force performance between the two preparation methods.

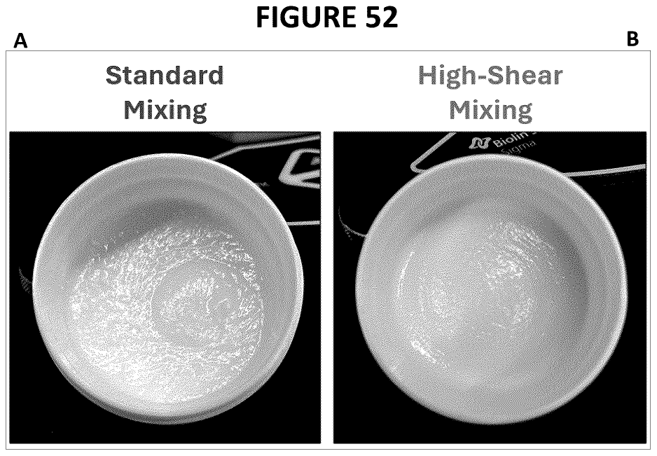

As shown in FIG. 52, the standard mixing approach yielded a concentrated suspension having visible powder agglomerates (FIG. 52A). In contrast, the approach of high-shear mixing and separation yielded a composition that appeared more consistently smooth (FIG. 52B), demonstrating effective agglomerate disruption while maintaining the viscous, non-pourable consistency characteristic of a concentrated suspension.

Injection forces for each of these two preparations were measured through 2.25 mL glass Luer-Lock syringes fitted with 25-gauge, 0.5-inch ultra-thin wall needles (JBP). To fill these test syringes, the concentrated suspension was first loaded into a larger 10-mL Luer-Lock syringe fitted with a blunt loading needle (14G). This larger “loading” syringe was then used to dispense the suspension into the test syringes, which were then fitted with the test needles, and injection force was evaluated using a texture analyzer with the linear probe speed set to achieve a volumetric flow rate of 50 μL/sec from the test syringes.

The composition prepared via the standard process quickly clogged the loading syringe (i.e., by occluding the loading needle) during attempts to fill the 2.25 mL test syringes. Since the loading needle (14G) had an approximately 1.6 mm (1600 μm) internal diameter, these results indicated the presence of large powder agglomerates that were not disrupted during the mixing process. As the test syringes could not be filled due to the clogging of the loading syringes, injection force measurements could not be performed for this composition.

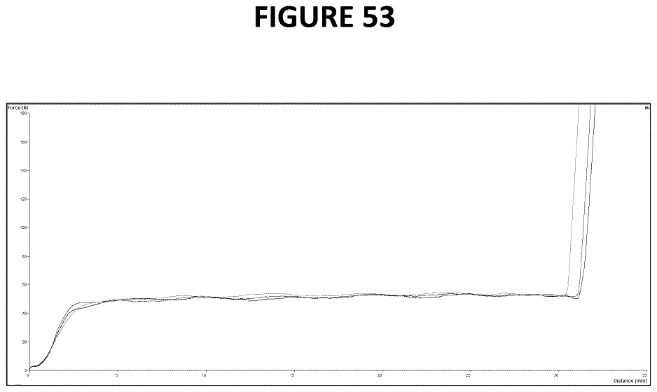

In contrast, the composition prepared using the high-shear mixing and separation approach was loaded into the test syringes without issue, and injection force was measured through the 25-gauge needle without any indication of clogging/occlusion (i.e., without injection force spikes). As shown in FIG. 53, which plots injection force (in Newtons) as a function of plunger travel distance (in millimeters), injection force measurements produced consistently smooth profiles across the entire dispensed volume of 1.75-2.00 mL, for N=3 replicate syringes, with a mean injection (glide) force of approximately 50 N. These results demonstrate that concentrated suspensions (e.g., viscoelastic compositions) having controlled rheological properties and improved syringeability for delivery through fine-gauge needles are suitably prepared using the methods described herein, including concentration from a dilute starting suspension (in this example, from approximately 3% to approximately 58% w/w, representing a greater than nineteen-fold increase in solids content) through the controlled removal of excess diluent in approximately one hour.

### Example 15: Concentrated Suspensions Prepared with Protein Powder

Having demonstrated the effectiveness of the methods described herein using spray-dried trehalose powder with intentionally created extensive agglomerates, the present inventors sought to evaluate the approach using a more pharmaceutically relevant powder composition. The following example demonstrates the application of the high-shear mixing and separation approach to a spray-dried protein powder formulation to produce a concentrated suspension (e.g., a viscoelastic composition), which represents a pharmaceutically relevant composition that may encounter agglomeration challenges during processing and handling.

By way of example and not limitation, the effectiveness of the methods described herein at disrupting powder agglomerates was demonstrated using a spray-dried protein powder composition. Approximately 80 g of spray-dried powder comprising an active pharmaceutical ingredient (in this example, immunoglobulin G at approximately 78-79% (w/w) protein content) and excipients (including trehalose, lactate, and polysorbate 80) was prepared from a feed solution having the composition listed in Table 33 and spray dried using a Yamato DL410 spray drying instrument with operating parameters listed in Table 34. The resulting powder was subjected to secondary drying under vacuum at approximately 5° C. overnight to reduce the moisture content to less than 1% (w/w).

TABLE 33
Feed Solution Composition

| Component | mg/mL | % w/w |
|---|---|---|
| IgG | 31.47 | 78.66% |
| Trehalose Dihydrate | 8.10 | 20.26% |
| Lactic Acid | 0.35 | 0.88% |
| Polysorbate 80 | 0.08 | 0.20% |
| pH | 3.8 | |
| Total | 40.00 | 100.00% |

TABLE 34
Spray Dryer Operating Parameters

| Parameter | Setting |
|---|---|
| Drying Gas Rate | 0.5 m³/min |
| Atomizing Air (Nitrogen) Pressure | 0.2 MPa |
| Liquid Feed Rate | 16 mL/min |
| Inlet Temperature | 80° C. |

The powder had a true density of approximately 1.34 g/cc (measured by helium pycnometry) and exhibited generally spherical morphology with a substantially smooth surface (assessed by scanning electron microscopy). The primary particle size distribution was characterized via laser diffraction as being generally in the range of approximately 1 to 10 micrometers, excluding agglomerates as defined herein. In contrast to the above example, this powder was not subjected to intentional agglomeration but was stored for approximately two weeks post-manufacture in a sealed container at 2-8° C. before use. A portion of this powder was used to prepare a concentrated suspension at approximately 62% (w/w) solids content using dispersion mixing with a planetary-centrifugal mixer (e.g., THINKY ARE-310 or similar equipment). The resulting composition exhibited visible agglomerates and, as discussed below, when subjected to injection force testing through fine-gauge needles, produced either partial or complete occlusion of the delivery path, as evidenced by elevated and irregular injection force profiles.

As an alternative approach demonstrating the present invention, approximately 25 g of the same powder was combined with approximately 300 g of a hydrophobic diluent (in this example, Miglyol 812, a medium-chain triglyceride) to create a dilute suspension having approximately 8% (w/w) solids content. This dilute suspension was then subjected to high-shear mixing using a rotor-stator mixer (e.g., Silverson L5M-A or similar equipment) for approximately 2 minutes at high speed (in this example, approximately 10,200 RPM) to disrupt powder agglomerates. Following high-shear mixing, the suspension was transferred to a separation apparatus comprising a main body having an internal diameter of approximately 2.2 inches and equipped with separation media (in this example, a 0.45 μm pore size PVDF membrane such as Durapore®) positioned to retain the powder while permitting passage of the excess diluent. Separation was accomplished by approximately 31 lb of applied weight to remove excess diluent and concentrate the suspension (FIGS. 47, 48).

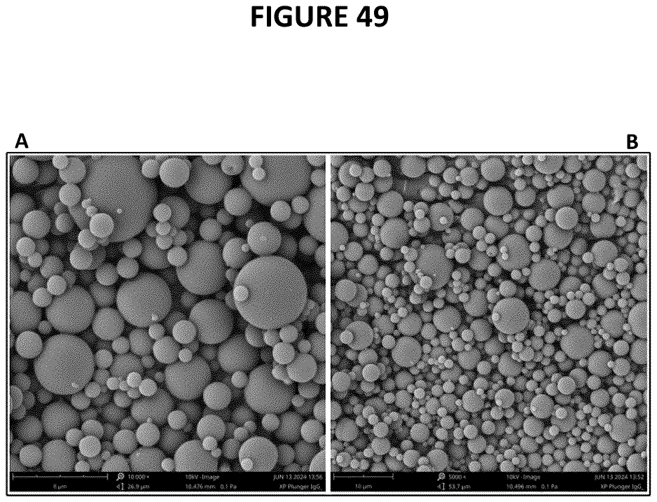

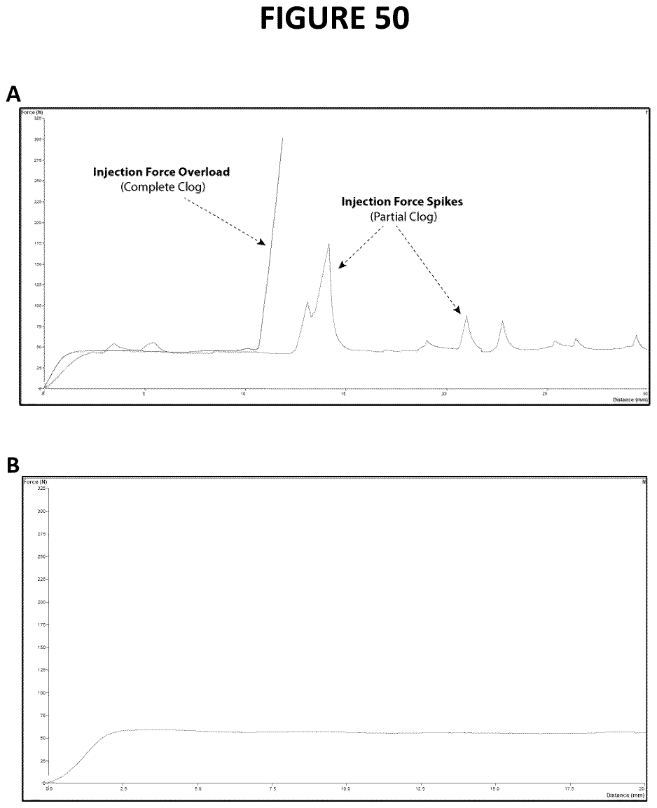

The resulting concentrated composition had a measured density of approximately 1.15 g/cc, corresponding to a calculated solids content of approximately 65% (w/w). Microscopic examination by scanning electron microscopy of the concentrated suspension confirmed that the high-shear mixing and separation process effectively disrupted agglomerates without damaging the primary particles, which retained their original spherical morphology and size distribution with no observable evidence of particle fracturing, distortion, or comminution (FIG. 49). The process successfully disrupted agglomerates (as evidenced by injection force profiling) while preserving the physical integrity of the individual powder particles. The concentrated viscoelastic composition exhibited a visually smooth consistency and demonstrated substantially improved syringeability compared to the composition prepared by dispersion mixing alone. As described above, when the composition mixed only by dispersion mixing was tested for injection through fine-gauge needles (e.g., 23-gauge, 8 mm ultra-thin wall), the presence of agglomerates was evident as notable spikes in the injection force profiles (indicating partial occlusion) or complete overloading of the force measurement system (indicating complete occlusion of the delivery path) (FIG. 50A). In contrast, when the same powder was processed using the high-shear mixing and separation approach described herein, the resulting viscoelastic composition was delivered smoothly through the same needle configuration without injection force spikes or delivery interruptions (FIG. 50B). These results demonstrate that the methods described herein are suitable for preparing concentrated suspensions from protein powders having controlled rheological properties and improved syringeability for parenteral delivery applications.

Taken together, these results confirm the conclusion from the studies above that high solids content pastes (or viscoelastic suspensions), which can then be injected at relatively low injection forces and therefore used therapeutically, are suitably prepared using the methods described herein.

### Example 16: Diluent Effect on Concentrated Suspensions Prepared by High-Shear Mixing and Separation

Having demonstrated in the preceding examples that the high-shear mixing and separation approach may be used to prepare concentrated suspensions (e.g., VES) having reduced agglomerate content and improved syringeability, the present inventors further sought to evaluate the influence of diluent selection on the injection forces observed when preparing concentrated suspensions using the methods and apparatus described herein. Specifically, spray dried powder was prepared from a feed solution and spray drying parameters described in Tables 33 and 34 from above.

TABLE 35
IgG Powder Characterization

| | Method | |
|---|---|---|
| Attribute | (Instrument) | Results |
| Particle Size | Laser Diffraction | D50 = 2.9 um |
| Distribution | (Anton Paar PSA 990) | D90 = 7.1 um |
| | | Mean Size = 3.6 um |
| | | Span = 2.4 |
| Powder Density | Helium Pycnometry | 1.33 g/cm³ |
| | (Anton Paar Ultrapyc 3000) | |
| Particle Shape | Scanning Electron Microscopy | Spherical |
| | (Thermo Scientific Pharos SEM) | |

Following spray drying, the powder was further dried under vacuum for 16 hours to lower the residual moisture content to less than 1% (w/w). This powder was combined with either Miglyol® 812 or Miglyol® 840 to form dilute suspensions at approximately 10% (w/w) solids content. Each dilute suspension was subjected to high-shear mixing for 2 minutes at approximately 10,200 RPM using a rotor-stator mixer (e.g., Silverson LM-5 or equivalent) to disrupt powder agglomerates. The mixed suspension was introduced into the interior volume of a separation apparatus of the type depicted in FIG. 47, comprising a cylindrical main body and separation media/assembly of the type depicted in FIG. 48 (e.g., a 0.45 μm PVDF membrane such as Durapore® or equivalent). The dilute suspension was then concentrated using approximately 40 lbs. of applied weight for 4 hours as described in Example 13, to remove excess diluent through the separation media while retaining the powder within the interior volume, thereby yielding a concentrated suspension (e.g., VES).

The solids content of each concentrated suspension was determined by helium pycnometry using the measured density of the composition in combination with the known densities of the powder (1.33 g/cm³) and the diluents (densities of approximately 0.92 g/mL and 0.94 g/mL for Miglyol 840 and 812, respectively), as described elsewhere herein. The Miglyol 840 concentrated suspension was approximately 69% (w/w) following the separation process, and was subsequently diluted to approximately 65.9% (w/w) by the addition of diluent using the methods described herein, such that its solids content was comparable to that of the Miglyol 812 concentrated suspension. Both compositions thus represented a greater than six-fold increase in solids content from the initial 10% (w/w) dilute suspension, demonstrating the capability of the apparatus and separation process to produce highly concentrated suspensions from dilute starting compositions regardless of diluent identity. Both concentrated suspensions exhibited a yield stress and did not flow under the force of gravity alone, consistent with viscoelastic suspension (VES) behavior as defined herein. Each concentrated suspension was filled into 1 mL cyclic olefin copolymer (Schott) Luer-Lock syringes and injection force was evaluated at two volumetric flow rates (50 and 100 μL/sec) through three different fine-gauge needles:
25G (UTW) 8 mm (Cadence)
25G (UTW) 13 mm (JBP)
27G (UTW) 8 mm (Cadence)

The measured injection force (provided as the average mean glide force in Newtons of N=3 replicate syringes) is shown in Table 36. All syringes for both diluent systems were administered without issue; no occlusions, injection force spikes, or other indications of delivery flow path obstruction were observed for any needle gauge, needle length, or volumetric flow rate tested. These results are consistent with the effective agglomerate disruption achieved by the high-shear mixing and separation approach described in Example 13.

For all evaluated syringes+needles+volumetric flow rates, Miglyol 840 yielded a much lower injection force relative to 812 (approximately 30-50% lower force for 840 relative to 812).

Notably, the diluent selection had a substantial impact on the injection forces of the resulting concentrated suspensions. For all evaluated syringes, needles, and volumetric flow rates, Miglyol 840 yielded substantially lower injection force relative to Miglyol 812 (approximately up to 50% lower force for 840 relative to 812). For the Schott syringe with 27G (UTW) 13 mm needle, the injection force for the 812 concentrated suspension was sufficient to eject the needle from the Luer-Lock syringe, and therefore no measurement was performed at that condition.

TABLE 36
Mean Glide Force (provided as the average of N = 3 replicate syringes)

| | 25G (XTW) | | 25G (UTW) | | 27G (UTW) | |
|---|---|---|---|---|---|---|
| | 8 mm | | 13 mm | | 8 mm | |
| | 50 | 100 | 50 | 100 | 50 | 100 |
| Diluent | (μL/s) | (μL/s) | (μL/s) | (μL/s) | (μL/s) | (μL/s) |
| Miglyol 812 | 31 | 44 | 52 | 82 | 59 | 94 |
| Miglyol 840 | 16 | 26 | 30 | 49 | 32 | 55 |

The difference in injection force between the two diluent systems is attributed to the difference in their respective viscosities, where the viscosity of Miglyol 840 (approximately 11 cP) is approximately one-third that of Miglyol 812 (approximately 30 cP). The approximately three-fold difference in diluent viscosity produced an approximately two-fold difference in injection force across the range of tested conditions, demonstrating that diluent selection may be important in optimizing the syringeability of concentrated suspensions, such as VES compositions, for delivery through fine-gauge needles. These results indicate that the choice of diluent, in addition to the high-shear mixing and separation methods described herein, may be used to modulate the injection force profile of a concentrated suspension to meet the requirements of the intended delivery device and clinical application.

The foregoing example is provided to illustrate the influence of diluent selection on the injection force of concentrated suspensions prepared using the high-shear mixing and separation approach described herein, and is not intended to limit the scope of the present disclosure to the specific diluents, powders, solids contents, needle configurations, or injection parameters described. It will be appreciated that other diluents, including without limitation other medium-chain triglycerides, propylene glycol diesters, ethyl oleate, benzyl benzoate, sesame oil, and other nonaqueous and/or hydrophobic diluents described elsewhere herein, may be employed in the methods and apparatus of the present disclosure, and that the relationship between diluent viscosity and injection force may vary depending on the specific powder, diluent, solids content, needle gauge, and flow rate employed.

### Example 17: Use of Diluent Additives T0 Modify the Injectability of Viscoelastic Suspensions for Delivery of Various Therapeutic Agents

The inclusion of diluent additives, such as PS80, can alleviate the problem of loss of yield stress in a viscoelastic suspension (VES) prepared for injection without markedly increasing the injection forces through narrow diameter needles used for parenteral injection of a diverse variety of therapeutic agents. Without wishing to be bound by theory, a viscoelastic suspension can be generally described as a suspension of a solid phase and a liquid phase, wherein the solid phase is present at such a high concentration in the liquid phase that when blended into a visibly homogeneous composition, the resulting suspension cannot be poured from the container at room temperature (e.g., from about 15° C. to 30° C.). The yield stress observed in a viscoelastic suspension causes the composition to transition between solid-like behavior (below the yield stress) and fluid-like behavior (above the yield stress). When a viscoelastic suspension is above the yield stress, it can be delivered from commercially available syringes and needles under applied injection force. Applied injection forces can include one of manual, assisted, or mechanical processes. The yield stress that is observed in a viscoelastic suspension may arise from the low interparticle distance between adjacent solid phase powder particles due to their high concentration in the diluent. This shortened interparticle distance between adjacent powder particles imparts a cohesive network throughout the composition. This interaction may arise from mechanical interlocking between particles and the capillary forces between particles.

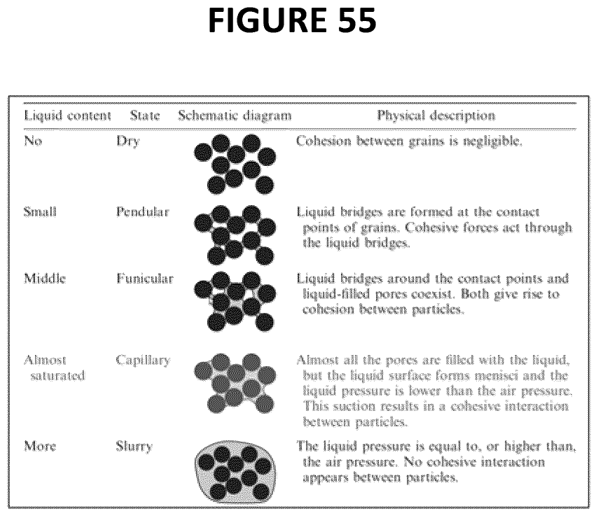

In general, the viscoelastic suspensions described herein can be described as being in the capillary regime, as shown in FIG. 55 describing liquid-solid mixtures, which demonstrates that nearly all the pores are filled with liquid. This results in a difference between the air pressure, which is higher, relative to the liquid pressure, which is lower. The pressure difference creates a cohesive particle interaction.

Increasing the solid phase powder content significantly beyond this region creates a wetted solid or clay-like composition that is no longer readily injectable through small diameter needles. Alternatively, decreasing the powder (solid phase) content significantly below the capillary regime will remove the yield stress due to decreasing the cohesive interaction between particles. This will allow the composition to be poured (e.g., under gravity), but will also allow particle settling in the suspension over time.

The solids content (defined as the % (w/w) of the powder in the composition) required to reach the capillary regime and produce a viscoelastic suspension will depend on the physical attributes of the powder (including but not limited to: powder density, size distribution, morphology, porosity, and specific surface area). For example, a low-density powder having a large specific surface could form a viscoelastic suspension at a much lower solids content relative to a higher-density particle having a smaller specific area. The solid phase of the compositions described herein may be a powder containing a therapeutic agent or diagnostic agent, while the liquid phase (also referred to as the diluent) does not fully solubilize the solid phase, and more preferably wherein the liquid phase is a non-solvent to the solid phase. An example of the latter would be a hydrophilic powder that is suspended in a hydrophobic diluent, wherein the hydrophilic powder should have minimal-to-no solubility in the hydrophobic diluent.

Examples of possible therapeutic agents, antigens, immunogens, or active pharmaceutical ingredients include but are not limited to peptides, antibodies, proteins, small molecules, and nucleic acids.

The additive(s) described in this approach may be introduced into the composition at different stages, including: addition to the diluent upstream of blending with the powder; addition the diluent during the blending phase; addition to the blended composition after initial diluent and powder blending, followed by subsequent processing to incorporate the additive uniformly across the composition. A benefit of this approach is that compositions that do have a yield stress are less susceptible to particle settling over time, which can promote content uniformity issues during filling and/or delivery issues if settling occurs in a pre-filled syringe. Further, for a given powder, the viscoelastic suspension represents the highest powder concentration that can be achieved while still enabling the composition to flow through commercial syringes and needles for injection. Generally, the promotion of a yield stress in the composition can depend on the properties of the particles. If the size distribution is not appropriate (i.e., insufficient fine particles to fill in the gaps between the larger particles and promote a cohesive capillary network) the composition may not exhibit a yield stress despite having a sufficiently high powder concentration. The inclusion of an additive at a low concentration (preferably below 5% (w/w) of the total composition, and most preferably below 2% (w/w) of the total composition) can restore the yield stress, visually “thickening” the suspension without markedly increasing the injection force.

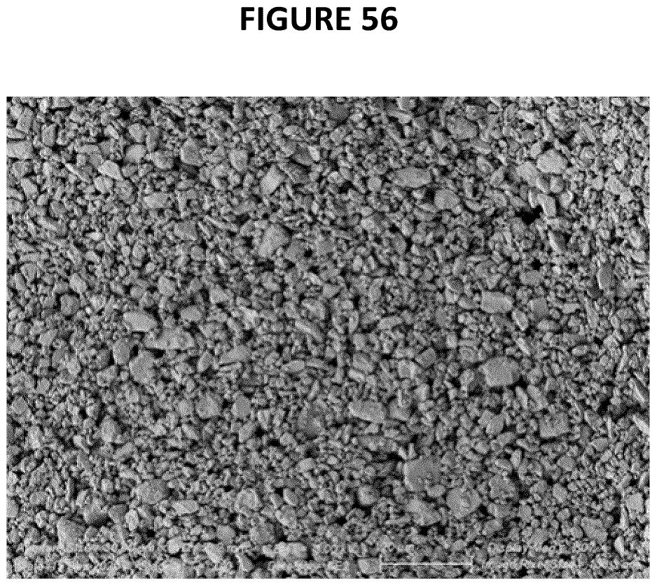

In the present studies, a viscoelastic suspension was prepared by blending micronized crystalline lactose (provided from DFE Pharma) with Miglyol 812 (Cremer Oleo) to 40% (w/w) solids content in a planetary-centrifugation mixer. The resulting viscoelastic suspension was split into two approximately equal aliquots. To one aliquot, an additional 0.5% (w/w) Miglyol 812 was added, and the composition was remixed. This aliquot served as control sample. To the second aliquot, 0.5% (w/w) Polysorbate 80 (PS80) was added, and then the composition was remixed. This aliquot served as the experimental sample. A scanning electron microscope was used to characterize the micronized lactose and revealed the particles to be irregular shaped with diameter generally below 10 μm, which can be seen in FIG. 56.

To measure the injection force profiles, each of the two VES samples were filled into 3-mL glass cartridges and sealed with elastomeric pistons. Injection force profiles were measured using a texture analyzer by delivering multiple doses through a 29G pen needle. For each measurement, the plunger was moved at a linear speed of 0.83 mm/sec in 5 mm increments.

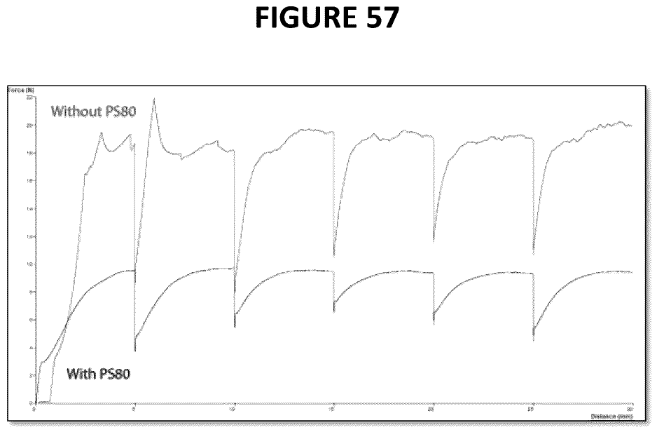

A comparison of injection forces for the control sample (red trace) and the experimental sample containing PS80 (blue trace) is shown in FIG. 57. The addition of the PS80 to the lactose-Miglyol 812 VES both lowered the measured injection force and provided a smoother injection force profile compared to the control (no additive) sample. This was the initial indication that the inclusion of an additive such as PS80 in a small quantity to a VES could potentially improve the injectability of VES formulations. At present, the mechanism responsible for the improvement is not known.

To examine the use of this approach with large-molecule peptides, samples of IgG were prepared by spray-drying from a solution containing the following components:

TABLE 36
Feed Solution for Spray-Dried IgG

| Component | mg/mL | % w/w |
|---|---|---|
| IgG | 31.47 | 78.66% |
| Trehalose Dihydrate | 8.10 | 20.26% |
| Lactic Acid | 0.35 | 0.88% |
| Polysorbate 80 | 0.08 | 0.20% |
| PH | 3.8 | |
| Total | 40.00 | 100.00% |

A Yamato spray dryer (model DL410) was used to spray-dry the solution, using the following settings:

TABLE 37
Spray-Dryer Settings

| Parameter | Spray Dryer Setting |
|---|---|
| Inlet Temperature | 80° C. |
| Feed Rate | 16 mL/min |
| Atomizing Pressure | 0.2 mPa |
| Drying Gas Flow Rate | 0.5 m³/min |

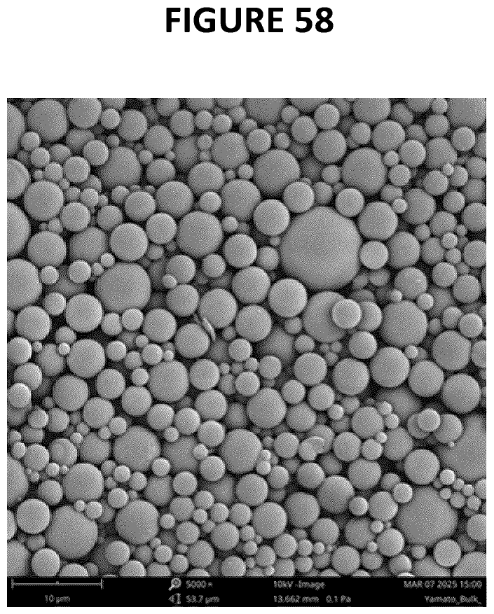

Other quantitative measurements were also taken. The spray dried powder was characterized for morphology (scanning electron microscope), particle size distribution (laser diffraction) and density (helium pycnometry). The particles appeared spherical with diameter generally below 10 μm, as shown in FIG. 58. For particle size distribution, the powder particles had the following parameters: D50=2.58 μm, D90=5.96 μm, Span=2.21 μm. The density of the powder, measured by helium pycnometry at room temperature (~23° C.), was 1.33 g/cm³.

For qualitative analysis, a viscoelastic suspension was prepared by blending the spray dried IgG powder with Miglyol 840 to yield a 60% (w/w) solids content VES. The powder and diluent were blended together using a planetary-centrifugation mixer. The resulting composition could be defined more of as a slurry than a viscoelastic suspension, as it did not have a notable yield stress and could be poured from the container (i.e., it could flow under gravity).

The VES was split into five aliquots, and polysorbate 80 was added to four of these aliquots to achieve either a 0.1%, 0.25%, 0.5% or 1.0% (w/w) content in the VES (excluding the PS80 content of the powder). The four aliquots were each mixed using the planetary-centrifugation mixer to disperse the added PS80 throughout the composition. The one aliquot that did not have PS80 added to it served as a comparative (control) sample in this study.

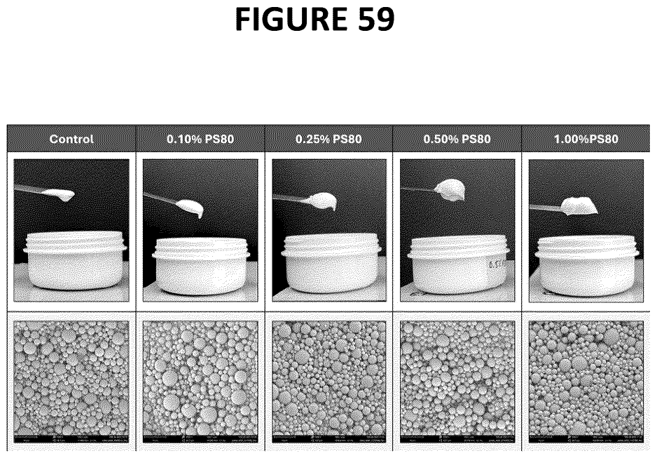

As shown in FIG. 59 (top row), the addition of even 0.1% (w/w) PS80 was sufficient to thicken the composition and restore the yield stress that was not seen in the control composition. Hence, the addition of PS80 appeared to thicken the consistency of the VES, which likely imparted the yield stress that was absent in the initial bulk sample. Interestingly, examination of these VES samples by SEM (FIG. 59, bottom row) indicated that the inclusion of PS80 did not appear to alter the morphology or size of the particles in the compositions.

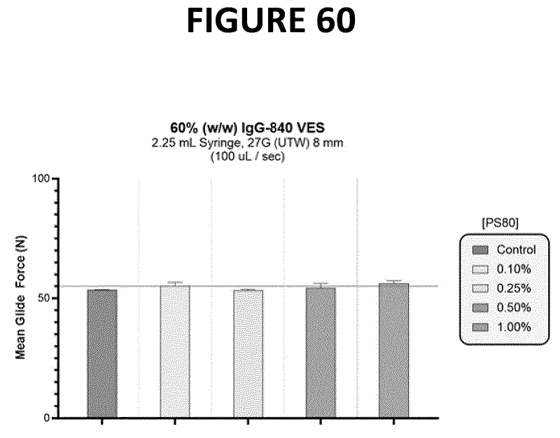

The addition of the surfactant did not appear to markedly increase the injection forces of the composition, despite the thicker appearance of the samples (FIG. 60). Injection force of the sample was measured using a texture analyzer (TA.XT Plus; Stable Microsystems). Samples were filled into 2.25-mL glass syringes having a staked needle (27G UTW, 8 mm) configuration. The linear speed of the texture analyzer probe was set to deliver a volumetric flow rate from the syringe of 100 μL/sec (0.1 mL/sec). FIG. 60 shows the measured mean glide forces for each of the test samples. While there did appear to be a slight increase across the samples, the 1% (w/w) PS80 sample was only about 3N higher than the control (no additional PS80) sample, which appeared to be within the standard deviation.

Without wishing to be bound by theory, the present inventors reasoned that including an additive) in the diluent phase of the viscoelastic suspension, particularly a minor quantity of a rheology modifier such as a surface-active agent (e.g., triacetin, or a surfactant such as Miglyol 810, Miglyol 812, Miglyol 820, or Miglyol 840) could potentially improve the injectability of the VES through small diameter needles. In this context, “improving injectability” of the VES could mean a reduction in injection force (plunger break-loose force and/or mean glide force) or smoother injection force curves, both of which would indicate that the powder particles within the VES are flowing past each other with reduced friction, inhibiting the formation of major and minor injection force spikes. The reduction in injection force could be a result of the diluent additive lowering the viscosity of the formulation by directly affecting the diluent and/or by functioning as a lubricant to improve the flow of powder particles during injection.

An additional benefit of including diluent additives can be to thicken the consistency of the composition without greatly increasing the injection forces of the composition through narrow diameter needles, as shown in the present Example. By thickening the consistency of the composition, an additive may restore the yield stress of a slurry or suspension, such that it may no longer be poured from a container. This could be beneficial as it would inhibit particle settling in the composition during short-term storage (important, since such particle settling could negatively impact dose content uniformity) and also over long-term storage in a relevant pharmaceutical container closure system such as a syringe or cartridge (also important, since particle settling over long storage periods could also negatively impact injectability). The invention described herein thus may be used to restore the yield stress of the suspensions to inhibit particle settling without markedly increasing injection forces through narrow diameter needles.

Alternative approaches to this problem typically focus on the use of suspensions to lower injection force relative to viscoelastic suspensions, where the suspensions do not have a yield stress that is characteristic of a viscoelastic suspension. In general, one can determine if a suspension has a yield stress if the suspension can be readily poured from the container (e.g., under gravity) following mixing and return to room temperature. If the suspension cannot be readily poured from the container, it is said to possess a yield stress and may be characterized as a viscoelastic suspension that exhibits both solid-like behavior (below the yield stress) and fluid-like behavior (above the yield stress). However, as the present invention shows, it is not sufficient to increase the yield stress of a suspension; to be useful in providing injectable formulations for therapeutic or diagnostic use, the resulting higher yield stress formulations must also be capable of being injected through narrow diameter needles without a substantial increase in injection force. The present invention provides such injectable formulations.

### Example 18: Apparatus for Producing Viscoelastic Suspensions by Separation of a Liquid Phase

The viscoelastic character of a viscoelastic suspension (VES) may be demonstrated or confirmed by any suitable measurement or combination of measurements, including without limitation: oscillatory shear rheometry (e.g., measurement of storage modulus (G′) and loss modulus (G″), wherein G′ exceeds G″ over at least a portion of the tested frequency or strain range); yield stress measurement (e.g., determination of the shear stress at which G′ and G″ cross over in an oscillatory amplitude sweep, or by other methods such as controlled stress ramp, tangent intersection, or model extrapolation); tack measurement (e.g., probe tack testing to determine the work-of-cohesion and/or work-of-adhesion of the composition, which may be indicative of the strength of the cohesive interparticle network); assessment of cohesive uniformity (e.g., the composition maintains structural coherence and may extrude as a substantially continuous, cohesive mass rather than as discrete crumbles, or fragments, or may not flow in a steady, continuous stream under the force of gravity alone); or measurement of complex viscosity or apparent viscosity at shear rates relevant to the intended application.

The foregoing characterization methods are not exhaustive; a VES may be identified by any measurable property or combination of properties that is indicative of the presence of a cohesive interparticle network arising from the interactions between the powder and the diluent at the solids content of the composition. The specific values of any rheological parameter at which a given powder-diluent combination exhibits viscoelastic suspension behavior are dependent on the physicochemical properties of the particular powder and diluent, the solids content of the composition, and the conditions of measurement, and are not limited to any particular numerical range or threshold.

A VES may exhibit shear-rate dependent viscosity, broadly characterized as non-Newtonian behavior. In many embodiments, the VES may exhibit shear-thinning behavior, wherein the apparent viscosity of the composition decreases with increasing shear rate. Such shear-thinning behavior may be further described as thixotropic, wherein the apparent viscosity decreases under sustained shear and recovers, partially or fully, upon cessation or reduction of shear. However, the compositions described herein are not limited to shear-thinning or thixotropic behavior, and a VES that exhibits shear-thickening, rheopectic, or other non-Newtonian rheological behavior is within the scope of the present disclosure. The term “viscoelastic suspension” is not limited to any particular solids content, viscosity, storage modulus, loss modulus, yield stress, or other rheological parameter, and encompasses compositions across a broad range of consistencies, from relatively thin, slowly flowing compositions to relatively thick compositions of high viscosity.

It is further noted that within the range of solids contents at which a given powder and diluent combination exhibits viscoelastic behavior, the consistency of the VES may vary considerably. At solids contents near the lower boundary of the viscoelastic range for a given powder and diluent combination, the VES may exhibit a relatively thin consistency, a lower yield stress, a lower apparent viscosity, and may be described as soft, spreadable, or flowable under moderate applied force, while still exhibiting measurable viscoelastic character (i.e., both a storage modulus (G′) and a loss modulus (G″), with the storage modulus exceeding the loss modulus at rest). At higher solids contents within the viscoelastic range, the VES may exhibit a progressively thicker consistency, a higher yield stress, a higher apparent viscosity, and may be described as stiff, dense, or resistant to deformation. At solids contents above the viscoelastic range, the composition may transition to a state in which the cohesive interparticle network is no longer maintained uniformly throughout the composition, and the material may exhibit behavior characteristic of a wetted solid, granular paste, or clay-like material rather than of a viscoelastic suspension. In all cases within the viscoelastic range, the composition remains a viscoelastic suspension as defined herein, provided that it exhibits both viscous and elastic behavior. The term “viscoelastic suspension” or “VES” as used herein is therefore not limited to compositions of any particular consistency, stiffness, yield stress, apparent viscosity, or other rheological property, and encompasses the full continuum of consistencies from relatively thin to relatively thick that a given powder and diluent combination may exhibit across the range of solids contents at which viscoelastic behavior is present. The target solids content and consistency of a VES for a particular application may be selected based on the intended end use of the composition, including but not limited to the route of administration, the delivery device (e.g., syringe and needle gauge), the acceptable injection force, the desired dose volume, and the stability and shelf-life requirements of the composition, and may be adjusted by the addition or removal of diluent using the methods and apparatus described herein.

As used herein, the term “target solids content” refers to a predetermined or desired proportion of the solid phase in the viscoelastic suspension, expressed as a weight fraction (w/w), volume fraction (w/v) or (v/v), percentage, or any other suitable measure. The target solids content may be selected based on the intended end use of the VES composition, and may vary or be changed at any time depending on the particular powder, diluent, and application. The target solids content need not be a single precise value, and may encompass one or more ranges or tolerance bands. As used herein, the term “compatible,” when used to describe a material of construction of the apparatus or any component thereof in relation to the suspension or its components, means that any interaction between the material and the suspension or its components, including but not limited to chemical reaction, degradation, leaching, absorption, adsorption, dissolution, fragmentation, shedding of particulates, or other physical or chemical interaction, does not occur to a degree that would render the apparatus unsuitable for its intended purpose, the separation process unable to achieve its intended function, or the resulting VES composition unsuitable for its intended end use. It is understood that minor or trace-level interactions between the material and the suspension may occur, and such interactions do not render the material incompatible provided that the resulting VES composition remains within applicable specifications for its intended end use, including but not limited to specifications relating to extractables and leachables, particulate matter, impurity levels, degradation products, potency, and stability. The determination of whether a material is “compatible” may be made with reference to the applicable regulatory requirements, pharmacopeial standards, compendia, or product specifications for the intended end use of the VES composition, or in the absence of such requirements, by a person of ordinary skill in the art exercising reasonable judgment.

Described herein is an apparatus configured to modify the solids content of a suspension comprising one or more powders and one or more diluents by the controlled separation and removal of a portion of the liquid phase from the suspension, for the purpose of producing a viscoelastic suspension (“VES”) as described elsewhere herein. The apparatus may be used at any and all stages of the VES production process, including but not limited to: following the application of high-shear mixing, low-shear mixing, blending, or any other process by which the powder and diluent have been combined into a suspension. The apparatus is not limited to use with any particular type of mixing or blending process, nor is it limited to any particular powder, diluent, powder-to-diluent ratio, or target solids content. The apparatus may be used to achieve a target solids content directly through the separation process or may be used in conjunction with one or more corrective steps, such as dilution of an over-pressed composition, with new or re-ised liquid phases, or re-pressing of an under-pressed or incorrectly pressed composition, as described elsewhere herein.

The apparatus is of a modular construction, comprising one or more components that may be assembled, disassembled, interchanged, and reconfigured in any number of to accommodate different suspension compositions, separation requirements, and target VES specifications. The modular construction allows individual components of the apparatus to be selected based on their suitability for the particular suspension being processed, the intended end use of the resulting VES composition, and any applicable regulatory or compatibility requirements. The principal components of the apparatus include a main body, any number of auxiliary bodies, any number of interchangeable bodies, any number of separation media, any number of pushing devices, any number of associated support structures, and any number of feedback systems, each of which is described in detail in the paragraphs that follow.

Referring now to the structure of the apparatus, the apparatus comprises a main body that is substantially hollow and that functions as a container or reservoir for receiving and retaining the suspension to be processed. The main body defines an interior volume sufficient to contain a working quantity of the suspension during the separation process. The main body may be formed of any material(s) that are compatible with the suspension components, capable of withstanding the forces and/or pressures encountered during the separation operation, and suitable for the intended end use of the resulting VES composition. In embodiments where the apparatus is used to produce a viscoelastic suspension intended for pharmaceutical, biomedical, veterinary, or other applications involving human or animal use, the main body may be formed of a material that is substantially inert with respect to the suspension components, such as stainless steel (e.g., 304, 316, or 316L grade), glass, or a suitable engineering plastic or polymer, and may be configured to comply with applicable regulatory requirements for materials intended for use in the manufacture of pharmaceutical or biomedical products.

The main body comprises at least one openings, each in fluid communication with the interior volume. The openings may serve one or more functions, including but not limited to the introduction of the suspension or its components into the interior volume, the connection of one or more auxiliary bodies to the main body, the exit of separated liquid from the interior volume, the insertion or removal of any number of pushing device (when present), the withdrawal of samples for analysis, and/or the venting or introduction of pressure. Any one or more of the openings may be sealed when not in active use. The openings may be sealed by any suitable means known in the art, including but not limited to sanitary fittings (e.g., tri-clamp fittings), gaskets, compressive sealing materials, threaded caps, plugs, or combinations thereof. The sealing means may permit the apparatus to be more selectively opened and closed as required at various stages of the separation process, and facilitate cleaning, sterilization, and maintenance of the apparatus between uses. The apparatus may be configured to be disassembled for cleaning and reassembled for subsequent use, and may be compatible with one or more sterilization methods known in the art, including but not limited to autoclaving, chemical sterilization, or irradiation, as may be required by the intended application.

The apparatus may further be comprised of one or more auxiliary bodies. Each auxiliary body may be disposed superiorly to (i.e., above, relative to the operational orientation of the apparatus), inferiorly to (i.e., below, relative to the operational orientation of the apparatus), laterally adjacent to, or in any other suitable spatial relationship relative to the main body. Each auxiliary body is configured to attach to the main body and/or to one or more other auxiliary bodies and is configured to perform or to aid in performing one or more functions associated with the separation process, as described in the following paragraphs.

In one embodiment, at least one of the auxiliary bodies provides a fluid, mechanical, or gravitational connection to at least one source of force and/or pressure configured to drive the separation process. The source of force or pressure may comprise, but is not limited to, a source of pneumatic pressure (e.g., compressed air or an inert gas), a source of hydraulic pressure (e.g., pressurized water or other liquid), a source of magnetic force, a mechanical actuator, gears, springs, pistons, plungers, vibrators, one or more weights configured to apply a gravitational force (e.g., dead weights, calibrated masses, or weighted plates disposed on or above the suspension), or any other source of force suitable for urging the diluent through at least one separation medium disposed within the apparatus. If a plurality of sources of force and/or pressure are used, each may be used in any combination of positioning, timing and sequencing in relation to all other sources of force and/or pressure. In cases where force is applied by one or more weights, weights may be used alone or in combination with any other source of force or pressure described herein. The connection between the auxiliary body and the source of force or pressure may comprise any suitable fitting, conduit, coupling, support surface, or retaining structure known in the art.

Any force or pressure applied to the suspension during the separation process may be applied according to any suitable profile over the duration of the separation. In one embodiment, the force or pressure is applied at a substantially constant magnitude throughout the separation process. In another embodiment, the force or pressure is applied according to a gradient profile, wherein the magnitude of the force or pressure is progressively increased from an initial value to a final value over the course of the separation, or progressively decreased from an initial value to a final value over the course of the separation. In another embodiment, the force or pressure may be steadily increased or decreased from a starting value until a target peak or trough value is reached, at which point the force or pressure may steadily increase or decrease until that starting value is reached In a further embodiment, the force or pressure is applied according to a stepped profile, wherein the magnitude of the force or pressure is changed at one or more predetermined intervals during the separation process, with each step comprising an increase or a decrease in magnitude relative to the preceding step. In yet another embodiment, the force or pressure is applied according to a fluctuating or oscillating profile, wherein the magnitude of the force or pressure is alternately increased and decreased over the course of the separation process, either periodically or aperiodically. The foregoing pressure profiles may be combined, such that different profiles are applied during different phases of the separation process. For example, the force or pressure may be applied at a constant magnitude during an initial phase of the separation and subsequently transitioned to a gradient, stepped, or oscillating profile during a later phase. The selection of a particular pressure profile may be determined by the operator, prescribed by a predetermined protocol, or controlled by the electronic feedback system described herein based on one or more measured parameters indicative of the status of the separation process. The apparatus is not limited to any particular pressure profile, and any profile or combination of profiles suitable for use in the separation process as described herein is within the scope of the present disclosure.

In another embodiment, one or more of the auxiliary bodies provides a connection between the main body and a source of one or more materials used in the preparation of the suspension prior to, during, or after separation. Such materials may comprise, but are not limited to, one or more diluents (e.g., water, hydrophobic liquids, aqueous solutions, organic solvents, or other liquids), one or more powders (e.g., active powders, filler powders, or blended powder compositions), pre-mixed suspensions or slurries, or any combination thereof. The connection may be configured to permit the selective and controlled introduction of these materials into the interior volume of the main body before, during, or after the separation process, as dictated by the requirements of the particular composition being prepared.

In a further embodiment, one or more of the auxiliary bodies is configured to relieve pressure from within the interior volume of the main body, to introduce additional pressure into the main body, or both. For example, an auxiliary body may comprise a pressure relief valve, a vent, or a bleed fitting to allow controlled release of excess pressure, or may comprise a port for connection to a supplemental pressure source. As another non-limiting example, an auxiliary body may comprise a controllable barrier to a source of mechanical force, such as a spring, weight, or mechanical actuator. The ability to selectively control the force and/or pressure within the main body provides improved process control during the separation operation and allows the operator or an automated control system to adjust separation conditions in response to the characteristics of the particular suspension being processed.

In a further embodiment, one or more auxiliary bodies may be configured to supply a force and/or pressure to apply agitation or movement to the suspension outside of the action of separation, either while the suspension is located inside the main body, has left the main body, or is in the process of transfer between any number of auxiliary bodies and/or the main body. As a non-limiting example, an auxiliary body may supply pressure that creates a turbulent flow in the suspension to encourage re-mixing.

The apparatus further comprises one or more interchangeable bodies. Each interchangeable body may be disposed superiorly to (i.e., above, relative to the operational orientation of the apparatus), inferiorly to (i.e., below, relative to the operational orientation of the apparatus), laterally adjacent to, or in any other suitable spatial relationship relative to the main body. Each interchangeable body is configured to attach to the main body and/or to one or more other interchangeable bodies. The interchangeable bodies may be removed, replaced, or reconfigured to accommodate different separation requirements, suspension compositions, or target VES specifications.

In one embodiment, an interchangeable body is configured to modify the flow of the suspension and/or the separated liquid (i.e., the diluent removed during the separation process) as it exits the main body. In another embodiment, an interchangeable body is configured to modify the shape, cross-sectional area of flux, direction of flow, and/or exit point of the suspension and/or any separated liquid passing through the apparatus. By selecting and configuring appropriate interchangeable bodies, the operator may tailor the flow characteristics of the apparatus to the requirements of the particular separation process being performed.

In a further embodiment, one or more of the interchangeable bodies is configured to support, retain, and/or secure one or more separation media within the apparatus. The separation media may comprise, but are not limited to, one or more sheets of filter paper, one or more filter plates, one or more semi-permeable membranes, one or more woven or non-woven filter cloths, one or more woven wire mesh screens or sieves, one or more sealing materials (e.g., gaskets, rubber sheets, elastomeric materials, plastics, or O-rings), or any combination thereof. The separation media, or components thereof, may be formed of any material that is compatible with the suspension being processed and suitable for the intended separation operation. By way of example and not limitation, membranes and filter sheets may be formed of polytetrafluoroethylene (PTFE), polyvinylidene fluoride (PVDF), polyethersulfone (PES), nylon (polyamide), polypropylene (PP), cellulose acetate, regenerated cellulose, mixed cellulose esters, glass fiber, or combinations thereof. Filter plates may be formed of polymeric materials (e.g., polypropylene, polyethylene, polyether ether ketone (PEEK), or other engineering plastics), metals or metal alloys (e.g., stainless steel such as 304 or 316L grade, sintered stainless steel, titanium, nickel, Hastelloy, brass, or phosphor bronze), ceramics, or combinations thereof. Woven wire mesh screens or sieves may be formed of stainless steel (e.g., 304 or 316 grade), brass, phosphor bronze, electroformed nickel, or other metals or alloys suitable for the intended application. Woven and non-woven filter cloths may be formed of synthetic fibers, including but not limited to polypropylene, polyester, nylon, polyphenylene sulfide (PPS), or polytetrafluoroethylene (PTFE), and may be constructed from monofilament, multifilament, or staple fiber yarns woven in plain, twill, or satin weave patterns, as known in the art.

The separation media may be characterized by a pore size, pore number, aperture size, mesh opening size, and/or other measure of permeability that is selected to be suitable for the retention of all, or substantially all, of the solid phase of the suspension during the separation process. The pore size, aperture size, or mesh opening size may be selected based on the particle size distribution of the powder(s) in the suspension and may be chosen such that the predominant fraction of the powder(s) is retained on or upstream of the separation media while the diluent (liquid phase?) is permitted to pass therethrough. It will be appreciated that in certain embodiments, a minor portion of fine particles within the powder(s) may pass through the separation media during the separation process. The passage of such fine particles does not render the separation media unsuitable for use in the apparatus or the process described herein, provided that the separation media retains a sufficient proportion of the powder(s) to achieve the intended concentration of the solid phase. As used herein, the retention of “substantially all” of the solid phase means that the separation media retains a sufficient quantity of the powder(s) such that the target solids content of the viscoelastic suspension may be achieved, whether directly through the separation process or through subsequent corrective steps such as dilution or re-pressing as described elsewhere herein. The apparatus and process described herein are not limited to any particular retention efficiency, pore size, aperture size, or mesh rating of the separation media.

The selection of a particular separation media, including its material composition, pore or aperture size, and physical configuration, may be based on one or more factors, including but not limited to the chemical compatibility of the material with the suspension components, the particle size and particle size distribution of the powder, the viscosity of the diluent, the target clarity of the separated liquid, the desired rate of separation, the operating temperature and pressure, the required cleanability or reusability of the media, and/or the acceptable level of fine particle passage through the media. The interchangeable nature of the bodies supporting and/or surrounding the separation media allows the operator to select and configure the appropriate media for each separation operation without requiring replacement of the entire apparatus. Similarly, the interchangeable nature of the bodies supporting and/or surrounding the separation media allows the operator to modify, replace and/or remove the separation media(s) while retaining at least partial functionality of the apparatus.

The apparatus may optionally further comprise one or more pushing devices disposed within or adjacent to the interior volume of the main body. The pushing devices may be configured to assist in the separation of the suspension by applying force to the suspension in a manner that may distribute the force more uniformly across the surface area of contact between the pushing device and the suspension. Such application of force may promote more even separation across the separation medium and may reduce the likelihood of channeling, preferential flow paths, stratification, heterogeneity, or other non-uniformities in the distribution of the solid phase within the resulting composition. In some embodiments, the pushing device may mitigate such non-uniformities relative to a separation process performed without the comparable pushing device, and may thereby improve the overall consistency, quality, and/or efficiency of the separation process. The pushing device need not eliminate all non-uniformity in the distribution of the solid phase, and the presence of some degree of non-uniformity in the resulting composition does not preclude the use of the pushing device or render the apparatus or process described herein unsuitable for its intended purpose. The pushing device may additionally or alternatively facilitate the movement, redistribution, or compression of the suspension within the main body at any stage of the separation process. The pushing device may be of any suitable shape or geometry, including but not limited to a disc, a plate, a cone, a dome, a cup, a dish, screw, a cylinder a plunger, a piston, or any other form capable of transmitting force to the suspension within the interior volume of the main body. This range of shapes and geometries may also be uniquely applicable to parts of the pushing device, including but not limited to its main body, connection to a potential source of force or pressure, or to its one or more faces of contact with the suspension or intermediate material.

A pushing device may be configured to apply force to the suspension before the initiation of separation, during separation, after the completion of a separation cycle, or during any combination of these stages. For example, a pushing device may be used to pre-compress the suspension prior to the application of external pressure, to maintain pressure on the suspension during active separation, or to expel residual diluent from the concentrated solids following the primary separation operation. a pushing device may be driven by any suitable source of force, including but not limited to manual force applied by an operator, mechanical force (e.g., via a threaded shaft, lead screw, cam, or lever mechanism), pneumatic force (e.g., via a pneumatic cylinder or air pressure acting on a pushing device), hydraulic force (e.g., via a hydraulic cylinder or pressurized liquid acting on a pushing device), gravitational force (e.g., via one or more weights disposed on or coupled to a pushing device), magnetic force, electromechanical force (e.g., via a linear actuator, stepper motor, or servo motor), or any combination thereof. The source of force used to drive each pushing device may be the same as, or different from, the source of force or pressure used to drive any other number of pushing devices, or the separation process through the separation media.

The pushing device may be configured to contact the suspension directly, such that a surface of the pushing device is in physical contact with the suspension during the application of force. Alternatively, the pushing device may be configured to apply force to the suspension indirectly, for example through one or more intermediate layers, barriers, membranes, liners, or other elements disposed between a pushing device and the suspension. The use of an intermediate element may serve to prevent direct contact between the pushing device and the suspension, which may be desirable in applications where contamination of the suspension by the pushing device, or fouling of the pushing device by the suspension, is to be avoided.

The pushing device may be formed of any material that is suitable for the intended application and compatible with the suspension being processed. In embodiments where the apparatus is used to produce a viscoelastic suspension intended for pharmaceutical, biomedical, veterinary, or other applications involving human or animal use, the pushing device may be formed of a material that is substantially inert with respect to the suspension components, such that the pushing device does not leach, dissolve, dissociate, degrade, shed particulates, or otherwise release material into the suspension during the separation process or at any other time during which the pushing device is in contact with or in proximity to the suspension. Suitable materials for the pushing device may include, but are not limited to, stainless steel (e.g., 304, 316, or 316L grade), titanium or titanium alloys, ceramics (e.g., alumina or zirconia), engineering plastics or polymers (e.g., polytetrafluoroethylene (PTFE), ultra-high molecular weight polyethylene (UHMWPE), polyether ether ketone (PEEK), polypropylene, or polyvinylidene fluoride (PVDF)), elastomeric materials (e.g., bromobutyl rubber, chlorobutyl rubber, butyl rubber, silicone rubber, ethylene propylene diene monomer (EPDM) rubber, fluoroelastomers such as Viton, polyurethane, polyisoprene, nitrile rubber, thermoplastic elastomers (TPE), or styrene butadiene rubber), glass, or combinations thereof. In embodiments where the pushing device comprises or incorporates an elastomeric material, the elastomeric material may form all or a portion of the pushing device, and may function as a sealing element, a contact surface, a compressible body, or any combination thereof. The elastomeric material may be selected to provide one or more of the following properties: chemical compatibility with the suspension components, low extractables and leachables, low gas and moisture permeability, resistance to sterilization processes (e.g., autoclaving, gamma irradiation, or ethylene oxide treatment), and/or suitable mechanical properties such as hardness, elasticity, and compression set resistance. The material of the pushing device may further be selected to comply with applicable regulatory requirements, including but not limited to those established by the U.S. Food and Drug Administration (FDA), the European Medicines Agency (EMA), the United States Pharmacopeia (USP), or other relevant regulatory bodies and pharmacopeial standards, for materials intended for use in the manufacture of pharmaceutical or biomedical products. The pushing device may optionally be coated, lined, laminated, or surface-treated with a compatible material (e.g., a fluoropolymer film, a barrier coating, or a silicone-based lubricant) to enhance chemical resistance, reduce surface adhesion, minimize extractables and leachables, inhibit or prevent the absorption and/or adsorption of one or more components of the liquid phase and/or solid phase of the suspension to or into the pushing device, or otherwise improve the suitability of the pushing device for the intended application.

The pushing device may be supported by an additional support structure configured to stabilize and guide the pushing device during operation. The support structure may contact at least one face of the pushing device and is configured to permit controlled movement of the pushing device along one or more axes relative to the main body. The support structure may comprise, but is not limited to, one or more guide rails with or without blocks, machined shafts or flats, linear bearings, spherical bearing or balls, bushings, bumpers, dampeners, magnetic guidance systems, threaded shafts, lead screws, pneumatic or hydraulic cylinders, crosshead guides, connecting rod, crankshaft or other mechanical elements suitable for constraining and directing the motion of the pushing device. The support structure provides for repeatable and precise positioning of the pushing device, enabling consistent application of force across multiple separation cycles.

The apparatus may further comprise a feedback system operatively coupled to one or more components of the apparatus. The feedback system may be temporal, tactile, sonic, electronic, mechanical, pneumatic, hydraulic, optical, manual, or any combination thereof, and is configured to measure, observe, or otherwise determine one or more parameters indicative of the status of the separation process, the status of any number of parts of the apparatus before, during or after the separation process, and to use the measured or observed parameters as input to direct, adjust, monitor, or terminate the separation process. Measured and observed parameters may also be used to create, modify and/or implement algorithms that may be used to: predict the amount of separation at any point, to calculate stopping points, to predict or provide insight into the status of the separation process or any number of parts of the apparatus, to alert to any malfunction at any number of areas in the apparatus including but not limited to a leak, tear, slip, shift or malformation of any number of parts, or any combination therein. In one embodiment, the feedback system is an electronic feedback system comprising one or more electronic sensors, transducers, controllers, processors, data storage elements, user interfaces, and/or communication interfaces. In other embodiments, the feedback system may comprise one or more non-electronic elements, such as mechanical indicators (e.g., graduated scales, sight glasses, mechanical gauges, or spring-loaded mechanisms), pneumatic or hydraulic control elements (e.g., pressure-sensitive valves, pressure regulators, or relief valves), or visual or auditory indicators that enable an operator to assess the status of the separation process and make process adjustments accordingly. The feedback system may comprise any combination of electronic and non-electronic elements. The feedback system may enable fully automated, semi-automated, or operator-assisted control of the separation operation, thereby improving process consistency, reproducibility, and throughput, while reducing reliance on subjective operator judgment. It will be appreciated that the degree of automation provided by the feedback system may vary depending on the particular components and configuration employed, and that a feedback system requiring some degree of operator involvement is within the scope of the present disclosure.

The status of the separation process may be assessed by measuring one or more of the following parameters, individually or in combination: (a) the mass of liquid removed from the suspension during separation, as measured, for example, by a load cell, a spring scale, multi-beam balance, gravimetric sensor, or flow meter positioned to quantify the collected separated liquid; (b) the volume of liquid removed from the suspension during separation, as measured, for example, by a volumetric flow meter, a graduated collection vessel, or a level sensor; (c) the position of the pushing device (when present) within the main body, as measured, for example, by a linear encoder, potentiometer, linear variable differential transformer (LVDT), or proximity sensor; (d) the force and/or pressure applied to the suspension within the interior volume of the main body, as measured, for example, by a pressure transducer, force gauge, strain gauge, or piezoelectric sensor; (e) the density or specific gravity of the composition within the main body, as measured, for example, by a densitometer, hydrometer, or ultrasonic density sensor; (f) the viscosity or one or more rheological properties of the composition within the main body, as measured, for example, by a viscometer, rheometer, or by monitoring the resistance to movement of the pushing device; (g) one or more optical properties of the separated liquid and/or the composition within the main body, including but not limited to turbidity, opacity, color, or light transmittance, as measured, for example, by a turbidimeter, spectrophotometer, colorimeter, or optical sensor; (h) the electrical conductivity or resistivity of the separated liquid and/or the composition within the main body; (i) the pH of the separated liquid and/or the composition within the main body; (j) the displacement or change in volume of the composition within the main body as liquid is removed; and/or (k) one or more acoustic or ultrasonic properties of the composition within the main body, as measured, for example, by an ultrasonic sensor configured to detect changes in composition density, homogeneity, or phase distribution. Additional parameters that may be monitored include, without limitation, temperature, separation time, liquid removal rate, and any other parameter from which the solids content, composition, or physical state of the suspension or the separated liquid may be inferred. The foregoing list of parameters is not exhaustive, and any measurable parameter that is indicative of or correlative to the progress or status of the separation process may be utilized by the feedback system described herein. The status of any number of parts of the apparatus before, during or after the separation process may similarly be measured by any or all of the parameters listed previously in this paragraph.

The one or more parameters described above may be measured in situ (i.e., within or on the apparatus during the separation process), in-line (i.e., by sensors or instruments integrated into or coupled to the apparatus that measure the parameter continuously or at intervals during the separation process), at-line (i.e., by instruments located in proximity to the apparatus to which a sample is transferred for measurement without significant delay), or off-line (i.e., by removing a sample of the composition or the separated liquid from the apparatus and measuring the parameter using external instrumentation at a location remote from the apparatus). In certain embodiments, one or more parameters may be measured by withdrawing an aliquot or sample of the composition from the interior volume of the main body, or an aliquot or sample of the separated liquid from a collection point downstream of the separation media and subjecting the aliquot or sample to analysis by any suitable instrument or method. The apparatus may comprise one or more sampling ports, valves, or other means configured to permit the withdrawal of an aliquot or sample at one or more stages of the separation process without interrupting or substantially disrupting the ongoing separation operation. In other embodiments, the withdrawal of a sample may require a temporary pause or cessation of the separation process, and the process may be resumed following sample analysis. The feedback system may receive measurement data from in situ, in-line, at-line, or off-line measurements, or any combination thereof, and may use the data to direct, adjust, or terminate the separation process as described herein. The foregoing list of parameters is not exhaustive, and any measurable parameter that is indicative of, or correlative to, the progress or status of the separation process may be utilized by the feedback system, regardless of the method or location of measurement.

As a non-limiting example, an electronic feedback system may utilize one or more of the measured parameters to determine when the target solids content of the VES has been substantially achieved, the action which may or may not use a developed algorithm to assist in this determination. Upon determining that the target solids content has been reached, that the ideal stopping point has been reached, and/or that a predetermined threshold value of one or more measured parameters has been met, the feedback system may automatically adjust the applied force or pressure, alter the rate of separation, alter or remove any remaining force or pressure in the apparatus, such as by venting, and/or terminate the separation process. The electronic feedback system may further comprise a controller, a processor, one or more data storage elements, one or more wireless communication elements such as Bluetooth capabilities, and/or a user interface for displaying process data and receiving operator input.

The feedback system may utilize one or more of the measured parameters to assess the status of the separation process, including but not limited to determining whether the solids content of the composition is approaching, has substantially reached, has exceeded, or has not yet reached the target solids content of the VES and/or the target stopping point for active separation. Upon determining that the target solids content has been substantially achieved, the feedback system may automatically adjust the applied force or pressure, alter the rate of separation, or terminate the separation process. Upon determining that the solids content has exceeded the target solids content (i.e., that the composition has been over-pressed), the feedback system may terminate the separation process and may initiate or signal the need for one or more corrective actions, such as the introduction of additional diluent into the composition and subsequent mixing to substantially achieve the target solids content, as described elsewhere herein. Upon determining that the solids content has not yet reached the target solids content (i.e., that the composition has been under-pressed or insufficiently pressed), the feedback system may continue or resume the application of force or pressure, adjust the magnitude or profile of the applied force or pressure, or initiate or signal the need for re-pressing of the composition to substantially achieve the target solids content. The feedback system may further be configured to generate one or more alerts, notifications, or output signals to inform the operator of the status of the separation process, including whether the process has reached completion, requires corrective action, or requires operator intervention. The feedback system may further comprise a controller, a processor, one or more data storage elements, a user interface for displaying process data and receiving operator input, and/or one or more communication interfaces for transmitting process data to external systems (e.g., a laboratory information management system (LIMS), a manufacturing execution system (MES), or a supervisory control and data acquisition (SCADA) system). In embodiments where the feedback system is non-electronic or partially non-electronic, the operator may perform one or more of the assessment, adjustment, or termination functions described above based on direct observation of mechanical indicators, gauges, graduated scales, or other non-electronic feedback elements.

It will be appreciated that the various components of the apparatus described herein, including the main body, the one or more auxiliary bodies, the one or more interchangeable bodies, the one or more separation media, the optional pushing device and its associated support structure, and the feedback system (whether electronic, mechanical, or any combination thereof), may be assembled in a variety of configurations and combinations to accommodate different suspension compositions, powder types, diluent types, separation requirements, manufacturing requirements, target solids contents, and VES specifications. The modular and interchangeable nature of the apparatus components provides flexibility in adapting the apparatus to the specific requirements of each separation operation, including but not limited to the selection of appropriate separation media materials and pore or aperture sizes, the selection of pushing device materials compatible with the suspension and its intended end use, the selection of appropriate force or pressure sources and pressure profiles, and the configuration of the feedback system to monitor and respond to one or more process parameters as described herein. The apparatus may be used in connection with the production of viscoelastic suspensions intended for pharmaceutical, biomedical, veterinary, industrial, or other applications, and the materials, components, and configurations of the apparatus may be selected to comply with applicable regulatory requirements for the intended application. Components may be added, removed, substituted, rearranged, or reconfigured without departing from the scope of the present disclosure.

The following non-limiting example describes the concentration of a suspension from a dilute suspension using an apparatus assembled in accordance with the present disclosure. The main body comprised a 304 stainless steel sanitary tube connector having an approximately 4-inch diameter and a length of approximately 18 inches, configured as a substantially hollow, generally cylindrical chamber defining an interior volume for receiving and retaining the suspension. The main body comprised an upper opening and a lower opening, each in fluid communication with the interior volume and each configured for connection to auxiliary and interchangeable bodies via sanitary tri-clamp fittings with elastomeric gaskets (e.g., silicone).

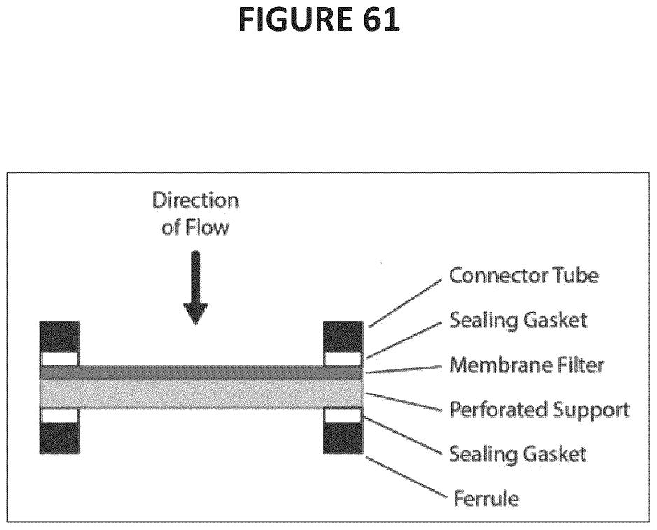

Referring to FIG. 61, the separation medium comprised a 0.65 μm pore size hydrophilic PVDF membrane filter (Millipore Durapore™ DVPP) cut to an outer diameter to be slightly larger than the internal diameter of the main body. The membrane filter was placed on top of an elastomeric sealing gasket disposed at the lower end of the main body, and a perforated support (in this example, a 304 stainless steel perforated sheet, approximately 5-inch diameter, approximately 0.16-inch hole size) was placed below the membrane to provide structural support during the application of pressure. A second sealing gasket was disposed below the perforated support. A 304 stainless steel sanitary tri-clamp fitting ferrule was inverted and placed below the second sealing gasket, and the components were compressed and secured using a 4-inch sanitary tri-clamp. In this configuration, the separation medium was oriented substantially horizontally at the lower end of the main body, spanning the cross-sectional area of the interior volume, and was configured to permit passage of the liquid phase (in this example, Miglyol® 840 was the diluent) therethrough while retaining substantially all of the solid phase (in this example, spray dried IgG powder) within the interior volume above the separation medium. Prior to the introduction of the suspension, the membrane filter was pre-wet with diluent.

The pushing device comprised a 3D-printed nylon cylindrical piston fitted with Viton® fluoroelastomer O-rings (FDA-compliant, size No. 235) disposed in circumferential slots on the piston body. The O-rings provided a substantially fluid-tight seal against the inner wall of the main body, permitting the pushing device to translate axially within the interior volume while minimizing bypass of the suspension around the piston. A venting tube (e.g., stainless steel, 0.028-inch outer diameter, 0.01-inch wall thickness) was temporarily inserted through the main body during insertion of the pushing device to vent trapped air from the interior volume below the piston. The venting tube was removed prior to the application of pneumatic pressure.

Pneumatic pressure was applied to the pushing device via an auxiliary body connected to the upper opening of the main body. The auxiliary body comprised, in series, a regulated compressed air supply, pneumatic tubing (6.5 mm inner diameter), a pneumatic pressure regulator (Pneumatic Plus model SAR200-N02BG), and a series of sanitary tri-clamp fittings and reducers (1.5-inch to 4-inch, 304 stainless steel) that adapted the pneumatic supply to the upper opening of the main body. The pressure regulator permitted controlled adjustment of the pneumatic pressure applied to the pushing device during the separation process.

The apparatus further comprised a feedback system for monitoring the progress of the separation process. The feedback system comprised a load cell coupled to an HX711 weight-to-digital conversion sensor and an Arduino Uno microcontroller, configured to measure and record the mass of separated diluent collected in the collection vessel at intervals of approximately 15 seconds throughout the separation process. A collection vessel comprising a 2000 mL plastic beaker was disposed below the separation medium on the load cell. The main body and separation medium assembly were supported by a support structure, and the apparatus was disposed on a laboratory bench.

A suspension was prepared by combining approximately 50 g of spray dried IgG powder with approximately 450 g of Miglyol® 840 (diluent), yielding approximately 500 g of a dilute suspension having an initial solids content of approximately 10% (w/w). The spray dried IgG powder (prepared by spray drying under the conditions described in Tables 33 and 34) had a generally spherical morphology of the type shown in FIG. 49 and a particle size volume distribution (measured by laser diffraction) having a D90 of less than 10 μm. The suspension was mixed using a high-shear mixer (Silverson model L5M-A, Silverson Machines, Inc.) at a mixing speed of approximately 10,200 RPM (which corresponded to the maximum speed setting of the Silverson L5M-A mixer) for approximately 2 minutes to disperse the powder in the diluent and to disrupt agglomerates. Following high-shear mixing, the suspension was introduced into the interior volume of the main body through the upper opening. The pushing device was inserted into the main body with the aid of the venting tube, and was advanced until the piston was substantially contained within the main body. In this configuration, an air space remained between the lower face of the pushing device and the upper surface of the suspension. The pneumatic auxiliary body was then connected to the upper opening of the main body and secured via a sanitary tri-clamp fitting.

Pneumatic pressure was applied to the pushing device according to a stepped pressure profile. Initially, the pressure regulator was gradually opened to deliver approximately 20 PSI to the pushing device, driving the liquid phase of the suspension through the separation medium and into the collection vessel as separated diluent. The pneumatic pressure applied above the pushing device drove the pushing device toward the suspension, compressing the air space between the pushing device and the suspension surface and thereby transmitting force to the suspension without requiring direct contact between the pushing device and the suspension. Accordingly, the apparatus may be operated with varying fill volumes relative to the total interior volume of the main body. The mass of separated diluent was monitored in real time via the load cell feedback system at 15-second intervals. When the rate of diluent removal decreased below approximately 2 g per 15-second interval, the pneumatic pressure was increased to approximately 30 PSI. As the separation approached the calculated target mass of separated diluent, the pneumatic pressure was turned off, and all residual pressure was vented through the regulator. Passive separation continued by gravity until the rate of diluent removal fell below approximately 0.4 g per 15-second interval, at which point the separation process was deemed complete. It will be appreciated that the endpoint of the separation process may be determined by any suitable criterion, including without limitation a target mass of separated diluent, a target solids content, a minimum flow rate threshold, a predetermined elapsed time, or a combination thereof.

The entire process was completed within 40 minutes. At the conclusion of the separation process, the concentrated composition remaining within the interior volume of the main body above the separation medium was recovered by disassembling the separation medium assembly and collecting the composition from the membrane filter surface (e.g., using a spatula, extrusion, or other suitable means). The density of the resulting concentrated composition was measured by helium pycnometry, and the final solids content was calculated using the density-based formula described elsewhere herein, using the known densities of the IgG powder (1.34 g/cm³ at 25° C.) and the Miglyol® 840 diluent (0.92 g/mL at 25° C.). The final solids content of the concentrated composition was approximately 63% (w/w), representing a greater than six-fold increase from the initial solids content of approximately 10% (w/w) through the controlled removal of the liquid phase, indicating that the apparatus may be used to produce highly concentrated suspensions from dilute starting compositions in a single separation operation. The resulting concentrated composition was observed to exhibit characteristics consistent with a concentrated suspension (e.g., VES) as described elsewhere herein, including a measurable yield stress and a consistency such that the composition was not pourable under gravity, though such characterization is not required in all embodiments and the apparatus is not limited to the production of compositions exhibiting any particular rheological profile. The resulting concentrated suspension may be suitable for subsequent processing, corrective adjustment (e.g., dilution or additional separation to a refined target solids content), testing, formulation, or use.

It will be appreciated that the foregoing process leverages the reduced viscosity of the dilute suspension to enable effective high-shear mixing and agglomerate disruption at a solids content (approximately 10% w/w) at which the suspension exhibits flow characteristics amenable to high-shear processing. The resulting concentrated composition (in this example, approximately 63% w/w for the IgG powder and Miglyol® 840 system) may constitute a concentrated suspension (e.g., VES) having a viscosity at which equivalent high-shear mixing may be more difficult, impractical, or less effective due to the resistance of the concentrated composition to flow, to the transmission of shear forces throughout the bulk material, and to the generation of localized heat (e.g., at the point of shear input), which may result in thermal degradation of heat-sensitive components such as proteins, peptides, or other biologic molecules. It will be appreciated that the solids content at which a suspension exhibits such concentrated behavior will vary depending on the specific powder, diluent, and their respective physical and chemical properties.

Accordingly, the apparatus and process described in this example may enable the production of concentrated suspensions (e.g., concentrated protein suspensions) having reduced agglomerate content by performing agglomerate disruption at a dilute stage and subsequently concentrating the suspension to a target solids content through the controlled removal of the liquid phase, which may permit the agglomerate disruption and concentration operations to be performed at different solids contents and under different rheological conditions, each of which may be independently selected or optimized for that operation (this may include optimization for removal of agglomerates, stability, ease of manufacturing, manufacturing yield, scale or cycle time). The concentrated suspension produced by this process may be characterized by reduced agglomerate content relative to concentrated suspensions prepared by direct mixing at equivalent solids content, or by other conventional approaches that may not perform the agglomerate disruption and concentration operations under independently selected conditions.

The foregoing example is provided to illustrate one embodiment of the apparatus and process described herein and is not intended to limit the scope of the present disclosure to the specific materials, components, dimensions, configurations, process parameters, or compositions described in this example. Furthermore, while this example employed a pushing device to transmit force to the suspension, the apparatus and process described herein may be practiced without a pushing device, for example by applying pneumatic pressure directly to the suspension surface. It will be appreciated that the apparatus and process described herein may be practiced with different main body materials and dimensions, different pushing device materials and configurations, different separation media (e.g., different membrane materials, pore sizes, filter types, or mesh screens), different sources of force or pressure (e.g., hydraulic, mechanical, electromechanical, or gravitational force in addition to or in place of pneumatic pressure), different pressure profiles (e.g., constant, gradient, oscillating, or other profiles), different mixing methods and parameters (e.g., different mixer types, shear rates, mixing durations, or mixing stages), different powders (including without limitation powders comprising any of the biologic molecules or small molecule therapeutics described elsewhere herein) and diluents, different initial and target solids contents, different feedback system configurations (e.g., open or closed loop controls; measuring applied force, mass/volume or flow rate of separated diluent, etc.), different corrective adjustment methods (e.g., dilution of an over-concentrated composition with additional diluent, additional separation of an under-concentrated composition, or blending of compositions having different solids contents), and different apparatus scales, all without departing from the scope of the present disclosure.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of certain specific embodiments will so fully reveal the general nature of the invention that others can, by applying knowledge within the skill of the art, readily modify and/or adapt for various applications such specific embodiments, without undue experimentation, without departing from the general concept of the present invention. Hence, in addition to those specifically described herein, other suitable embodiments of the invention will be readily apparent to one of ordinary skill in the art based upon the foregoing description and examples, and upon knowledge generally available in the relevant arts. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All references cited herein, including U.S. patents and published patent applications, international patents and patent applications, and journal references or other publicly available documents, are incorporated herein by reference in their entireties to the same extent as if each reference had been specifically cited for the portion or portions of such reference applicable to the section of this application to which it is relevant.

1. A method of preparing a therapeutic composition comprising a concentrated suspension, said composition having a solids concentration of greater than about 350 mg/mL, wherein the concentrated suspension comprises one or more active pharmaceutical ingredients, one or more pharmaceutically acceptable excipients, at least one rheology modifier, and one or more non-solvent fluids, wherein said paste is capable of being injected subcutaneously, intracutaneously, intradermally, or intramuscularly into an animal in a volume of 3 mL or less at a flow rate of at least 30 μL/s using a commercially available needle/syringe combination, the method comprising (a) obtaining a spray-dried powder comprising said one or more active pharmaceutical ingredients and one or more pharmaceutically acceptable excipients; (b) combining said powder with one or more diluents to form a suspension of said powder in said one or more diluents; (c) mixing the suspension to evenly distribute the powder in the diluent; (d) removing excess diluent from said suspension, thereby forming a paste; and (e) mixing said paste to form an injectable high solids content paste.

2. The method of claim 1, wherein said suspension is a dilute suspension that is mixed via high shear mixing, thereby disrupting any agglomerates that are present in the dilute suspension, or wherein said suspension is mixed via low shear mixing.

3.-6. (canceled)

7. The method of claim 1, wherein said active pharmaceutical ingredient is a peptide or protein therapeutic, selected from the group consisting of an enzyme, an antithrombin agent, a thrombolytic agent, a peptide hormone, a bone-active peptide, a diabetic-active peptide, an antibody, a non-antibody antineoplastic agent, a fertility agent, and an immunosuppressive agent.

8. (canceled)

9. The method of claim 7, wherein said antibody is a monoclonal antibody, a polyclonal antibody, or a fragment thereof.

10. (canceled)

11. The method of claim 1, wherein the solids concentration of said concentrated suspension is greater than about 500 mg/mL.

12. (canceled)

13. The method of claim 1, wherein said one or more pharmaceutically acceptable excipients are selected from the group consisting of a saccharide, a surfactant, an amino acid, and a buffering agent.

14. The method of claim 13, wherein said saccharide is selected from the group consisting of trehalose, dextrose, sucrose, mannose, and fructose.

15. The method of claim 1, wherein said excipient is selected from the group consisting of triacetin, polysorbate 20, polysorbate 80, caprylic/capric triglyceride, and propylene glycol dicaprylate/dicaprate.

16. (canceled)

17. The method of claim 1, wherein said amino acid is tryptophan, phenylalanine, arginine, histidine, proline, or cysteine.

18. (canceled)

19. The method of claim 13, wherein said buffering agent is selected from the group consisting of histidine, citrate, succinate, and lactate.

20. The method of claim 1, wherein said non-solvent fluid is at least one rheology modifier.

21. The method of claim 20, wherein said rheology modifier is a surfactant.

22. (canceled)

23. The method of claim 21, wherein said surfactant is a non-ionic surfactant comprising at least one fatty acid, selected from the group consisting of lauric acid, palmitic acid, stearic acid, tristearic acid, oleic acid, and trioleic acid.

24.-25. (canceled)

26. A method of treating, preventing, ameliorating, or diagnosing a disease or disorder in an animal or human suffering from or predisposed to said disease or disorder, comprising injecting an effective dose of the therapeutic composition prepared according to the method of claim 1 into said animal or human subcutaneously, intracutaneously, intradermally, or intramuscularly.

27. The method of claim 26, wherein said active pharmaceutical ingredient is one or more of a small molecule, biologic, peptide, oligonucleotide, or radiopharmaceutical.

28. The method of claim 26, wherein said active pharmaceutical ingredient is an antibody or a fragment thereof.

29. The method of claim 28, wherein said antibody or fragment thereof is selected from the group consisting of a monoclonal antibody or fragment thereof, a polyclonal antibody or fragment thereof, a multi-specific antibody, an Fc-engineered antibody, a fusion antibody, a conjugated antibody, a multimeric antibody, an antibody based on a non-immunoglobulin scaffold, a specialized engineered antibody, and a synthetic antibody-like molecule.

30.-39. (canceled)

40. An apparatus for increasing the solids content of a suspension comprising a powder and a diluent, the apparatus comprising:
(a) a main body that is substantially hollow and defines an interior volume configured to receive and retain the suspension, the main body comprising at least two openings in fluid communication with the interior volume, wherein at least one of the openings is configured to be temporarily sealed;
(b) any number of auxiliary bodies, each auxiliary body being attachable to the main body or to another auxiliary body, wherein at least one auxiliary body is configured to provide a connection to a source of force or pressure for driving a separation process in which a portion of the diluent is removed from the suspension while the powder is substantially retained;
(c) any number of interchangeable bodies, each interchangeable body being attachable to the main body, to one or more auxiliary bodies or to one or more other interchangeable body, wherein at least one interchangeable body is configured to support one or more separation media; and
(d) one or more separation medium disposed within the apparatus and configured to permit passage of the diluent therethrough while retaining substantially all of the powder, upon application of the force and/or pressure.

41. The apparatus of claim 40, further comprising one or more pushing devices disposed within or adjacent to the interior volume of the main body, the pushing device being configured to apply force to the suspension, wherein the pushing device may contact the suspension directly or may apply force to the suspension indirectly through an intermediate element disposed between the pushing device and the suspension; and optionally a support structure configured to stabilize and guide the pushing device, the support structure contacting at least one face of the pushing device and permitting controlled movement of the pushing device along one or more axes relative to the main body, wherein the pushing device is driven by at least one of manual force, mechanical force, pneumatic force, hydraulic force, gravitational force, magnetic force, electromechanical force, or a combination thereof.

42.-46. (canceled)

47. An apparatus for producing a viscoelastic suspension from a suspension comprising a powder and a diluent, the apparatus comprising:
(a) a main body that is substantially hollow and defines an interior volume for receiving the suspension, the main body comprising at least two openings in fluid communication with the interior volume;
(b) any number of auxiliary bodies attachable to the main body, wherein at least one auxiliary body provides a connection to a source of force or pressure configured to drive a separation process in which a portion of the diluent is removed from the suspension;
(c) any number interchangeable bodies attachable to the main body, wherein at least one interchangeable body supports one or more separation media configured to retain substantially all of the powder while permitting passage of the diluent;
(d) a pushing device disposed within or adjacent to the interior volume and configured to apply force to the suspension; and
(e) one or more feedback systems operatively coupled to the apparatus, the feedback systems being configured to measure, observe, or otherwise determine one or more parameters indicative of the status of the separation process, and to use the one or more parameters to direct, adjust, monitor, or terminate the separation process, wherein the feedback system is electronic, mechanical, or any combination thereof.

48. The apparatus of claim 47, wherein the one or more parameters comprise at least one of: a mass or volume of liquid removed from the suspension during separation; a position of the pushing device within the main body; a force or pressure applied to the suspension within the interior volume; a density or specific gravity of the composition within the main body; a viscosity or rheological property of the composition within the main body; an optical property of the separated liquid or the composition within the main body; an electrical conductivity or resistivity of the separated liquid or the composition; a pH of the separated liquid or the composition; or an acoustic or ultrasonic property of the composition within the main body.

49.-50. (canceled)

51. A method of producing a viscoelastic suspension, the method comprising:
(a) introducing a suspension comprising one or more powders and one or more diluents into an interior volume of a main body of an apparatus, the main body being substantially hollow and comprising at least two openings in fluid communication with the interior volume;
(b) applying a force or pressure to the suspension within the interior volume via so as to cause a portion of the diluent to pass through one or more separation media supported by at least one interchangeable body attached to the main body, while retaining substantially all of the powder within the interior volume, thereby concentrating the powder relative to its concentration in the suspension prior to the application of the force or pressure; and
(c) monitoring one or more parameters indicative of the status of the separation and/or the status of one or more parts of the apparatus using a feedback system; and optionally
(d) determining a solids content of the composition following the application of the force or pressure; and
(e) upon determining that the solids content exceeds a target solids content, diluting the composition with additional diluent and mixing the composition to substantially achieve the target solids content of the viscoelastic suspension, wherein the mixing is accomplished by manual mixing, low-shear mixing, planetary-centrifugal mixing, or by high-shear mixing if said suspension is determined to contain agglomerates; or
(f) upon determining that the solids content is below a target solids content, reapplying force or pressure to the composition to remove additional diluent until the target solids content is substantially achieved.

52. The method of claim 51, wherein the force or pressure is applied according to at least one of a substantially constant profile, a gradient profile, a stepped profile, or a fluctuating or oscillating profile, or a combination of profiles applied during different phases of the separation.

53.-56. (canceled)
